(19) United States
(12) Patent Application Publication
(10) Pub. No.: US 2023/0021837 A1
(43) Pub. Date: Jan. 26, 2023

(54) METHODS AND KITS FOR DETECTING AUTOIMMUNE DISEASES

(71) Applicant: MESO SCALE TECHNOLOGIES, LLC., Rockville, MD (US)

(72) Inventors: Anu MATHEW, North Potomac, MD (US); Mingyue WANG, Potomac, MD (US); Christopher CAMPBELL, Columbia, MD (US); Katarzyna HAYNESWORTH, Buckeystown, MD (US); Nikhil PADMANABHAN, McLean, VA (US); Martin STENGELIN, Gaithersburg, MD (US); Paridhi GUPTA, Boyds, MD (US); Rashmi SRIRAM, Gaithersburg, MD (US)

(21) Appl. No.: 17/779,900

(22) PCT Filed: Nov. 25, 2020
(86) PCT No.: PCT/US2020/062362

§ 371 Date: May 25, 2022

Related U.S. Application Data
(60) Provisional application No. 63/105,716, filed on Oct. 26, 2020.
(60) Provisional application No. 62/940,730, filed on Nov. 26, 2019.

(51) Int. Cl. G01N 33/564; G01N 33/543

(52) U.S. Cl. CPC G01N 33/564; G01N 33/543; G01N 2800/042; G01N 2800/104; G01N 2800/24; G01N 2800/52

(57) ABSTRACT

The invention relates to assay methods and kits for assessing autoimmune diseases in a human subject. In embodiments, the present disclosure provides assay methods and kits for diagnosing a subject with or predicting that a subject will develop Type 1 diabetes. In embodiments, the present disclosure provides assay methods and kits for assessing responsiveness of a subject having Type 1 diabetes to treatment with alefacept. In embodiments, the present disclosure provides assay methods and kits for diagnosing a subject with systemic lupus erythematosus. In embodiments, the present disclosure provides assay methods and kits for determining if a subject is at risk of a systemic lupus erythematosus flare. In embodiments, the present disclosure provides assay methods and kits for diagnosing a subject with celiac disease.

### CROSS REFERENCE TO RELATED APPLICATIONS

This patent application claims priority under 35 U.S.C. § 119(e) to U.S. provisional patent application No. 62/940,730, filed on Nov. 26, 2019 and application No. 63/105,716, filed on Oct. 26, 2020, the disclosures of which are hereby incorporated by reference in their entireties.

### STATEMENT REGARDING FEDERALLY-SPONSORED RESEARCH

This invention was made with federal support under grants U24AI118660, U24AI118663 and 5R43DK096967-02 awarded by the Department of Health and Human Services. The U.S. government has certain rights in the invention.

### FIELD OF THE INVENTION

The invention relates to assay methods and kits for assessing autoimmune diseases.

### BACKGROUND

Alefacept is a genetically engineered immunosuppressive drug that has recently shown promise in the treatment of Type 1 diabetes. M. R. Rigby, et al, J Clin Invest. 2015 Aug. 3; 125(8):3285-96. Alefacept is dimeric fusion protein consisting of the leukocyte function antigen-3 protein and Fc portion of human IgG1. It would be advantageous to non-invasively monitor treatment in subjects with Type 1 diabetes receiving alefacept. It would similarly be advantageous to non-invasively determine whether a subject having Type 1 diabetes is amenable to treatment with alefacept in determining whether or not to treat the subject with alefacept. In addition, there is a need to prevent the life-threatening consequences of undiagnosed Type 1 diabetes (T1D) before the onset of T1D by identifying individuals likely to develop T1D prior to onset.

Systemic lupus erythematosus (SLE), sometimes referred to as lupus, is an autoimmune disease in which the immune system attacks various tissues in the body. Common symptoms of lupus include painful and swollen joints, fever, chest pain, hair loss, mouth ulcers, swollen lymph nodes, exhaustion, and a red rash on the face. Often there are periods of illness for SLE, called flares, and periods of remission during which there are few symptoms. While there is no cure for SLE, symptoms can be treated using corticosteroids and certain anti-malarial drugs. As long term use of either of these treatments is not desirable, it would be advantageous to develop a non-invasive method to diagnose SLE and to predict when the symptoms of the disease might flare.

Celiac disease is an autoimmune disorder that primarily affects the small intestine. Celiac disease is caused by a reaction to gluten in foods. Consumption of gluten in celiac patients causes an abnormal immune response that can affect a number of different organs, including causing an inflammatory reaction in the small intestine that may lead to shortening of its villi lining. Celiac disease can occur at any age. The diagnosis of celiac disease is complicated by a lack of consistently reliable biomarkers and by the fact that villi shortening is different between patients and may not be detectable until the patient has been suffering from the disease for a long period of time. As following a gluten free diet is the only known treatment for celiac disease, a non-invasive method to diagnose celiac disease before more serious symptoms arise would allow the patient to switch to a gluten free diet before more damage to the villi is done.

### SUMMARY OF THE INVENTION

In embodiments, the disclosure provides a multiplexed immunoassay method comprising, quantifying the amounts of at least two human biomarkers in a biological sample, wherein the at least two biomarkers comprise (a) anti-IA2 IgG and (b) anti-beta2glycoprotein IgG, wherein the quantifying comprises measuring the concentrations of the at least two biomarkers in a multiplexed assay format to simultaneously measure the concentrations of the least two biomarkers in the biological sample wherein the multiplexed immunoassay comprises: (a) combining, in one or more steps: (i) the biological sample; (ii) at least a first and second binding reagent, wherein the first and second binding reagent is a binding partner of anti-IA2 IgG and anti-beta2glycoprotein IgG, respectively; (b) forming at least a first and second binding complex comprising the binding reagents and the biomarkers; and (c) measuring the concentration of the biomarkers in each of the binding complexes.

In embodiments, the disclosure provides a multiplexed immunoassay method comprising, quantifying the amounts of at least five human biomarkers in a biological sample, wherein the at least five biomarkers comprise (a) anti-IA2 IgG, (b) anti-beta2glycoprotein IgG, (c) anti-DGP IgG, (d) anti-IA2 IgM and (e) anti-MPO IgA, wherein the quantifying comprises measuring the concentrations of the at least five biomarkers in a multiplexed assay format to simultaneously measure the concentrations of the least five biomarkers in the biological sample wherein the multiplexed immunoassay comprises: (a) combining, in one or more steps: (i) the biological sample; (ii) at least a first, second, third, fourth and fifth binding reagent, wherein the first, second, third, fourth and fifth binding reagent is a binding partner of anti-IA2 IgG, anti-beta2glycoprotein IgG, anti-DGP IgG, anti-IA2 IgM, and anti-MPO IgA, respectively; (b) forming at least a first, second, third, fourth and fifth binding complex comprising the binding reagents and the biomarkers; and (c) measuring the concentration of the biomarkers in each of the binding complexes.

In embodiments, the disclosure provides a multiplexed first assay method comprising, simultaneously detecting at least two human biomarkers in a biological sample in a multiplexed assay format, wherein the at least two biomarkers comprise (a) anti-IA2 IgG and (b) anti-beta2glycoprotein IgG, wherein the multiplexed assay comprises: (a) combining, in one or more steps: (i) the biological sample; (ii) at least a first and second binding reagent, wherein the first and second binding reagent is a binding partner of anti-IA2 IgG and anti-beta2glycoprotein IgG, respectively; (b) forming at least a first and second binding complex comprising the binding reagents and the biomarkers; and (c) detecting the biomarkers in each of the binding complexes.

In embodiments, the disclosure provides a multiplexed second assay method comprising, detecting at least four human biomarkers in a biological sample in a multiplexed assay format, wherein the at least four biomarkers comprise (a) anti-IA2 IgG, (b) anti-beta2glycoprotein IgG, (c) anti-DGP IgG and (d) anti-IA2 IgM, wherein the multiplexed assay comprises: (a) combining, in one or more steps: (i) the biological sample; (ii) at least a first, second, third and fourth binding reagent, wherein the first, second, third and fourth binding reagent is a binding partner of anti-IA2 IgG, anti-beta2glycoprotein IgG, anti-DGP IgG and anti-IA2 IgM, respectively; (b) forming at least a first, second, third and fourth binding complex comprising the binding reagents and the biomarkers; and (c) detecting the biomarkers in each of the binding complexes.

In further embodiments, the present disclosure provides methods wherein the biomarkers are antibodies and the binding reagents and detection reagents are antigens to the antibodies. In further embodiments, the present disclosure provides methods wherein the biomarkers are antibodies, the binding reagents are antigens to the biomarker antibodies, and the detection reagents are antigens and/or detection antibodies that bind the biomarker antibodies. In further embodiments, the present disclosure provides methods comprising detecting, in the multiplexed assay format, at least one additional biomarker in the biological sample, wherein the at least one additional biomarker is anti-proinsulin IgG, anti-MPO IgM, anti-ZnT8 IgM or a combination thereof.

In embodiments, the disclosure provides a third assay method comprising quantifying the amount of a human biomarker in a biological sample, wherein the biomarker is selected from anti-IA2 IgG, anti-beta2glycoprotein IgG, anti-DGP IgG, anti-IA2 IgM anti-MPO IgA, anti-proinsulin IgG, anti-MPO IgM, anti-ZnT8 IgM, or combinations thereof, wherein the quantifying comprises (a) contacting the biological sample with (i) a capture reagent that specifically binds to the biomarker in a binding domain on a surface, wherein the binding domain further comprises an anchoring reagent; (ii) a first detection reagent that specifically binds to the biomarker and is linked to a first nucleic acid probe; and (iii) a second detection reagent that specifically binds to the biomarker and is linked to a second nucleic acid probe, thereby forming a complex on the surface comprising the capture reagent, the first biomarker, and the first and second detection reagents; (b) using an extension process that requires the first and second probes to be in proximity, extending the second probe to form an extended sequence comprising an anchoring region that binds the anchoring reagent; (c) binding the extended sequence to the anchoring reagent; and (d) measuring the amount of extended sequence bound to the binding domain, thereby quantifying the amount of the biomarker.

In further embodiments, the disclosure provides methods wherein the biomarker is selected from anti-IA2 IgG and anti-beta2glycoprotein IgG. In further embodiments, the disclosure provides methods wherein the biomarkers are antibodies and the binding reagents and detection reagents are antigens to the antibodies. In further embodiments, the disclosure provides methods wherein the biomarkers are antibodies, the binding reagents are antigens to the biomarker antibodies, and the detection reagents are detection antibodies that bind the biomarker antibodies.

In embodiments, the disclosure provides a multiplexed fourth assay method comprising, detecting at least three human biomarkers in a biological sample in a multiplexed assay format, wherein the at least three biomarkers are selected from (a) anti-Smith IgG, (b) anti-RoSSA60 IgG, (c) anti-U1 RNPA IgG, (d) anti-insulin IgM and (e) anti-RoSSA52 IgG, wherein the multiplexed assay comprises combining, in one or more steps: the biological sample; at least a first, second and third binding reagent, wherein the first, second and third binding reagent is a binding partner of anti-Smith IgG, anti-RoSSA60 IgG and anti-U1 RNPA IgG anti-insulin IgM or anti-RoSSA52 IgG, respectively; forming at least a first, second and third binding complex comprising the binding reagents and the biomarkers; and detecting the biomarkers in each of the binding complexes. In embodiments, the method further comprises detecting, in a multiplexed assay format, at least one additional biomarker in the biological sample, wherein the at least one additional biomarker is anti-GAD65 IgM, anti-ZnT8 IgG, anti-RoSSA60 IgA, anti-RoSSA52 IgM, anti-aNCA PR3 IgG, anti-Jo1 IgA, anti-Smith IgA, anti-U1 RNPA IgM, anti-MPO IgA, anti-U1RNPC IgG, anti-GAD65 IgG, anti-LaSSb IgA, anti-TPO IgG, anti-MPO IgG, anti-insulin IgA, anti-TPO IgM, anti-ZnT8 IgM, anti-IF IgG, anti-Smith IgG, TNF-alpha, IL-15, MIP1a, IL-10, NFL, IL-8, IL-6, IL-2, IL-21, IL-23 or a combination thereof.

In embodiments, the disclosure provides a fifth assay method comprising quantifying the amount of a human biomarker in a biological sample, wherein the biomarker is selected from anti-Smith IgG, anti-RoSSA60 IgG, anti-U1 RNPA IgG, anti-insulin IgM, anti-RoSSA52 IgG, anti-GAD65 IgM, anti-ZnT8 IgG, anti-RoSSA60 IgA, anti-RoSSA52 IgM, anti-aNCA PR3 IgG, anti-Jo1 IgA, anti-Smith IgA, or combinations thereof, wherein the quantifying comprises (a) contacting the biological sample with (i) a capture reagent that specifically binds to the biomarker in a binding domain on a surface, wherein the binding domain further comprises an anchoring reagent; (ii) a first detection reagent that specifically binds to the biomarker and is linked to a first nucleic acid probe; and (iii) a second detection reagent that specifically binds to the biomarker and is linked to a second nucleic acid probe, thereby forming a complex on the surface comprising the capture reagent, the first biomarker, and the first and second detection reagents; (b) using an extension process that requires the first and second probes to be in proximity, extending the second probe to form an extended sequence comprising an anchoring region that binds the anchoring reagent; (c) binding the extended sequence to the anchoring reagent; and (d) measuring the amount of extended sequence bound to the binding domain, thereby quantifying the amount of the biomarker.

In embodiments, the disclosure provides a multiplexed sixth assay method comprising, detecting at least five human biomarkers in a biological sample in a multiplexed assay format, wherein the at least five biomarkers are selected from: (a) anti-insulin IgM, (b) anti-MPO IgA, (c) anti-Jo1 IgA, (d) anti-ZnT8 IgM, (e) anti-GAD65 IgG, (f) anti-Smith IgA, (g) anti-IA2 IgA, (h) anti-GAD65 IgM, (i) anti-Jo1 IgG, (j) anti-beta2glycoprotein IgA, (k) anti-ZnT8 IgG, and (1) anti-insulin IgA, wherein the multiplexed assay comprises combining, in one or more steps: the biological sample; at least a first, second, third, fourth and fifth binding reagent, wherein the first, second, third, fourth and fifth binding reagent is a binding partner of a biomarker selected from anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA, respectively; forming at least a first, second, third, fourth and fifth binding complex comprising the binding reagents and the biomarkers; and detecting the biomarkers in each of the binding complexes.

In embodiments, the disclosure provides a multiplexed seventh assay method comprising, detecting at least two human biomarkers in a biological sample in a multiplexed assay format, wherein the at least two biomarkers are selected from: (a) anti-insulin IgM and (b) anti-MPO IgA, wherein the multiplexed assay comprises combining, in one or more steps: the biological sample; at least a first and second binding reagent, wherein the first and second binding reagent is a binding partner of a biomarker selected from anti-insulin IgM and anti-MPO IgA, respectively; forming at least a first and second binding complex comprising the binding reagents and the biomarkers; and detecting the concentration of the biomarkers in each of the binding complexes. In embodiments, the method further comprises detecting, in a multiplexed assay format, at least one additional biomarker in the biological sample, wherein the at least one additional biomarker is TARC, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, anti-insulin IgA or a combination thereof.

In embodiments, the disclosure provides an eighth assay method comprising quantifying the amount of a human biomarker in a biological sample, wherein the biomarker is selected from anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, anti-insulin IgA, or combinations thereof, wherein the quantifying comprises (a) contacting the biological sample with (i) a capture reagent that specifically binds to the biomarker in a binding domain on a surface, wherein the binding domain further comprises an anchoring reagent; (ii) a first detection reagent that specifically binds to the biomarker and is linked to a first nucleic acid probe; and (iii) a second detection reagent that specifically binds to the biomarker and is linked to a second nucleic acid probe, thereby forming a complex on the surface comprising the capture reagent, the first biomarker, and the first and second detection reagents; (b) using an extension process that requires the first and second probes to be in proximity, extending the second probe to form an extended sequence comprising an anchoring region that binds the anchoring reagent; (c) binding the extended sequence to the anchoring reagent; and (d) measuring the amount of extended sequence bound to the binding domain, thereby quantifying the amount of the biomarker.

In embodiments, the disclosure provides a multiplexed ninth assay method comprising, detecting at least two human biomarkers in a biological sample in a multiplexed assay format, wherein the at least two biomarkers are selected from: (a) anti-DGP IgA, (b) anti-DGP IgG, (c) anti-DGP IgM, (d) anti-TGM2 IgA, (e) anti-TGM2 IgG and (f) anti-TGM2 IgM, wherein the multiplexed assay comprises combining, in one or more steps: the biological sample; at least a first and second binding reagent, wherein the first and second binding reagent is a binding partner of a biomarker selected from anti-DGP IgA, anti-DGP IgG, anti-DGP IgM, anti-TGM2 IgA, anti-TGM2 IgG and anti-TGM2 IgM, respectively; forming at least a first and second binding complex comprising the binding reagents and the biomarkers; and detecting the concentration of the biomarkers in each of the binding complexes.

In embodiments, the disclosure provides a multiplexed tenth assay method comprising, detecting at least three human biomarkers in a biological sample in a multiplexed assay format, wherein the at least three biomarkers are selected from: (a) anti-DGP IgA, (b) anti-DGP IgG, (c) anti-TGM2 IgA, (d) anti-TGM2 IgG, (e) anti-Jo1 IgA, (f) anti-beta2glycoprotein IgG, (g) anti-CCP IgG, (h) anti-CENP B IgG, (i) anti-GAD65 IgA, (j) anti-GAD65 IgG, (k) anti-IA2 IgM, (1) anti-proinsulin IgA, (m) anti-proinsulin IgM, (n) anti-U1RNPA IgA, (o) anti-ZnT8 IgA, (p) anti-Sc170 IgA, (q) anti-Smith IgA, and (r) anti-RoSSA60 IgG, wherein the multiplexed assay comprises combining, in one or more steps: the biological sample; at least a first, second and third, binding reagent, wherein the first, second and third, binding reagent is a binding partner of one of anti-DGP IgA, anti-DGP IgG, anti-TGM2 IgA, anti-TGM2 IgG, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, or anti-RoSSA60 IgG, respectively; forming at least a first, second, and third binding complex comprising the binding reagents and the biomarkers; and detecting the biomarkers in each of the binding complexes. In further embodiments, the present disclosure provides methods comprising detecting, in the multiplexed assay format, at least one additional biomarker in the biological sample, wherein the at least one additional biomarker is anti-beta2glycoprotein IgA, anti-IA2 IgG, anti-MPO IgA, anti-MPO IgM, anti-RNP68/70 IgM, anti-RoSSA52 IgG, anti-RoSSA52 IgM, anti-RoSSA60 IgM or a combination thereof.

In embodiments, the disclosure provides an eleventh assay method comprising quantifying the amount of a human biomarker in a biological sample, wherein the biomarker is selected from anti-DGP IgA, anti-DGP IgG, anti-DGP IgM, anti-TGM2 IgA, anti-TGM2 IgG, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, or anti-RoSSA60 IgG, anti-beta2glycoprotein IgA, anti-IA2 IgG, anti-MPO IgA, anti-MPO IgM, anti-RNP68/70 IgM, anti-RoSSA52 IgG, anti-RoSSA52 IgM and anti-RoSSA60 IgM, wherein the quantifying comprises (a) contacting the biological sample with (i) a capture reagent that specifically binds to the biomarker in a binding domain on a surface, wherein the binding domain further comprises an anchoring reagent; (ii) a first detection reagent that specifically binds to the biomarker and is linked to a first nucleic acid probe; and (iii) a second detection reagent that specifically binds to the biomarker and is linked to a second nucleic acid probe, thereby forming a complex on the surface comprising the capture reagent, the first biomarker, and the first and second detection reagents; (b) using an extension process that requires the first and second probes to be in proximity, extending the second probe to form an extended sequence comprising an anchoring region that binds the anchoring reagent; (c) binding the extended sequence to the anchoring reagent; and (d) measuring the amount of extended sequence bound to the binding domain, thereby quantifying the amount of the biomarker.

In further embodiments of any of the above methods, the disclosure provides methods wherein the biomarkers are antibodies and the binding reagents and detection reagents are antigens to the antibodies. In further embodiments, the disclosure provides methods wherein the biomarkers are antibodies, the binding reagents are antigens to the biomarker antibodies, and the detection reagents are antigens and/or detection antibodies that bind the biomarker antibodies.

In embodiments, the disclosure provides a method of determining if treatment of a human subject having Type 1 diabetes with alefacept is effective, comprising (a) conducting the first, second or third assay methods above on a biological sample of the human taken at a timepoint following the beginning of treatment with alefacept; (b) detecting the concentration of at least one of anti-IA2 IgG, anti-beta2glycoprotein IgG, anti-DGP IgG, anti-IA2 IgM, anti-MPO IgA, anti-proinsulin IgG, anti-MPO IgM, anti-ZnT8 IgM or a combination thereof; and (c) determining: (i) if the concentration of at least one of anti-IA2 IgG, anti-beta2glycoprotein IgG, anti-DGP IgG, anti-IA2 IgM and anti-MPO IgA is higher compared to a control, or (ii) if the concentration of at least one of anti-proinsulin IgG, anti-MPO IgM, anti-ZnT8 IgM is lower compared to levels in the individual before treatment with alefacept: (i) if the concentration of at least one of anti-IA2 IgG, anti-beta2glycoprotein IgG, anti-DGP IgG, anti-IA2 IgM and anti-MPO IgA is higher than the control, or (ii) if the concentration of at least one of anti-proinsulin IgG, anti-MPO IgM, anti-ZnT8 IgM is lower compared to levels in the individual before treatment with alefacept, reporting that the treatment with alefacept is effective.

In further embodiments, the disclosure provides methods wherein the biological sample is taken at a timepoint 0 weeks, 11 weeks, 26 weeks or 30 weeks following the beginning of treatment with alefacept.

In embodiments, the disclosure provides a method of determining if a human subject having Type 1 diabetes is a candidate for treatment with alefacept, comprising (a) conducting the first, second or third assay methods above on a biological sample of the human; (b) detecting the concentration of at least one of anti-IA2 IgG, anti-beta2glycoprotein IgG, anti-DGP IgG, anti-IA2 IgM, anti-MPO IgA or a combination thereof; and (c) determining if the concentration of at least one of anti-IA2 IgG, anti-beta2glycoprotein IgG, anti-DGP IgG, anti-IA2 IgM and anti-MPO IgA is higher compared to a control, wherein the control is a human subject that is not a candidate for treatment with alefacept or a human subject that does not have Type 1 diabetes; wherein if the concentration of at least one of anti-IA2 IgG, anti-beta2glycoprotein IgG, anti-DGP IgG, anti-IA2 IgM and anti-MPO IgA is higher than the control, reporting that the human is a candidate for treatment with alefacept.

In embodiments, the disclosure provides a method of determining if a human subject has systemic lupus erythematosus, comprising conducting the fourth or fifth assay methods above on a biological sample of the human; detecting the concentration of at least one of anti-Smith IgG, anti-RoSSA60 IgG, anti-U1 RNPA IgG, anti-insulin IgM, anti-RoSSA52 IgG, anti-GAD65 IgM, anti-ZnT8 IgG, anti-RoSSA60 IgA, anti-RoSSA52 IgM, anti-aNCA PR3 IgG, anti-Jo 1 IgA, anti-Smith IgA or a combination thereof; and determining if the concentration of at least one of anti-Smith IgG, anti-RoSSA60 IgG, anti-U1 RNPA IgG, anti-insulin IgM, anti-RoSSA52 IgG, anti-GAD65 IgM, anti-ZnT8 IgG, anti-RoSSA60 IgA, anti-RoSSA52 IgM, anti-aNCA PR3 IgG, anti-Jo1 IgA, and anti-Smith IgA is higher compared to a control, wherein the control is a human subject that does not have systemic lupus erythematosus; wherein if the concentration of at least one of anti-Smith IgG, anti-RoSSA60 IgG, anti-U1 RNPA IgG, anti-insulin IgM, anti-RoSSA52 IgG, anti-GAD65 IgM, anti-ZnT8 IgG, anti-RoSSA60 IgA, anti-RoSSA52 IgM, anti-aNCA PR3 IgG, anti-Jo1 IgA, and anti-Smith IgA is higher than the control, reporting that the human subject has systemic lupus erythematosus.

In embodiments, the disclosure provides a method of determining if a human subject is at risk of a systemic lupus erythematosus flare, comprising conducting the sixth, seventh or eighth assay methods above on a biological sample of the human; detecting the concentration of at least one of anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, anti-insulin IgA or a combination thereof; and determining if the concentration of at least one of anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA is higher compared to a control, wherein the control is a human subject that does not have systemic lupus erythematosus; wherein if the concentration of at least one of anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA is higher than the control, reporting that the human subject is at risk of a systemic lupus erythematosus flare.

In embodiments, the disclosure provides a method of determining if a human subject has celiac disease, comprising conducting the ninth, tenth or eleventh assay methods above on a biological sample of the human; detecting the concentration of at least one of anti-DGP IgA, anti-DGP IgG, anti-DGP IgM, anti-TGM2 IgA, anti-TGM2 IgG, anti-TGM2 IgM, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, anti-RoSSA60 IgG or a combination thereof; and determining if the concentration of at least one of anti-DGP IgA, anti-DGP IgG, anti-TGM2 IgA, anti-TGM2 IgG, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, or anti-RoSSA60 IgG is higher compared to a control, wherein the control is a human subject that does not have celiac disease; wherein if the concentration of at least one of anti-DGP IgA, anti-DGP IgG, anti-DGP IgM, anti-TGM2 IgA, anti-TGM2 IgG, anti-TGM2 IgM, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, or anti-RoSSA60 IgG is higher than the control, reporting that the human subject has celiac disease.

In embodiments, the disclosure provides a kit comprising, in one or more vials, containers, or compartments: (a) a surface comprising at least a first and second binding reagent immobilized on an associated first and second binding domain, wherein the first and second binding reagent is a binding partner of anti-IA2 IgG and anti-beta2glycoprotein IgG, respectively; (b) a detection reagent that specifically binds to anti-IA2 IgG; and (c) a detection reagent that specifically binds to anti-beta2glycoprotein IgG.

In embodiments, the disclosure provides a kit comprising, in one or more vials, containers, or compartments: (a) a surface comprising at least a first, second, third, fourth and fifth binding reagent immobilized on an associated first, second, third, fourth and fifth binding domain, wherein the first, second, third, fourth and fifth binding reagent is a binding partner of anti-IA2 IgG, anti-beta2glycoprotein IgG, anti-DGP IgG, anti-IA2 IgM and anti-MPO IgA, respectively; (b) a detection reagent that specifically binds to anti-IA2 IgG; (c) a detection reagent that specifically binds to anti-beta2glycoprotein IgG; (d) a detection reagent that specifically binds to anti-DGP IgG; (e) a detection reagent that specifically binds to anti-IA2 IgM; and (f) a detection reagent that specifically binds to anti-MPO IgA.

In embodiments, the disclosure provides a kit comprising, in one or more vials, containers, or compartments: (a) a surface comprising at least a first, second and third binding reagent immobilized on an associated first, second and third binding domain, wherein the first, second and third binding reagent is a binding partner of anti-Smith IgG, anti-RoSSA60 IgG and anti-U1 RNPA IgG, respectively; (b) a detection reagent that specifically binds to anti-Smith IgG; (c) a detection reagent that specifically binds to anti-RoSSA60 IgG; and (d) a detection reagent that specifically binds to anti-U1 RNPA IgG.

In embodiments, the disclosure provides a kit comprising, in one or more vials, containers, or compartments: (a) a surface comprising at least a first, second, third, fourth and fifth binding reagent immobilized on an associated first, second, third, fourth and fifth binding domain, wherein the first, second, third, fourth and fifth binding reagent is a binding partner of anti-insulin IgM, anti-MPO IgA, anti-Jo1 IgA, anti-ZnT8 IgM, anti-GAD65 IgG, respectively; (b) a detection reagent that specifically binds to anti-insulin IgM; (c) a detection reagent that specifically binds to anti-MPO IgA; (d) a detection reagent that specifically binds to anti-Jo1 IgA (e) a detection reagent that specifically binds to anti-ZnT8 IgM; and (f) a detection reagent that specifically binds to anti-GAD65 IgG.

In embodiments, the disclosure provides a kit comprising, in one or more vials, containers, or compartments: (a) a surface comprising at least a first and second binding reagent immobilized on an associated first and second binding domain, wherein the first and second binding reagent is a binding partner of anti-insulin IgM and anti-MPO IgA, respectively; (b) a detection reagent that specifically binds to anti-insulin IgM; and (c) a detection reagent that specifically binds to anti-MPO IgA.

In embodiments, the disclosure provides a kit comprising, in one or more vials, containers, or compartments: (a) a surface comprising at least a first, second, third, fourth, fifth and sixth binding reagent immobilized on an associated first, second, third, fourth, fifth and sixth binding domain, wherein the first, second, third, fourth, fifth and sixth binding reagent is a binding partner of anti-DGP IgA, anti-DGP IgG, anti-DGP IgM, anti-TGM2 IgA, anti-TGM2 IgG, and anti-TGM2 IgM, respectively; (b) a detection reagent that specifically binds to anti-DGP IgA; (c) a detection reagent that specifically binds to anti-DGP IgG; (d) a detection reagent that specifically binds to anti-DGP IgM; (e) a detection reagent that specifically binds to anti-TGM2 IgA; (f) a detection reagent that specifically binds to TGM2 IgG; and (g) a detection reagent that specifically binds to anti-TGM2 IgM.

In embodiments, the disclosure provides a kit comprising, in one or more vials, containers, or compartments: (a) a surface comprising at least a first, second, third, fourth, fifth and sixth binding reagent immobilized on an associated first, second, third, fourth, fifth and sixth binding domain, wherein the first, second, third, fourth, fifth and sixth binding reagent is a binding partner of a biomarker selected from anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, and anti-ZnT8 IgA, respectively; and (b) detection reagents that specifically binds to six of the biomarkers selected from anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, and anti-ZnT8 IgA, respectively.

In embodiments, the disclosure provides an assay system comprising at least one assay panel selected from: a) an assay panel comprising anti-insulin, anti-proinsulin, and anti-ZnT8 autoantibodies for IgG, IgA, and IgM isotypes; b) an assay panel comprising anti-GAD65 and anti-Intrinsic Factor autoantibodies for IgG, IgA, and IgM isotypes; c) an assay panel comprising anti-IA2 and anti-Jo-1 autoantibodies for IgG, IgA, and IgM isotypes; d) an assay panel comprising anti-IA2 and anti-Jo-1 autoantibodies for IgG, IgA, and IgM isotypes; e) an assay panel comprising anti-Smith, anti-Thyroglobulin, anti-MPO, anti-DGP, and anti-TGM2 autoantibodies for IgG, IgA, and IgM isotypes; f) an assay panel comprising anti-TPO, anti-U1RNPA, anti-RoSSA52, and anti-aNCA PR3 autoantibodies for IgG, IgA, and IgM isotypes; g) an assay panel comprising anti-CENPB, anti-Sc170, anti-CCP, anti-MPO, anti-RoSSA60, anti-U1RNPC, anti-Smith, and anti-RNP68/70 autoantibodies for IgG, IgA, and IgM isotypes; h) an assay panel comprising anti-LaSSB and anti-beta 2-glycoprotein autoantibodies for IgG, IgA, and IgM isotypes; i) an assay panel comprising anti-insulin IgM, anti-MPO IgA, anti-Jo-1 IgA and anti-GAD65 IgM autoantibodies, and MIP-1a, TARC and IL-7 antigens; j) an assay panel comprising anti-insulin IgM, anti-MPO IgA, anti-Jo-1 IgA and anti-GAD65 IgM autoantibodies, and MIP-1a, TARC, IL-7 and Eotaxin antigens; or k) an assay panel comprising anti-insulin IgM, anti-MPO IgA, anti-MPO IgM, TARC, anti-Jo-1 IgA and anti-GAD65 IgM autoantibodies, and MIP-1a, IL-7 and Eotaxin antigens.

In embodiments, the disclosure provides an assay system comprising at least one assay panel selected from: a) an assay panel comprising anti-IA2 IgG and anti-beta2glycoprotein IgG; b) an assay panel comprising at least two of anti-IA2 IgG, anti-beta2glycoprotein IgG, anti-DGP IgG, anti-IA2 IgM, anti-proinsulin IgG, anti-MPO IgM or anti-ZnT8 IgM; c) an assay panel comprising at least two of anti-Smith IgG, anti-RoSSA60 IgG, anti-U1 RNPA IgG, anti-insulin IgM and anti-RoSSA52 IgG, anti-GAD65 IgM, anti-ZnT8 IgG, anti-RoSSA60 IgA, anti-RoSSA52 IgM, anti-aNCA PR3 IgG, anti-Jo1 IgA, anti-Smith IgA, anti-U1 RNPA IgM, anti-MPO IgA, anti-U1RNPC IgG, anti-GAD65 IgG, anti-LaSSb IgA, anti-TPO IgG, anti-MPO IgG, anti-insulin IgA, anti-TPO IgM, anti-ZnT8 IgM, anti-IF IgG, anti-Smth IgG, TNF-alpha, IL-15, MIP1a, IL-10, NFL, IL-8, IL-6, IL-2, IL-21 or IL-23; d) an assay panel comprising at least two of anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG or anti-insulin IgA; e) as assay panel comprising at anti-insulin IgM and anti-MPO IgA; f) an assay panel comprising at least two of anti-DGP IgA, anti-DGP IgG, anti-DGP IgM, anti-TGM2 IgA, anti-TGM2 IgG, anti-TGM2 IgM, anti-Smith IgA, or anti-insulin IgA; or g) an assay panel comprising at least two of anti-DGP IgA, anti-DGP IgG, anti-TGM2 IgA, anti-TGM2 IgG, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, anti-RoSSA60 IgG, anti-beta2glycoprotein IgA, anti-IA2 IgG, anti-MPO IgA, anti-MPO IgM, anti-RNP68/70 IgM, anti-RoSSA52 IgG, anti-RoSSA52 IgM or anti-RoSSA60 IgM.

In embodiments of the assay systems disclosed herein, the assay system comprises simultaneous bridging assays, sequential bridging assays, classical serology assays or combinations thereof. In embodiments of the assay systems disclosed herein, the assay system comprises at least two, at least three, at least four, at least five, at least six or at least seven of the assay panels.

In embodiments, the disclosure provides an assay method comprising detecting, quantifying, or both, at least two human biomarkers in a biological sample, wherein the biomarker is (a) anti-IA2 IgG and (b) anti-beta2glycoprotein IgG, wherein the detecting, quantifying, or both, comprises: (a) contacting the biological sample with (i) a capture reagent that specifically binds to the biomarker in a binding domain on a surface, wherein the binding domain further comprises an anchoring reagent; (ii) a first detection reagent that specifically binds to the biomarker and is linked to a first nucleic acid probe; and (iii) a second detection reagent that specifically binds to the biomarker and is linked to a second nucleic acid probe, thereby forming a complex on the surface comprising the capture reagent, the first biomarker, and the first and second detection reagents; (b) using an extension process that requires the first and second probes to be in proximity, extending the second probe to form an extended sequence comprising an anchoring region that binds the anchoring reagent; (c) binding the extended sequence to the anchoring reagent; and (d) measuring the extended sequence bound to the binding domain, thereby detecting, quantifying, or both, the biomarkers.

In embodiments, the disclosure provides an assay method comprising detecting, quantifying, or both, at least four human biomarkers in a biological sample, wherein at least four biomarkers comprise (a) anti-IA2 IgG, (b) anti-beta2glycoprotein IgG, (c) anti-DGP IgG and (d) anti-IA2 IgM, wherein the quantifying, detecting, or both, comprises (a) contacting the biological sample with (i) a capture reagent that specifically binds to the biomarker in a binding domain on a surface, wherein the binding domain further comprises an anchoring reagent; (ii) a first detection reagent that specifically binds to the biomarker and is linked to a first nucleic acid probe; and (iii) a second detection reagent that specifically binds to the biomarker and is linked to a second nucleic acid probe, thereby forming a complex on the surface comprising the capture reagent, the first biomarker, and the first and second detection reagents; (b) using an extension process that requires the first and second probes to be in proximity, extending the second probe to form an extended sequence comprising an anchoring region that binds the anchoring reagent; (c) binding the extended sequence to the anchoring reagent; and (d) measuring the extended sequence bound to the binding domain, thereby detecting, quantifying, or both, the biomarkers.

In embodiments, the disclosure provides an assay method comprising detecting, quantifying, or both, at least three human biomarkers in a biological sample, wherein the at least three biomarkers comprise (a) anti-Smith IgG, (b) anti-RoSSA60 IgG, (c) anti-U1 RNPA IgG, (d) anti-insulin IgM and (e) anti-RoSSA52 IgG, wherein the quantifying, detecting, or both, comprises (a) contacting the biological sample with (i) a capture reagent that specifically binds to the biomarker in a binding domain on a surface, wherein the binding domain further comprises an anchoring reagent; (ii) a first detection reagent that specifically binds to the biomarker and is linked to a first nucleic acid probe; and (iii) a second detection reagent that specifically binds to the biomarker and is linked to a second nucleic acid probe, thereby forming a complex on the surface comprising the capture reagent, the first biomarker, and the first and second detection reagents; (b) using an extension process that requires the first and second probes to be in proximity, extending the second probe to form an extended sequence comprising an anchoring region that binds the anchoring reagent; (c) binding the extended sequence to the anchoring reagent; and (d) measuring the extended sequence bound to the binding domain, thereby detecting, quantifying, or both, the biomarkers.

In embodiments, the disclosure provides an assay method comprising detecting, quantifying, or both, at least five human biomarkers in a biological sample, wherein the at least five biomarkers are selected from: (a) anti-insulin IgM, (b) anti-MPO IgA, (c) anti-Jo1 IgA, (d) anti-ZnT8 IgM, (e) anti-GAD65 IgG, (f) anti-Smith IgA, (g) anti-IA2 IgA, (h) anti-GAD65 IgM, (i) anti-Jo1 IgG, (j) anti-beta2glycoprotein IgA, (k) anti-ZnT8 IgG, and (1) anti-insulin IgA, wherein the quantifying, detecting, or both, comprises (a) contacting the biological sample with (i) a capture reagent that specifically binds to the biomarker in a binding domain on a surface, wherein the binding domain further comprises an anchoring reagent; (ii) a first detection reagent that specifically binds to the biomarker and is linked to a first nucleic acid probe; and (iii) a second detection reagent that specifically binds to the biomarker and is linked to a second nucleic acid probe, thereby forming a complex on the surface comprising the capture reagent, the first biomarker, and the first and second detection reagents; (b) using an extension process that requires the first and second probes to be in proximity, extending the second probe to form an extended sequence comprising an anchoring region that binds the anchoring reagent; (c) binding the extended sequence to the anchoring reagent; and (d) measuring the extended sequence bound to the binding domain, thereby detecting, quantifying, or both, the biomarkers.

In embodiments, the disclosure provides a multiplexed assay method comprising, simultaneously detecting at least five human biomarkers in a biological sample in a multiplexed assay format, wherein the at least two biomarkers comprise (a) anti-TGM2, (b) anti-GAD65, (c) anti-ZnT8, (d) anti-insulin, and (e) anti-IA2, wherein the multiplexed assay comprises: combining, in one or more steps: the biological sample; at least a first and second binding reagent, wherein the first and second binding reagent is a binding partner of (a) TGM2, (b) GAD65, (c) ZnT8, (d) insulin, and (e) IA-2, respectively; forming at least a first and second binding complex comprising the binding reagents and the biomarkers; and detecting the biomarkers in each of the binding complexes. In embodiments, the assay cut-points are from about 10.0-13.0 U/mL for anti-TGM2, from about 12.0-15.0 IU/mL for anti-GAD65, from about 6.0-9.0 U/mL for anti-ZnT8, from about 0.1-2.0 U/mL for anti-insulin or from about 0.5-3.0 IU/mL for anti-IA2. In embodiments, the assay cut-points are from about 7.0-10.0 U/mL for anti-TGM2, from about 10.0-15.0 U/mL for anti-GAD65, from about 5.0-9.0 U/mL for anti-ZnT8, from about 1.0-3.0 U/mL for anti-insulin or from about 1.5-3.5 U/mL for anti-IA2. In embodiments, the biological sample is from a same human subject at ages selected from the group consisting of about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, and combinations thereof. In embodiments, the biological sample is from a same human subject every 2, 3, 4, 5, 6, 7, 8, 9, 10, 11, 12, 13, 14, 15, 16, 17, 18, 19, 20, 21, 22, 23 or 24 months.

In embodiments, the disclosure provides a kit comprising, in one or more vials, containers, or compartments: a surface comprising at least a first, second, third, fourth and fifth binding reagent immobilized on an associated first, second, third, fourth and fifth binding domain, wherein the first, second, third, fourth and fifth binding reagent is a binding partner of TGM2, GAD65, ZnT8, insulin, and IA-2, respectively; a detection reagent that specifically binds to TGM2; a detection reagent that specifically binds to GAD65; a detection reagent that specifically binds to ZnT8; a detection reagent that specifically binds to insulin; and a detection reagent that specifically binds to IA-2.

In embodiments of any of the multiplexed methods herein, the biomarkers are located on separate plates. In other embodiments of any of the multiplexed methods herein, the biomarkers are located on the same plate.

In embodiments, the disclosure provides an assay method comprising detecting at least two human biomarkers in a biological sample, wherein the at least two biomarkers comprise (a) anti-IA2 IgG and (b) anti-beta2glycoprotein IgG, wherein the assay comprises: contacting, in one or more steps: the biological sample; at least a first and second binding reagent, wherein the first and second binding reagent is a binding partner of anti-IA2 IgG and anti-beta2glycoprotein IgG, respectively; forming at least a first and second binding complex comprising the binding reagents and the biomarkers; and detecting the biomarkers in each of the binding complexes.

In embodiments, the disclosure provides an assay method comprising detecting at least four human biomarkers in a biological sample, wherein the at least four biomarkers comprise (a) anti-IA2 IgG, (b) anti-beta2glycoprotein IgG, (c) anti-DGP IgG and (d) anti-IA2 IgM, wherein the assay comprises: contacting, in one or more steps: the biological sample; at least a first, second, third and fourth binding reagent, wherein the first, second, third and fourth binding reagent is a binding partner of anti-IA2 IgG, anti-beta2glycoprotein IgG, anti-DGP IgG and anti-IA2 IgM, respectively; forming at least a first, second, third and fourth binding complex comprising the binding reagents and the biomarkers; and detecting the biomarkers in each of the binding complexes.

In embodiments, the disclosure provides an assay method comprising detecting at least three human biomarkers in a biological sample, wherein the at least three biomarkers comprise (a) anti-Smith IgG, (b) anti-RoSSA60 IgG, (c) anti-U1 RNPA IgG, (d) anti-insulin IgM and (e) anti-RoSSA52 IgG, wherein the assay comprises: contacting, in one or more steps: the biological sample; at least a first, second and third binding reagent, wherein the first, second and third binding reagent is a binding partner selected from anti-Smith IgG, anti-RoSSA60 IgG, anti-U1 RNPA IgG, anti-insulin IgM and anti-RoSSA52 IgG, respectively; forming at least a first, second and third binding complex comprising the binding reagents and the biomarkers; and detecting the biomarkers in each of the binding complexes.

In embodiments, the disclosure provides an assay method comprising detecting at least five human biomarkers in a biological sample, wherein the at least five biomarkers are selected from: (a) anti-insulin IgM, (b) anti-MPO IgA, (c) anti-Jo1 IgA, (d) anti-ZnT8 IgM, (e) anti-GAD65 IgG, (f) anti-Smith IgA, (g) anti-IA2 IgA, (h) anti-GAD65 IgM, (i) anti-Jo1 IgG, (j) anti-beta2glycoprotein IgA, (k) anti-ZnT8 IgG, and (1) anti-insulin IgA, wherein the assay comprises: contacting, in one or more steps: the biological sample; at least a first, second, third, fourth and fifth binding reagent, wherein the first, second, third, fourth and fifth binding reagent is a binding partner of a biomarker selected from anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA, respectively; forming at least a first, second, third, fourth and fifth binding complex comprising the binding reagents and the biomarkers; and detecting the biomarkers in each of the binding complexes.

In embodiments, the disclosure provides an assay method comprising detecting at least two human biomarkers in a biological sample, wherein the at least two biomarkers are selected from: (a) anti-insulin IgM and (b) anti-MPO IgA, wherein the assay comprises: contacting, in one or more steps: the biological sample; at least a first and second binding reagent, wherein the first and second, binding reagent is a binding partner of a biomarker selected from anti-insulin IgM and anti-MPO IgA, respectively; forming at least a first and second binding complex comprising the binding reagents and the biomarkers; and detecting the biomarkers in each of the binding complexes.

In embodiments, the disclosure provides an assay method comprising detecting at least two human biomarkers in a biological sample, wherein the at least two biomarkers are selected from: (a) anti-DGP IgA, (b) anti-DGP IgG, (c) anti-DGP IgM, (d) anti-TGM2 IgA, (e) anti-TGM2 IgG and (f) anti-TGM2 IgM, wherein the assay comprises: contacting, in one or more steps: the biological sample; at least a first and second binding reagent, wherein the first and second binding reagent is a binding partner of a biomarker selected from anti-DGP IgA, anti-DGP IgG, anti-DGP IgM, anti-TGM2 IgA, anti-TGM2 IgG and anti-TGM2 IgM, respectively; forming at least a first and second binding complex comprising the binding reagents and the biomarkers; and detecting the biomarkers in each of the binding complexes.

### BRIEF DESCRIPTION OF THE DRAWINGS

The following drawings form part of the present specification and are included to further demonstrate exemplary embodiments of certain aspects of the present invention.

FIGS. 1 and 2 relate to Example 2. FIGS. 1A and 1B are plots of average (FIG. 1A) and median (FIG. 1B) anti-ZnT8 antibody isotype concentrations over time following treatment with alefacept or placebo, measured as described in Example 1.

FIGS. 2A and 2B are plots of average (FIG. 2A) and median (FIG. 2B) LaSSB autoantibody isotype concentrations over time following treatment with alefacept or placebo, measured as described in Example 2.

FIGS. 3-10 relate to Example 3. FIG. 3 shows plots of results obtained for embodiments of anti-IA2 IgG assays as described in Example 3. Each plotted line represents an individual patient at all available time points. The graphs are sorted based on treatment group (Placebo or Alefacept), and treatment outcome (Positive or Negative). The data are shown in terms of sample signals and calculated concentrations (top half). The ratios of signals or concentrations to each patient's 0 time point sample values are shown as well (bottom half).

FIG. 4 shows a summary plot of median values for all treated patients for anti-IA2 IgG and IgM in the positive and negative response groups in an embodiment of the invention described in Example 3. Higher levels of anti-IA2 IgG and IgM are seen in the positive response group relative to negative response group at earlier time points.

FIG. 5 shows plots of results obtained for anti-beta2glycoprotein IgA assays in an embodiment of the invention described in Example 3. Each plotted line represents an individual patient at all available time points. The graphs are sorted based on treatment group (Placebo or Alefacept), and treatment outcome (Positive or Negative). The data are shown in terms of sample signals and calculated concentrations (top half). The ratios of signals or concentrations to each patient's 0 time point sample values are shown as well (bottom half).

FIG. 6 shows a summary plot of median values for all treated patients for anti-beta2glycoprotein IgA in the positive and negative response groups in an embodiment as described in Example 3.

FIG. 7 shows plots of results obtained for embodiments of anti-DGP IgG assays as described in Example 3. Each plotted line represents an individual patient at all available time points. The graphs are sorted based on treatment group (Placebo or Alefacept), and treatment outcome (Positive or Negative). The data are shown in terms of sample signals and calculated concentrations (top half). The ratios of signals or concentrations to each patient's 0 time point sample values are shown as well (bottom half).

FIG. 8 shows a summary plot of median values for all treated patients for anti-DGP IgG in the positive and negative response groups in an embodiment of the invention as described in Example 3.

FIG. 9 shows plots of results obtained for embodiments of anti-IA2 IgM assays as described in Example 3. Each plotted line represents an individual patient at all available time points. The graphs are sorted based on treatment group (Placebo or Alefacept), and treatment outcome (Positive or Negative). The data are shown in terms of sample signals and calculated concentrations (top half). The ratios of signals or concentrations to each patient's 0 time point sample values are shown as well (bottom half).

FIG. 10 shows plots of results obtained for anti-MPO IgA assays in embodiments as described in Example 3. Each plotted line represents an individual patient at all available time points. The graphs are sorted based on treatment group (Placebo or Alefacept), and treatment outcome (Positive or Negative). The ratios of signals or concentrations to each patient's 0 time point sample values are shown.

FIG. 11 relates to Example 4. FIG. 11 shows whisker box plots of the biomarker concentrations detected for each sample in the SLE flare and non-flare groups as described in Example 4. FIG. 11A shows concentrations for anti-insulin IgM. FIG. 11B shows concentrations for anti-MPO IgA.

FIGS. 12-15 relate to Example 5. FIG. 12 shows a plot of the SLE disease versus control LASSO regulation described in Example 5, displaying cross validated deviance, as determined by the cost function, for different lambda values.

FIG. 13 shows a plot of the ROC curves generated from outcome probability equations for the Panel of 8 and the Panel of 9 as applied to the original dataset. The equations are defined in Example 5. The AUC for both curves is 1.0.

FIG. 14 shows a plot of the SLE flare versus non-flare LASSO regulation described in Example 5, displaying cross validated deviance, as determined by the cost function, for different lambda values.

FIG. 15 shows a plot of the ROC curves generated from outcome probability equations for the Panel of 7, Panel of 8 and the Panel of 9 as applied to the original dataset. The equations are defined in Example 5. The AUC for each curve is greater than 0.98.

FIGS. 16-18 relate to Example 7. FIG. 16 shows whisker-box plots of the concentration of anti-TGM2 and anti-DGP antibodies of the IgA, IgG and IgM isotypes as shown. Samples are tested from non-celiac patients (NC), treated celiac patients and untreated celiac patients as described in Example 7.

FIG. 17 shows whisker-box plots of the concentration of anti-Smith IgG, anti-U1RNPA IgG and anti-RoSSA60 IgG autoantibodies in control and Lupus (SLE) patients, measured as described in Example 7.

FIG. 18 shows a plot of the concentration of anti-insulin IgM and anti-MPO IgA autoantibodies in the same patient during either SLE flare or non-flare, measured as described in Example 7. Each line represents one patient, connecting the flare and non-flare marker concentrations.

FIG. 19 illustrates an exemplary assay surface described in embodiments herein. Shown is a schematic of a well of an exemplary 96-well assay plate, comprising ten distinct binding domains (“spots”).

FIGS. 20 and 21 relate to Example 8. FIG. 20 shows a plot of the measured concentration of each biomarker of the panel of five described in Example 8 for each one of the 72 “normal” samples tested. The top horizontal line in each column represents the 98th percentile while the bottom horizontal line in each column represents the 90th percentile.

FIG. 21 shows a plot of the measured concentration of each biomarker of the panel of five described in Example 8 for each of the samples tested. A total of 172 Type 1 Diabetes (T1D) samples were tested for each biomarker. The horizontal line in each column represents the 98th percentile cut-point.

### DETAILED DESCRIPTION OF THE INVENTION

In embodiments, the present disclosure provides assays for quantifying amounts of at least one biomarker in a sample. In embodiments, the present disclosure provides assays for quantifying amounts of at least two, at least three, at least four or at least five biomarkers in a sample. In embodiments, the disclosure also provides kits for performing the assays.

### I. DEFINITIONS

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

As used herein, the term “simultaneous” or “simultaneously” in reference to one or more events (e.g., detecting analytes as described herein) means that the events occur at exactly the same time or at substantially the same time, e.g., simultaneous events described herein can occur less than or about 10 minutes apart, less than or about 5 minutes apart, less than or about 2 minutes apart, less than or about 1 minute apart, or less than or about 30 seconds apart.

As used herein, the terms “multiplex” and “multiplexed” as they refer to assays, formats and systems, refer to assays, formats and systems that simultaneously detect more than one analyte or allow for simultaneous detection of more than one analyte.

The terms “anti-insulin,” “insulin autoantibodies” and “anti-IAA,” as used herein, all refer to antibodies to insulin, including antibodies of certain isotypes where indicated. The terms “anti-proinsulin,” “proinsulin autoantibodies,” “anti-proIAA” as used herein, all refer to antibodies to proinsulin, including antibodies of certain isotypes where indicated.

### II. OVERVIEW

Detection of the presence of biomarkers and/or the measurement of biomarker values and levels before and after a particular event, e.g., cellular, environmental or treatment event, may be used to gain information regarding an individual's response to the event. For example, samples or model organisms can be subjected to stress- or disease-inducing conditions, or a treatment or prevention regimen, and a particular biomarker can then be detected and quantitated in order to determine its changes in response to the condition or regimen. In one example, detection and quantitation of biomarkers can be used to determine a subject's response to treatment with a therapeutic agent such as a drug or biologic.

While single biomarkers generally do not provide sufficient information, e.g., for prediction and/or diagnosis of a disease or condition, certain combinations of biomarkers may be used to provide a strong prediction and/or diagnosis. Although a linear combination of biomarkers (i.e., the combination comprises biomarkers that individually provide a relatively strong correlation) can be utilized, linear combinations may not be available in many situations, for example, when there are not enough biomarkers available and/or with strong correlation. In alternative approaches, a biomarker combination is selected such that the combination is capable of achieving improved performance (i.e., prediction or diagnosis) compared with any of the individual biomarkers, each of which may not be a strong correlator on its own. Biomarkers for inclusion in a biomarker combination can be selected for based on their performance in different individuals, e.g., patients, wherein the same biomarker may not have the same performance in different individuals, but when combined with the remaining biomarkers, provide an unexpectedly strong correlation for prediction or diagnosis in a population. For example, Bansal et al., Statist Med 32: 1877-1892 (2013) describe methods of determining biomarkers to include in such a combination, noting in particular that optimal combinations may not be obvious to one of skill in the art, especially when subgroups are present or when individual biomarker correlations are different between cases and controls. Thus, selecting a combination of biomarkers for providing a consistent and accurate prediction and/or diagnosis can be particularly challenging and unpredictable.

Even when a suitable combination of biomarkers is determined, utilizing the combination of biomarkers in an assay poses its own set of difficulties. For example, detecting and/or quantitating each biomarker in the combination in its own separate assay may not be feasible with small samples, and using a separate assay to measure each biomarker in a sample may not provide consistent and comparable results. Furthermore, running an individual assay for each biomarker in a combination can be a cumbersome and complex process that can be inefficient and costly.

A multiplexed assay that can simultaneously measure the concentrations of multiple biomarkers can provide reliable results while reducing processing time and cost. Challenges of developing a multi-biomarker assay (such as, e.g., a multiplexed assay described in embodiments herein) include, for example, determining compatible reagents for all of the biomarkers (e.g., capture and detection reagents described herein should be highly specific and not be cross-reactive; all assays should perform well in the same diluents); determining concentration ranges of the reagents for consistent assay (e.g., comparable capture and detection efficiency for the assays described herein); having similar levels in the condition and sample type of choice such that the levels of all of the biomarkers fall within the dynamic range of the assays at the same dilution; minimizing non-specific binding between the biomarkers and binding reagents thereof or other interferents; and accurately and precisely detecting a multiplexed output measurement.

The assays described herein have improved specificity, sensitivity, dynamic range, scalability, and, in embodiments, the ability to multiplex compared with conventional assays. In embodiments, the assays described herein provide an accurate measurement of low-abundance biomarkers in a sample. In embodiments, the assays described herein provide accurate measurements of high- and low-abundance biomarkers in the same sample.

### III. ASSAY PANELS

In embodiments, the disclosure provides for assay systems, as described herein, that comprise at least one assay panel. An “assay panel,” as used herein, is a collection of reagents that bind a biomarker that are used together in the analysis of a sample. An assay panel may be grouped together using methods known in the art. In embodiments, the reagents of the assay panel may be grouped together on a surface, such as a multi-well plate. In embodiments, the reagents of the assay panel may be grouped together on a chip. In embodiments, the reagents of the assay panel may be grouped together in a single vial. In embodiments, the reagents of the assay panel may be grouped together in multiple vials.

The disclosure provides for assay panels that can be used for analysis of samples. In embodiments, the assay panel is used in a bridging, simultaneous assay. In embodiments, the assay panel is used in a bridging, sequential assay. In embodiments, the assay panel is used in a classical serology assay. In embodiments, the assay panel is used in a sandwich immunoassay. In embodiments, the assay panel is used in more than one type of assay.

In embodiments, the assay panel comprises biomarkers selected from anti-insulin, anti-proinsulin, anti-ZnT8, anti-GAD65, anti-Intrinsic Factor, anti-IA2, anti-Jo-1, anti-Smith, anti-Thyroglobulin, anti-MPO, anti-DGP, anti-TGM2, anti-TPO, anti-U1RNPA, anti-RoSSA52, anti-aNCA PR3, anti-CENPB, anti-Sc170, anti-CCP, anti-RoSSA60, anti-U1RNPC, anti-RNP68/70, anti-LaSSB, anti-beta2glycoprotein, autoantibodies for IgG, IgA, and IgM isotypes, and MIP-1a, IL-7 and Eotaxin antigens.

In embodiments, the assay panel comprises biomarkers selected from anti-insulin, anti-proinsulin, anti-ZnT8, anti-GAD65, anti-Intrinsic Factor, anti-IA2, anti-Jo-1, anti-Smith, anti-Thyroglobulin, anti-MPO, anti-DGP, anti-TGM2, anti-TPO, anti-U1RNPA, anti-RoSSA52, anti-aNCA PR3, anti-CENPB, anti-Sc170, anti-CCP, anti-RoSSA60, anti-U1RNPC, anti-RNP68/70, anti-LaSSB and anti-beta2glycoprotein autoantibodies for IgG, IgA, and IgM isotypes. In embodiments, the assay panel comprises 2, 3, 4, 5, 6, 7, 8, 9, 10, 11, 12, 13, 14, 15, 16, 17, 18, 19, 20, 21, 22, 23 or 24 of the biomarkers.

In embodiments, the assay panel comprises biomarkers selected from anti-insulin, anti-proinsulin, anti-ZnT8, anti-GAD65, anti-Intrinsic Factor, anti-IA2, anti-Jo-1, anti-Smith, anti-Thyroglobulin, anti-MPO, anti-DGP, anti-TGM2, anti-TPO, anti-U1RNPA, anti-RoSSA52, anti-aNCA PR3, anti-CENPB, anti-Sc170, anti-CCP, anti-RoSSA60, anti-U1RNPC, anti-RNP68/70, anti-LaSSB, and anti-beta2glycoprotein, autoantibodies for IgG, IgA, and IgM isotypes, and MIP-1a, IL-7 and Eotaxin antigens. In embodiments, the assay panel comprises 2, 3, 4, 5, 6, 7, 8, 9, 10, 11, 12, 13, 14, 15, 16, 17, 18, 19, 20, 21, 22, 23, 24, 25, 26 or 27 of the biomarkers.

In embodiments, the assay panel comprises anti-insulin, anti-proinsulin, and anti-ZnT8 autoantibodies for IgG, IgA, and IgM isotypes.

In embodiments, the assay panel is a simultaneous bridging assay comprising anti-insulin, anti-proinsulin, and anti-ZnT8 autoantibodies for IgG, IgA, and IgM isotypes.

In embodiments, the assay panel comprises anti-GAD65 and anti-Intrinsic Factor autoantibodies for IgG, IgA, and IgM isotypes.

In embodiments, the assay panel is a simultaneous bridging assay comprising anti-GAD65 and anti-Intrinsic Factor autoantibodies for IgG, IgA, and IgM isotypes.

In embodiments, the assay panel comprises anti-IA2 and anti-Jo-1 autoantibodies for IgG, IgA, and IgM isotypes.

In embodiments, the assay panel is a simultaneous bridging assay comprising anti-IA2 and anti-Jo-1 autoantibodies for IgG, IgA, and IgM isotypes.

In embodiments, the assay panel comprises anti-Smith, anti-Thyroglobulin, anti-MPO, anti-DGP, and anti-TGM2 autoantibodies for IgG, IgA, and IgM isotypes.

In embodiments, the assay panel is a sequential bridging assay comprising anti-Smith, anti-Thyroglobulin, anti-MPO, anti-DGP, and anti-TGM2 autoantibodies for IgG, IgA, and IgM isotypes.

In embodiments, the assay panel comprises anti-TPO, anti-U1RNPA, anti-RoSSA52, and anti-aNCA PR3 autoantibodies for IgG, IgA, and IgM isotypes.

In embodiments, the assay panel is a sequential bridging assay comprising anti-TPO, anti-U1RNPA, anti-RoSSA52, and anti-aNCA PR3 autoantibodies for IgG, IgA, and IgM isotypes.

In embodiments, the assay panel comprises anti-CENPB, anti-Sc170, anti-CCP, anti-MPO, anti-RoSSA60, anti-U1RNPC, anti-Smith, and anti-RNP68/70 autoantibodies for IgG, IgA, and IgM isotypes.

In embodiments, the assay panel is a classical serology assay comprising anti-CENPB, anti-Sc170, anti-CCP, anti-MPO, anti-RoSSA60, anti-U1RNPC, anti-Smith, and anti-RNP68/70 autoantibodies for IgG, IgA, and IgM isotypes.

In embodiments, the assay panel comprises anti-LaSSB and anti-beta 2-glycoprotein autoantibodies for IgG, IgA, and IgM isotypes.

In embodiments, the assay panel is a classical serology assay comprising anti-LaSSB and anti-beta 2-glycoprotein autoantibodies for IgG, IgA, and IgM isotypes.

In embodiments, the assay panel comprises anti-TGM2, anti-GAD65, anti-ZnT8, anti-insulin and anti-IA2 autoantibodies.

In embodiments, the assay panel is a simultaneous bridging assay comprising anti-TGM2, anti-GAD65, anti-ZnT8, anti-insulin and anti-IA2 autoantibodies.

In embodiments, the assay panel is a sequential bridging assay comprising anti-TGM2, anti-GAD65, anti-ZnT8, anti-insulin and anti-IA2 autoantibodies.

In embodiments, the assay panel comprises anti-insulin IgM, anti-MPO IgA, TARC, anti-Jo-1 IgA and anti-GAD65 IgM autoantibodies, and MIP-1a, and IL-7 antigens.

In embodiments, the assay panel comprises anti-insulin IgM, anti-MPO IgA, TARC, anti-Jo-1 IgA and anti-GAD65 IgM autoantibodies, and MIP-1a, IL-7 and Eotaxin antigens.

In embodiments, the assay panel comprises anti-insulin IgM, anti-MPO IgA, MPO IgM, TARC, anti-Jo-1 IgA and anti-GAD65 IgM autoantibodies, and MIP-1a, IL-7 and Eotaxin antigens.

In embodiments, the assay panel comprises anti-IA2 IgG and anti-beta2glycoprotein IgG.

In embodiments, the assay panel comprises biomarkers selected from at least 2 of anti-IA2 IgG, anti-beta2glycoprotein IgG, anti-DGP IgG, anti-IA2 IgM anti-proinsulin IgG, anti-MPO IgM or anti-ZnT8 IgM. In embodiments, the assay panel comprises at least 3 or at least 4 of the biomarkers.

In embodiments, the assay panel comprises biomarkers selected from at least 2 of anti-Smith IgG, anti-RoSSA60 IgG, anti-U1 RNPA IgG, anti-insulin IgM and anti-RoSSA52 IgG, anti-GAD65 IgM, anti-ZnT8 IgG, anti-RoSSA60 IgA, anti-RoSSA52 IgM, anti-aNCA PR3 IgG, anti-Jo1 IgA, anti-Smith IgA, anti-U1 RNPA IgM, anti-MPO IgA, anti-U1RNPC IgG, anti-GAD65 IgG, anti-LaSSb IgA, anti-TPO IgG, anti-MPO IgG, anti-insulin IgA, anti-TPO IgM, anti-ZnT8 IgM, anti-IF IgG, anti-Smth IgG, TNF-alpha, IL-15, MIP1a, IL-10, NFL, IL-8, IL-6, IL-2, IL-21 or IL-23. In embodiments, the assay panel comprises at least 3, 4, 5, 6, 7, 8, 9, 10, 11, 12, 13, 14, 15, 16, 17, 18, 19 or 20 of the biomarkers.

In embodiments, the assay panel comprises biomarkers selected from at least 2 of anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG or anti-insulin IgA. In embodiments, the assay panel comprises at least 3, 4, 5, 6, 7, 8, 9, 10, 11 or 12 of the biomarkers.

In embodiments, the assay panel comprises anti-insulin IgM and anti-MPO IgA.

In embodiments, the assay panel comprises at least 2 biomarkers selected from anti-DGP IgA, anti-DGP IgG, anti-DGP IgM, anti-TGM2 IgA, anti-TGM2 IgG, anti-TGM2 IgM, anti-Smith IgA, or anti-insulin IgA. In embodiments, the assay panel comprises at least 3, 4, 5, 6, 7 or 8 of the biomarkers.

In embodiments, the assay panel comprises at least 2 biomarkers selected from anti-DGP IgA, anti-DGP IgG, anti-TGM2 IgA, anti-TGM2 IgG, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, anti-RoSSA60 IgG, anti-beta2glycoprotein IgA, anti-IA2 IgG, anti-MPO IgA, anti-MPO IgM, anti-RNP68/70 IgM, anti-RoSSA52 IgG, anti-RoSSA52 IgM or anti-RoSSA60 IgM. In embodiments, the assay panel comprises at least 3, 4, 5, 6, 7, 8, 9, 10, 11, 12, 13, 14, 15, 16, 17, 18, 19, 20, 21, 22, 23, 24, 25 or 26 of the biomarkers.

In embodiments, the assay panel comprises at least 2 biomarkers selected from anti-CCP IgG, anti-beta2glycoprotein IgG, anti-CENP B IgG, anti-GAD65 IgA, anti-GAD65 IgG, anti-IA2 IgM, anti-Jo1 IgA, anti-proinsulin IgA, anti-proinsulin IgM, anti-TGM2 IgA, anti-U1RNPA IgA or anti-ZnT8 IgA. In embodiments, the assay panel comprises at least 3, 4, 5, 6, 7, 8, 9, 10, 11 or 12 of the biomarkers.

In embodiments, the assay panel comprises at least 2 biomarkers selected from anti-aNCAPR3 IgG, anti-beta2glycoprotein IgA, anti-beta2glycoprotein IgG, anti-CENP B IgG, anti-GAD65 IgA, anti-GAD65 IgG, anti-IA2 IgG, anti-IA2 IgM, anti-Jo1 IgA, anti-MPO IgA, anti-MPO IgM, anti-proinsulin IgM, anti-RNP68/70 IgM, anti-RoSSA52 IgG, anti-RoSSA52 IgM, anti-RoSSA60 IgM, anti-Smith IgG or anti-U1RNPC IgA. In embodiments, the assay panel comprises at least 3, 4, 5, 6, 7, 8, 9, 10, 11, 12, 13, 14, 15, 16, 17 or 18 of the biomarkers.

In embodiments, the assay system comprises at least two assay panels. In embodiments, the assay system comprises at least three assay panels. In embodiments, the assay system comprises at least four assay panels. In embodiments, the assay system comprises at least five assay panels. In embodiments, the assay system comprises at least six assay panels. In embodiments, the assay system comprises at least seven assay panels. In embodiments, the assay system comprises at least eight assay panels. In embodiments, the assay system comprises at least nine assay panels. In embodiments, the assay system comprises at least ten assay panels.

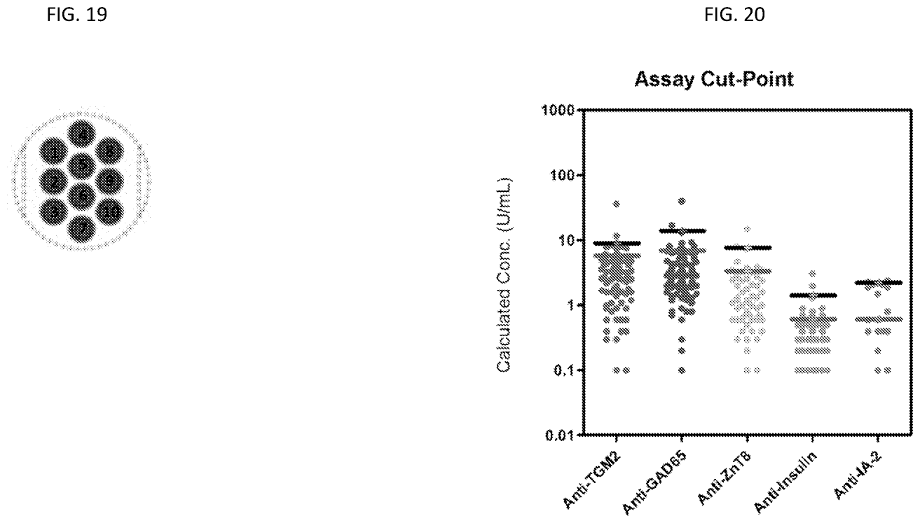

In embodiments, the assay panel is comprised in a well on an assay plate as described herein. In embodiments, the assay panel is comprised in a well on a 96-well assay plate as described herein. An embodiment of a well in a 96-well assay plate, comprising ten binding domains (“spots”), is shown in FIG. 19.

In embodiments, the assay panel is comprised in a well comprising ten binding domains (“spots”) as exemplified in FIG. 19, wherein one, two, three, four, five, six, seven, eight, nine or ten of the spots comprises a biomarker selected from anti-insulin, anti-proinsulin, anti-ZnT8, anti-GAD65, anti-Intrinsic Factor, anti-IA2, anti-Jo-1, anti-Smith, anti-Thyroglobulin, anti-MPO, anti-DGP, anti-TGM2, anti-TPO, anti-U1RNPA, anti-RoSSA52, anti-aNCA PR3, anti-CENPB, anti-Sc170, anti-CCP, anti-RoSSA60, anti-U1RNPC, anti-RNP68/70, anti-LaSSB and anti-beta 2-glycoprotein. In embodiments of the assay panel, spots that do not comprise one of the biomarkers listed above comprise immobilized bovine serum albumin (BSA).

In embodiments, the assay panel is comprised in a well comprising ten spots as exemplified in FIG. 19, wherein one, two or three of the spots comprises a biomarker selected from anti-insulin, anti-proinsulin, and anti-ZnT8. In embodiments, the assay panel is comprised in a well comprising ten spots as exemplified in FIG. 19, wherein Spot 1 comprises anti-insulin, Spot 2 comprises anti-proinsulin and Spot 5 comprises anti-ZnT8.

In embodiments, the assay panel is comprised in a well comprising ten spots as exemplified in FIG. 19, wherein one or two of the spots comprises a biomarker selected from anti-GAD65 and anti-Intrinsic Factor. In embodiments, the assay panel is comprised in a well comprising ten spots as exemplified in FIG. 19, wherein Spot 1 comprises anti-GAD65 and Spot 8 comprises anti-Intrinsic Factor.

In embodiments, the assay panel is comprised in a well comprising ten spots as exemplified in FIG. 19, wherein one or two of the spots comprises a biomarker selected from anti-IA2 and anti-Jo-1. In embodiments, the assay panel is comprised in a well comprising ten spots as exemplified in FIG. 19, wherein Spot 4 comprises anti-IA2 and Spot 6 comprises anti-Jo-1.

In embodiments, the assay panel is comprised in a well comprising ten spots as exemplified in FIG. 19, wherein one, two, three, four or five of the spots comprises a biomarker selected from anti-Smith, anti-Thyroglobulin, anti-MPO, anti-DGP, and anti-TGM2. In embodiments, the assay panel is comprised in a well comprising ten spots as exemplified in FIG. 19, wherein Spot 2 comprises anti-Thyroglobulin, Spot 3 comprises anti-TGM2, Spot 4 comprises anti-MPO, Spot 8 comprises anti-Smith and Spot 9 comprises anti-DGP.

In embodiments, the assay panel is comprised in a well comprising ten spots as exemplified in FIG. 19, wherein one, two, three or four of the spots comprises a biomarker selected from anti-TPO, anti-U1RNPA, anti-RoSSA52, and anti-aNCA PR3. In embodiments, the assay panel is comprised in a well comprising ten spots as exemplified in FIG. 19, wherein Spot 1 comprises anti-TPO, Spot 2 comprises anti-U1RNPA, Spot 3 comprises anti-RoSSA52 and Spot 4 comprises anti-aNCA PR3.

In embodiments, the assay panel is comprised in a well comprising ten spots as exemplified in FIG. 19, wherein one, two, three, four, five, six, seven or eight of the spots comprises a biomarker selected from anti-CENPB, anti-Sc170, anti-CCP, anti-MPO, anti-RoSSA60, anti-U1RNPC, anti-Smith and anti-RNP68/70. In embodiments, the assay panel is comprised in a well comprising ten spots as exemplified in FIG. 19, wherein Spot 1 comprises anti-CENPB, Spot 2 comprises anti-Sc170, Spot 3 comprises anti-CCP, Spot 4 comprises anti-MPO, Spot 5 comprises anti-RoSSA60, Spot 6 comprises anti-U1RNPC, Spot 8 comprises anti-Smith and Spot 9 comprises anti-RNP68/70

In embodiments, the assay panel is comprised in a well comprising ten spots as exemplified in FIG. 19, wherein one or two of the spots comprises a biomarker selected from anti-LaSSB and anti-beta 2-glycoprotein. In embodiments, the assay panel is comprised in a well comprising ten spots as exemplified in FIG. 19, wherein Spot 3 comprises anti-LaSSB and Spot 6 comprises anti-beta 2-glycoprotein.

In embodiments, the assay panel is comprised in a well comprising ten spots as exemplified in FIG. 19, wherein one, two, three, four or five of the spots comprises a biomarker selected from anti-TGM2, anti-GAD65, anti-ZnT8, anti-insulin and anti-IA2. In embodiments, the assay panel is comprised in a well comprising ten spots as exemplified in FIG. 19, wherein Spot 1 comprises anti-TGM2, Spot 2 comprises anti-GAD65, Spot 3 comprises anti-ZnT8, Spot 8 comprises anti-insulin and Spot 10 comprises anti-IA2.

### IV. TYPE 1 DIABETES

In embodiments, the present disclosure provides a biomarker assay for determining whether a subject having Type 1 diabetes is a candidate for treatment with the biologic therapeutic agent alefacept. For example, a subject having a biomarker profile as identified herein may be more responsive to treatment of Type 1 diabetes with alefacept than a subject having a different biomarker profile. Such a biomarker profile may be determined prior to the beginning of treatment with alefacept. In embodiments, if the subject is determined to be a candidate for treatment with alefacept, the subject is treated with alefacept. In embodiments, if the subject is determined not to be a candidate for treatment with alefacept, the subject is treated with another diabetes treatment that is not alefacept.

In embodiments, the present disclosure provides a biomarker assay for determining the response of a subject having Type 1 diabetes to treatment with the biologic therapeutic agent alefacept. For example, the biomarker assay can determine whether the subject is responding to treatment with alefacept before any clinical indicators show that the subject is responding to treatment.

In embodiments, the present disclosure provides a biomarker assay for determining whether a subject has Type 1 diabetes or is at risk of developing Type 1 diabetes. In embodiments, if the subject is determined to have Type 1 diabetes, the subject is treated for Type 1 diabetes. In embodiments, if the subject is determined to be at risk for Type 1 diabetes, the subject is treated for Type 1 diabetes.

Type 1 diabetes, also known as juvenile diabetes or insulin-dependent diabetes, results from destruction of pancreatic β cells by autoreactive effector T cells. Pancreatic β cells produce insulin which regulates the metabolism of carbohydrates, fats and protein by promoting the absorption of carbohydrates, especially glucose from the blood into liver, fat and skeletal muscle cells. Type 1 diabetics do not produce enough insulin and insulin administration is required for survival.

Alefacept was previously marketed under the tradename Amevive® for the treatment of psoriasis and was voluntarily discontinued by the manufacturer in 2011. Alefacept is a dimeric fusion protein consisting of the leukocyte function antigen-3 protein and Fc portion of human IgG1. It was recently discovered that alefacept was able to slow or halt the destruction of insulin-producing β cells in newly-diagnosed Type 1 diabetes patients. Preliminary studies indicated that subjects treated with alefacept had reduced insulin dependence along with a reduced number of hypoglycemic effects improved immune profiles. See, e.g., M. R. Rigby, et al, J Clin Invest. 2015 Aug. 3; 125(8):3285-96. Clinical trials regarding the treatment of Type 1 diabetes with alefacept are completed. See information on clinical trials NCT00965458 and NCT02734277 at clinicaltrials.gov. The Immune Tolerance Network Trial ID No. for these trials is ITN045AI and further information can also be found at t1dal.org. Alefacept has shown the ability to improve clinical indicators of Type 1 diabetes, including reduced frequency of insulin administration, reduced frequency of hypoglycemic or hyperglycemic events, increased blood levels of C-peptide (a protein released into the blood in equal amounts to insulin) and decreased levels of depleted CD4⁺ and CD8⁺ central memory T cells and effector memory T cells (Tem). See, M. R. Rigby, et al, J Clin Invest. 2015 Aug. 3; 125(8):3285-96 and the results of the above clinical trials.

While the detection of the above traditional clinical indicators is helpful in predicting the effectiveness of the treatment of diabetes with alefacept, detection of one or more of the biomarkers identified herein allows for another type of prediction of the effectiveness of treatment. Further, detection of the biomarkers identified herein allows for an earlier determination of the effectiveness of alefacept treatment than traditional clinical indicators. The ability to quickly and accurately determine the effectiveness of treatment is especially important with an immune regulating therapeutic such as alefacept.

In embodiments, the assays described herein detect the presence of and/or measure the concentration of biomarkers related to whether a subject having Type 1 diabetes responds to treatment with alefacept. In embodiments, the assays described herein are used for predicting the effectiveness of alefacept treatment in subjects having Type 1 diabetes and/or determination of whether a subject having Type 1 diabetes is a candidate for, e.g., expected to be responsive to, treatment with alefacept. In embodiments, the subjects are human subjects. In embodiments, the biomarkers identified herein are low-abundance and highly specific for responsiveness to treatment of Type 1 diabetes with alefacept. In embodiments, the biomarkers identified herein are present in plasma of patients being treated with alefacept or who are candidates for treatment with alefacept. In embodiments, the biomarkers identified herein allow for determination of whether a subject is responsive to treatment of Type 1 diabetes with alefacept earlier than a determination of effective treatment using traditional clinical indicators for Type 1 diabetes.

In embodiments, the assays described herein detect the presence of and/or measure the concentration of biomarkers related to whether a subject has Type 1 diabetes or is at risk of developing Type 1 diabetes.

In embodiments, the assays described herein are used in methods for screening human subjects for Type 1 diabetes biomarkers over time. In embodiments, the subjects screened are adults. In embodiments, the subjects screened are children ages 0-18. In embodiments, the subjects screened are children ages 1-13. In embodiments, the subjects screened are children ages 2-5. In embodiments, the subjects screened are children about 1, 2, 3, 4 or 5 years of age. In embodiments, the subjects screened are not known to have Type 1 diabetes and are not considered to be at risk of developing Type 1 diabetes.

In embodiments, the subject is tested every few months, for example, every 2, 3, 4, 5, 6, 7, 8, 9, 10, 11, 12, 13, 14, 15, 16, 17, 18, 19, 20, 21, 22, 23 or 24 months. In embodiments, the subject is tested every 6 months. In embodiments, the subject is tested every 12 months. In embodiments, the subject is tested every 24 months. In embodiments, the subject is tested every 3, 4, 5, 6, 7, 8, 9 or 10 years. In embodiments, if the subject shows high concentrations of one or more Type 1 diabetes biomarkers, the subject may be tested more frequently to monitor potential progression of the disease.

In embodiments, screening methods start by testing a child between the ages of about 2 and about 5 for Type 1 diabetes biomarkers. In embodiments, the child is tested every few months, for example, every 2, 3, 4, 5, 6, 7, 8, 9, 10, 11, 12, 13, 14, 15, 16, 17, 18, 19, 20, 21, 22, 23 or 24 months. In embodiments, the child is tested every 6 months. In embodiments, the child is tested every 12 months. In embodiments, the child is tested every 24 months. In embodiments, if the child shows high concentrations of one or more Type 1 diabetes biomarkers, the child may be tested more frequently to monitor potential progression of the disease. In embodiments, the child is tested as part of a routine pediatric checkup.

The invention thus provides a multiplexed assay method comprising, simultaneously detecting at least five human biomarkers in a biological sample in a multiplexed assay format, wherein the at least two biomarkers comprise (a) anti-TGM2, (b) anti-GAD65, (c) anti-ZnT8, (d) anti-insulin, and (e) anti-IA2, wherein the multiplexed assay comprises: combining, in one or more steps: the biological sample; at least a first and second binding reagent, wherein the first and second binding reagent is a binding partner of (a) anti-TGM2, (b) anti-GAD65, (c) anti-ZnT8, (d) anti-insulin, and (e) anti-IA-2, respectively; forming at least a first and second binding complex comprising the binding reagents and the biomarkers; and detecting the biomarkers in each of the binding complexes, wherein the biological sample is from a same human subject at ages selected from the group consisting of about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, and combinations thereof. As used with respect to this embodiment “about” is within 5 months of the indicated age. In embodiments, the biological sample is from a same human subject every 2, 3, 4, 5, 6, 7, 8, 9, 10, 11, 12, 13, 14, 15, 16, 17, 18, 19, 20, 21, 22, 23 or 24 months.

In embodiments, screening methods are performed on individuals older than 18, e.g., as part of annual physicals. Such individuals may be 19-100 years old.

In embodiments, the assay methods used herein can be used in early detection studies such as the Fr1da-/Fr1da-Plus-Study, described at clinicaltrials.gov/ct2/show/NCT04039945.

In embodiments, the screening methods described herein have the benefit that they allow treatment of Type 1 diabetes before life-threatening symptoms, such as ketoacidosis, begin to develop. In embodiments, the screening methods described herein have the benefit that they allow for the collection of data on the prevalence of Type 1 diabetes in certain populations or geographical areas.

In embodiments, the disclosure provides a method comprising detecting at least two biomarkers in a biological sample, wherein the at least two biomarkers are any combination of anti-IA2 IgG, anti-beta2glycoprotein IgG, anti-DGP IgG, anti-IA2 IgM, anti-MPO IgA, anti-proinsulin IgG, anti-MPO IgM and anti-ZnT8 IgM. In embodiments, the disclosure provides a method comprising quantifying the amounts of at least two biomarkers in a biological sample, wherein the at least two biomarkers are any combination of anti-IA2 IgG, anti-beta2glycoprotein IgG, anti-DGP IgG, anti-IA2 IgM, anti-MPO IgA, anti-proinsulin IgG, anti-MPO IgM and anti-ZnT8 IgM wherein the quantifying comprises measuring the concentration of each of the at least two biomarkers in an assay. In embodiments, the disclosure provides a method comprising detecting at least two biomarkers in a biological sample, wherein the at least two biomarkers are any combination of anti-IA2 IgG, anti-beta2glycoprotein IgG, anti-DGP IgG, anti-IA2 IgM, and anti-MPO IgA. In embodiments, the disclosure provides a method comprising quantifying the amounts of at least two biomarkers in a biological sample, wherein the at least two biomarkers are any combination of anti-IA2 IgG, anti-beta2glycoprotein IgG, anti-DGP IgG, anti-IA2 IgM, and anti-MPO IgA, wherein the quantifying comprises measuring the concentration of each of the at least two biomarkers in an assay. In embodiments, the disclosure provides a method comprising detecting two biomarkers in a biological sample, wherein the at least two biomarkers are anti-IA2 IgG and anti-beta2glycoprotein IgG. In embodiments, the disclosure provides a method comprising quantifying the amounts of two biomarkers in a biological sample, wherein the at least two biomarkers are anti-IA2 IgG and anti-beta2glycoprotein IgG, wherein the quantifying comprises measuring the concentration of each of the at least two biomarkers in an assay.

In embodiments, the disclosure provides a method comprising quantifying the amounts of at least two biomarkers disclosed herein and determining if that biomarker is present in the sample in a higher or lower concentration compared to a control sample. In embodiments, the control sample is from a human subject that is not a candidate for treatment with alefacept or from a human subject that does not have Type 1 diabetes.

### V. SYSTEMIC LUPUS ERYTHEMATOSUS

In embodiments, the disclosure provides a biomarker assay for determining whether a subject has systemic lupus erythematosus (SLE). For example, a subject having a biomarker profile as identified herein can be identified as having SLE, or as having an increased risk of developing SLE. In embodiments, a subject identified as having SLE using the non-invasive methods herein can then be further tested using other methods of diagnosing SLE, including clinical methods and use of the Systemic Lupus Erythematosus Disease Activity Index (SLEDAI) described below. In embodiments, if the subject is determined to have SLE, the subject is treated for SLE. In embodiments, if the subject is determined to be at risk for SLE, the subject is treated for SLE.

In embodiments, the disclosure provides a biomarker assay for determining whether a subject will experience a SLE flare. In embodiments, an “SLE flare” as used herein refers to an increase, e.g., flare up, in SLE symptoms including fever, malaise, joint pains, muscle pains, and fatigue. In embodiments, the disclosure provides a biomarker assay for determining whether a subject is at risk for a SLE flare. For example, in embodiments a subject having a biomarker profile as identified herein is identified as a subject who will experience an SLE flare in the near future, or is a subject at risk of experiencing a SLE flare in the near future. In embodiments, a subject identified as a subject who will experience a SLE flare or who is at risk of experiencing a SLE flare can be pre-medicated using appropriate treatment, including administering corticosteroids and/or antimalarial drugs, in order to prevent or reduce the symptoms of the flare.

In embodiments, the assays described herein are used in methods for screening human subjects for SLE biomarkers over time. In embodiments, the subjects screened are adults. In embodiments, the subjects screened are children ages 0-18. In embodiments, the subjects screened are children ages 1-13. In embodiments, the subjects screened are children ages 2-5. In embodiments, the subjects screened are children about 1, 2, 3, 4 or 5 years of age. In embodiments, the subjects screened are not known to have SLE and are not considered to be at risk of developing SLE.

In embodiments, the subject is tested every few months, for example, every 2, 3, 4, 5, 6, 7, 8, 9, 10, 11, 12, 13, 14, 15, 16, 17, 18, 19, 20, 21, 22, 23 or 24 months. In embodiments, the subject is tested every 6 months. In embodiments, the subject is tested every 12 months. In embodiments, the subject is tested every 24 months. In embodiments, the subject is tested every 3, 4, 5, 6, 7, 8, 9 or 10 years. In embodiments, if the subject shows high concentrations of one or more SLE biomarkers, the subject may be tested more frequently to monitor potential progression of the disease.

In embodiments, screening methods start by testing a child between the ages of about 2 and about 5 for SLE biomarkers. In embodiments, the child is tested every few months, for example, every 2, 3, 4, 5, 6, 7, 8, 9, 10, 11, 12, 13, 14, 15, 16, 17, 18, 19, 20, 21, 22, 23 or 24 months. In embodiments, the child is tested every 6 months. In embodiments, the child is tested every 12 months. In embodiments, the child is tested every 24 months. In embodiments, if the child shows high concentrations of one or more SLE biomarkers, the child may be tested more frequently to monitor potential progression of the disease. In embodiments, the child is tested as part of a routine pediatric checkup.

In embodiments, screening methods are performed on individuals older than 18, e.g., as part of annual physicals. Such individuals may be 19-100 years old.

In embodiments, the disclosure provides a biomarker assay that predicts an increased Systemic Lupus Erythematosus Disease Activity Index (SLEDAI) score for a subject. SLEDAI is a diagnostic test given to SLE patients that uses a weighted score based on symptoms as shown in Table 1 below. The SLEDAI test is described in detail at www.lupusil.org/what-are-the-sledai-and-bilag-evaluations.html, the contents of which are hereby incorporated by reference herein.

TABLE 1

| Weight | SCORE | Descriptor | Definition |
|---|---|---|---|
| 8 | □ | Seizure | Recent onset, exclude metabolic, infectious or drug |
| | | | causes. |
| 8 | □ | Psychosis | Altered ability to function in normal activity due to |
| | | | severe disturbance in the perception of reality. |
| | | | Include hallucination, incoherence, marked loose |
| | | | associations, impoverished thought content, marked |
| | | | illogical thinking, bizarre, disorganized, or catatonic |
| | | | behavior. Exclude uremia and drug causes. |
| 8 | □ | Organic brain | Altered mental function with impaired orientation, |
| | | syndrome | memory or other intellectual function, with rapid |
| | | | onset and fluctuating clinical features, inability to |
| | | | sustain attention to environment, plus at least 2 of |
| | | | the following: perceptual disturbance, incoherent |
| | | | speech, insomnia or daytime drowsiness, or |
| | | | increased or decreased psychomotor activity. |
| | | | Exclude metabolic, infectious or drug causes. |
| 8 | □ | Visual disturbance | Retinal changes of SLE. Include cytoid bodies, |
| | | | retinal hemorrhages, serous exudate or hemorrhages |
| | | | in the choroid, or optic neuritis. Exclude |
| | | | hypertension, infection, or drug causes. |
| 8 | □ | Cranial nerve | New onset of sensory or motor neuropathy |
| | | disorder | involving cranial nerves. |
| 8 | □ | Lupus headache | Severe, persistent headache; may be migrainous, but |
| | | | must be nonresponsive to narcotic analgesia. |
| 8 | □ | CVA | New onset of cerebrovascular accident(s). Exclude |
| | | | arteriosclerosis. |
| 8 | □ | Vasculitis | Ulceration, gangrene, tender finger nodules, |
| | | | periungual infarction, splinter hemorrhages, or |
| | | | biopsy or angiogram proof vasculitis. |
| 4 | □ | Arthritis | ≥2 joints with pain and signs of inflammation (i.e., |
| | | | tenderness, swelling or effusion). |
| 4 | □ | Myositis | Proximal muscle aching/weakness, associated with |
| | | | elevated creatine phosphokinase/aldolase or |
| | | | electromyogram changes or a biopsy showing |
| | | | myositis. |
| 4 | □ | Urinary casts | Heme-granular or red blood cell casts. |
| 4 | □ | Hematuria | >5 red blood cells/high power field. Exclude stone, |
| | | | infection or other cause. |
| 4 | □ | Proteinuria | >0.5 gram/24 hours. |
| 4 | □ | Pyuria | >5 white blood cells/high power field. Exclude |
| | | | infection. |
| 2 | □ | Rash | Inflammatory type rash |
| 2 | □ | Alopecia | Abnormal, patchy or diffuse loss of hair. |
| 2 | □ | Mucosal ulcers | Oral or nasal ulcerations. |
| 2 | □ | Pleurisy | Pleuritic chest pain with pleural rub or effusion, or |
| | | | pleural thickening. |
| 2 | □ | Pericarditis | Pericardial pain with at least 1 of the following: rub, |
| | | | effusion, or electrocardiogram or echocardiogram |
| | | | confirmation. |
| 2 | □ | Low complement | Decrease in CH50, C3, or C4 below the lower limit |
| | | | of normal for testing laboratory |
| 2 | □ | Increased DNA | Increased DNA binding by Farr assay above normal |
| | | binding | range for testing laboratory. |
| 1 | □ | Fever | >38° C. Exclude infectious cause. |
| 1 | □ | Thrombocytopenia | <100,000 platelets/×10⁹/L, exclude drug causes. |

SLEDAI scores range from 0 to 105, with a higher score indicating worse symptoms. Scores greater than 6 are associated with active disease requiring therapy. Scores greater than 20 are rare.

In embodiments, the disclosure provides a biomarker assay that predicts an increased SLEDAI score. In embodiments, the disclosure provides a biomarker assay that determines if a subject will have a SLEDAI score of greater than 6. In embodiments, the disclosure provides a biomarker assay that can determine if a subject will have a SLEDAI score of greater than 20.

In embodiments, subjects are classified as having SLE if the subject has a SLEDAI score of greater than 6, 7, 8, 9, 10, 11, 12, 13, 14, 15, 16, 17, 18, 19 or 20.

In embodiments, the disclosure provides a biomarker assay for determining whether a subject has SLE comprising detecting the presence of at least two biomarkers using a bridging assay as described herein. In embodiments, the disclosure provides a biomarker assay for determining whether a subject has SLE comprising quantifying the amounts of at least two biomarkers using a bridging assay as described herein. In embodiments, the bridging assay is a regular bridging assay. In embodiments, the disclosure is a stepwise bridging assay (sequential bridging assay).

In embodiments, the disclosure provides a biomarker assay for determining whether a subject will experience an SLE flare comprising detecting the presence of at least two biomarkers using a bridging assay as described herein. In embodiments, the disclosure provides a biomarker assay for determining whether a subject will experience an SLE flare comprising quantifying the amounts of at least two biomarkers using a bridging assay as described herein. In embodiments, the bridging assay is a regular bridging assay. In embodiments, the bridging assay is a stepwise bridging assay (sequential bridging assay). In embodiments, the assay is a classical serology assay.

In embodiments, the disclosure provides a method for determining whether a subject has SLE comprising detecting at least two biomarkers in a biological sample, wherein the at least two biomarkers are any combination of anti-Smith IgG, anti-RoSSA60 IgG, anti-U1 RNPA IgG, anti-insulin IgM, anti-Ro SSA52 IgG, anti-GAD65 IgM, anti-ZnT8 IgG, anti-RoSSA60 IgA and anti-Ro SSA52 IgM. In embodiments, the disclosure provides a method for determining whether a subject has SLE comprising quantifying the amounts of at least two biomarkers in a biological sample, wherein the at least two biomarkers are any combination of anti-Smith IgG, anti-RoSSA60 IgG, anti-U1 RNPA IgG, anti-insulin IgM, anti-Ro SSA52 IgG, anti-GAD65 IgM, anti-ZnT8 IgG, anti-RoSSA60 IgA, and anti-Ro SSA52 IgM, wherein the quantifying comprises measuring the concentration of each of the at least two biomarkers in an assay. In embodiments, the disclosure provides a method comprising detecting or quantifying at least three, at least four at least five, at least six, at least seven, at least eight, at least nine or at least ten of the above biomarkers. In embodiments, the disclosure provides a method for determining whether a subject has SLE comprising detecting at least two biomarkers in a biological sample, wherein the at least two biomarkers are any combination of anti-Smith IgG, anti-RoSSA60 IgG and anti-U1 RNPA IgG. In embodiments, the disclosure provides a method for determining whether a subject has SLE comprising quantifying the amounts of at least two biomarkers in a biological sample, wherein the at least two biomarkers are any combination of anti-Smith IgG, anti-RoSSA60 IgG and anti-U1 RNPA IgG.

In embodiments, the disclosure provides a method for determining whether a subject will experience SLE flare comprising detecting at least two biomarkers in a biological sample, wherein the at least two biomarkers are any combination of anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA. In embodiments, the disclosure provides a method for determining whether a subject will experience SLE flare comprising quantifying the amounts of at least two biomarkers in a biological sample, wherein the at least two biomarkers are any combination of anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA, wherein the quantifying comprises measuring the concentration of each of the at least two biomarkers in an assay. In embodiments, the disclosure provides a method comprising detecting or quantifying at least three, at least four at least five, at least six, at least seven, at least eight, at least nine or at least ten of the above biomarkers. In embodiments, the disclosure provides a method for determining whether a subject will experience SLE flare comprising detecting at least two biomarkers in a biological sample, wherein the at least two biomarkers are anti-insulin IgM and anti-MPO IgA. In embodiments, the disclosure provides a method for determining whether a subject will experience SLE flare comprising quantifying the amounts of at least two biomarkers in a biological sample, wherein the at least two biomarkers are anti-insulin IgM and anti-MPO IgA.

In embodiments, the disclosure provides a method comprising quantifying the amounts of at least two biomarkers disclosed herein and determining if that biomarker is present in the sample in a higher or lower concentration compared to a control sample. In embodiments, the control sample is from a human subject that does not have SLE. In embodiments, the control sample is from a human subject that has not been diagnosed with SLE. In embodiments, the control sample is from a human subject that has SLE and is not experiencing SLE flare. In embodiments, the control sample is from a human subject that has just recovered from SLE flare.

### VI. CELIAC DISEASE

In embodiments, the disclosure provides a biomarker assay for determining whether a subject has celiac disease. For example, a subject having a biomarker profile as identified herein can be identified as having celiac disease, or having an increased risk of developing celiac disease. In embodiments, a subject identified as having celiac disease using the non-invasive methods herein can then be further tested using other methods of diagnosing celiac disease, including clinical methods such as villi biopsy. In embodiments, if the subject is determined to have celiac disease, the subject is treated for celiac disease. In embodiments, if the subject is determined to be at risk for celiac disease, the subject is treated for celiac disease. In embodiments, if the subject is determined to have celiac disease, the subject is put on a gluten free diet. In embodiments, if the subject is determined to be at risk for celiac disease, the subject is put on a gluten free diet.

In embodiments, the assays described herein are used in methods for screening human subjects for celiac disease biomarkers over time. In embodiments, the subjects screened are adults. In embodiments, the subjects screened are children ages 0-18. In embodiments, the subjects screened are children ages 1-13. In embodiments, the subjects screened are children ages 2-5. In embodiments, the subjects screened are children about 1, 2, 3, 4 or 5 years of age. In embodiments, the subjects screened are not known to have celiac disease and are not considered to be at risk of developing celiac disease.

In embodiments, the subject is tested every few months, for example, every 2, 3, 4, 5, 6, 7, 8, 9, 10, 11, 12, 13, 14, 15, 16, 17, 18, 19, 20, 21, 22, 23 or 24 months. In embodiments, the subject is tested every 6 months. In embodiments, the subject is tested every 12 months. In embodiments, the subject is tested every 24 months. In embodiments, the subject is tested every 3, 4, 5, 6, 7, 8, 9 or 10 years. In embodiments, if the subject shows high concentrations of one or more celiac disease biomarkers, the subject may be tested more frequently to monitor potential progression of the disease.

In embodiments, screening methods start by testing a child between the ages of about 2 and about 5 for celiac disease biomarkers. In embodiments, the child is tested every few months, for example, every 2, 3, 4, 5, 6, 7, 8, 9, 10, 11, 12, 13, 14, 15, 16, 17, 18, 19, 20, 21, 22, 23 or 24 months. In embodiments, the child is tested every 6 months. In embodiments, the child is tested every 12 months. In embodiments, the child is tested every 24 months. In embodiments, if the child shows high concentrations of one or more celiac disease biomarkers, the child may be tested more frequently to monitor potential progression of the disease. In embodiments, the child is tested as part of a routine pediatric checkup.

In embodiments, screening methods are performed on individuals older than 18, e.g., as part of annual physicals. Such individuals may be 19-100 years old.

In embodiments, the disclosure provides a method for determining whether a subject has celiac disease comprising detecting at least two biomarkers in a biological sample, wherein the at least two biomarkers are any combination of anti-DGP IgA, anti-DGP IgG, anti-TGM2 IgA, anti-TGM2 IgG, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, anti-RoSSA60 IgG, anti-beta2glycoprotein IgA, anti-IA2 IgG, anti-MPO IgA, anti-MPO IgM, anti-RNP68/70 IgM, anti-RoSSA52 IgG, anti-RoSSA52 IgM and anti-RoSSA60 IgM. In embodiments, the disclosure provides a method for determining whether a subject has celiac disease comprising quantifying the amounts of at least two biomarkers in a biological sample, wherein the at least two biomarkers are any combination of anti-DGP IgA, anti-DGP IgG, anti-TGM2 IgA, anti-TGM2 IgG, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, anti-RoSSA60 IgG, anti-beta2glycoprotein IgA, anti-IA2 IgG, anti-MPO IgA, anti-MPO IgM, anti-RNP68/70 IgM, anti-RoSSA52 IgG, anti-RoSSA52 IgM and anti-RoSSA60 IgM, wherein the quantifying comprises measuring the concentration of each of the at least two biomarkers in an assay. In embodiments, the disclosure provides a method comprising detecting or quantifying at least three, at least four at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty one, at least twenty two, at least twenty three, at least twenty four, at least twenty five or twenty six of the above biomarkers. In embodiments, the disclosure provides a method for determining whether a subject has celiac disease comprising detecting at least two biomarkers in a biological sample, wherein the at least two biomarkers are any combination of anti-DGP IgA, anti-DGP IgG, anti-DGP IgM, anti-TGM2 IgA, anti-TGM2 IgG and anti-TGM2 IgM. In embodiments, the disclosure provides a method for determining whether a subject has celiac disease comprising quantifying the amounts of at least two biomarkers in a biological sample, wherein the at least two biomarkers are any combination of anti-DGP IgA, anti-DGP IgG, anti-DGP IgM, anti-TGM2 IgA, anti-TGM2 IgG and anti-TGM2 IgM, wherein the quantifying comprises measuring the concentration of each of the at least two biomarkers in an assay.

In embodiments, the disclosure provides a method for determining whether a subject has celiac disease comprising detecting at least two biomarkers in a biological sample, wherein the at least two biomarkers are any combination of anti-CCP IgG, anti-beta2glycoprotein IgG, anti-CENP B IgG, anti-GAD65 IgA, anti-GAD65 IgG, anti-IA2 IgM, anti-Jo1 IgA, anti-proinsulin IgA, anti-proinsulin IgM, anti-TGM2 IgA, anti-U1RNPA IgA and anti-ZnT8 IgA. In embodiments, the disclosure provides a method comprising detecting or quantifying at least three, at least four at least five, at least six, at least seven, at least eight, at least nine, at least ten, at least eleven or at least twelve or these biomarkers. In embodiments, the method for determining whether a subject has celiac disease further comprises detecting one or more additional biomarker selected from anti-beta2glycoprotein IgA, anti-IA2 IgG, anti-MPO IgA, anti-MPO IgM, anti-RNP68/70 IgM, anti-RoSSA52 IgG, anti-RoSSA52 IgM, anti-RoSSA60 IgM or a combination thereof.

In embodiments, the disclosure provides a method comprising quantifying the amounts of at least two biomarkers disclosed herein and determining if that biomarker is present in the sample in a higher or lower concentration compared to a control sample. In embodiments, the control sample is from a human subject that does not have Celiac disease. In embodiments, the control sample is from a human subject that has not been diagnosed with Celiac disease.

### VII. BIOMARKERS AND SAMPLES

As used herein, the term “biomarker” refers to a biological substance that is indicative of a normal or abnormal process, e.g., disease, infection, or environmental exposure. Biomarkers can be small molecules such as ligands, signaling molecules, or peptides, or macromolecules such as antibodies, receptors, or proteins and protein complexes. A change in the levels of a biomarker can correlate with the risk or progression of a disease or abnormality or with the susceptibility or responsiveness of the disease or abnormality to a given treatment. A biomarker can be useful in the diagnosis of disease risk or the presence of disease in an individual, or to tailor treatments for the disease in an individual (e.g., choices of drug treatment or administration regimes). In evaluating potential drug therapies, a biomarker can be used as a surrogate for a natural endpoint such as survival or irreversible morbidity. If a treatment alters a biomarker that has a direct connection to improved health, the biomarker serves as a “surrogate endpoint” for evaluating clinical benefit. Biomarkers are further described in, e.g., Mayeux, NeuroRx 1(2): 182-188 (2004); Strimbu et al., Curr Opin HIV AIDS 5(6): 463-466 (2010); and Bansal et al., Statist Med 32: 1877-1892 (2013). The term “biomarker,” when used in the context of a specific organism (e.g., human, nonhuman primate or another animal), refers to the biomarker native to that specific organism. Unless specified otherwise, the biomarkers referred to in embodiments herein encompass human biomarkers.

In embodiments of the disclosure, the biomarker is an antibody. IgA, IgG, IgM, IgE, and IgD are different subclasses (also called isotypes) of antibodies that have different immunological properties and functional locations. For example, IgA is typically found in the mucosal areas, such as the respiratory and gastrointestinal tracts, saliva, and tears and can prevent colonization by pathogens. IgG, the most abundant antibody subclass, is found in all bodily fluids and provides the majority of antibody-based immunity against pathogens. IgM is mainly found in the blood and lymph fluid and is typically the first antibody made by the body to fight a new infection. IgE is mainly associated with allergic reactions and is found in the lungs, skin, and mucous membranes. IgD mainly functions as an antigen receptor on B cells and may activate basophils and mast cells to produce antimicrobial factors. In embodiments, the multiplexed assay method is capable of quantifying the amount of each subclass of antibodies, e.g., IgG, IgA, and IgM, present in the biological sample.

In some embodiments of the disclosure, the biomarker is an antigen, e.g., a moiety that is bound by an antibody, such as a protein, peptide, nucleic acid or macromolecule.

As used herein, the term “level” in the context of a biomarker refers to the amount, concentration, or activity of a biomarker. The term “level” can also refer to the rate of change of the amount, concentration, or activity of a biomarker. A level can be represented, for example, by the amount or synthesis rate of messenger RNA (mRNA) encoded by a gene, the amount or synthesis rate of polypeptide corresponding to a given amino acid sequence encoded by a gene, or the amount or synthesis rate of a biochemical form of a biomarker accumulated in a cell, including, for example, the amount of particular post-synthetic modifications of a biomarker such as a polypeptide, nucleic acid or small molecule. “Level” can also refer to an absolute amount of a biomarker in a sample or to a relative amount of the biomarker, including amount or concentration determined under steady-state or non-steady-state conditions. “Level” can further refer to an assay signal that correlates with the amount, concentration, activity or rate of change of a biomarker. The level of a biomarker can be determined relative to a control marker in a sample.

A. Biomarkers for Type 1 Diabetes

In embodiments, the biomarker for assessing whether a subject has Type 1 diabetes or assessing the responsiveness of a subject having Type 1 diabetes to treatment with alefacept is an autoimmune biomarker, such as an antibody or other autoimmune protein. In embodiments, the method comprises quantifying a combination of the biomarkers described herein in a sample, e.g., a biological sample. In embodiments, the sample is obtained from a subject having Type 1 diabetes, at risk of developing Type 1 diabetes, or suspected of having Type 1 diabetes. In embodiments, the responsiveness of a subject having Type 1 diabetes or suspected of having Type 1 diabetes, to treatment with alefacept is assessed based on the quantitated amounts of the biomarkers in the combination. In embodiments, quantifying the biomarker combination provides a more accurate and precise diagnosis of Type 1 diabetes, or determination of responsiveness of a subject having Type 1 diabetes to treatment with alefacept, compared with quantifying each biomarker in the combination individually.

In embodiments, the biomarker for assessing whether a subject has Type 1 diabetes or assessing the responsiveness of a subject having Type 1 diabetes to treatment with alefacept is selected from one or more of the following antibodies: beta2glycoprotein.IgA, IA2 (insulinoma-2), IgM, IA2.IgG, MPO (myeloperoxidase), IgA (MPO serology), DGP (deamidated forms of gliadin peptides), IgG, TGM2.IgG, Prolnsulin.IgG (proinsulin autoantibody or proIAA, IgG), aNCA (anti-neutrophil cytoplasmic antibodies).PR3.IgG, U1RNPC.IgG, MPO.IgM (MPO bridging serology), Sm (Smith), IgG (Smith serology), Jo.1.IgA, Thyroglobulin.IgG, IA2.IgA, Insulin.IgG, RNP68.70.IgG, U1.RNPA.IgM, Jo.1.IgM, IF.IgA, RoSSA60.IgG, RNP68.70.IgA, Thyroglobulin.IgA, Insulin.IgM, ZnT8 (zinc transporter 8 protein), IgM, CCP (cyclic citrullinated peptide; also known as anti-citrullinated protein/peptide antibody (ACPA)), IgA, RoSSA60.IgM, U1.RNPA.IgA, La.SSb.IgG, CENP.B (centromere protein B), IgA, La.SSb.IgA, MPO.IgG (MPO classical serology), IF.IgM, ZnT8.IgG, Prolnsulin.IgM (proinsulin autoantibody, IgM), Thyroglobulin.IgM, Jo.1.IgG, MPO.IgG (MPO bridging serology), CCP.IgG, Sm.IgA (Smith serology), TPO (thyroid peroxidase), IgA, TGM2 (tissue transglutaminase), IgM, Sc1.70 (topoisomerase I), IgM, aNCA.PR3.IgA, RNP68.70.IgM, Ro.SSA52.IgA, DGP.IgA, U1RNPC.IgA, Smith.IgM (Smith bridging serology), La.SSb.IgM, DGP.IgM, MPO.IgM (MPO serology), ZnT8.IgA, GAD65 (glutamic acid decarboxylase), IgM, beta2glycoprotein.IgM, Insulin.IgA, TGM2.IgA, CENP.B.IgM, TPO.IgG, Sc1.70.IgG, U1RNPC.IgM, Ro.SSA52.IgG, TPO.IgM, Prolnsulin.IgA (proinsulin autoantibody, IgA), Sm.IgM (Smith serology), IF.IgG, Smith.IgA (Smith bridging serology), MPO.IgA (MPO bridging serology), U1.RNPA.IgG, GAD65.IgA, GAD65.IgG, aNCA.PR3.IgM, Smith.IgG (Smith bridging serology), beta2glycoprotein.IgG, CENP.B.IgG, RoSSA60.IgA, CCP.IgM, Sc1.70.IgA or Ro.SSA52.IgM.

In embodiments, the method comprises quantifying the amount of at least 2, at least 3, at least 4, at least 5, at least 6, at least 7 or at least 8 biomarkers described herein, in a sample, e.g., a biological sample. In embodiments, the sample is obtained from a subject having Type 1 diabetes, at risk of developing Type 1 diabetes, or suspected of having Type 1 diabetes. In embodiments, the method comprises quantifying the amount of at least two biomarkers selected from an immunoglobulin G isotype antibody to Islet Antigen 2 (anti-IA2 IgG), an immunoglobulin G isotype antibody to beta2glycoprotein (anti-beta2glycoprotein IgG), an immunoglobulin G isotype antibody to Deamidated Gliadin Peptide (anti-DGP IgG), an immunoglobulin M isotype antibody to Islet Antigen 2 (anti-IA2 IgM), an immunoglobulin A isotype antibody to Myeloperoxidase (anti-MPO IgA), an immunoglobulin G isotype proinsulin autoantibody (anti-proinsulin IgG), an immunoglobulin M isotype antibody to Myeloperoxidase (anti-MPO IgM) and an immunoglobulin M isotype zinc transporter 8 autoantibody (anti-ZnT8 IgM). In embodiments, quantifying the amount of at least two biomarkers described herein provides a more accurate and precise assessment of whether a subject has Type 1 diabetes or assessment of the responsiveness of a subject having Type 1 diabetes to treatment with alefacept, compared with quantifying a single biomarker described herein.

In embodiments, the disclosure provides a method comprising quantifying the amounts of at least two biomarkers in a biological sample, wherein the at least two biomarkers are any combination of anti-IA2 IgG, anti-beta2glycoprotein IgG, anti-DGP IgG, anti-IA2 IgM, anti-MPO IgA, anti-proinsulin IgG, anti-MPO IgM and anti-ZnT8 IgM wherein the quantifying comprises measuring the concentration of each of the at least two biomarkers in an assay. In embodiments, the disclosure provides a method comprising quantifying the amounts of at least three biomarkers in a biological sample, wherein the at least three biomarkers are any combination of anti-IA2 IgG, anti-beta2glycoprotein IgG, anti-DGP IgG, anti-IA2 IgM, anti-MPO IgA, anti-proinsulin IgG, anti-MPO IgM and anti-ZnT8 IgM wherein the quantifying comprises measuring the concentration of each of the at least three biomarkers in an assay. In embodiments, the disclosure provides a method comprising quantifying the amounts of at least four biomarkers in a biological sample, wherein the at least four biomarkers are any combination of anti-IA2 IgG, anti-beta2glycoprotein IgG, anti-DGP IgG, anti-IA2 IgM, anti-MPO IgA, anti-proinsulin IgG, anti-MPO IgM and anti-ZnT8 IgM wherein the quantifying comprises measuring the concentration of each of the at least four biomarkers in an assay. In embodiments, the disclosure provides a method comprising quantifying the amounts of at least five biomarkers in a biological sample, wherein the at least five biomarkers are any combination of anti-IA2 IgG, anti-beta2glycoprotein IgG, anti-DGP IgG, anti-IA2 IgM, anti-MPO IgA, anti-proinsulin IgG, anti-MPO IgM and anti-ZnT8 IgM wherein the quantifying comprises measuring the concentration of each of the at least five biomarkers in an assay. In embodiments, quantifying the amount of at least five biomarkers described herein provides a more accurate and precise assessment of whether a subject has Type 1 diabetes or assessment of the responsiveness of a subject having Type 1 diabetes to treatment with alefacept, compared with quantifying less than 5 of the biomarkers described herein.

In embodiments, the disclosure provides a method comprising quantifying the amounts of at least two biomarkers in a biological sample, wherein the at least two biomarkers are any combination of anti-IA2 IgG, anti-beta2glycoprotein IgG, anti-DGP IgG, anti-IA2 IgM, and anti-MPO IgA, wherein the quantifying comprises measuring the concentration of each of the at least two biomarkers in an assay. In embodiments, the disclosure provides a method comprising quantifying the amounts of at least three biomarkers in a biological sample, wherein the at least three biomarkers are any combination of anti-IA2 IgG, anti-beta2glycoprotein IgG, anti-DGP IgG, anti-IA2 IgM, and anti-MPO IgA, wherein the quantifying comprises measuring the concentration of each of the at least three biomarkers in an assay. In embodiments, the disclosure provides a method comprising quantifying the amounts of at least three biomarkers in a biological sample, wherein the at least three biomarkers are any combination of anti-IA2 IgG, anti-beta2glycoprotein IgG, anti-DGP IgG, anti-IA2 IgM, and anti-MPO IgA, wherein the quantifying comprises measuring the concentration of each of the at least three biomarkers in an assay. In embodiments, the disclosure provides a method comprising quantifying the amounts of at least four biomarkers in a biological sample, wherein the at least four biomarkers are any combination of anti-IA2 IgG, anti-beta2glycoprotein IgG, anti-DGP IgG, anti-IA2 IgM, and anti-MPO IgA, wherein the quantifying comprises measuring the concentration of each of the at least four biomarkers in an assay. In embodiments, the disclosure provides a method comprising quantifying the amounts of five biomarkers in a biological sample, wherein the five biomarkers are a combination of anti-IA2 IgG, anti-beta2glycoprotein IgG, anti-DGP IgG, anti-IA2 IgM, and anti-MPO IgA, wherein the quantifying comprises measuring the concentration of each of the five biomarkers in an assay.

In embodiments, the disclosure provides a method comprising quantifying the amounts of two biomarkers in a biological sample, wherein the at least two biomarkers are anti-IA2 IgG and anti-beta2glycoprotein IgG, wherein the quantifying comprises measuring the concentration of each of the at least two biomarkers in an assay.

In embodiments, the biomarker combination comprises an immunoglobulin G isotype antibody to Islet Antigen 2 (anti-IA2 IgG). In embodiments, anti-IA2 IgG levels are higher in a subject that is responsive to treatment of Type 1 diabetes with alefacept compared with a subject who is not responsive to treatment of Type 1 diabetes with alefacept or who does not have Type 1 diabetes. In embodiments, anti-IA2 IgG levels in a subject that is responsive to treatment of Type 1 diabetes with alefacept are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% higher compared with a subject not responsive to treatment of Type 1 diabetes with alefacept or who does not have Type 1 diabetes.

In embodiments, the biomarker combination comprises an immunoglobulin G isotype antibody to beta2glycoprotein (anti-beta2glycoprotein IgG). In embodiments, anti-beta2glycoprotein IgG levels are higher in a subject that is responsive to treatment of Type 1 diabetes with alefacept compared with a subject who is not responsive to treatment of Type 1 diabetes with alefacept or who does not have Type 1 diabetes. In embodiments, anti-beta2glycoprotein IgG levels in a subject that is responsive to treatment of Type 1 diabetes with alefacept are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% higher compared with a subject not responsive to treatment of Type 1 diabetes with alefacept or who does not have Type 1 diabetes.

In embodiments, the biomarker combination comprises an immunoglobulin G isotype antibody to Deamidated Gliadin Peptide (anti-DGP IgG). In embodiments, anti-DGP IgG levels are higher in a subject that is responsive to treatment of Type 1 diabetes with alefacept compared with a subject who is not responsive to treatment of Type 1 diabetes with alefacept or who does not have Type 1 diabetes. In embodiments, anti-DGP IgG levels in a subject that is responsive to treatment of Type 1 diabetes with alefacept are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% higher compared with a subject not responsive to treatment of Type 1 diabetes with alefacept or who does not have Type 1 diabetes.

In embodiments, the biomarker combination comprises an immunoglobulin M isotype antibody to Islet Antigen 2 (anti-IA2 IgM). In embodiments, anti-IA2 IgM levels are higher in a subject that is responsive to treatment of Type 1 diabetes with alefacept compared with a subject who is not responsive to treatment of Type 1 diabetes with alefacept or who does not have Type 1 diabetes. In embodiments, anti-IA2 IgM levels in a subject that is responsive to treatment of Type 1 diabetes with alefacept are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% higher compared with a subject not responsive to treatment of Type 1 diabetes with alefacept or who does not have Type 1 diabetes.

In embodiments, the biomarker combination comprises an immunoglobulin A isotype antibody to Myeloperoxidase (anti-MPO IgA). In embodiments, anti-MPO IgA levels are higher in a subject that is responsive to treatment of Type 1 diabetes with alefacept compared with a subject who is not responsive to treatment of Type 1 diabetes with alefacept or who does not have Type 1 diabetes. In embodiments, anti-MPO IgA levels in a subject that is responsive to treatment of Type 1 diabetes with alefacept are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% higher compared with a subject not responsive to treatment of Type 1 diabetes with alefacept or who does not have Type 1 diabetes.

In embodiments, the biomarker combination comprises an immunoglobulin G isotype proinsulin autoantibody (anti-proinsulin IgG). In embodiments, anti-proinsulin IgG levels are lower in a subject that is responsive to treatment of Type 1 diabetes with alefacept compared with a subject who is not responsive to treatment of Type 1 diabetes with alefacept. In embodiments, anti-proinsulin IgG levels in a subject that is responsive to treatment of Type 1 diabetes with alefacept are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% lower compared with a subject not responsive to treatment of Type 1 diabetes with alefacept.

In embodiments, the biomarker combination an immunoglobulin M isotype antibody to Myeloperoxidase (anti-MPO IgM). In embodiments, anti-MPO IgM levels are lower in a subject that is responsive to treatment of Type 1 diabetes with alefacept compared with a subject who is not responsive to treatment of Type 1 diabetes with alefacept. In embodiments, anti-MPO IgM levels in a subject that is responsive to treatment of Type 1 diabetes with alefacept are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% lower compared with a subject not responsive to treatment of Type 1 diabetes with alefacept.

In embodiments, the biomarker combination an immunoglobulin M isotype zinc transporter 8 autoantibody (anti-ZnT8 IgM). In embodiments, anti-ZnT8 IgM levels are lower in a subject that is responsive to treatment of Type 1 diabetes with alefacept compared with a subject who is not responsive to treatment of Type 1 diabetes with alefacept. In embodiments, anti-ZnT8 IgM levels in a subject that is responsive to treatment of Type 1 diabetes with alefacept are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% lower compared with a subject not responsive to treatment of Type 1 diabetes with alefacept.

In embodiments, samples are obtained from subjects prior to treatment with alefacept or concurrently with the beginning of treatment with alefacept. In embodiments, samples are obtained from subjects at one or more timepoints following the beginning of treatment with alefacept. In embodiments, the timepoints for obtaining samples may be 1, 5, 10, 15, 20, 25, 30 or 35 days following the beginning of treatment with alefacept. In embodiments, the timepoints for obtaining samples may be less than one week or 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 weeks following the beginning of treatment with alefacept. In embodiments, the timepoints for obtaining samples may be less than one week or 1, 10, 15, 20, 25, 30, 35, 40, 45, 50 or 52 weeks following the beginning of treatment with alefacept. In embodiments, the timepoints for obtaining samples may be 11, 26, or 30 weeks following the beginning of treatment with alefacept. In other embodiments, the timepoints may be at a certain number of days, weeks, months or years following the beginning of treatment with alefacept as is known to be common in the art of biomarker analysis.

In embodiments, the disclosure provides a method of determining whether the treated group appears to have lower anti-ZnT8, anti-Sc170, and anti-LaSSB levels than in a subject with Type 1 diabetes prior to beginning treatment with alefacept (i.e. at 0 weeks). In embodiments, alefacept treatment results in decrease in anti-ZnT8 IgA, IgG, and IgM levels during the course of treatment.

In embodiments, a method comprising quantifying a combination of the biomarkers provided herein has improved sensitivity in determining the responsiveness of a subject having Type 1 diabetes to treatment with alefacept, compared with methods in which only a single biomarker is quantified. In embodiments, a method comprising quantifying a combination of the biomarkers provided herein has improved dynamic range for different stages and degrees of responsiveness of a subject having Type 1 diabetes to treatment with alefacept, compared with methods in which a single biomarker is quantified.

B. Biomarkers to SLE

In embodiments, the biomarker for determining if a subject has SLE or will experience SLE flare is an autoimmune biomarker, such as an antibody or other autoimmune protein. In embodiments, the method comprises quantifying a combination of the biomarkers described herein in a sample, e.g., a biological sample. In embodiments, the sample is obtained from a subject having SLE, at risk of developing SLE, or suspected of having SLE. In embodiments, the sample is obtained from a subject experiencing SLE flare or at risk of developing SLE flare. In embodiments, the biomarkers are predictive of a subject's SLEDAI score.

In embodiments, the biomarker for determining if a subject has SLE or will experience SLE flare is selected from one or more of the following antibodies: beta2glycoprotein.IgA, IA2.IgM, IA2.IgG, MPO.IgA (MPO serology), DGP.IgG, TGM2.IgG, ProInsulin.IgG (proinsulin autoantibody, IgG), aNCA.PR3.IgG, U1RNPC.IgG, MPO.IgM (MPO bridging serology), Sm.IgG (Smith serology), Jo.1.IgA, Thyroglobulin.IgG, IA2.IgA, Insulin.IgG, RNP68.70.IgG, U1.RNPA.IgM, Jo.1.IgM, IF.IgA, RoSSA60.IgG, RNP68.70.IgA, Thyroglobulin.IgA, Insulin.IgM, ZnT8.IgM, CCP.IgA, RoSSA60.IgM, U1.RNPA.IgA, La.SSb.IgG, CENP.B.IgA, La.SSb.IgA, MPO.IgG (MPO serology), IF.IgM, ZnT8.IgG, Prolnsulin.IgM (proinsulin autoantibody, IgM), Thyroglobulin.IgM, Jo.1.IgG, MPO.IgG (MPO bridging serology), CCP.IgG, Sm.IgA (Smith serology), TPO.IgA, TGM2.IgM, Sc1.70.IgM, aNCA.PR3.IgA, RNP68.70.IgM, Ro.SSA52.IgA, DGP.IgA, U1RNPC.IgA, Smith.IgM (Smith bridging serology), La.SSb.IgM, DGP.IgM, MPO.IgM (MPO serology), ZnT8.IgA, GAD65.IgM, beta2glycoprotein.IgM, Insulin.IgA, TGM2.IgA, CENP.B.IgM, TPO.IgG, Sc1.70.IgG, U1RNPC.IgM, Ro.SSA52.IgG, TPO.IgM, Prolnsulin.IgA (proinsulin autoantibody, IgA), Sm.IgM (Smith serology), IF.IgG, Smith.IgA (Smith bridging serology), MPO.IgA (MPO bridging serology), U1.RNPA.IgG, GAD65.IgA, GAD65.IgG, aNCA.PR3.IgM, Smith.IgG (Smith bridging serology), beta2glycoprotein.IgG, CENP.B.IgG, RoSSA60.IgA, CCP.IgM, Sc1.70.Ig, Ro.SSA52.IgM TNF-alpha, IL-15, MIP1a, IL-10, NFL, IL-8, IL-6, IL-2, IL-21 or IL-23.

In embodiments, the method of determining if a subject has SLE comprises quantifying the amount of at least 2, at least 3, at least 4, at least 5, at least 6, at least 7, at least 8, at least 9, at least 10, at least 11 or at least 12 biomarkers, in a sample, e.g., a biological sample. In embodiments, the sample is obtained from a subject having SLE, at risk of developing SLE, or suspected of having SLE. In embodiments, the method comprises quantifying the amount of at least two biomarkers selected from immunoglobulin G isotype antibody to Smith antigen (anti-Smith IgG), immunoglobulin G isotype antibody to Sjögren's-syndrome-related antigen A/Ro 60 (anti-RoSSA60 IgG), immunoglobulin G isotype antibody to U1 ribonucleoprotein A (anti-U1 RNPA IgG), immunoglobulin M isotype antibody to insulin (anti-insulin IgM), immunoglobulin M isotype antibody to proinsulin (anti-proinsulin IgM), immunoglobulin G isotype antibody to Sjögren's-syndrome-related antigen A/Ro 52 (anti-Ro SSA52 IgG), immunoglobulin M isotype antibody to glutamic acid decarboxylase (anti-GAD65 IgM), immunoglobulin G isotype zinc transporter 8 autoantibody (anti-ZnT8 IgG), immunoglobulin A isotype antibody to Sjögren's-syndrome-related antigen A/Ro 60 (anti-RoSSA60 IgA), immunoglobulin M isotype antibody to Sjögren's-syndrome-related antigen A/Ro 52 (anti-Ro SSA52 IgM), immunoglobulin G isotype antineutrophil cytoplasmic autoantibodies (anti-aNCA PR3 IgG), immunoglobulin A isotype antibody to Jo1 (anti-Jo1 IgA), immunoglobulin A isotype antibody to Smith antigen (anti-Smith IgA), immunoglobulin M isotype antibody to U1 ribonucleoprotein A (anti-U1 RNPA IgM), immunoglobulin A isotype antibody to Myeloperoxidase (anti-MPO IgA), immunoglobulin G isotype antibody to U1 ribonucleoprotein C (anti-U1RNPC IgG), immunoglobulin G isotype antibody to glutamic acid decarboxylase (anti-GAD65 IgG), immunoglobulin A isotype antibody to La ribonucleoprotein SSB antigen (Sjögren's syndrome antigen B) (anti-LaSSb IgA), immunoglobulin G isotype antibody to thyroid peroxidase (anti-TPO IgG), immunoglobulin G isotype antibody to Myeloperoxidase (anti-MPO IgG), immunoglobulin A isotype antibody to insulin (anti-insulin IgA), immunoglobulin A isotype antibody to proinsulin (anti-proinsulin IgA), immunoglobulin M isotype antibody to thyroid peroxidase (anti-TPO IgM), immunoglobulin M isotype zinc transporter 8 autoantibody (anti-ZnT8 IgM), immunoglobulin G isotype antibody to intrinsic factor (anti-IF IgG), immunoglobulin G isotype antibody to Smith antigen (anti-Smith IgG), tumor necrosis factor alpha antigen (TNF-alpha), interleukin 15 antigen (IL-15), macrophage inflammatory protein 1 alpha antigen (MIP1a), interleukin 10 antigen (IL-10), neurofilament-L antigen (NFL), interleukin 8 antigen (IL-8), interleukin 6 antigen (IL-6), interleukin 2 antigen (IL-2), interleukin 21 antigen (IL-21) and interleukin 23 antigen (IL-23). In embodiments, quantifying the amount of at least two biomarkers described herein provides a more accurate and precise assessment of the determination that the subject has SLE, compared with quantifying a single biomarker described herein.

In embodiments, the method of determining whether a subject has SLE comprises quantifying the amount of at least three biomarkers selected from anti-Smith IgG, anti-RoSSA60 IgG, anti-U1 RNPA IgG, anti-insulin IgM, anti-Ro SSA52 IgG, anti-GAD65 IgM, anti-ZnT8 IgG, anti-RoSSA60 IgA, and anti-Ro SSA52 IgM. In embodiments, the method comprises quantifying the amount of at least four biomarkers selected from anti-Smith IgG, anti-RoSSA60 IgG, anti-U1 RNPA IgG, anti-insulin IgM, anti-Ro SSA52 IgG, anti-GAD65 IgM, anti-ZnT8 IgG, anti-RoSSA60 IgA, and anti-Ro SSA52 IgM. In embodiments, the method comprises quantifying the amount of at least five biomarkers selected from anti-Smith IgG, anti-RoSSA60 IgG, anti-U1 RNPA IgG, anti-insulin IgM, anti-Ro SSA52 IgG, anti-GAD65 IgM, anti-ZnT8 IgG, anti-RoSSA60 IgA, and anti-Ro SSA52 IgM. In embodiments, the method comprises quantifying the amount of at least six biomarkers selected from anti-Smith IgG, anti-RoSSA60 IgG, anti-U1 RNPA IgG, anti-insulin IgM, anti-Ro SSA52 IgG, anti-GAD65 IgM, anti-ZnT8 IgG, anti-RoSSA60 IgA, and anti-Ro SSA52 IgM. In embodiments, the method comprises quantifying the amount of at least seven biomarkers selected from anti-Smith IgG, anti-RoSSA60 IgG, anti-U1 RNPA IgG, anti-insulin IgM, anti-Ro SSA52 IgG, anti-GAD65 IgM, anti-ZnT8 IgG, anti-RoSSA60 IgA, and anti-Ro SSA52 IgM. In embodiments, the method comprises quantifying the amount of at least eight biomarkers selected from anti-Smith IgG, anti-RoSSA60 IgG, anti-U1 RNPA IgG, anti-insulin IgM, anti-Ro SSA52 IgG, anti-GAD65 IgM, anti-ZnT8 IgG, anti-RoSSA60 IgA, and anti-Ro SSA52 IgM. In embodiments, the method comprises quantifying the amount of at least nine biomarkers selected from anti-Smith IgG, anti-RoSSA60 IgG, anti-U1 RNPA IgG, anti-insulin IgM, anti-Ro SSA52 IgG, anti-GAD65 IgM, anti-ZnT8 IgG, anti-RoSSA60 IgA, and anti-Ro SSA52 IgM. In embodiments, the method comprises quantifying the amount of ten biomarkers selected from anti-Smith IgG, anti-RoSSA60 IgG, anti-U1 RNPA IgG, anti-insulin IgM, anti-Ro SSA52 IgG, anti-GAD65 IgM, anti-ZnT8 IgG, anti-RoSSA60 IgA, and anti-Ro SSA52 IgM.

In embodiments, the method of determining whether a subject has SLE comprises quantifying the amount of at least one biomarker selected from anti-Smith IgG, anti-RoSSA60 IgG and anti-U1 RNPA IgG. In embodiments, the method of determining whether a subject has SLE comprises quantifying the amount of at least two biomarkers selected from anti-Smith IgG, anti-RoSSA60 IgG and anti-U1 RNPA IgG. In embodiments, the method of determining whether a subject has SLE comprises quantifying the amount of at least three biomarkers selected from anti-Smith IgG, anti-RoSSA60 IgG and anti-U1 RNPA IgG.

In embodiments, the method of determining if a subject will experience SLE flare comprises quantifying the amount of at least 2, at least 3, at least 4, at least 5, at least 6, at least 7, at least 8, at least 9, at least 10, at least 11 or at least 12 biomarkers in a sample, e.g., a biological sample. In embodiments, the sample is obtained from a subject experiencing SLE flare or at risk of experiencing SLE flare. In embodiments, the method comprises quantifying the amount of at least two biomarkers selected from anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA.

In embodiments, the method comprises quantifying the amount of at least three biomarkers selected from anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA. In embodiments, the method comprises quantifying the amount of at least four biomarkers selected from anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA. In embodiments, the method comprises quantifying the amount of at least five biomarkers selected from anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA. In embodiments, the method comprises quantifying the amount of at least six biomarkers selected from anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA. In embodiments, the method comprises quantifying the amount of at least seven biomarkers selected from anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA. In embodiments, the method comprises quantifying the amount of at least eight biomarkers selected from anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA. In embodiments, the method comprises quantifying the amount of at least nine biomarkers selected from anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA. In embodiments, the method comprises quantifying the amount of at least ten biomarkers selected from anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA. In embodiments, the method comprises quantifying the amount of at least eleven biomarkers selected from anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA. In embodiments, the method comprises quantifying the amount of twelve biomarkers selected from anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA.

In embodiments, the method of determining if a subject will experience SLE flare comprises quantifying the amount of at least one of a biomarker selected from anti-insulin IgM and anti-MPO IgA. In embodiments, the method of determining if a subject will experience SLE flare comprises quantifying the amounts of the biomarkers anti-insulin IgM and anti-MPO IgA.

In embodiments, the amount of the biomarker is increased in a subject experiencing SLE flare compared to the amount of the biomarker in a subject not experiencing SLE flare. In embodiments, the amount of the biomarker is decreased in a subject experiencing SLE flare compared to the amount of the biomarker in a subject not experiencing SLE flare.

In embodiments, a method comprising quantifying a combination of the biomarkers provided herein has improved sensitivity in determining if a subject has SLE, or predicting SLE flare, compared with methods in which only a single biomarker is quantified. In embodiments, a method comprising quantifying a combination of the biomarkers provided herein has improved dynamic range for different stages and degrees of SLE severity, or SLE flare severity, compared with methods in which a single biomarker is quantified.

C. Biomarkers to Celiac Disease

In embodiments, the biomarker for determining if a subject has Celiac disease is an autoimmune biomarker, such as an antibody or other autoimmune protein. In embodiments, the method comprises quantifying a combination of the biomarkers described herein in a sample, e.g., a biological sample. In embodiments, the sample is obtained from a subject having Celiac disease, at risk of developing Celiac disease, or suspected of having Celiac disease.

In embodiments, the biomarker for determining if a subject has Celiac disease is selected from one or more of the following antibodies: beta2glycoprotein.IgA, IA2.IgM, IA2.IgG, MPO.IgA (MPO serology), DGP.IgG, TGM2.IgG, Prolnsulin.IgG (proinsulin autoantibody, IgG), aNCA.PR3.IgG, U1RNPC.IgG, MPO.IgM (MPO bridging serology), Sm.IgG (Smith serology), Jo.1.IgA, Thyroglobulin.IgG, IA2.IgA, Insulin.IgG, RNP68.70.IgG, U1.RNPA.IgM, Jo.1.IgM, IF.IgA, RoSSA60.IgG, RNP68.70.IgA, Thyroglobulin.IgA, Insulin.IgM, ZnT8.IgM, CCP.IgA, RoSSA60.IgM, U1.RNPA.IgA, La.SSb.IgG, CENP.B.IgA, La. SSb.IgA, MPO.IgG (MPO serology), IF.IgM, ZnT8.IgG, ProInsulin.IgM (proinsulin autoantibody, IgM), Thyroglobulin.IgM, Jo.1.IgG, MPO.IgG (MPO bridging serology), CCP.IgG, Sm.IgA (Smith serology), TPO.IgA, TGM2.IgM, Sc1.70.IgM, aNCA.PR3.IgA, RNP68.70.IgM, Ro.SSA52.IgA, DGP.IgA, U1RNPC.IgA, Smith.IgM (Smith bridging serology), La.SSb.IgM, DGP.IgM, MPO.IgM (MPO serology), ZnT8.IgA, GAD65.IgM, beta2glycoprotein.IgM, Insulin.IgA, TGM2.IgA, CENP.B.IgM, TPO.IgG, Sc1.70.IgG, U1RNPC.IgM, Ro.SSA52.IgG, TPO.IgM, Prolnsulin.IgA (proinsulin autoantibody or IAA, IgA), Sm.IgM (Smith serology), IF.IgG, Smith.IgA (Smith bridging serology), MPO.IgA (MPO bridging serology), U1.RNPA.IgG, GAD65.IgA, GAD65.IgG, aNCA.PR3.IgM, Smith.IgG (Smith bridging serology), beta2glycoprotein.IgG, CENP.B.IgG, RoSSA60.IgA, CCP.IgM, Sc1.70.Ig, and Ro.SSA52.IgM.

In embodiments, the method of determining if a subject has Celiac disease comprises quantifying the amount of at least 2, at least 3, at least 4, at least 5, at least 6, at least 7, at least 8, at least 9, at least 10, at least 11 or at least 12 biomarkers described herein, in a sample, e.g., a biological sample. In embodiments, the sample is obtained from a subject having Celiac disease, at risk of developing Celiac disease, or suspected of having Celiac disease. In embodiments, the method comprises quantifying the amount of at least two biomarkers selected from immunoglobulin A isotype antibody to deamidated gliadin peptide (anti-DGP IgA), immunoglobulin G isotype antibody to deamidated gliadin peptide (anti-DGP IgG), immunoglobulin M isotype antibody to deamidated gliadin peptide (anti-DGP IgM), immunoglobulin A isotype antibody to transglutaminase 2 (anti-TGM2 IgA), immunoglobulin G isotype antibody to transglutaminase 2 (anti-TGM2 IgG), immunoglobulin M isotype antibody to transglutaminase 2 (anti-TGM2 IgM), immunoglobulin A isotype antibody to Smith antigen (anti-Smith IgA), and immunoglobulin A isotype antibody to proinsulin (anti-proinsulin IgA).

In embodiments, the method comprises quantifying the amount of at least three biomarkers selected from anti-DGP IgA, anti-DGP IgG, anti-TGM2 IgA, anti-TGM2 IgG, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, anti-RoSSA60 IgG, anti-beta2glycoprotein IgA, anti-IA2 IgG, anti-MPO IgA, anti-MPO IgM, anti-RNP68/70 IgM, anti-RoSSA52 IgG, anti-RoSSA52 IgM and anti-RoSSA60 IgM. In embodiments, the method comprises quantifying the amount of at least four biomarkers selected from anti-DGP IgA, anti-DGP IgG, anti-TGM2 IgA, anti-TGM2 IgG, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, anti-RoSSA60 IgG, anti-beta2glycoprotein IgA, anti-IA2 IgG, anti-MPO IgA, anti-MPO IgM, anti-RNP68/70 IgM, anti-RoSSA52 IgG, anti-RoSSA52 IgM and anti-RoSSA60 IgM. In embodiments, the method comprises quantifying the amount of at least five biomarkers selected from anti-DGP IgA, anti-DGP IgG, anti-TGM2 IgA, anti-TGM2 IgG, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, anti-RoSSA60 IgG, anti-beta2glycoprotein IgG, anti-IA2 IgG, anti-MPO IgA, anti-MPO IgM, anti-RNP68/70 IgM, anti-RoSSA52 IgG, anti-RoSSA52 IgM and anti-RoSSA60 IgM. In embodiments, the method comprises quantifying the amount of at least six biomarkers selected from anti-DGP IgA, anti-DGP IgG, anti-TGM2 IgA, anti-TGM2 IgG, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, anti-RoSSA60 IgG, anti-beta2glycoprotein IgA, anti-IA2 IgG, anti-MPO IgA, anti-MPO IgM, anti-RNP68/70 IgM, anti-RoSSA52 IgG, anti-RoSSA52 IgM and anti-RoSSA60 IgM.

In embodiments, the method comprises quantifying the amount of at least three biomarkers selected from anti-DGP IgA, anti-DGP IgG, anti-DGP IgM, anti-TGM2 IgA, anti-TGM2 IgG, and anti-TGM2 IgM. In embodiments, the method comprises quantifying the amount of at least four biomarkers selected from anti-DGP IgA, anti-DGP IgG, anti-DGP IgM, anti-TGM2 IgA, anti-TGM2 IgG, and anti-TGM2 IgM. In embodiments, the method comprises quantifying the amount of at least five biomarkers selected from anti-DGP IgA, anti-DGP IgG, anti-DGP IgM, anti-TGM2 IgA, anti-TGM2 IgG, and anti-TGM2 IgM. In embodiments, the method comprises quantifying the amount of six biomarkers selected from anti-DGP IgA, anti-DGP IgG, anti-DGP IgM, anti-TGM2 IgA, anti-TGM2 IgG, and anti-TGM2 IgM.

In embodiments, the method comprises quantifying the amount of at least three biomarkers selected from anti-CCP IgG, anti-beta2glycoprotein IgG, anti-CENP B IgG, anti-GAD65 IgA, anti-GAD65 IgG, anti-IA2 IgM, anti-Jo1 IgA, anti-proinsulin IgA, anti-proinsulin IgM, anti-TGM2 IgA, anti-U1RNPA IgA and anti-ZnT8 IgA. In embodiments, the method comprises quantifying at least four, at least five, at least six, at least seven, at least eight, at least nine, at least ten, at least eleven or twelve of these biomarkers. In embodiments, the method further comprises quantifying one or more additional biomarker selected from anti-beta2glycoprotein IgA, anti-IA2 IgG, anti-MPO IgA, anti-MPO IgM, anti-RNP68/70 IgM, anti-RoSSA52 IgG, anti-RoSSA52 IgM, anti-RoSSA60 IgM or a combination thereof.

In embodiments the disclosure provides a method comprising quantifying a combination of the biomarkers provided herein has improved sensitivity in determining if a subject has Celiac disease, compared with methods in which only a single biomarker is quantified. In embodiments, a method comprising quantifying a combination of the biomarkers provided herein has improved dynamic range for different stages and degrees of Celiac disease, compared with methods in which a single biomarker is quantified.

D. Samples

In embodiments, the biomarkers described herein are detected and/or measured in a sample, e.g., a biological sample. In embodiments, the sample comprises a mammalian fluid, secretion, or excretion. In embodiments, the sample is a purified mammalian fluid, secretion, or excretion. In embodiments, the mammalian fluid, secretion, or excretion is whole blood, plasma, serum, sputum, lachrymal fluid, lymphatic fluid, synovial fluid, pleural effusion, urine, sweat, cerebrospinal fluid, ascites, milk, stool, bronchial lavage, saliva, amniotic fluid, nasal secretions, vaginal secretions, a surface biopsy, sperm, semen/seminal fluid, wound secretions and excretions, or an extraction, purification therefrom, or dilution thereof. Further exemplary biological samples include but are not limited to physiological samples, samples containing suspensions of cells such as mucosal swabs, tissue aspirates, tissue homogenates, cell cultures, and cell culture supernatants. In embodiments, the biological sample is whole blood, serum, plasma, cerebrospinal fluid, urine, saliva, or an extraction or purification therefrom, or dilution thereof. In embodiments, the biological sample is serum or plasma. In embodiments, the plasma is in EDTA, heparin, or citrate.

In embodiments, the methods assay systems disclosed herein are designed so that minimal sample volume is needed. Use of minimal sample volume is advantageous as it requires less sample to be obtained from the subject to be analyzed. In embodiments, multiple measurements can be made from the sample volume when using multiplexed assays. In embodiments, the sample volumes range from about 5 μL to about 500 μL. In embodiments, the sample volumes collected are from about 10 μL to about 200 μL. In embodiments, the sample volumes collected for a bridging, simultaneous assay are about 5 μL, about 10 μL, about 15 μL, about 20 μL, about 25 μL, about 30 μL, about 35 μL, about 40 μL, about 45 μL, about 50 μL, about 60 μL about 70 μL, about 80 μL, about 90 μL, about 100 μL, about 110 μL, about 120 μL, about 125 μL, about 130 μL, about 140 μL, about 150 μL, about 160 μL, about 170 μL, about 175 μL about 180 μL, about 190 μL or about 200 μL.

In embodiments, the sample is obtained from a subject, e.g., a human. In embodiments, the sample comprises a plasma (e.g., in EDTA, heparin, or citrate) sample from a subject. In embodiments, the sample comprise a serum sample from a subject. In embodiments, the sample is obtained from a healthy subject. In embodiments, the sample is obtained from a subject who does not have Type 1 diabetes. In embodiments, the sample is obtained from a subject who does not have SLE. In embodiments, the sample is obtained from a subject who is not exhibiting SLE flare. In embodiments, the sample is obtained from a subject who does not have Celiac disease. In embodiments, the sample is obtained from a subject that has Type 1 diabetes or is suspected of having Type 1 diabetes. In embodiments, the sample is obtained from a subject that has SLE or is suspected of having SLE. In embodiments, the sample is obtained from a subject who is exhibiting SLE flare, e.g., a subject who is exhibiting one or more physiological symptoms of SLE. In embodiments, the sample is obtained from a subject that has Celiac disease or is suspected of having Celiac disease. Samples may be obtained from a single source described herein, or may contain a mixture from two or more sources, e.g., pooled from one or more subjects. The subjects may be adult subjects or pediatric subjects.

E. Methods of Treatment

1. Treatment of Type 1 Diabetes

In embodiments, the methods disclosed herein further comprise providing one or more treatments for Type 1 diabetes to the subject. In embodiments, the treatment is alefacept. In embodiments, the treatment is one or more of short-acting (regular) insulin, rapid-acting insulin, intermediate-acting (NPH) insulin and long-acting insulin. In embodiments, the treatment is one or more of aspirin, a high blood pressure medication and a cholesterol-lowering drug. In embodiments, the treatment comprises prescribing the subject a specific diet. In embodiments, the treatments above are used in combination.

2. Treatment of SLE or SLE Flare

In embodiments, the methods disclosed herein further comprise providing one or more treatments for SLE or SLE flare. In embodiments, the treatment is administration of a corticosteroid. In embodiments, the corticosteroid is prednisolone, methylprednisolone or prednisone. In embodiments, the treatment is administration of an anti-malarial agent. In embodiments, the anti-malarial agent is hydroxychloroquine or chloroquine.

3. Treatment of Celiac Disease

In embodiments, the methods disclosed herein further comprise providing one or more treatments for Celiac disease. In embodiments, the treatment is a gluten free diet.

### VIII. MEASUREMENT METHODS

In some embodiments, the methods disclosed herein comprise detecting biomarkers by measuring the level, e.g., the concentration, of the biomarker in the sample. As described herein, measuring the concentration of a biomarker may also be referred to as “quantifying” or “quantifying the level” of a biomarker. Levels of the biomarkers described herein can be measured using a number of techniques available to a person of ordinary skill in the art, e.g., direct physical measurements (e.g., mass spectrometry) or binding assays (e.g., assays, agglutination assays and immunochromatographic assays). Biomarkers identified herein can be measured by any suitable assay method, including but not limited to, ELISA, microsphere-based assay methods, lateral flow test strips, antibody based dot blots or western blots. The method can also comprise measuring a signal that results from a chemical reaction, e.g., a change in optical absorbance, a change in fluorescence, the generation of chemiluminescence or electrochemiluminescence, a change in reflectivity, refractive index or light scattering, the accumulation or release of detectable labels from the surface, the oxidation or reduction of redox species, an electrical current or potential, changes in magnetic fields, etc. Suitable detection techniques can detect binding events by measuring the participation of labeled binding reagents through the measurement of the labels via their photoluminescence (e.g., via measurement of fluorescence, time-resolved fluorescence, evanescent wave fluorescence, up-converting phosphors, multi-photon fluorescence, etc.), chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). Alternatively, detection techniques can be used that do not require the use of labels, e.g., techniques based on measuring mass (e.g., surface acoustic wave measurements), refractive index (e.g., surface plasmon resonance measurements), or the inherent luminescence of a biomarker.

Binding assays for measuring biomarker levels can use solid phase or homogenous formats. Suitable assay methods include sandwich or competitive binding assays. Examples of sandwich assays are described in U.S. Pat. Nos. 4,168,146 and 4,366,241. Examples of competitive assays include those disclosed in U.S. Pat. Nos. 4,235,601, 4,442,204, and 5,208,535.

A. Ultrasensitive Assays

In embodiments, the assay for measuring the concentration of the biomarkers is an ultrasensitive assay. Ultrasensitive assays are described, e.g., in U.S. Pat. No. 9,618,510; U.S. Publication No. 2017/0168047; and U.S. Provisional Application No. 62/812,928, filed Mar. 1, 2019.

In embodiments, the assay comprises: (a) contacting the biological sample with (i) a capture reagent that specifically binds to a first biomarker of the at least two biomarkers in a binding domain on a surface, wherein the binding domain further comprises an anchoring reagent; and (ii) a detection reagent that specifically binds to the first biomarker and linked to a nucleic acid probe, thereby forming a complex on the surface comprising the capture reagent, the first biomarker, and the detection reagent; (b) extending the probe to form an extended sequence comprising an anchoring region that binds the anchoring reagent; (c) binding the extended sequence to the anchoring reagent; and (d) measuring the amount of extended sequence bound to the binding domain, thereby quantifying the amount of the first biomarker.

In embodiments, the assay comprises: (a) contacting the biological sample with (i) a capture reagent that specifically binds to the first biomarker in a binding domain on a surface, wherein the binding domain further comprises an anchoring reagent; (ii) a first detection reagent that specifically binds to the first biomarker linked to a first nucleic acid probe; and (iii) a second detection reagent that specifically binds to the first biomarker and linked to a second nucleic acid probe, thereby forming a complex on the surface comprising the capture reagent, the first biomarker, and the first and second detection reagents; (b) using an extension process that requires the first and second probes to be in proximity, extending the second probe to form an extended sequence comprising an anchoring region that binds the anchoring reagent; (c) binding the extended sequence to the anchoring reagent; and (d) measuring the amount of extended sequence bound to the binding domain, thereby quantifying the amount of the first biomarker.

In embodiments, the capture reagent is an antibody, antigen, ligand, receptor, oligonucleotide, hapten, epitope, mimotope, or an aptamer. In embodiments, the capture reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the capture reagent comprises at least one heavy or light chain complementarity determining region (CDR) of an antibody. In embodiments, the capture reagent comprises at least two CDRs from one or more antibodies.

In embodiments comprising a detection reagent, the detection reagent is an antibody, antigen, ligand, receptor, oligonucleotide, hapten, epitope, mimotope, or an aptamer. In embodiments, the detection reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the detection reagent comprises at least one heavy or light chain complementarity determining region (CDR) of an antibody. In embodiments, the detection reagent comprises at least two CDRs from one or more antibodies.

In embodiments comprising first and second detection reagents, the first and second detection reagents are independently an antibody, antigen, ligand, receptor, oligonucleotide, hapten, epitope, mimotope, or an aptamer. In embodiments, the first and second detection reagents are each an antibody or a variant thereof, including an antigen/epitope-binding portion thereof, an antibody fragment or derivative, an antibody analogue, an engineered antibody, or a substance that binds to antigens in a similar manner to antibodies. In embodiments, the first and second detection reagents each comprises at least one heavy or light chain complementarity determining region (CDR) of an antibody. In embodiments, the first and second detection reagents each comprises at least two CDRs from one or more antibodies.

In embodiments comprising a detection reagent, the extending step comprises binding the probe to a template oligonucleotide and extending the probe by polymerase chain reaction (PCR). In embodiments, the extending step comprises binding the probe to a template oligonucleotide, forming a circular template oligonucleotide (e.g., by ligation of a linear template oligonucleotide to form a circle), and extending the circular oligonucleotide by rolling circle amplification. In embodiments, the extending step comprises PCR, ligase chain reaction (LCR), strand displacement amplification (SDA), self-sustained synthetic reaction (3 SR), or isothermal amplification, e.g., helicase-dependent amplification and rolling circle amplification (RCA).

In embodiments comprising first and second detection reagents, the extending step comprises binding the first and second probes to a template oligonucleotide and extending the probe by polymerase chain reaction (PCR). In embodiments, the extending step comprises binding the first and second probes to a template oligonucleotide, forming a circular template oligonucleotide, and extending the circular template oligonucleotide by rolling circle amplification. In embodiments, the extending step comprises PCR, ligase chain reaction (LCR), strand displacement amplification (SDA), self-sustained synthetic reaction (3 SR), or isothermal amplification, e.g., helicase-dependent amplification and rolling circle amplification (RCA).

In embodiments comprising first and second detection reagents, the extending step comprises contacting the complex comprising the capture reagent, the first biomarker, and the first and second detection reagents with a connector sequence comprising (i) an interior sequence complementary to the second probe and (ii) two end sequences complementary to non-overlapping regions of the first probe. In embodiments, the method further comprises ligating the two end sequences of the connector oligonucleotide to form a circular template oligonucleotide that is hybridized to both the first and second probes. In embodiments, the extending step comprises contacting the complex with (i) a first connector oligonucleotide comprising a first connector probe sequence complementary to a first region of the first probe and a first region on the second probe, and (ii) a second connector oligonucleotide comprising a second connector probe sequence complementary to a second non-overlapping region of the first probe and a second non-overlapping region of the second probe. In embodiments, the method further comprises ligating the first and second connector oligonucleotides to form a circular template oligonucleotide that is hybridized to both the first and second probes.

In embodiments, the anchoring reagent comprises an oligonucleotide, aptamer, aptamer ligand, antibody, antigen, ligand, receptor, hapten, epitope, or a mimotope. In embodiments, the anchoring reagent comprises an aptamer ligand, and the anchoring region comprises an aptamer. In embodiments, the anchoring reagent comprises an oligonucleotide-binding protein, and the anchoring region comprises an oligonucleotide sequence. In embodiments, the anchoring reagent and the anchoring region comprise complementary oligonucleotide sequences. In embodiments, the anchoring reagent comprises an anchoring oligonucleotide sequence. In embodiments, the extended sequence comprises an anchoring oligonucleotide complement that is complementary to the anchoring oligonucleotide sequence.

In embodiments, binding the extended sequence to the anchoring reagent comprises forming a triple helix between the anchoring reagent and the anchoring region. In embodiments, binding the extended sequence to the anchoring reagent comprises denaturing the anchoring region to expose a single stranded sequence prior to the binding step; exposing the anchoring region to helicase activity prior to the binding step; and/or exposing the anchoring region to nuclease treatment prior to the binding step, wherein the anchoring region comprises one or more hapten-modified bases and the anchoring reagent comprises one or more antibodies specific for the hapten; and/or the anchoring region comprises one or more ligand-modified bases and the anchoring reagent comprises one or more receptors specific for the ligand.

In embodiments, the extended sequence comprises a detection sequence complement, and measuring the amount of extended sequence comprises contacting the extended sequence with a labeled probe complementary to the detection sequence complement. In embodiments, the extended sequence comprises a detection sequence complement that is complementary to a detection oligonucleotide sequence and measuring the amount of extended sequence comprises contacting the extended sequence with a labeled probe comprising the detection oligonucleotide sequence. In embodiments, the extended sequence comprises a modified base, and measuring the amount of extended sequence comprises contacting the extended sequence with a detectable moiety capable of binding to the modified base. In embodiments, the modified base comprises an aptamer, aptamer ligand, antibody, antigen, ligand, receptor, hapten, epitope, or a mimotope, and the detectable moiety comprises a binding partner of the modified base and a detectable label. In embodiments, the modified base comprises streptavidin, and the detectable moiety comprises biotin and a detectable label. In embodiments, the modified base comprises avidin, and the detectable moiety comprises biotin and a detectable label. In embodiments, the modified base comprise biotin, and the detectable moiety comprises avidin and a detectable label.

In embodiments, the labeled probe is measured by a measurement of light scattering, optical absorbance, fluorescence, chemiluminescence, electrochemiluminescence, bioluminescence, phosphorescence, radioactivity, magnetic field, or combinations thereof. In embodiments, the labeled probe comprises an electrochemiluminescent (ECL) label, and measuring the extended sequence comprises measuring an ECL signal. In embodiments, the labeled probe comprises multiple ECL labels. In embodiments, the labeled probe comprises ruthenium. In embodiments, measuring the concentration of the biomarkers comprises measuring the presence and/or amount of the labeled probe by electrochemiluminescence.

In embodiments, the surface comprises a particle. In embodiments, the surface comprises a well of a multi-well plate. In embodiments, the surface comprises a plurality of distinct binding domains, and the capture reagent and anchoring reagent are located on two distinct binding domains on the surface. In embodiments, the surface comprises a plurality of distinct binding domains, and the capture reagent and the anchoring reagent are located on two distinct binding domains within the well. In embodiments, the surface comprises a plurality of distinct binding domains, and the capture reagent and the anchoring reagent are located in the same binding domain on the surface.

In embodiments, the surface comprises an electrode. In embodiments, the electrode is a carbon ink electrode. In embodiments, measuring the amount of extended sequence comprises applying a voltage waveform (e.g., a potential) to the electrode to general an ECL signal. In embodiments, the surface comprises a particle, and the method comprises collecting the particle on an electrode and applying a voltage waveform (e.g., a potential) to the electrode to generate an ECL signal.

In embodiments, the method further comprises repeating one or more of steps (a) to (d) described herein for one or more additional biomarkers of the at least two biomarkers, wherein each biomarker binds to a different capture reagent in a different binding domain on one or more surfaces, thereby quantifying the amount of the one or more additional biomarkers. In embodiments, a different detection reagent is used for each biomarker of the at least two biomarkers.

B. Multiplexed Assays

In embodiments, the binding of each biomarker to its corresponding capture reagent is performed in parallel by contacting the one or more surfaces with the biological sample comprising the at least two biomarkers, e.g., at least 3, at least 4, at least 5, at least 6, at least 7, or at least 8 biomarkers. In embodiments, each method step is performed for each biomarker in parallel.

In embodiments, a multiplexed assay is used to perform each step of the method in parallel. Such multiplexed assays can provide consistent and reliable results while reducing processing time and cost. Challenges of developing a multi-biomarker assay (such as, e.g., a multiplexed assay described in embodiments herein) include, for example, determining compatible reagents for all of the biomarkers (e.g., capture and detection reagents described herein should be highly specific and not be cross-reactive; all assays should perform well in the same diluents); determining concentration ranges of the reagents for consistent assay (e.g., comparable capture and detection efficiency for the assays described herein); having similar levels in the condition and sample type of choice such that the levels of all of the biomarkers fall within the dynamic range of the assays at the same dilution; minimizing non-specific binding between the biomarkers and binding reagents thereof or other interferents; and accurately and precisely detecting a multiplexed output.

A multiplexed assay format can include, e.g., multiplexing through the use of binding reagent arrays, multiplexing using spectral discrimination of labels, multiplexing of flow cytometric analysis of binding assays carried out on particles, e.g., using the LUMINEX® system. Suitable multiplexing methods include array based binding assays using patterned arrays of immobilized antibodies directed against the biomarkers of interest. Various approaches for conducting multiplexed assays have been described (see, e.g., US 2003/0113713; US 2003/0207290; US 2004/0022677; US 2004/0189311; US 2005/0052646; US 2005/0142033; US 2006/0069872; U.S. Pat. Nos. 6,977,722; 7,842,246; 10,189,023; and 10,201,812). One approach to multiplexing binding assays involves the use of patterned arrays of binding reagents, e.g., as described in U.S. Pat. Nos. 5,807,522 and 6,110,426; Delehanty, “Printing functional protein microarrays using piezoelectric capillaries,” Methods Mol Bio 278: 135-144 (2004); Lue et al., “Site-specific immobilization of biotinylated proteins for protein microarray analysis,” Methods Mol Biol 278: 85-100 (2004); Lovett, “Toxicogenomics: Toxicologists Brace for Genomics Revolution,” Science 289: 536-537 (2000); Berns, “Cancer: Gene expression in diagnosis,” Nature 403: 491-492 (2000); Walt, “Molecular Biology: Bead-based Fiber-Optic Arrays,” Science 287: 451-452 (2000). Another approach involves the use of binding reagents coated on beads that can be individually identified and interrogated. See, e.g., WO 99/26067, which describes the use of magnetic particles that vary in size to assay multiple analytes; particles belonging to different distinct size ranges are used to assay different analytes. The particles are designed to be distinguished and individually interrogated by flow cytometry. Vignali, “Multiplexed Particle-Based Flow Cytometric Assays,” J Immunol Meth 243: 243-255 (2000) has described a multiplex binding assay in which 64 different bead sets of microparticles are employed, each having a uniform and distinct proportion of two. A similar approach involving a set of 15 different beads of differing size and fluorescence has been disclosed as useful for simultaneous typing of multiple pneumococcal serotypes (Park et al., “A Latex Bead-Based Flow Cytometric Immunoassay Capable of Simultaneous Typing of Multiple Pneumococcal Serotypes (Multibead Assay),” Clin Diag Lab Immunol 7: 4869 (2000)). Bishop et al. have described a multiplex sandwich assay for simultaneous quantification of six human cytokines (Bishop et al., “Simultaneous Quantification of Six Human Cytokines in a Single Sample Using Microparticle-based Flow Cytometric Technology,” Clin Chem 45:1693-1694 (1999)).

A diagnostic test can be conducted in a single assay chamber, such as a single well of an assay plate or an assay chamber that is an assay chamber of a cartridge. The assay modules, e.g., assay plates or cartridges or multi-well assay plates, methods and apparatuses for conducting assay measurements suitable for the present invention, are described, e.g., in US 2004/0022677; US 2004/0189311; US 2005/0052646; and US 2005/0142033. Assay plates and plate readers are commercially available (MULTI-SPOT® and MULTI-ARRAY® plates and SECTOR® instruments, MESO SCALE DISCOVERY®, a division of Meso Scale Diagnostics, LLC, Rockville, Md.).

In embodiments, different capture reagents are used for each of the biomarkers of the at least two biomarkers. In embodiments, the biological sample is simultaneously combined with at least a first capture reagent for a first biomarker of the at least two biomarkers, and at least a second capture reagent for a second biomarker of the at least two biomarkers. In embodiments, the biological sample is sequentially combined with at least a first capture reagent for a first biomarker of the at least two biomarkers, and at least a second capture reagent for a second biomarker of the at least two biomarkers

In embodiments, the binding of each biomarker to its corresponding capture reagent is performed in parallel by contacting the one or more surfaces with a single liquid volume comprising a plurality of biomarkers. In embodiments, the plurality of biomarkers includes the at least two biomarkers described herein.

In embodiments, each of the different capture reagents are immobilized on separate binding domains on the surface. In embodiments, the at least first capture reagent and the at least second capture reagent are immobilized on associated first and second binding domains. In embodiments, each binding domain comprises a targeting agent capable of binding to a targeting agent complement, wherein the targeting agent complement is connected to a linking agent, and each capture reagent comprises a supplemental linking agent capable of binding to the linking agent. Thus, in embodiments, the capture reagent is immobilized on the binding domain by: (1) binding each capture reagent to the targeting agent complement via the supplemental linking agent and the linking agent; and (2) binding each product of step (1) to a binding domain comprising the targeting agent, wherein (i) each binding domain comprises a different targeting agent, and (ii) each targeting agent selectively binds to one of the targeting agent complements, thereby immobilizing each capture reagent to its associated binding domain.

In embodiments, an optional bridging agent, which is a binding partner of both the linking agent and the supplemental linking agent, bridges the linking agent and supplemental linking agent, such that each capture reagent bound to its respective targeting agent complement binds with its respective targeting agent in a respective binding domain, via the bridging agent, the targeting agent complement on each of the capture reagents, and the targeting agent on each of the binding domains.

In embodiments, the targeting agent and targeting agent complement are two members of a binding partner pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the targeting agent and targeting agent complement are cross-reactive moieties, e.g., thiol and maleimide or iodoacetamide; aldehyde and hydrazide; or azide and alkyne or cycloalkyne. In embodiments, the targeting agent is biotin, and the targeting agent complement is avidin or streptavidin.

In embodiments, the linking agent and supplemental linking agent are two members of a binding partner pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the linking agent and supplemental linking agent are cross-reactive moieties, e.g., thiol and maleimide or iodoacetamide; aldehyde and hydrazide; or azide and alkyne or cycloalkyne. In embodiments, the linking agent is avidin or streptavidin, and the supplemental linking agent is biotin. In embodiments, the targeting agent and targeting agent complement are complementary oligonucleotides. In embodiments, the targeting agent complement is streptavidin, the targeting agent is biotin, and the linking agent and the supplemental linking agent are complementary oligonucleotides.

In embodiments that include the optional bridging agent, the bridging agent is streptavidin or avidin, and the linking agents and the supplemental linking agents are each biotin.

In embodiments, each binding domain is an element of an array of binding elements. In embodiments, the binding domains are on a surface. In embodiments, the surface is a plate. In embodiments, the surface is a well in a multi-well plate. In embodiments, the array of binding elements is located within a well of a multi-well plate. In embodiments, the surface is a particle. In embodiments, each binding domain is positioned on one or more particles. In embodiments, the particles are in a particle array. In embodiments, the particles are coded to allow for identification of specific particles and distinguish between each binding domain.

In embodiments, a method for performing the multiplexed assays described herein includes:
1. Coupling the supplemental linking agent to the capture reagent. In embodiments, the supplemental linking agent is biotin, and the capture reagent is an antigen. Methods of biotinylating proteins, e.g., antibodies, are known to the skilled artisan. The coupling may include agitation, e.g., vortexing or shaking, and incubation, e.g., for about 10 minutes to about 2 hours, about 20 minutes to about 1 hour, or about 30 minutes. After the incubation, the coupling reaction can be stopped by adding a stop solution followed by agitation (e.g., vortex), and incubation for about 10 minutes to about 2 hours, about 20 minutes to 1 hour, or about 30 minutes. In embodiments, the stop solution comprises a reagent that inactivates one or more reagents in the coupling reaction. In embodiments, the coupling further comprises contacting the capture reagent comprising the supplemental linking agent with a linking agent connected to a targeting agent complement or with a bridging agent linked to a linking agent connected to a targeting agent complement. In embodiments, each unique capture reagent is contacted with a linking agent connected to a unique targeting agent complement. In embodiment, the targeting agent complement is an oligonucleotide.
2. Mixing capture reagents for each of the biomarkers in a solution. In embodiments, the mixture of binding reagents comprises at least a first capture reagent for a first biomarker of the at least two biomarkers and at least a second capture reagent for a second biomarker of the at least two biomarkers.
3. Coating the binding domains with the mixture of capture reagents. In embodiments, the binding domains are arranged on a surface. In embodiments, the surface is a well of a multi-well plate. In embodiments, the mixture of capture reagents is added to the well. In embodiments, each binding domain comprises a targeting agent for one of the unique targeting agent complements. In embodiments, the targeting agent is a complementary oligonucleotide of the targeting agent complement. In embodiments, the mixture of capture reagents is added to the well and incubated for about 10 minutes to about 4 hours, about 30 minutes to about 2 hours, or about 1 hour. In embodiments, the incubation is at 20° C. to about 30° C., about 22° C. to about 28° C., or about 24° C. to about 26° C. In embodiments, the incubation is performed with agitation, e.g., shaking. In embodiments, the surface comprising the binding domains, e.g., the plate, is washed after incubation to remove excess capture reagent.
4. Contacting the surface comprising the binding domains with the detection reagent(s) for each biomarker and the sample comprising the biomarkers, calibration reagent, or control reagent. In embodiments, the detection reagents are added before or after the other assay components. In embodiments, the detection reagents are added at a volume of about 10 μL to about 50 μL, about 20 μL to about 30 μL, or about 25 μL. In embodiments, the sample, calibration reagent, or control reagent is added at a volume of about 10 μL to about 50 μL, about 20 μL to about 30 μL, or about 25 μL. In embodiments, the volume of the detection reagents and sample, calibration reagent, or control reagent is such that the final assay reaction volume is about 50 μL. In embodiments, the assay reactions are incubated for about 10 minutes to about 4 hours, about 30 minutes to about 2 hours, or about 1 hour. In embodiments, the incubation is at 20° C. to about 30° C., about 22° C. to about 28° C., or about 24° C. to about 26° C. In embodiments, the incubation is performed with agitation, e.g., shaking. In embodiments, the surface comprising the binding domains, e.g., the plate, is washed after incubation to remove excess detection reagent and unbound components of the sample.
5. Adding read buffer and reading the assay immediately. In embodiments, the read buffer comprises an ECL co-reactant. In embodiments, the read buffer is 2×MSD Read Buffer T. In embodiments, the read buffer is a read buffer provided in, e.g., U.S. Provisional Application No. 62/787,892, filed on Jan. 3, 2019. In embodiments, the read buffer is added at a volume of about 50 μL to about 200 μL, about 100 μL to about 180 μL, or about 150 μL.

C. Validated Assays

In embodiments, the binding of each biomarker to its corresponding capture reagent is performed using a validated assay. In embodiments, a validated assay is an assay with a high standard of reproducibility, such as an assay with a coefficient of variation between assays of less than about 20%, less than about 15%, less than about 10%, less than about 5%, less than about 3%, less than about 2% or less than about 1%. Embodiments of validated assays include those described in US Published Patent Application No. 2018/0045720, which is hereby incorporated by reference herein.

D. Detection of Antibody Biomarkers

In embodiments, the biomarkers to be detected are antibodies. Antibody biomarkers can be detected using any type of assay described herein, including multiplexed assays. In embodiments, the assays are serology assays, e.g., assays of serum or other body fluids as described herein.

In embodiments, antibody biomarkers are detected using a bridging assay, e.g., a bridging serology assay. In a bridging assay, both the binding reagent and the detection reagent are an antigen that is bound by the antibody biomarker. As the antibody biomarkers are typically bivalent, the antibody biomarker will bind both the binding reagent antigen and the detection reagent antigen. In other embodiments of bridging assays, antibody biomarkers are detected using detection reagent antibodies. In these embodiments, the detection reagent antibody can be an anti-human antibody that binds human antibody biomarkers. In embodiments, the detection reagent antibody can be an anti-human IgG, an anti-human IgM or an anti-human IgA isotype antibody.

In embodiments, the binding reagent may be immobilized on a surface and/or conjugated to a molecule such as biotin or streptavidin. In embodiments, where the binding reagent is conjugated to biotin, the binding reagent may be immobilized on a surface coated with streptavidin, such as a streptavidin plate.

In embodiments, the detection regent is conjugated to a detectable label and/or conjugated to a molecule such as biotin or streptavidin. The detectable label may be any label described herein. In embodiments, the detectable label is SULFO-TAG™, an electrochemiluminescent label, as described in International Application Publication No. WO2003022028A2.

In embodiments, antibody biomarkers are detected using a regular bridging assay. In a regular bridging assay the antibody biomarker, binding reagent antigen and detection reagent antigen are incubated together to form a complex where the antibody biomarker bivalently binds both the binding reagent antigen and the detection reagent antigen, e.g., a bridged complex. The incubation can be performed in any appropriate container, for example, in the well of a polypropylene plate. In embodiments where the binding reagent antigen is conjugated to an anchor molecule such as biotin, the bridged complex solution can be transferred to contact a surface such as a streptavidin plate. In this embodiment, the biotin conjugated to the binding reagent antigen binds to the streptavidin plate, causing the entire bridged complex to be immobilized on the streptavidin plate.

In embodiments, antibody biomarkers are detected using a stepwise bridging assay (a sequential bridging assay). In a first step of a stepwise bridging assay, the binding reagent antigen is first immobilized on a surface. In embodiments where the binding reagent antigen is conjugated to biotin, the binding reagent antigen can be immobilized on a streptavidin plate. In a second step, after the binding reagent antigen is immobilized on the surface, a solution containing the antibody biomarker is contacted with the surface, allowing the first bivalent position on the antibody biomarker to bind the binding reagent antibody. In a third step, the detection reagent antigen is then contacted with the surface, allowing the second bivalent position on the antibody to bind the detection reagent antibody. In this stepwise method, the bridging complex is formed stepwise on the surface, rather than forming the entire bridging complex before immobilization, as is done in the regular bridging assay described above. In the stepwise bridging assay, the surface may optionally be rinsed or washed between any of the steps.

In a non-limiting example of the above bridging assay embodiments, using one of the biomarkers identified herein as an antibody biomarker, the antibody to be detected is anti-Islet Antigen 2 (IA2) IgG. In this example, IA2 is used as both the binding reagent antigen and the detection reagent antigen. The binding reagent IA2 can be conjugated to a molecule that allows the binding reagent IA2 to be immobilized on a surface, such as a biotin conjugated IA2 immobilized on a streptavidin plate. The detection reagent IA2 can be conjugated to a detectable label. Upon addition of the biomarker anti-IA2 IgG, the bivalent antibody biomarker will bind both the binding reagent IA2 and the detection reagent IA2, allowing for detection of biomarker anti-IA2 IgG.

In either of the regular bridging assay or stepwise bridging assay, a method may be used where the detectable label is not directly conjugated to the detection reagent antigen but is instead attached to the detection antigen reagent using a binding complex such as streptavidin/biotin or other binding pair. The advantage of using this method is that it is not necessary to prepare separately conjugated binding reagent antigen and detection reagent antigen. In a non-limiting example of this method, a biotin conjugated antigen is prepared. Some of this biotin conjugated antigen is then incubated with a detectable label conjugated with streptavidin. The binding of biotin to streptavidin causes the detectable label to become attached to the biotin conjugated antigen, creating a detection reagent antigen having a detectable label as follows:

Antigen-biotin-streptavidin-detectable label

In embodiments, additional free biotin is added to the antigen-detectable label reagent to fully occupy the streptavidin binding sites and prevent other biotin conjugates from binding to the antigen-detectable label reagent. An additional amount of the biotin conjugated antigen, which is not attached to a detectable label, is then used as the binding reagent antigen. Binding reagent antigen and detection reagent antigen prepared in this way may be used in any of the assay methods embodied above.

In embodiments, antibody biomarkers are detected using a serology assay. In embodiments of a serology assay, the binding reagent is an antigen that is bound by the antibody biomarker. After the antibody biomarker is captured (e.g., bound by) the binding reagent antigen, the complex is detected using a detection reagent antibody that binds the antibody biomarker. In embodiments, the detection reagent antibody can be an anti-human antibody that binds human antibody biomarkers. In embodiments, the detection reagent antibody can be an anti-human IgG, an anti-human IgM or an anti-human IgA isotype antibody.

In a non-limiting example of the above serology assay embodiments, using one of the biomarkers identified herein as an antibody biomarker, the antibody to be detected is anti-Islet Antigen 2 (IA2) IgG. In this example, IA2 is used as the binding reagent antigen. The binding reagent IA2 can be conjugated to a molecule that allows the binding reagent IA2 to be immobilized on a surface, such as a biotin conjugated IA2 immobilized on a streptavidin plate. Upon addition of the biomarker anti-IA2 IgG, the bivalent antibody biomarker will bind the binding reagent IA2. To this complex is added a detection reagent antibody that binds to the biomarker anti-IA2 IgG, for example, an anti-human IgG antibody. The detection reagent antibody is conjugated to a detectable label. When the detection reagent antibody binds to the biomarker antibody, the formation of this complex can then be observed by detection of the detectable label, allowing for detection of biomarker anti-IA2 IgG.

E. Establishment of Cut-Points

In embodiments, cut-points, also known as clinical cut-points or cut-offs, can be established for assays. In embodiments, a cut-point is a detected biomarker concentration value at or above which the biomarker is assessed as significant for the assay. In embodiments, a cut-point is a detected biomarker concentration value at or below which the biomarker is assessed as significant for the assay. For example, if a biomarker is measured to have a concentration in a sample that is the same, greater, or below the cut-point concentration established for that biomarker, then the sample is considered to be significant for that biomarker.

In embodiments, a clinical cut-point is a detected biomarker concentration at or above which is assessed as significant to be associated with a positive diagnosis. In embodiments, a clinical cut-point is a detected biomarker concentration below which is assessed as significant to be associated with a positive diagnosis.

In embodiments, a cut-point for a biomarker is established at the 90^{th} percentile. In embodiments, the cut-point for a biomarker is established at the 95^{th} percentile. In embodiments, the cut point for a biomarker is established at the 98^{th} percentile. In embodiments, the cut-point is established by determining the median concentration value of the “normal” samples and the interquartile ranges of the concentration values of the “normal” samples, and then setting the cut-point at a value equal to Median+(2.2 multiplied by Interquartile Range).

In embodiments, the cut-point concentrations are determined by analyzing a group of samples from “normal” subjects, e.g., subjects that are not known to have the disease that will be assayed for. For example, in development of an assay for Type 1 diabetes, the “normal” subjects will be those that are not known to have Type 1 diabetes and/or do not show clinical symptoms of Type 1 diabetes. In embodiments, if a “normal” sample provides an assay reading about the cut-point concentration for 2 or more biomarkers, it is removed from the cut-point determination as it is possible that the sample is not “normal” for the assay.

In embodiments, the group of “normal” samples contains about 10, 20, 30, 40, 50, 60, 70, 80, 90, 100, 110, 120, 130, 140, 150, 160, 170, 180, 190 or 200 or more samples. In embodiments, the group of “normal” samples contains more than about 50 samples. In embodiments, the group of “normal” samples contains more than about 75 samples. In embodiments, the group of “normal” samples contains more than about 100 samples. In embodiments, the group of “normal” samples contains more than about 150 samples. In embodiments, the group of “normal” samples contains more than about 200 samples.

In embodiments, the “normal” samples are assayed for the presence or absence of the biomarker for which the cut-point is to be established. In embodiments, the cut-point is then determined as the concentration at which the percentile of biomarkers fall below that concentration. In embodiments, if the cut-point is to be established at the 98^{th} percentile, then the concentration the biomarker in each “normal” sample is determined and the cut-point is established at a concentration of the biomarker which is greater than the concentration of the biomarker in 98% of the “normal” samples. In embodiments, if the cut-point is to be established at the 95^{th} percentile, then the concentration the biomarker in each “normal” sample is determined and the cut-point is established at a concentration of the biomarker which is greater than the concentration of the biomarker in 95% of the “normal” samples. In embodiments, if the cut-point is to be established at the 90^{th} percentile, then the concentration the biomarker in each “normal” sample is determined and the cut-point is established at a concentration of the biomarker which is greater than the concentration of the biomarker in 90% of the “normal” samples.

In embodiments, the assay panel comprises anti-TGM2, anti-GAD65, anti-ZnT8, anti-insulin and anti-IA2 autoantibodies, wherein the assay cut-points are from about 4.0-7.0 U/mL for anti-TGM2, from about 5.0-8.0 U/mL for anti-GAD65, from about 2.0-5.0 U/mL for anti-ZnT8, from about 0.1-2.0 U/mL for anti-insulin or from about 0.1-2.0 U/mL for anti-IA2.

In embodiments, the assay panel comprises anti-TGM2, anti-GAD65, anti-ZnT8, anti-insulin and anti-IA2 autoantibodies, wherein the assay cut-points are about 5.7 U/mL for anti-TGM2, about 6.8 U/mL for anti-GAD65, about 3.3 U/mL for anti-ZnT8, about 0.6 U/mL for anti-insulin or about 0.6 U/mL for anti-IA2.

In embodiments, the assay panel comprises anti-TGM2, anti-GAD65, anti-ZnT8, anti-insulin and anti-IA2 autoantibodies, wherein the assay cut-points are from about 7.0-10.0 U/mL for anti-TGM2, from about 10.0-15.0 U/mL for anti-GAD65, from about 5.0-9.0 U/mL for anti-ZnT8, from about 1.0-3.0 U/mL for anti-insulin or from about 1.5-3.5 U/mL for anti-IA2.

In embodiments, the assay panel comprises anti-TGM2, anti-GAD65, anti-ZnT8, anti-insulin and anti-IA2 autoantibodies, wherein the assay cut-points are about 8.8 U/mL for anti-TGM2, about 13.6 U/mL for anti-GAD65, about 7.5 U/mL for anti-ZnT8, about 1.4 U/mL for anti-insulin or about 2.2 U/mL for anti-IA2.

In embodiments, the assay panel comprises anti-TGM2, anti-GAD65, anti-ZnT8, anti-insulin and anti-IA2 autoantibodies, wherein the assay cut-points are from about 10.0-13.0 U/mL for anti-TGM2, from about 12.0-15.0 IU/mL for anti-GAD65, from about 6.0-9.0 U/mL for anti-ZnT8, from about 0.1-2.0 U/mL for anti-insulin or from about 0.5-3.0 IU/mL for anti-IA2.

In embodiments, the assay panel comprises anti-TGM2, anti-GAD65, anti-ZnT8, anti-insulin and anti-IA2 autoantibodies, wherein the assay cut-points are about 11.9 U/mL for anti-TGM2, about 13.1 IU/mL for anti-GAD65, about 7.9 U/mL for anti-ZnT8, about 0.65 U/mL for anti-insulin or about 1.2 IU/mL for anti-IA2.

F. Assay Components

The concentration of various reagents used in the assays described herein may be selected during assay optimization. In embodiments, the concentration of each capture reagent in the solution used to coat (i.e., coating solution) the binding domain is about 0.05 μg/mL to about 5 μg/mL; about 0.1 μg/mL to about 1 μg/mL; about 0.2 μg/mL to about 0.5 μg/mL; or about 0.25 to about 0.3 μg/mL. In embodiments, the concentration of each capture reagent in the solution used to coat the binding domain is about 0.1 μg/mL, about 0.11 μg/mL, about 0.12 μg/mL, about 0.13 μg/mL, about 0.14 μg/mL, about 0.15 μg/mL, about 0.16 μg/mL, about 0.17 μg/mL, about 0.18 μg/mL, about 0.19 μg/mL, about 0.2 μg/mL, about 0.21 μg/mL, about 0.22 μg/mL, about 0.23 μg/mL, about 0.24 μg/mL, about 0.25 μg/mL, about 0.26 μg/mL, about 0.27 μg/mL, about 0.28 μg/mL, about 0.29 μg/mL, about 0.3 μg/mL, about 0.31 μg/mL, about 0.32 μg/mL, about 0.33 μg/mL, about 0.34 μg/mL, about 0.35 μg/mL, about 0.36 μg/mL, about 0.37 μg/mL, about 0.38 μg/mL, about 0.39 μg/mL, about 0.4 μg/mL, about 0.5 μg/mL, about 0.6 μg/mL, about 0.7 μg/mL, about 0.8 μg/mL, about 0.9 μg/mL, or about 1 μg/mL. In embodiments, the amount of capture reagent per reaction (e.g., per well on a plate) is about 0.01 pmol to about 5 pmol, about 0.05 pmol to about 3 pmol, or about 0.1 pmol to about 1 pmol.

In embodiments, the working concentration of each detection reagent is about 0.5 μg/mL to about 20 μg/mL; about 1 μg/mL to about 10 μg/mL; or about 2 μg/mL to about 5 μg/mL. In embodiments, the working concentration of each detection reagent is about 0.5 μg/mL, about 0.6 μg/mL, about 0.7 μg/mL, about 0.8 μg/mL, about 0.9 μg/mL, about 1 μg/mL, about 1.1 μg/mL, about 1.2 μg/mL, about 1.3 μg/mL, about 1.4 μg/mL, about 1.5 μg/mL, about 1.6 μg/mL, about 1.7 μg/mL, about 1.8 μg/mL, about 1.9 μg/mL, about 2 μg/mL, about 3 μg/mL, about 4 μg/mL, about 5 μg/mL, about 6 μg/mL, about 7 μg/mL, about 8 μg/mL, about 9 μg/mL, or about 10 μg/mL. In embodiments, the amount of detection reagent per reaction (e.g., per well on a plate) is about 0.01 pmol to about 5 pmol, about 0.05 pmol to about 3 pmol, or about 0.1 pmol to about 1 pmol.

In embodiments, the assay described herein further comprises measuring the concentration of one or more calibration reagents. In embodiments, a calibration reagent comprises a known concentration of a biomarker. In embodiments, the calibration reagent comprises a mixture of known concentrations of multiple biomarkers, e.g., the at least two biomarkers. In embodiments, the assay further comprises measuring the concentration of multiple calibration reagents comprising a range of concentrations for one or more biomarkers. In embodiments, the multiple calibration reagents comprise concentrations of one or more biomarkers near the upper and lower limits of quantitation for the assay. In embodiments, the multiple concentrations of the calibration reagent spans the entire dynamic range of the assay. In embodiments, the calibration reagent is a negative control, i.e., containing no biomarkers.

In embodiments, the concentration of biomarker in the calibration reagent is about 0.01 pg/mL to about 5 μg/mL; about 0.05 pg/mL to about 4 μg/mL; about 0.1 pg/mL to about 3 μg/mL; about 0.2 pg/mL to about 1 μg/mL; about 0.3 pg/mL to about 0.5 μg/mL; about 0.4 pg/mL to about 0.1 μg/mL; about 0.5 pg/mL to about 90 ng/mL; about 0.6 pg/mL to about 80 ng/mL; about 0.7 pg/mL to about 70 ng/mL; about 0.8 pg/mL to about 60 ng/mL; about 0.9 pg/mL to about 50 ng/mL; about 1 pg/mL to about 40 ng/mL; about 2 pg/mL to about 30 ng/mL; about 3 pg/mL to about 20 ng/mL; about 4 pg/mL to about 10 ng/mL; about 5 pg/mL to about 5 ng/mL; about 6 pg/mL to about 4 ng/mL; about 7 pg/mL to about 3 ng/mL; about 8 pg/mL to about 2 ng/mL; about 9 pg/mL to about 1 ng/mL; about 10 pg/mL to about 700 pg/mL; about 20 pg/mL to about 600 pg/mL; about 30 pg/mL to about 500 pg/mL; about 40 pg/mL to about 400 pg/mL; about 50 pg/mL to about 300 pg/mL; about 60 pg/mL to about 200 pg/mL; about 70 pg/mL to about 150 pg/mL; about 80 pg/mL to about 120 pg/mL; or about 90 pg/mL to about 100 pg/mL. In embodiments, the calibrator reagent is a known biomarker from a sample obtained from a subject that exhibits high reactivity to an antigen to one or more of the biomarker antibodies identified herein and who may or may not have Type 1 diabetes. In embodiments, the calibrator reagent is a known biomarker from a sample obtained from a subject that exhibits high reactivity to an antigen to one or more of the biomarker antibodies identified herein and who may or may not have SLE. In embodiments, the calibrator reagent is a known biomarker from a sample obtained from a subject that exhibits high reactivity to an antigen to one or more of the biomarker antibodies identified herein and who may or may not be experiencing SLE flare. In embodiments, the calibrator reagent is a known biomarker from a sample obtained from a subject that exhibits high reactivity to an antigen to one or more of the biomarker antibodies identified herein and who may or may not have Celiac disease.

In embodiments, the concentration of biomarkers in the negative control reagent is about 1 pg/mL to about 1000 ng/mL, or about 10 pg/mL to about 500 ng/mL, or about 50 pg/mL to about 100 ng/mL, or about 100 pg/mL to about 50 ng/mL, or about 200 pg/mL to about 10 ng/mL, or about 500 pg/mL to about 1 ng/mL, depending on the biomarker. In embodiments, the concentration of biomarkers in the positive control reagent is about 10 pg/mL to about 1000 ng/mL, about 30 pg/mL to about 500 ng/mL, about 50 ng/mL to about 100 ng/mL, about 100 pg/mL to about 10 ng/mL, or about 500 pg/mL to about 1 ng/mL, depending on the particular biomarker. In embodiments, the negative control reagent is made from pooled sera and or plasma from a large number of normal samples, e.g., samples taken from subjects that do not have Type 1 diabetes. In embodiments, the negative control reagent is made from pooled sera and or plasma from a large number of normal samples, e.g., samples taken from subjects that do not have SLE. In embodiments, the negative control reagent is made from pooled sera and or plasma from a large number of normal samples, e.g., samples taken from subjects that are not exhibiting a SLE flare. In embodiments, the negative control reagent is made from pooled sera and or plasma from a large number of normal samples, e.g., samples taken from subjects that do not have Celiac disease.

In embodiments, the positive control reagent is made from sample obtained from a subject that exhibits high reactivity to an antigen to one or more of the biomarker antibodies identified herein and who may or may not have Type 1 diabetes. In embodiments, the positive control reagent is made from sample obtained from a subject that exhibits high reactivity to an antigen to one or more of the biomarker antibodies identified herein and who may or may not have SLE. In embodiments, the positive control reagent is made from sample obtained from a subject that exhibits high reactivity to an antigen to one or more of the biomarker antibodies identified herein and who may or may not be exhibiting a SLE flare. In embodiments, the positive control reagent is made from sample obtained from a subject that exhibits high reactivity to an antigen to one or more of the biomarker antibodies identified herein and who may or may not have Celiac disease.

In embodiments, the dynamic range of the assays described herein is about 0.01 fg/mL to about 10 μg/mL. In embodiments, the concentration of each biomarker detected in the biological sample is within a range of about 0.01 fM to about 10 μM; about 0.03 fM to about 1 μM; about 0.05 fM to about 0.1 μM, about 0.1 fM to about 10 nM, about 1 fM to about 1 nM, about 10 fM to about 0.1 nM, or about 0.1 pM to about 10 pM. In embodiments, the concentration of each biomarker detected in the biological sample is less than about 100 fM, less than about 50 fM, less than about 10 fM, less than about 5 fM, less than about 3 fM, less than about 1 fM, less than about 1 fM, less than about 0.5 fM, less than about 0.3 fM, less than about 0.1 fM, less than about 0.05 fM, less than about 0.03 fM, or less than about 0.01 fM. In embodiments, the assay is capable of simultaneously detecting biomarkers with concentrations differing by at least one order of magnitude in the sample, e.g., a biomarker present at less than 100 fM and a biomarker present at greater than 1 pM in the sample.

G. Automated and/or Ultra-High Throughput Methods

Methods disclosed herein may be performed manually, using automated technology, or both. Automated technology may be partially automated, e.g., one or more modular instruments, or a fully integrated, automated instrument. Exemplary automated systems are discussed and described in International Patent Publication Nos. WO 2018/017156, WO 2017/015636, and WO 2016/164477.

Automated systems (modules and fully integrated) on which the methods herein may be carried out may comprise the following automated subsystems: computer subsystem(s) that may comprise hardware (e.g., personal computer, laptop, hardware processor, disc, keyboard, display, printer), software (e.g., processes such as drivers, driver controllers, and data analyzers), and database(s); liquid handling subsystem(s), e.g., sample handling and reagent handling, e.g., robotic pipetting head, syringe, stirring apparatus, ultrasonic mixing apparatus, magnetic mixing apparatus; sample, reagent, and consumable storing and handling subsystem(s), e.g., robotic manipulator, tube or lid or foil piercing apparatus, lid removing apparatus, conveying apparatus such as linear and circular conveyors and robotic manipulators, tube racks, plate carriers, trough carriers, pipet tip carriers, plate shakers; centrifuges, assay reaction subsystem(s), e.g., fluid-based and consumable-based (such as tube and multi well plate); container and consumable washing subsystem(s), e.g., plate washing apparatus; magnetic separator or magnetic particle concentrator subsystem(s), e.g., flow cell, tube, and plate types; cell and particle detection, classification and separation subsystem(s), e.g., flow cytometers and Coulter counters; detection subsystem(s) such as colorimetric, nephelometric, fluorescence, and ECL detectors; temperature control subsystem(s), e.g., air handling, air cooling, air warming, fans, blowers, water baths; waste subsystem(s), e.g., liquid and solid waste containers; global unique identifier (GUI) detecting subsystem(s) e.g., 1D and 2D bar-code scanners such as flat bed and wand types; sample identifier detection subsystem(s), e.g., 1D and 2D bar-code scanners such as flat bed and wand types. Analytical subsystem(s), e.g., chromatography systems such as high-performance liquid chromatography (HPLC), fast-protein liquid chromatography (FPLC), and mass spectrometer can also be modules or fully integrated.

Systems or modules that perform sample identification and preparation may be combined with (or be adjoined to or adjacent to or robotically linked or coupled to) systems or modules that perform assays and that perform detection or that perform both. Multiple modular systems of the same kind may be combined to increase throughput. Modular system(s) may be combined with module(s) that carry out other types of analysis such as chemical, biochemical, and nucleic acid analysis.

The automated system may allow batch, continuous, random-access, and point-of-care workflows and single, medium, and high sample throughput.

The system can comprise, for example, one or more of the following devices: plate sealer (e.g., Zymark), plate washer (e.g., BioTek, TECAN), reagent dispenser and/or automated pipetting station and/or liquid handling station (e.g., TECAN, Zymark, Labsystems, Beckman, Hamilton), incubator (e.g., Zymark), plate shaker (e.g., Q. Instruments, Inheco, Thermo Fisher Scientific), compound library or sample storage and/or compound and/or sample retrieval module. One or more of these devices can be coupled to the apparatus via a robotic assembly such that the entire assay process can be performed automatically. In embodiments, containers (e.g., plates) are manually moved between the apparatus and various devices (e.g., stacks of plates).

The automated system can be configured to perform one or more of the following functions: (a) moving consumables such as plates into, within, and out of the detection subsystem, (b) moving consumables between other subsystems, (c) storing the consumables, (d) sample and reagent handling (e.g., adapted to mix reagents and/or introduce reagents into consumables), (e) consumable shaking (e.g., for mixing reagents and/or for increasing reaction rates), (f) consumable washing (e.g., washing plates and/or performing assay wash steps (e.g., well aspirating)), (g) measuring ECL in a flow cell or a consumable such as a tube or a plate. The automated system may be configured to handle individual tubes placed in racks, multi-well plates such as 96 or 384 well plates.

Methods for integrating components and modules in automated systems as described herein are further described in, e.g., Sargeant et al., “Platform Perfection,” Medical Product Outsourcing, May 17, 2010.

In embodiments, the automated system is fully automated, is modular, is computerized, performs in vitro quantitative and qualitative tests on a wide range of analytes and performs photometric assays, ion-selective electrode measurements, and/or electrochemiluminescence (ECL) assays. In embodiments, the system comprises the following hardware units: a control unit, a core unit and at least one analytical module.

In embodiments, the control unit uses a graphical user interface to control all instrument functions, and is comprised of a readout device, such as a monitor, an input device(s), such as keyboard and mouse, and a personal computer using, e.g., a Windows operating system. In embodiments, the core unit is comprised of several components that manage conveyance of samples to each assigned analytical module. The actual composition of the core unit depends on the configuration of the analytical modules, which can be configured by one of skill in the art using methods known in the art. In embodiments, the core unit comprises at least the sampling unit and one rack rotor as main components. Conveyor line(s) and a second rack rotor are possible extensions. Several other core unit components can include the sample rack loader/unloader, a port, a barcode reader (for racks and samples), a water supply and a system interface port. In embodiments, the analytical module conducts ECL assays and comprises a reagent area, a measurement area, a consumables area and a pre-clean area.

In embodiments, the disclosure further provides an automated version of the methods of the invention using an ultra high-throughput robotic liquid handling system. This system allows simultaneous preparation of up to 1,520 samples with accuracy and reproducibility unmatched by a human operator. In embodiments, the automated system is a free-standing, fully integrated system for carrying out assays using ECL technology. This system, capable of simultaneously running up to twenty 96-well assay plates, includes a robotic lab automation workstation for liquid handling and plate manipulation, physically integrated with an ECL reader. In embodiments, the workflow conducts the methods described herein, e.g., the multiplexed assays, with minimal human intervention. In embodiments, the ultra-high throughput system produces results for about 1,520 samples in about 30 minutes to about 300 minutes, or about 60 minutes to about 150 minutes, or about 70 minutes to about 130 minutes. The ultra-high throughput system described herein is capable of processing about 10,000 single samples in a day, or about 5,000 duplicate samples in a day.

### IX. KITS

A. Autoimmune Disease Assay Kits

In embodiments, the present disclosure also provides kits that are used in diagnosing autoimmune disease. In embodiments where the kits comprise autoantibodies, the autoantibodies can be of the IgG, IgA or IgM isotypes.

In embodiments, the present disclosure further provides a kit comprising, in one or more vials, containers, or compartments: (a) a surface comprising at least a first, second and third binding reagent immobilized on an associated first, second and third binding domain, wherein the first, second and third binding reagent is a binding partner of anti-Insulin, anti-proinsulin, and anti-ZnT8, respectively; (b) a detection reagent that specifically binds to anti-insulin; (c) a detection reagent that specifically binds to anti-proinsulin and (d) a detection reagent that specifically binds to anti-ZnT8.

In embodiments, the present disclosure further provides a kit comprising, in one or more vials, containers, or compartments: (a) a surface comprising at least a first and second binding reagent immobilized on an associated first and second binding domain, wherein the first and second binding reagent is a binding partner of anti-GAD65 and anti-Intrinsic Factor, respectively; (b) a detection reagent that specifically binds to anti-GAD65; and (c) a detection reagent that specifically binds to anti-Intrinsic Factor.

In embodiments, the present disclosure further provides a kit comprising, in one or more vials, containers, or compartments: (a) a surface comprising at least a first and second binding reagent immobilized on an associated first and second binding domain, wherein the first and second binding reagent is a binding partner of anti-IA2 and anti-Jo-1, respectively; (b) a detection reagent that specifically binds to anti-IA2; and (c) a detection reagent that specifically binds to anti-Jo-1.

In embodiments, the present disclosure further provides a kit comprising, in one or more vials, containers, or compartments: (a) a surface comprising at least a first, second, third, fourth and fifth binding reagent immobilized on an associated first, second, third, fourth and fifth binding domain, wherein the first, second, third, fourth and fifth binding reagent is a binding partner of anti-Smith, anti-Thyroglobulin, anti-MPO, anti-DGP, and anti-TGM2, respectively; (b) a detection reagent that specifically binds to anti-Smith; (c) a detection reagent that specifically binds to anti-Thyroglobulin, (d) a detection reagent that specifically binds to anti-MPO, (e) a detection reagent that specifically binds to anti-DGP, and (f) a detection reagent that specifically binds to anti-TGM2.

In embodiments, the present disclosure further provides a kit comprising, in one or more vials, containers, or compartments: (a) a surface comprising at least a first, second, third and fourth binding reagent immobilized on an associated first, second, third and fourth binding domain, wherein the first, second, third and fourth binding reagent is a binding partner of anti-TPO, anti-U1RNPA, anti-RoSSA52, and anti-aNCA PR3, respectively; (b) a detection reagent that specifically binds to anti-TPO; (c) a detection reagent that specifically binds to anti-U1RNPA, (d) a detection reagent that specifically binds to anti-RoSSA52, and (e) a detection reagent that specifically binds to anti-aNCA PR3.

In embodiments, the present disclosure further provides a kit comprising, in one or more vials, containers, or compartments: (a) a surface comprising at least a first, second, third, fourth, fifth, sixth, seventh and eighth binding reagent immobilized on an associated first, second, third, fourth, fifth, sixth, seventh and eighth binding domain, wherein the first, second, third, fourth, fifth, sixth, seventh and eighth binding reagent is a binding partner of anti-CENPB, anti-Sc170, anti-CCP, anti-MPO, anti-RoSSA60, anti-U1RNPC, anti-Smith, and anti-RNP68/70, respectively; (b) a detection reagent that specifically binds to anti-CENPB; (c) a detection reagent that specifically binds to anti-Sc170, (d) a detection reagent that specifically binds to anti-CCP, (e) a detection reagent that specifically binds to anti-MPO, (f) a detection reagent that specifically binds to anti-RoSSA60, (g) a detection reagent that specifically binds to anti-U1RNPC, (h) a detection reagent that specifically binds to anti-Smith, and (h) a detection reagent that specifically binds to anti-RNP68/70.

In embodiments, the present disclosure further provides a kit comprising, in one or more vials, containers, or compartments: (a) a surface comprising at least a first and second binding reagent immobilized on an associated first and second binding domain, wherein the first and second binding reagent is a binding partner of anti-LaSSB and anti-beta2-glycoprotein, respectively; (b) a detection reagent that specifically binds to anti-LaSSB; and (c) a detection reagent that specifically binds to anti-beta2-glycoprotein.

In embodiments, the present disclosure further provides a kit comprising, in one or more vials, containers, or compartments: (a) a surface comprising at least a first, second, third, fourth and fifth binding reagent immobilized on an associated first, second, third, fourth and fifth binding domain, wherein the first, second, third, fourth and fifth binding reagent is a binding partner of anti-TGM2, anti-GAD65, anti-ZnT8, anti-Insulin and anti-IA2, respectively; (b) a detection reagent that specifically binds to anti-TGM2; (c) a detection reagent that specifically binds to anti-GAD65, (d) a detection reagent that specifically binds to anti-ZnT8, (e) a detection reagent that specifically binds to anti-Insulin, and (f) a detection reagent that specifically binds to anti-IA2.

In embodiments, the present disclosure further provides a kit comprising, in one or more vials, containers, or compartments: (a) a surface comprising at least a first, second, third, fourth, fifth, sixth and seventh binding reagent immobilized on an associated first, second, third, fourth, fifth, sixth and seventh binding domain, wherein the first, second, third, fourth, fifth, sixth, and seventh binding reagent is a binding partner of anti-insulin, anti-MPO, TARC, anti-Jo-1, anti-GAD65, MIP-1a, and IL-7, respectively; (b) a detection reagent that specifically binds to anti-insulin; (c) a detection reagent that specifically binds to anti-MPO, (d) a detection reagent that specifically binds to TARC, (e) a detection reagent that specifically binds to anti-Jo-1, (f) a detection reagent that specifically binds to anti-GAD65, (g) a detection reagent that specifically binds to MIP-1a, and (h) a detection reagent that specifically binds to IL-7.

In embodiments, the present disclosure further provides a kit comprising, in one or more vials, containers, or compartments: (a) a surface comprising at least a first, second, third, fourth, fifth, sixth, seventh and eighth binding reagent immobilized on an associated first, second, third, fourth, fifth, sixth, seventh and eighth binding domain, wherein the first, second, third, fourth, fifth, sixth, seventh and eighth binding reagent is a binding partner of anti-insulin, anti-MPO, TARC, anti-Jo-1, anti-GAD65, MIP-1a, IL-7 and Eotaxin, respectively; (b) a detection reagent that specifically binds to anti-insulin; (c) a detection reagent that specifically binds to anti-MPO, (d) a detection reagent that specifically binds to TARC, (e) a detection reagent that specifically binds to anti-Jo-1, (f) a detection reagent that specifically binds to anti-GAD65, (g) a detection reagent that specifically binds to MIP-1a, and (h) a detection reagent that specifically binds to IL-7, and (i) a detection reagent that specifically binds to Eotaxin.

B. Type 1 Diabetes Kits

In embodiments, the present disclosure further provides a kit comprising, in one or more vials, containers, or compartments: (a) a surface comprising at least a first and second binding reagent immobilized on an associated first and second binding domain, wherein the first and second binding reagent is a binding partner of anti-IA2 IgG and anti-beta2glycoprotein IgG, respectively; (b) a detection reagent that specifically binds to anti-IA2 IgG; and (c) a detection reagent that specifically binds to anti-beta2glycoprotein IgG.

In embodiments, the present disclosure further provides a kit comprising, in one or more vials, containers, or compartments: (a) a surface comprising at least a first, second, third, fourth and fifth binding reagent immobilized on an associated first, second, third, fourth and fifth binding domain, wherein the first, second, third, fourth and fifth binding reagent is a binding partner of anti-IA2 IgG, anti-beta2glycoprotein IgG, anti-DGP IgG, anti-IA2 IgM and anti-MPO IgA, respectively; (b) a detection reagent that specifically binds to anti-IA2 IgG; (c) a detection reagent that specifically binds to anti-beta2glycoprotein IgG; (d) a detection reagent that specifically binds to anti-DGP IgG; (e) a detection reagent that specifically binds to anti-IA2 IgM; and (f) a detection reagent that specifically binds to anti-MPO IgA.

In embodiments, the present disclosure further provides a kit comprising, in one or more vials, containers, or compartments: (a) a surface comprising at least a first, second, third, fourth and fifth binding reagent immobilized on an associated first, second, third, fourth and fifth binding domain, wherein the first, second, third, fourth and fifth binding reagent is a binding partner of anti-TGM2, anti-GAD65, antiZnT8, anti-insulin and anti-IA2, respectively; (b) a detection reagent that specifically binds to anti-TGM2; (c) a detection reagent that specifically binds to anti-GAD65; (d) a detection reagent that specifically binds to anti-ZnT8; (e) a detection reagent that specifically binds to anti-insulin; and (f) a detection reagent that specifically binds to anti-IA2.

The biomarkers anti-IA2 IgG, anti-beta2glycoprotein IgG, anti-DGP IgG, anti-IA2 IgM, anti-MPO IgA, anti-TGM2, anti-GAD65, antiZnT8, anti-insulin and anti-IA2, capture and detection reagents therefor, and anchoring reagents are described herein. In embodiments, the capture and detection reagents are each an antibody. In embodiments where the biomarkers are an antibody, the capture reagents can be an antigen to the antibody. In embodiments, the detection reagents are lyophilized. In embodiments, the detection reagents are provided in solution. In embodiments, the anchoring reagent comprises an anchoring oligonucleotide sequence as described herein. In embodiments, the reagents and other components of the kit are provided separately. In embodiments, the reagents and other components are provided separately according to their optimal shipping or storage temperatures.

C. SLE Kits

In embodiments, the present disclosure further provides a kit comprising, in one or more vials, containers, or compartments: (a) a surface comprising at least a first, second and third binding reagent immobilized on an associated first, second and third binding domain, wherein the first, second and third binding reagent is a binding partner of anti-Smith IgG, anti-RoSSA60 IgG and anti-U1 RNPA IgG, respectively; (b) a detection reagent that specifically binds to anti-Smith IgG; (c) a detection reagent that specifically binds to anti-RoSSA60 IgG; and (d) a detection reagent that specifically binds to anti-U1 RNPA IgG. In embodiments, the kit is used to detect SLE.

The biomarkers anti-Smith IgG, anti-RoSSA60 IgG, anti-U1 RNPA IgG, anti-insulin IgM and anti-RoSSA52 IgG, capture and detection reagents therefor, and anchoring reagents are described herein. In embodiments, the capture and detection reagents are each an antibody. In embodiments where the biomarkers are an antibody, the capture reagents can be an antigen to the antibody. In embodiments, the detection reagents are lyophilized. In embodiments, the detection reagents are provided in solution. In embodiments, the anchoring reagent comprises an anchoring oligonucleotide sequence as described herein. In embodiments, the reagents and other components of the kit are provided separately. In embodiments, the reagents and other components are provided separately according to their optimal shipping or storage temperatures.

In embodiments, the present disclosure further provides a kit comprising, in one or more vials, containers, or compartments: (a) a surface comprising at least a first, second, third, fourth and fifth binding reagent immobilized on an associated first, second, third, fourth and fifth binding domain, wherein the first, second, third, fourth and fifth binding reagent is a binding partner of anti-insulin IgM, anti-MPO IgA, anti-Jo1 IgA, anti-ZnT8 IgM, anti-GAD65 IgG, respectively; (b) a detection reagent that specifically binds to anti-insulin IgM; (c) a detection reagent that specifically binds to anti-MPO IgA; (d) a detection reagent that specifically binds to anti-Jo1 IgA; (e) a detection reagent that specifically binds to anti-ZnT8 IgM; and (f) a detection reagent that specifically binds to anti-GAD65 IgG. In embodiments, the kit is used to detect SLE flare.

In embodiments, the disclosure provides a kit comprising, in one or more vials, containers, or compartments: (a) a surface comprising at least a first and second binding reagent immobilized on an associated first and second binding domain, wherein the first and second binding reagent is a binding partner of anti-insulin IgM and anti-MPO IgA, respectively; (b) a detection reagent that specifically binds to anti-insulin IgM; and (c) a detection reagent that specifically binds to anti-MPO IgA. In embodiments, the kit is used to detect SLE flare.

The biomarkers anti-insulin IgM, anti-MPO IgA, anti-Jo1 IgA, anti-ZnT8 IgM, anti-GAD65 IgG, capture and detection reagents therefor, and anchoring reagents are described herein. In embodiments, the capture and detection reagents are each an antibody. In embodiments where the biomarkers are an antibody, the capture reagents can be an antigen to the antibody. In embodiments, the detection reagents are lyophilized. In embodiments, the detection reagents are provided in solution. In embodiments, the anchoring reagent comprises an anchoring oligonucleotide sequence as described herein. In embodiments, the reagents and other components of the kit are provided separately. In embodiments, the reagents and other components are provided separately according to their optimal shipping or storage temperatures.

D. Celiac Disease Kits

In embodiments, the present disclosure further provides a kit comprising, in one or more vials, containers, or compartments: (a) a surface comprising at least a first, second, third, fourth, fifth and sixth binding reagent immobilized on an associated first, second, third, fourth, fifth and sixth binding domain, wherein the first, second, third, fourth, fifth and sixth binding reagent is a binding partner of anti-DGP IgA, anti-DGP IgG, anti-DGP IgM, anti-TGM2 IgA, anti-TGM2 IgG and anti-TGM2 IgM, respectively; (b) a detection reagent that specifically binds to anti-DGP IgA; (c) a detection reagent that specifically binds to anti-DGP IgG; (d) a detection reagent that specifically binds to anti-DGP IgM; (e) a detection reagent that specifically binds to anti-TGM2 IgA; (f) a detection reagent that specifically binds to TGM2 IgG; and and (g) a detection reagent that specifically binds to anti-TGM2 IgM. In embodiments, the kit can be used to detect celiac disease.

In embodiments, the disclosure provides a kit comprising, in one or more vials, containers, or compartments: (a) a surface comprising at least a first, second, third, fourth, fifth and sixth binding reagent immobilized on an associated first, second, third, fourth, fifth and sixth binding domain, wherein the first, second, third, fourth, fifth and sixth binding reagent is a binding partner of a biomarker selected from anti-TGM2 IgA, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, and anti-ZnT8 IgA, respectively; and (b) detection reagents that specifically binds to six of the biomarkers selected from anti-TGM2 IgA, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, and anti-ZnT8 IgA, respectively. In embodiments, the kit further comprises, in one or more vials containers or compartments at least a seventh, eighth, ninth, tenth, eleventh or twelfth binding reagent which is a binding partner of the listed biomarker and further comprises detection reagents that specifically bind to seven, eight, nine, ten, eleven or twelve of the listed biomarkers.

The biomarkers anti-DGP IgA, anti-DGP IgG, anti-DGP IgM, anti-TGM2 IgA, anti-TGM2 IgG anti-TGM2 IgM, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, and anti-ZnT8 IgA, capture and detection reagents therefor, and anchoring reagents are described herein. In embodiments, the capture and detection reagents are each an antibody. In embodiments where the biomarkers are an antibody, the capture reagents can be an antigen to the antibody. In embodiments, the detection reagents are lyophilized. In embodiments, the detection reagents are provided in solution. In embodiments, the anchoring reagent comprises an anchoring oligonucleotide sequence as described herein. In embodiments, the reagents and other components of the kit are provided separately. In embodiments, the reagents and other components are provided separately according to their optimal shipping or storage temperatures.

E. Kit Components and Properties

For all kits described herein, reagents and methods for immobilizing binding reagents to surfaces, e.g., via targeting agents/targeting agent complements, linking agents/supplemental linking agents, and bridging agents are described herein. In embodiments, the surface is a plate. In embodiments, the surface is a multi-well plate. In embodiments, the surface is a particle. In embodiments, the surface is a cartridge. In embodiments, the surface comprises an electrode. In embodiments, the electrode is a carbon ink electrode.

In embodiments, the kit further comprises a calibration reagent, a control reagent, or both. In embodiments, the calibration reagent comprises a known quantity of a biomarker of interest, e.g., a known quantity of anti-IA2 IgG, anti-beta2glycoprotein IgG, anti-DGP IgG, anti-IA2 IgM and anti-MPO IgA. In embodiments, multiple calibration reagents comprise a range of concentrations of the biomarker. In embodiments, the multiple calibration reagents comprise concentrations of a biomarker near the upper and lower limits of quantitation for the assay. In embodiments, the multiple concentrations of the calibration reagent span the entire dynamic range of the assay. In embodiments, the negative control reagent comprises a sample obtained from an individual not having Type 1 diabetes or not responsive to treatment of Type 1 diabetes with alefacept. In embodiments, the positive control reagent comprises a sample obtained from a subject that exhibits high reactivity to an antigen to one or more of the above biomarker antibodies and who may or may not have Type 1 diabetes. In embodiments, the control reagents are used to provide a basis of comparison for the biological sample to be tested with the methods of the present disclosure. In embodiments, the calibration reagent, the control reagent, or both, are lyophilized. In embodiments, the calibration reagent, the control reagent, or both, are provided in solution.

In embodiments, the kit further comprises a diluent for one or more of the various reagents in the kit. In embodiments, the diluent is subjected to heat during manufacture. In embodiments, the diluent is subjected to a temperature of about 50° C. to about 80° C., about 55° C. to about 75° C., about 60° C. to about 70° C., about 61° C. to about 65° C., or about 62° C. to about 64° C. during manufacture. In embodiments, heat treatment of the diluent reduces interference and/or non-specific binding when performing assays with the kit components.

In embodiments, the kit further comprises one or more of a buffer, e.g., assay buffer, reconstitution buffer, storage buffer, read buffer, and the like; an assay consumable, e.g., assay modules, vials, tubes, liquid handling and transfer devices such as pipette tips, covers and seals, racks, labels, and the like; an assay instrument; and/or instructions for carrying out the assay.

In embodiments, the kit comprises lyophilized reagents, e.g., detection reagent, non-immobilized competing reagent, calibration reagent, and control reagent. In embodiments, the kit comprises one or more solutions to reconstitute the lyophilized reagents.

In embodiments, a kit comprising the components above include stock concentrations of the components that are 5×, 10×, 20×, 30×, 40×, 50×, 60×, 70×, 80×, 90×, 100×, 125×, 150× or higher fold concentrations of the concentrations (e.g., coating, working, calibration, and control concentrations) set forth above.

All references cited herein, including patents, patent applications, papers, textbooks and the like, and the references cited therein, to the extent that they are not already, are hereby incorporated herein by reference in their entirety.

### X. EXAMPLES

### Example 1. Summary of Sample Analysis

Samples were obtained from one study on the effects of alefacept in Type 1 diabetes patients (ITN T1DAL), one study on Celiac disease patient and control samples (BIDMC), and lupus patient and control samples (U Minnesota), as well as up to 73 commercially purchased normal samples, as summarized in Table 2. Alefacept is a dimeric fusion protein consisting of the leukocyte function antigen-3 protein and Fc portion of human IgG1 that blocks the T-cell CD2 receptor thus preventing T-cell proliferation, a key mechanism in psoriasis. It also induces apoptosis of effector memory T cells. Blocking T-cell activity and proliferation is theorized to prevent pancreatic β cell depletion in early stage Type 1 diabetes patients.

TABLE 2

| | No. | No. of | | | |
|---|---|---|---|---|---|
| | of | Commercially | | | Days |
| | Sam- | purchased | No. of | No. of | to |
| Study | ples | normal sera | Isotype | panels | testing |
| ITN T1DAL - T1D | 184 | 44 | 3 | 7 | 6 |
| Ciaran Kelly | 60 | | 3 | 7 | 2 |
| (BIDMC) - Celiac | | | | | |
| Disease | | | | | |
| Brian Fife (Univ. | 45 | 29 | 3 | 7 | 2 |
| Minnesota) - Lupus | | | | | |
| Total | 289 | 73 | | | 10 |

The ITN T1DAL trial was conducted as a multi-center, prospective, double-blind, placebo-controlled, 50-patient, 2:1 randomized, phase II clinical trial for individuals with recent-onset Type 1 diabetes mellitus (T1DM) aged 12-35 years. Participants received weekly IM injections of alefacept (15 mg) or placebo for 12 weeks, followed by a 12-week pause before resuming another 12 weeks of dosing, for a total course of 24 weeks of alefacept or placebo.

The purpose of this project is to measure autoantibodies during Alefacept treatment and stratify with response to see if there is any correlation. Samples were provided blinded to treatment group and outcome. Data were partially unblinded by mining the Immune Tolerance Network (ITN) web portal information for this study.

Placebo—negative response n=11

Placebo—positive response n=1

Alefacept—negative response n=21

Alefacept—positive response n=9

289 total samples from the three studies along with 73 normal samples were tested over 10 days on 7 panels with 24 assays for IgG, IgA and IgM antibody isotypes. 38 samples were tested on each plate along with calibrator, positive and negative controls. Panels tested and the type of assays performed for each antigen are shown in Table 3.

TABLE 3

| | Assay Format | | | | | | |
|---|---|---|---|---|---|---|---|
| | Bridging - Simultaneous | | | Bridging - Sequential | | Classical | |
| Panels | Panel 1 | Panel 2 | Panel 3 | Panel 4 | Panel 5 | Panel 6 | Panel 7 |
| | Insulin | GAD65 | IA2 | TGM2 | TPO | Scl 70 | Beta2glycoprotein |
| | ZnT8 | Intrinsic | Jo1 | DGP | U1 RNP A | ACPA | La/SSB |
| | | factor | | | | (CCP) | |
| | | (IF) | | | | | |
| | Proinsulin | | | Thryroglobulin | Ro/SSA-52 | U1 RNP C | |
| | | | | MPO | aNCA-PR3 | CENP B | |
| | | | | Smith | | Ro/SSA 60 | |
| | | | | | | RNP68/70 | |
| | | | | | | MPO | |
| | | | | | | Smith | |
| In well - | 6X | 6X | 6X | 30X | 30X | 30X | 30X |
| Sample | | | | | | | |
| Dilution | | | | | | | |

The panels shown in Table 3 were assembled by determining optimal assay format and sample dilutions to use for each assay and determining compatibility with other assays in the panel.

As shown in Table 3, panels 1-3 were analyzed using a simultaneous, or regular, bridging assay. Panels 4 and 5 were analyzed using a sequential, or stepwise, bridging assay. Panels 6 and 7 were analyzed using a classical, or serological assay. These assays were generally performed using standard assay techniques as described herein.

Unless described otherwise, the multiplex assays described herein are performed on multi-well plates. Calibrators were made from screened samples positive for specific reactivities that were pooled to create mixed calibrators for each panel. Capture proteins (i.e., binding reagents) were conjugated with biotin according to known methods. For bridging assays, detection antigens were used. Detection antigens were conjugated to oligonucleotides according to known methods. For classical assays, detection antibodies were used. Detection antibodies were conjugated to MesoScale Diagnostics's SULFO-TAG label according to known methods. Negative controls were made from pooled samples of “normal” subjects not having Type 1 diabetes. Positive controls were made from samples obtained from subjects showing high reactivity to one or more antigens.

For each of the following Examples, calibrators and controls were prepared from screened human serum/plasma samples. Multiple individual patient samples were sourced and tested for reactivity to each antigen, to identify ones with high levels of autoantibodies. In most cases, each isotype (IgA, IgG, and IgM) required unique samples. The sample signals had to be high enough such that following pooling of samples for individual reactivities in a panel of assays, sufficient dynamic range remained for calibrator materials. No recombinant material is available for use as calibrator or control. Calibrators and controls were defined for all 72 assays. The performance of these were assessed in a dry run test prior to use in testing of the samples.

For each of the following Examples, calibrators and controls were tested on each assay plate in duplicate. Serum based controls were run on each plate. Concentrations were derived for samples for each reactivity and are presented as arbitrary units/mL (U/mL) unless it is indicated that standard International Unit (IU) concentrations were used. International units are used for the determination of biological material in an internationally agreed upon, consistent manner. The calculated concentrations for each isotype reactivity to a given antigen are derived from unique and separate calibrators for each isotype (e.g. U/mL concentration of anti GAD65 IgG cannot be directly compared to U/mL concentrations of anti GAD65 IgA or IgM reactivities). Concentrations that were at or below the assay detection limits were assigned detection limit values. In many cases, sample signals were high, above the short dynamic ranges of some calibration curves. Hence, the calibrator curves had to be extrapolated beyond the top calibrator, to derive sample concentrations that were above the top calibrator concentration. Samples were tested in duplicate.

### Example 2. Analysis of Treated Vs. Untreated Patients (Ignoring Treatment Outcomes)

For the T1DAL sample cohort, autoimmune biomarkers from both alefacept treated and untreated patients were analyzed at 0 weeks (at the beginning of treatment), 11 weeks, 26 weeks and 30 weeks. Biomarkers with a Mann-Whitney test value below a threshold of 0.05 were considered significant (shown in gray in the tables below). As the final Receiver Operating Characteristic curve (ROC) analysis contained 810 tests (81 biomarkers/table×10 tables). A p-value of 6.2×10⁻⁵ will remain significant at the level of 0.05 after applying a Bonferroni adjustment as a conservative correction for multiple comparisons. Thus, a threshold of 0.05 was chosen.

A summary of the biomarker significance at all timepoints combined when comparing treated versus untreated (placebo) groups, irrespective of treatment outcomes, is provided in Table 4.

TABLE 4

| | Mann- | | | | treated | placebo | geometric |
|---|---|---|---|---|---|---|---|
| | Whitney | | treated | placebo | geometric | geometric | mean |
| Biomarker | Test | AUC | count | count | mean | mean | ratio |
| ZnT8.IgG | 7.70E−09 | 0.223 | 120 | 52 | 11 | 301 | 0.04 |
| Scl.70.IgG | 1.27E−08 | 0.225 | 117 | 52 | 37 | 116 | 0.32 |
| beta2glycoprotein.IgG | 5.84E−06 | 0.281 | 117 | 52 | 51 | 271 | 0.19 |
| ZnT8.IgA | 7.58E−06 | 0.288 | 120 | 52 | 171 | 468 | 0.37 |
| La.SSb.IgG | 1.61E−05 | 0.295 | 117 | 52 | 0.8 | 4.2 | 0.19 |
| ZnT8.IgM | 9.20E−05 | 0.386 | 120 | 52 | 299 | 1630 | 0.18 |
| La.SSb.IgM | 0.00012 | 0.881 | 117 | 52 | 2840 | 3200 | 0.89 |
| U1RNPC.IgG | 0.00132 | 0.345 | 117 | 52 | 226 | 370 | 0.61 |
| RoSSA60.IgG | 0.00208 | 0.363 | 117 | 52 | 0.3 | 0.6 | 0.54 |
| U1.RNPA.IgG | 0.00483 | 0.602 | 117 | 52 | 1.9 | 3.0 | 0.63 |
| GAD65.IgG | 0.00492 | 0.365 | 120 | 52 | 2.9 | 7.6 | 0.39 |
| DGP.IgG | 0.00514 | 0.365 | 117 | 52 | 110 | 225 | 0.49 |
| Scl.70.IgA | 0.00577 | 0.367 | 117 | 52 | 34 | 65 | 0.52 |
| RoSSA60.IgM | 0.00584 | 0.786 | 117 | 52 | 1380 | 1460 | 0.95 |
| DGP.IgM | 0.00871 | 0.659 | 117 | 52 | 920 | 908 | 1.01 |
| TPO.IgG | 0.00938 | 0.440 | 117 | 52 | 3.1 | 11 | 0.29 |
| CENP.B.IgM | 0.01112 | 0.748 | 117 | 52 | 2420 | 2590 | 0.93 |
| IF.IgG | 0.01581 | 0.654 | 120 | 52 | 0.31 | 0.37 | 0.82 |
| MPO.IgG | 0.01800 | 0.386 | 117 | 52 | 120 | 163 | 0.74 |
| aNCA.PR3.IgG | 0.02170 | 0.702 | 117 | 52 | 86 | 166 | 0.52 |
| Sm.IgG | 0.03188 | 0.396 | 117 | 52 | 29 | 42 | 0.71 |
| Jo.1.IgM | 0.04513 | 0.579 | 120 | 52 | 34 | 40 | 0.84 |
| CCP.IgM | 0.04821 | 0.688 | 117 | 52 | 2870 | 3050 | 0.94 |
| aNCA.PR3.IgA | 0.04973 | 0.485 | 117 | 52 | 637 | 1060 | 0.60 |
| IA2.IgM | 0.05185 | 0.700 | 120 | 52 | 282 | 220 | 1.28 |
| CCP.IgG | 0.05302 | 0.407 | 117 | 52 | 30 | 39 | 0.76 |
| MPO.IgG | 0.06582 | 0.416 | 117 | 52 | 29 | 37 | 0.78 |
| Ro.SSA52.IgG | 0.07093 | 0.802 | 117 | 52 | 47 | 56 | 0.83 |
| GAD65.IgA | 0.09126 | 0.420 | 120 | 52 | 26 | 33 | 0.79 |
| MPO.IgM | 0.09184 | 0.778 | 117 | 52 | 869 | 915 | 0.95 |
| RoSSA60.IgA | 0.09342 | 0.420 | 117 | 52 | 3.9 | 4.7 | 0.82 |
| U1.RNPA.IgA | 0.09367 | 0.608 | 117 | 52 | 204 | 281 | 0.73 |
| TGM2.IgM | 0.10047 | 0.790 | 117 | 52 | 1170 | 1320 | 0.89 |
| U1RNPC.IgM | 0.10498 | 0.696 | 117 | 52 | 1230 | 1370 | 0.90 |
| MPO.IgA | 0.11363 | 0.870 | 117 | 52 | 720 | 743 | 0.97 |
| MPO.IgM | 0.11684 | 0.739 | 117 | 52 | 981 | 1060 | 0.93 |
| Scl.70.IgM | 0.12679 | 0.605 | 117 | 52 | 2220 | 2360 | 0.94 |
| DGP.IgA | 0.13262 | 0.427 | 117 | 52 | 122 | 169 | 0.72 |
| Ro.SSA52.IgM | 0.13697 | 0.981 | 117 | 52 | 1500 | 1830 | 0.82 |
| RNP68.70.IgM | 0.13939 | 0.720 | 117 | 52 | 977 | 1140 | 0.86 |
| TGM2.IgA | 0.14937 | 0.931 | 117 | 52 | 4.0 | 2.8 | 1.42 |
| RNP68.70.IgA | 0.15724 | 0.436 | 117 | 52 | 0.0001 | 0.0002 | 0.50 |
| Thyroglobulin.IgM | 0.15836 | 0.874 | 117 | 52 | 2590 | 3190 | 0.81 |
| ProIAA.IgG | 0.16123 | 0.475 | 120 | 52 | 54 | 62 | 0.86 |
| Sm.IgA | 0.16984 | 0.434 | 117 | 52 | 205 | 336 | 0.61 |
| beta2glycoprotein.IgM | 0.17844 | 0.540 | 117 | 52 | 10800 | 15300 | 0.71 |
| CENP.B.IgA | 0.18800 | 0.436 | 117 | 52 | 39 | 61 | 0.64 |
| TPO.IgM | 0.19276 | 0.839 | 117 | 52 | 1610 | 1920 | 0.84 |
| Smith.IgM | 0.20131 | 0.772 | 117 | 52 | 975 | 1180 | 0.83 |
| ProIAA.IgA | 0.20858 | 0.602 | 120 | 52 | 405 | 493 | 0.82 |
| Sm.IgM | 0.21100 | 0.649 | 117 | 52 | 2060 | 2400 | 0.86 |
| Thyroglobulin.IgA | 0.24282 | 0.834 | 117 | 52 | 2680 | 3510 | 0.76 |
| CENP.B.IgG | 0.24751 | 0.444 | 117 | 52 | 11 | 10 | 1.14 |
| U1.RNPA.IgM | 0.24845 | 1.000 | 117 | 52 | 1200 | 1430 | 0.84 |
| IAA.IgG | 0.28739 | 0.451 | 120 | 52 | 77 | 93 | 0.83 |
| IAA.IgM | 0.29590 | 0.570 | 120 | 52 | 315 | 267 | 1.18 |
| Jo.1.IgA | 0.29978 | 0.450 | 120 | 52 | 86 | 89 | 0.97 |
| IAA.IgA | 0.31066 | 0.452 | 120 | 52 | 232 | 257 | 0.90 |
| TGM2.IgG | 0.32624 | 0.658 | 117 | 52 | 10 | 8.0 | 1.26 |
| Smith.IgG | 0.37188 | 0.698 | 117 | 52 | 93 | 103 | 0.91 |
| Thyroglobulin.IgG | 0.37846 | 0.767 | 117 | 52 | 879 | 816 | 1.08 |
| aNCA.PR3.IgM | 0.37885 | 0.947 | 117 | 52 | 1340 | 1550 | 0.86 |
| Jo.1.IgG | 0.39800 | 0.541 | 120 | 52 | 8.0 | 6.7 | 1.19 |
| ProIAA.IgM | 0.49531 | 0.629 | 120 | 52 | 692 | 602 | 1.15 |
| IF.IgM | 0.55610 | 0.505 | 120 | 52 | 39 | 40 | 0.98 |
| Ro.SSA52.IgA | 0.58548 | 0.981 | 117 | 52 | 11 | 12 | 0.97 |
| IA2.IgA | 0.61817 | 0.476 | 120 | 52 | 248 | 236 | 1.05 |
| CCP.IgA | 0.63953 | 0.477 | 117 | 52 | 87 | 102 | 0.85 |
| beta2glycoprotein.IgA | 0.63953 | 0.477 | 117 | 52 | 2730 | 3700 | 0.74 |
| IA2.IgG | 0.65866 | 0.521 | 120 | 52 | 19 | 14 | 1.36 |
| GAD65.IgM | 0.66029 | 0.556 | 120 | 52 | 42 | 43 | 0.97 |
| RNP68.70.IgG | 0.72315 | 0.483 | 117 | 52 | 8.9 | 10 | 0.89 |
| TPO.IgA | 0.79097 | 0.608 | 117 | 52 | 454 | 724 | 0.63 |
| MPO.IgA | 0.85593 | 0.533 | 117 | 52 | 423 | 456 | 0.93 |
| La.SSb.IgA | 0.89028 | 0.507 | 117 | 52 | 1.8 | 1.6 | 1.11 |
| Smith.IgA | 0.89220 | 0.712 | 117 | 52 | 139 | 158 | 0.88 |
| U1RNPC.IgA | 0.92127 | 0.511 | 117 | 52 | 181 | 201 | 0.90 |
| IF.IgA | 0.92799 | 0.518 | 120 | 52 | 60 | 53 | 1.13 |

A summary of the biomarker significance 0 weeks after the start of treatment with alefacept when comparing treated versus untreated (placebo) groups, irrespective of treatment outcomes, is provided in Table 5.

TABLE 5

| | Mann- | | | | treated | placebo | geometric |
|---|---|---|---|---|---|---|---|
| | Whitney | | treated | placebo | geometric | geometric | mean |
| Biomarker | Test | AUC | count | count | mean | mean | ratio |
| Scl.70.IgG | 0.00858 | 0.254 | 26 | 15 | 27 | 124 | 0.22 |
| ZnT8.IgG | 0.02557 | 0.292 | 29 | 15 | 24 | 287 | 0.08 |
| La.SSb.IgG | 0.03959 | 0.308 | 26 | 15 | 0.9 | 4.2 | 0.22 |
| Scl.70.IgA | 0.04278 | 0.308 | 26 | 15 | 23 | 82 | 0.28 |
| La.SSb.IgM | 0.05190 | 0.877 | 26 | 15 | 1470 | 3200 | 0.46 |
| beta2glycoprotein.IgG | 0.05220 | 0.315 | 26 | 15 | 41 | 263 | 0.16 |
| U1.RNPA.IgG | 0.11185 | 0.579 | 26 | 15 | 1.8 | 3.1 | 0.58 |
| ZnT8.IgA | 0.13083 | 0.363 | 29 | 15 | 216 | 402 | 0.54 |
| IAA.IgM | 0.14001 | 0.678 | 29 | 15 | 248 | 177 | 1.40 |
| CENP.B.IgA | 0.14156 | 0.359 | 26 | 15 | 22 | 55 | 0.40 |
| RNP68.70.IgA | 0.14725 | 0.367 | 26 | 15 | 0.0002 | 0.0001 | 2.00 |
| U1RNPC.IgG | 0.14915 | 0.362 | 26 | 15 | 138 | 345 | 0.40 |
| RoSSA60.IgA | 0.15140 | 0.364 | 26 | 15 | 2.9 | 4.6 | 0.62 |
| ZnT8.IgM | 0.15715 | 0.428 | 29 | 15 | 420 | 1580 | 0.27 |
| beta2glycoprotein.IgM | 0.17151 | 0.474 | 26 | 15 | 4940 | 15300 | 0.32 |
| RoSSA60.IgG | 0.17437 | 0.390 | 26 | 15 | 0.3 | 0.5 | 0.69 |
| aNCA.PR3.IgM | 0.18729 | 1.000 | 26 | 15 | 753 | 1540 | 0.49 |
| MPO.IgG | 0.20110 | 0.377 | 26 | 15 | 80 | 174 | 0.46 |
| MPO.IgM | 0.20364 | 0.792 | 26 | 15 | 500 | 895 | 0.56 |
| Ro.SSA52.IgM | 0.20554 | 0.933 | 26 | 15 | 835 | 1870 | 0.45 |
| DGP.IgM | 0.21143 | 0.638 | 26 | 15 | 524 | 889 | 0.59 |
| IF.IgG | 0.23085 | 0.609 | 29 | 15 | 0.4 | 0.4 | 0.87 |
| Sm.IgA | 0.27894 | 0.397 | 26 | 15 | 100 | 298 | 0.34 |
| GAD65.IgG | 0.30214 | 0.402 | 29 | 15 | 4.8 | 9.2 | 0.52 |
| TPO.IgG | 0.31109 | 0.462 | 26 | 15 | 3.5 | 10 | 0.35 |
| Sm.IgG | 0.31407 | 0.403 | 26 | 15 | 22 | 39 | 0.56 |
| Thyroglobulin.IgM | 0.32063 | 0.838 | 26 | 15 | 1410 | 3150 | 0.45 |
| DGP.IgG | 0.32726 | 0.405 | 26 | 15 | 89 | 209 | 0.43 |
| U1.RNPA.IgA | 0.33234 | 0.592 | 26 | 15 | 124 | 266 | 0.47 |
| CCP.IgG | 3.41E−01 | 0.408 | 26 | 15 | 22 | 36 | 0.60 |
| aNCA.PR3.IgG | 3.79E−01 | 0.705 | 26 | 15 | 63 | 157 | 0.40 |
| RNP68.70.IgM | 0.37898 | 0.744 | 26 | 15 | 555 | 1130 | 0.49 |
| Jo.1.IgG | 0.39047 | 0.582 | 29 | 15 | 11 | 7.5 | 1.49 |
| TGM2.IgA | 0.40472 | 0.941 | 26 | 15 | 3.9 | 2.8 | 1.42 |
| Thyroglobulin.IgG | 0.42247 | 0.749 | 26 | 15 | 622 | 862 | 0.72 |
| IAA.IgA | 0.42784 | 0.579 | 29 | 15 | 186 | 169 | 1.10 |
| TPO.IgA | 0.42834 | 0.677 | 26 | 15 | 299 | 707 | 0.42 |
| MPO.IgG | 0.44007 | 0.426 | 26 | 15 | 24 | 43 | 0.57 |
| RoSSA60.IgM | 0.44021 | 0.746 | 26 | 15 | 745 | 1500 | 0.50 |
| IF.IgM | 0.44274 | 0.467 | 29 | 15 | 36 | 41 | 0.87 |
| Jo.1.IgM | 0.45698 | 0.572 | 29 | 15 | 37 | 41 | 0.897 |
| La.SSb.IgA | 0.46488 | 0.572 | 26 | 15 | 1.9 | 1.5 | 1.297 |
| IA2.IgM | 0.47262 | 0.657 | 29 | 15 | 344 | 266 | 1.293 |
| Ro.SSA52.IgA | 0.47838 | 1.000 | 26 | 15 | 9.1 | 12 | 0.790 |
| U1.RNPA.IgM | 0.47838 | 1.000 | 26 | 15 | 676 | 1430 | 0.473 |
| Thyroglobulin.IgA | 0.49503 | 0.813 | 26 | 15 | 1430 | 3550 | 0.403 |
| Ro.SSA52.IgG | 0.52328 | 0.815 | 26 | 15 | 35 | 56 | 0.623 |
| CENP.B.IgM | 0.53172 | 0.695 | 26 | 15 | 1210 | 2670 | 0.453 |
| CCP.IgA | 5.43E−01 | 0.441 | 26 | 15 | 46 | 91 | 0.508 |
| ProIAA.IgM | 0.56400 | 0.664 | 29 | 15 | 699 | 567 | 1.233 |
| MPO.IgA | 0.59150 | 0.885 | 26 | 15 | 426 | 740 | 0.576 |
| CENP.B.IgG | 0.60173 | 0.449 | 26 | 15 | 9.1 | 10 | 0.936 |
| TPO.IgM | 0.61269 | 0.823 | 26 | 15 | 923 | 1880 | 0.491 |
| ProIAA.IgA | 0.62192 | 0.715 | 29 | 15 | 397 | 450 | 0.882 |
| Smith.IgA | 0.62778 | 0.828 | 26 | 15 | 88 | 144 | 0.608 |
| DGP.IgA | 0.63958 | 0.454 | 26 | 15 | 82 | 141 | 0.584 |
| RNP68.70.IgG | 0.64541 | 0.456 | 26 | 15 | 6.9 | 10 | 0.701 |
| IA2.IgG | 0.65583 | 0.543 | 29 | 15 | 34 | 20 | 1.660 |
| GAD65.IgA | 0.65942 | 0.457 | 29 | 15 | 31 | 36 | 0.861 |
| IF.IgA | 0.67291 | 0.554 | 29 | 15 | 58 | 49 | 1.185 |
| beta2glycoprotein.IgA | 0.67837 | 0.541 | 26 | 15 | 1450 | 2730 | 0.531 |
| MPO.IgM | 0.68031 | 0.687 | 26 | 15 | 555 | 1100 | 0.505 |
| GAD65.IgM | 0.70473 | 0.515 | 29 | 15 | 48 | 50 | 0.974 |
| CCP.IgM | 7.41E−01 | 0.654 | 26 | 15 | 1400 | 3150 | 0.444 |
| U1RNPC.IgA | 0.75524 | 0.479 | 26 | 15 | 111 | 203 | 0.547 |
| Smith.IgG | 0.76100 | 0.744 | 26 | 15 | 68 | 102 | 0.663 |
| Sm.IgM | 0.78962 | 0.559 | 26 | 15 | 1060 | 2520 | 0.421 |
| ProIAA.IgG | 0.79870 | 0.545 | 29 | 15 | 46 | 42 | 1.091 |
| IA2.IgA | 0.82364 | 0.522 | 29 | 15 | 282 | 243 | 1.160 |
| Scl.70.IgM | 0.82735 | 0.554 | 26 | 15 | 1120 | 2460 | 0.455 |
| TGM2.IgM | 0.86226 | 0.782 | 26 | 15 | 634 | 1300 | 0.488 |
| TGM2.IgG | 0.86559 | 0.659 | 26 | 15 | 7.8 | 7.2 | 1.080 |
| Jo.1.IgA | 0.87214 | 0.485 | 29 | 15 | 88 | 87 | 1.003 |
| U1RNPC.IgM | 0.88556 | 0.603 | 26 | 15 | 668 | 1420 | 0.470 |
| Smith.IgM | 0.92166 | 0.749 | 26 | 15 | 539 | 1220 | 0.442 |
| aNCA.PR3.IgA | 9.89E−01 | 0.569 | 26 | 15 | 385 | 965 | 0.399 |
| IAA.IgG | 0.99011 | 0.503 | 29 | 15 | 47 | 39 | 1.202 |
| MPO.IgA | 1.00000 | 0.526 | 26 | 15 | 251 | 432 | 0.581 |

A summary of the biomarker significance 11 weeks after the start of treatment with alefacept when comparing treated versus untreated (placebo) groups, irrespective of treatment outcomes, is provided in Table 6.

TABLE 6

| | Mann- | | | | treated | placebo | geometric |
|---|---|---|---|---|---|---|---|
| | Whitney | | treated | placebo | geometric | geometric | mean |
| Biomarker | Test | AUC | count | count | meat | mean | ratio |
| ZnT8.IgG | 0.00659 | 0.244 | 31 | 14 | 17 | 383 | 0.04 |
| beta2glycoprotein.IgG | 0.00853 | 0.256 | 31 | 14 | 58 | 326 | 0.18 |
| Scl.70.IgG | 0.01074 | 0.263 | 31 | 14 | 47 | 126 | 0.37 |
| ZnT8.IgM | 0.02743 | 0.346 | 31 | 14 | 396 | 2220 | 0.18 |
| DGP.IgG | 0.03960 | 0.306 | 31 | 14 | 97 | 270 | 0.36 |
| ZnT8.IgA | 0.04438 | 0.311 | 31 | 14 | 203 | 541 | 0.38 |
| La.SSb.IgG | 0.05425 | 0.318 | 31 | 14 | 0.8 | 3.4 | 0.25 |
| GAD65.IgG | 0.07951 | 0.334 | 31 | 14 | 2.9 | 10 | 0.30 |
| IF.IgG | 0.10800 | 0.558 | 31 | 14 | 0.3 | 0.4 | 0.74 |
| Jo.1.IgM | 0.10877 | 0.537 | 31 | 14 | 32 | 42 | 0.76 |
| Smith.IgG | 0.12658 | 0.618 | 31 | 14 | 99 | 110 | 0.90 |
| U1.RNPA.IgG | 0.13460 | 0.620 | 31 | 14 | 1.9 | 2.5 | 0.78 |
| beta2glycoprotein.IgM | 0.17501 | 0.484 | 31 | 14 | 13100 | 15600 | 0.84 |
| Thyroglobulin.IgM | 0.20649 | 0.929 | 31 | 14 | 3240 | 3130 | 1.04 |
| GAD65.IgA | 0.22024 | 0.385 | 31 | 14 | 26 | 36 | 0.73 |
| DGP.IgM | 0.22997 | 0.654 | 31 | 14 | 1100 | 928 | 1.19 |
| MPO.IgG | 0.23410 | 0.392 | 31 | 14 | 28 | 36 | 0.79 |
| CENP.B.IgM | 0.24045 | 0.740 | 31 | 14 | 3000 | 2630 | 1.14 |
| La.SSb.IgM | 0.24600 | 0.823 | 31 | 14 | 3510 | 3260 | 1.08 |
| Scl.70.IgA | 0.26816 | 0.394 | 31 | 14 | 42 | 63 | 0.68 |
| Thyroglobulin.IgG | 0.28881 | 0.836 | 31 | 14 | 1090 | 650 | 1.68 |
| U1RNPC.IgG | 0.30131 | 0.401 | 31 | 14 | 286 | 349 | 0.82 |
| RoSSA60.IgG | 0.30259 | 0.412 | 31 | 14 | 0.4 | 0.6 | 0.59 |
| RoSSA60.IgM | 0.31062 | 0.735 | 31 | 14 | 1630 | 1510 | 1.08 |
| Ro.SSA52.IgA | 0.35403 | 1.000 | 31 | 14 | 13 | 12 | 1.10 |
| DGP.IgA | 0.40214 | 0.419 | 31 | 14 | 127 | 177 | 0.72 |
| IA2.IgM | 0.41272 | 0.659 | 31 | 14 | 312 | 269 | 1.16 |
| Sm.IgG | 0.41600 | 0.422 | 31 | 14 | 33 | 41 | 0.79 |
| MPO.IgG | 0.43013 | 0.424 | 31 | 14 | 144 | 168 | 0.86 |
| ProIAA.IgM | 0.43962 | 0.654 | 31 | 14 | 739 | 592 | 1.25 |
| TGM2.IgA | 0.44272 | 0.933 | 31 | 14 | 3.6 | 3.1 | 1.17 |
| IF.IgM | 0.44911 | 0.454 | 31 | 14 | 38 | 44 | 0.87 |
| TPO.IgA | 0.45292 | 0.712 | 31 | 14 | 546 | 611 | 0.89 |
| U1.RNPA.IgA | 0.45591 | 0.622 | 31 | 14 | 239 | 264 | 0.91 |
| CCP.IgG | 4.59E−01 | 0.429 | 31 | 14 | 34 | 41 | 0.83 |
| GAD65.IgM | 0.47234 | 0.521 | 31 | 14 | 40 | 53 | 0.75 |
| Smith.IgA | 0.47308 | 0.624 | 31 | 14 | 162 | 178 | 0.91 |
| CCP.IgM | 4.75E−01 | 0.636 | 31 | 14 | 3420 | 3170 | 1.08 |
| Jo.1.IgA | 0.50470 | 0.435 | 31 | 14 | 85 | 90 | 0.95 |
| U1.RNPA.IgM | 0.53261 | 1.000 | 31 | 14 | 1430 | 1430 | 1.00 |
| MPO.IgM | 0.56664 | 0.726 | 31 | 14 | 1160 | 1100 | 1.05 |
| CENP.B.IgG | 0.58542 | 0.447 | 31 | 14 | 13 | 11 | 1.19 |
| ProIAA.IgG | 0.58608 | 0.479 | 31 | 14 | 58 | 67 | 0.87 |
| MPO.IgA | 0.58650 | 0.869 | 31 | 14 | 810 | 758 | 1.07 |
| ProIAA.IgA | 0.59450 | 0.590 | 31 | 14 | 422 | 482 | 0.88 |
| U1RNPC.IgA | 0.61501 | 0.555 | 31 | 14 | 226 | 194 | 1.16 |
| IAA.IgA | 0.61513 | 0.452 | 31 | 14 | 257 | 274 | 0.94 |
| aNCA.PR3.IgA | 6.19E−01 | 0.544 | 31 | 14 | 767 | 972 | 0.79 |
| La.SSb.IgA | 0.61926 | 0.548 | 31 | 14 | 2.0 | 1.2 | 1.68 |
| TPO.IgM | 0.63710 | 0.862 | 31 | 14 | 1890 | 1890 | 1.00 |
| Sm.IgM | 0.65331 | 0.652 | 31 | 14 | 2470 | 2440 | 1.01 |
| TPO.IgG | 0.66664 | 0.535 | 31 | 14 | 3.8 | 5.3 | 0.71 |
| RNP68.70.IgA | 0.66785 | 0.461 | 31 | 14 | 0.0001 | 0.0001 | 1.00 |
| RoSSA60.IgA | 0.67152 | 0.459 | 31 | 14 | 4.4 | 4.6 | 0.95 |
| aNCA.PR3.IgG | 6.72E−01 | 0.802 | 31 | 14 | 105 | 121 | 0.87 |
| Scl.70.IgM | 0.70239 | 0.565 | 31 | 14 | 2710 | 2490 | 1.09 |
| CCP.IgA | 7.07E−01 | 0.537 | 31 | 14 | 110 | 84 | 1.30 |
| RNP68.70.IgG | 0.70726 | 0.463 | 31 | 14 | 10 | 11 | 0.88 |
| RNP68.70.IgM | 0.70943 | 0.689 | 31 | 14 | 1140 | 1160 | 0.98 |
| TGM2.IgG | 0.73592 | 0.509 | 31 | 14 | 11 | 10 | 1.11 |
| Sm.IgA | 0.74364 | 0.468 | 31 | 14 | 264 | 323 | 0.82 |
| MPO.IgM | 0.75897 | 0.677 | 31 | 14 | 1040 | 926 | 1.12 |
| IAA.IgG | 0.76859 | 0.472 | 31 | 14 | 90 | 105 | 0.85 |
| beta2glycoprotein.IgA | 0.79925 | 0.525 | 31 | 14 | 3570 | 2970 | 1.20 |
| Smith.IgM | 0.81395 | 0.707 | 31 | 14 | 1160 | 1210 | 0.96 |
| TGM2.IgM | 0.83993 | 0.675 | 31 | 14 | 1410 | 1440 | 0.98 |
| U1RNPC.IgM | 0.84178 | 0.666 | 31 | 14 | 1440 | 1420 | 1.01 |
| IA2.IgG | 0.85587 | 0.518 | 31 | 14 | 27 | 22 | 1.20 |
| IAA.IgM | 0.87315 | 0.495 | 31 | 14 | 326 | 324 | 1.01 |
| CENP.B.IgA | 0.87492 | 0.516 | 31 | 14 | 54 | 57 | 0.94 |
| Jo.1.IgG | 0.88305 | 0.516 | 31 | 14 | 8.8 | 7.4 | 1.19 |
| Ro.SSA52.IgG | 0.88454 | 0.864 | 31 | 14 | 52 | 54 | 0.96 |
| IF.IgA | 0.95099 | 0.516 | 31 | 14 | 64 | 55 | 1.16 |
| Thyroglobulin.IgA | 0.95970 | 0.809 | 31 | 14 | 3570 | 3500 | 1.02 |
| MPO.IgA | 0.97038 | 0.532 | 31 | 14 | 482 | 472 | 1.02 |
| IA2.IgA | 0.97066 | 0.495 | 31 | 14 | 263 | 252 | 1.04 |
| aNCA.PR3.IgM | 1.00E+00 | 0.933 | 31 | 14 | 1590 | 1550 | 1.03 |
| Ro.SSA52.IgM | NA | 1.000 | 31 | 14 | 1810 | 1810 | 1.00 |

A summary of the biomarker significance 26 weeks after the start of treatment with alefacept when comparing treated versus untreated (placebo) groups, irrespective of treatment outcomes, is provided in Table 7.

TABLE 7

| | Mann- | | | | treated | placebo | geometric |
|---|---|---|---|---|---|---|---|
| | Whitney | | treated | placebo | geometric | geometric | mean |
| Biomarker | Test | AUC | count | count | mean | mean | ratio |
| ZnT8.IgG | 1.69E−03 | 0.186 | 30 | 12 | 7.3 | 322 | 0.02 |
| Scl.70.IgG | 0.00658 | 0.233 | 30 | 12 | 40 | 100 | 0.40 |
| ZnT8.IgA | 0.01424 | 0.258 | 30 | 12 | 150 | 485 | 0.31 |
| La.SSb.IgM | 0.02039 | 0.922 | 30 | 12 | 3580 | 3140 | 1.14 |
| RoSSA60.IgM | 0.03147 | 0.867 | 30 | 12 | 1710 | 1390 | 1.23 |
| DGP.IgM | 0.03462 | 0.758 | 30 | 12 | 1170 | 783 | 1.49 |
| TGM2.IgM | 0.03913 | 0.922 | 30 | 12 | 1470 | 1210 | 1.21 |
| CENP.B.IgM | 0.05559 | 0.806 | 30 | 12 | 3120 | 2410 | 1.29 |
| La.SSb.IgG | 0.05983 | 0.322 | 30 | 12 | 0.7 | 4.1 | 0.17 |
| aNCA.PR3.IgG | 0.05991 | 0.617 | 30 | 12 | 86 | 200 | 0.43 |
| aNCA.PR3.IgA | 0.07214 | 0.389 | 30 | 12 | 721 | 1140 | 0.63 |
| TPO.IgG | 0.07946 | 0.422 | 30 | 12 | 2.3 | 16 | 0.14 |
| beta2glycoprotein.IgG | 0.08179 | 0.325 | 30 | 12 | 55 | 212 | 0.26 |
| CCP.IgM | 0.08832 | 0.756 | 30 | 12 | 3730 | 2850 | 1.31 |
| GAD65.IgG | 0.09236 | 0.331 | 30 | 12 | 2.8 | 8.0 | 0.35 |
| RoSSA60.IgG | 0.10308 | 0.344 | 30 | 12 | 0.3 | 0.6 | 0.49 |
| ZnT8.IgM | 0.10481 | 0.442 | 30 | 12 | 246 | 1630 | 0.15 |
| IA2.IgM | 0.10498 | 0.792 | 30 | 12 | 257 | 166 | 1.55 |
| U1RNPC.IgG | 0.11664 | 0.342 | 30 | 12 | 246 | 333 | 0.74 |
| MPO.IgM | 0.13173 | 0.814 | 30 | 12 | 1230 | 1010 | 1.22 |
| U1RNPC.IgM | 0.13908 | 0.750 | 30 | 12 | 1530 | 1300 | 1.18 |
| Ro.SSA52.IgG | 0.14425 | 0.767 | 30 | 12 | 50 | 58 | 0.87 |
| MPO.IgM | 0.14685 | 0.819 | 30 | 12 | 1050 | 892 | 1.18 |
| Smith.IgM | 0.14828 | 0.861 | 30 | 12 | 1190 | 1120 | 1.06 |
| Scl.70.IgM | 0.16245 | 0.661 | 30 | 12 | 2960 | 2260 | 1.31 |
| RNP68.70.IgM | 0.20593 | 0.764 | 30 | 12 | 1210 | 1090 | 1.11 |
| Sm.IgM | 2.37E−01 | 0.686 | 30 | 12 | 2570 | 2260 | 1.14 |
| DGP.IgG | 0.24032 | 0.381 | 30 | 12 | 139 | 230 | 0.60 |
| Sm.IgG | 0.25171 | 0.383 | 30 | 12 | 32 | 42 | 0.76 |
| U1.RNPA.IgG | 0.25486 | 0.619 | 30 | 12 | 1.7 | 3.1 | 0.56 |
| ProIAA.IgG | 0.26427 | 0.431 | 30 | 12 | 45 | 65 | 0.69 |
| MPO.IgA | 0.30296 | 0.861 | 30 | 12 | 887 | 755 | 1.17 |
| IAA.IgG | 0.33670 | 0.403 | 30 | 12 | 73 | 111 | 0.66 |
| Thyroglobulin.IgM | 0.35009 | 0.917 | 30 | 12 | 3130 | 3130 | 1.00 |
| MPO.IgG | 3.56E−01 | 0.406 | 30 | 12 | 126 | 138 | 0.91 |
| IAA.IgA | 0.36545 | 0.408 | 30 | 12 | 233 | 274 | 0.85 |
| ProIAA.IgA | 0.37166 | 0.617 | 30 | 12 | 381 | 476 | 0.80 |
| TPO.IgM | 0.39687 | 0.844 | 30 | 12 | 1890 | 1960 | 0.96 |
| IF.IgG | 0.39753 | 0.689 | 30 | 12 | 0.29 | 0.34 | 0.85 |
| MPO.IgG | 4.09E−01 | 0.428 | 30 | 12 | 29 | 34 | 0.86 |
| IF.IgM | 0.42522 | 0.600 | 30 | 12 | 48 | 33 | 1.47 |
| IAA.IgM | 0.42683 | 0.592 | 30 | 12 | 349 | 266 | 1.31 |
| Scl.70.IgA | 0.48293 | 0.428 | 30 | 12 | 39 | 51 | 0.76 |
| CCP.IgG | 0.53609 | 0.436 | 30 | 12 | 33 | 36 | 0.93 |
| DGP.IgA | 0.53609 | 0.436 | 30 | 12 | 160 | 182 | 0.88 |
| Smith.IgG | 0.54785 | 0.622 | 30 | 12 | 107 | 105 | 1.02 |
| Ro.SSA52.IgA | 0.56208 | 1.000 | 30 | 12 | 12 | 12 | 1.03 |
| U1.RNPA.IgM | 0.56208 | 1.000 | 30 | 12 | 1450 | 1430 | 1.01 |
| MPO.IgA | 0.56683 | 0.575 | 30 | 12 | 534 | 451 | 1.18 |
| Jo.1.IgM | 0.59195 | 0.647 | 30 | 12 | 33 | 36 | 0.92 |
| Jo.1.IgA | 0.61114 | 0.447 | 30 | 12 | 88 | 88 | 1.00 |
| RoSSA60.IgA | 0.67609 | 0.461 | 30 | 12 | 4.0 | 4.0 | 1.01 |
| beta2glycoprotein.IgM | 0.74186 | 0.533 | 30 | 12 | 14300 | 15200 | 0.94 |
| Sm.IgA | 7.49E−01 | 0.467 | 30 | 12 | 245 | 320 | 0.77 |
| U1.RNPA.IgA | 0.75761 | 0.683 | 30 | 12 | 228 | 263 | 0.87 |
| GAD65.IgM | 0.75895 | 0.639 | 30 | 12 | 45 | 35 | 1.29 |
| GAD65.IgA | 0.75939 | 0.469 | 30 | 12 | 25 | 29 | 0.87 |
| TGM2.IgG | 0.76579 | 0.586 | 30 | 12 | 12 | 9.3 | 1.28 |
| IA2.IgG | 0.76999 | 0.531 | 30 | 12 | 15 | 10 | 1.46 |
| CENP.B.IgG | 0.77309 | 0.469 | 30 | 12 | 12 | 8.4 | 1.47 |
| RNP68.70.IgA | 0.78060 | 0.475 | 30 | 12 | 0.0001 | 0.0002 | 0.50 |
| TPO.IgA | 0.78431 | 0.564 | 30 | 12 | 487 | 827 | 0.59 |
| Jo.1.IgG | 0.79420 | 0.528 | 30 | 12 | 7.1 | 6.6 | 1.07 |
| IF.IgA | 0.82256 | 0.500 | 30 | 12 | 56 | 53 | 1.07 |
| aNCA.PR3.IgM | 0.82693 | 0.925 | 30 | 12 | 1600 | 1550 | 1.03 |
| Thyroglobulin.IgA | 0.82726 | 0.917 | 30 | 12 | 3090 | 3370 | 0.92 |
| CENP.B.IgA | 0.83685 | 0.478 | 30 | 12 | 43 | 56 | 0.78 |
| Thyroglobulin.IgG | 0.85171 | 0.717 | 30 | 12 | 844 | 902 | 0.94 |
| Smith.IgA | 0.85819 | 0.728 | 30 | 12 | 156 | 160 | 0.98 |
| U1RNPC.IgA | 0.86727 | 0.522 | 30 | 12 | 204 | 197 | 1.04 |
| TGM2.IgA | 0.87279 | 0.850 | 30 | 12 | 4.3 | 2.9 | 1.49 |
| CCP.IgA | 0.87998 | 0.517 | 30 | 12 | 106 | 99 | 1.07 |
| beta2glycoprotein.IgA | 0.90168 | 0.486 | 30 | 12 | 3190 | 3560 | 0.90 |
| RNP68.70.IgG | 0.90168 | 0.514 | 30 | 12 | 9.5 | 10 | 0.99 |
| La.SSb.IgA | 0.92346 | 0.489 | 30 | 12 | 1.7 | 1.6 | 1.06 |
| IA2.IgA | 0.94529 | 0.492 | 30 | 12 | 233 | 223 | 1.04 |
| ProIAA.IgM | 0.95374 | 0.583 | 30 | 12 | 640 | 614 | 1.04 |
| Ro.SSA52.IgM | NA | 1.000 | 30 | 12 | 1810 | 1810 | 1.00 |

A summary of the biomarker significance 30 weeks after the start of treatment with alefacept when comparing treated versus untreated (placebo) groups, irrespective of treatment outcomes, is provided in Table 8.

TABLE 8

| | Mann- | | | | treated | placebo | geometric |
|---|---|---|---|---|---|---|---|
| | Whitney | | treated | placebo | geometric | geometric | mean |
| Biomarker | Test | AUC | count | count | mean | mean | ratio |
| Scl.70.IgG | 0.00008 | 0.121 | 30 | 11 | 36 | 110 | 0.33 |
| ZnT8.IgG | 1.66E−03 | 0.176 | 30 | 11 | 4.6 | 220 | 0.02 |
| ZnT8.IgA | 0.00310 | 0.197 | 30 | 11 | 130 | 461 | 0.28 |
| beta2glycoprotein.IgG | 0.00640 | 0.224 | 30 | 11 | 53 | 295 | 0.18 |
| La.SSb.IgG | 0.00842 | 0.227 | 30 | 11 | 0.7 | 5.7 | 0.12 |
| U1RNPC.IgG | 0.01361 | 0.248 | 30 | 11 | 261 | 493 | 0.53 |
| TGM2.IgG | 0.02791 | 0.939 | 30 | 11 | 10 | 6.0 | 1.71 |
| RoSSA60.IgG | 0.03275 | 0.285 | 30 | 11 | 0.3 | 0.7 | 0.42 |
| La.SSb.IgM | 0.03381 | 0.909 | 30 | 11 | 3420 | 3200 | 1.07 |
| ZnT8.IgM | 0.04240 | 0.367 | 30 | 11 | 196 | 1140 | 0.17 |
| TPO.IgG | 0.04425 | 0.315 | 30 | 11 | 3.1 | 17 | 0.18 |
| beta2glycoprotein.IgA | 0.09090 | 0.324 | 30 | 11 | 3260 | 7770 | 0.42 |
| MPO.IgG | 9.09E−02 | 0.324 | 30 | 11 | 142 | 170 | 0.84 |
| U1RNPC.IgM | 0.09680 | 0.782 | 30 | 11 | 1520 | 1300 | 1.17 |
| ProIAA.IgA | 0.10614 | 0.455 | 30 | 11 | 422 | 598 | 0.71 |
| Ro.SSA52.IgA | 0.11040 | 0.909 | 30 | 11 | 12 | 12 | 0.96 |
| aNCA.PR3.IgA | 0.11468 | 0.400 | 30 | 11 | 755 | 1220 | 0.62 |
| Thyroglobulin.IgA | 0.11786 | 0.821 | 30 | 11 | 3150 | 3610 | 0.87 |
| IAA.IgA | 0.12238 | 0.339 | 30 | 11 | 256 | 393 | 0.65 |
| Scl.70.IgA | 0.13111 | 0.342 | 30 | 11 | 33 | 63 | 0.51 |
| RoSSA60.IgM | 0.13680 | 0.812 | 30 | 11 | 1700 | 1400 | 1.21 |
| RoSSA60.IgA | 0.14523 | 0.348 | 30 | 11 | 4.3 | 6.1 | 0.72 |
| CENP.B.IgA | 0.14712 | 0.348 | 30 | 11 | 44 | 82 | 0.53 |
| TGM2.IgA | 0.16226 | 1.000 | 30 | 11 | 4.3 | 2.7 | 1.62 |
| U1.RNPA.IgG | 0.18141 | 0.585 | 30 | 11 | 2.0 | 3.4 | 0.58 |
| Ro.SSA52.IgG | 0.20473 | 0.745 | 30 | 11 | 52 | 59 | 0.87 |
| aNCA.PR3.IgG | 0.20795 | 0.670 | 30 | 11 | 97 | 222 | 0.43 |
| CCP.IgG | 0.21108 | 0.370 | 30 | 11 | 32 | 47 | 0.69 |
| Sm.IgG | 0.21453 | 0.370 | 30 | 11 | 33 | 47 | 0.70 |
| U1.RNPA.IgA | 0.23619 | 0.533 | 30 | 11 | 250 | 356 | 0.70 |
| MPO.IgM | 0.24360 | 0.848 | 30 | 11 | 1010 | 957 | 1.06 |
| CENP.B.IgM | 0.24693 | 0.752 | 30 | 11 | 2970 | 2620 | 1.13 |
| CCP.IgM | 0.24885 | 0.724 | 30 | 11 | 3710 | 3000 | 1.24 |
| Sm.IgM | 2.52E−01 | 0.715 | 30 | 11 | 2590 | 2320 | 1.12 |
| TPO.IgA | 0.27023 | 0.427 | 30 | 11 | 526 | 806 | 0.65 |
| CENP.B.IgG | 0.27428 | 0.385 | 30 | 11 | 11 | 11 | 1.01 |
| DGP.IgA | 0.27428 | 0.385 | 30 | 11 | 132 | 189 | 0.70 |
| GAD65.IgA | 0.28274 | 0.391 | 30 | 11 | 23 | 30 | 0.75 |
| DGP.IgG | 0.30100 | 0.391 | 30 | 11 | 120 | 192 | 0.63 |
| Sm.IgA | 3.01E−01 | 0.391 | 30 | 11 | 265 | 437 | 0.61 |
| GAD65.IgG | 0.31497 | 0.394 | 30 | 11 | 2.0 | 3.9 | 0.51 |
| Scl.70.IgM | 0.31959 | 0.648 | 30 | 11 | 2640 | 2200 | 1.20 |
| IAA.IgG | 0.35391 | 0.403 | 30 | 11 | 113 | 218 | 0.52 |
| IA2.IgA | 0.35930 | 0.403 | 30 | 11 | 220 | 223 | 0.99 |
| CCP.IgA | 0.39087 | 0.409 | 30 | 11 | 102 | 154 | 0.66 |
| La.SSb.IgA | 0.39087 | 0.409 | 30 | 11 | 1.5 | 2.5 | 0.60 |
| ProIAA.IgG | 0.39713 | 0.439 | 30 | 11 | 70 | 94 | 0.74 |
| IA2.IgM | 0.41586 | 0.718 | 30 | 11 | 228 | 180 | 1.27 |
| Smith.IgM | 0.42313 | 0.791 | 30 | 11 | 1180 | 1150 | 1.03 |
| RNP68.70.IgA | 0.44355 | 0.427 | 30 | 11 | 0.0001 | 0.0004 | 0.25 |
| Jo.1.IgM | 0.45908 | 0.594 | 30 | 11 | 34 | 41 | 0.82 |
| MPO.IgA | 0.47010 | 0.858 | 30 | 11 | 858 | 715 | 1.20 |
| IF.IgG | 0.50102 | 0.827 | 30 | 11 | 0.27 | 0.30 | 0.90 |
| MPO.IgM | 0.51048 | 0.742 | 30 | 11 | 1140 | 1010 | 1.13 |
| Jo.1.IgG | 0.51348 | 0.570 | 30 | 11 | 5.9 | 5.1 | 1.15 |
| TPO.IgM | 0.53452 | 0.830 | 30 | 11 | 1980 | 1970 | 1.01 |
| TGM2.IgM | 0.58954 | 0.779 | 30 | 11 | 1370 | 1310 | 1.05 |
| Jo.1.IgA | 0.61160 | 0.445 | 30 | 11 | 85 | 90 | 0.95 |
| Smith.IgG | 0.65501 | 0.848 | 30 | 11 | 103 | 93 | 1.11 |
| IAA.IgM | 0.66887 | 0.470 | 30 | 11 | 343 | 370 | 0.93 |
| beta2glycoprotein.IgM | 0.66946 | 0.691 | 30 | 11 | 14100 | 15100 | 0.93 |
| IA2.IgG | 0.67407 | 0.545 | 30 | 11 | 9.2 | 6.3 | 1.47 |
| RNP68.70.IgM | 0.69264 | 0.688 | 30 | 11 | 1170 | 1200 | 0.98 |
| Smith.IgA | 0.69507 | 0.645 | 30 | 11 | 164 | 152 | 1.08 |
| MPO.IgA | 0.71201 | 0.479 | 30 | 11 | 484 | 473 | 1.02 |
| DGP.IgM | 0.74529 | 0.558 | 30 | 11 | 1040 | 1070 | 0.97 |
| Thyroglobulin.IgM | 0.75341 | 0.818 | 30 | 11 | 3040 | 3380 | 0.90 |
| U1RNPC.IgA | 0.76852 | 0.473 | 30 | 11 | 203 | 214 | 0.95 |
| IF.IgM | 0.82255 | 0.530 | 30 | 11 | 35 | 43 | 0.82 |
| MPO.IgG | 8.25E−01 | 0.479 | 30 | 11 | 35 | 35 | 0.99 |
| Thyroglobulin.IgG | 0.86918 | 0.770 | 30 | 11 | 1030 | 907 | 1.14 |
| RNP68.70.IgG | 0.87335 | 0.518 | 30 | 11 | 9.3 | 9.3 | 1.00 |
| aNCA.PR3.IgM | 0.88640 | 0.918 | 30 | 11 | 1640 | 1560 | 1.05 |
| ProIAA.IgM | 0.88904 | 0.591 | 30 | 11 | 691 | 654 | 1.06 |
| IF.IgA | 0.90596 | 0.524 | 30 | 11 | 60 | 55 | 1.09 |
| GAD65.IgM | 0.98796 | 0.570 | 30 | 11 | 37 | 35 | 1.05 |
| Ro.SSA52.IgM | NA | 1.000 | 30 | 11 | 1810 | 1810 | 1.00 |
| U1.RNPA.IgM | NA | 1.000 | 30 | 11 | 1430 | 1430 | 1.00 |

Key results of the comparison of treated vs. untreated (ignoring treatment outcomes) are summarized as follows:
The treated group appears to have lower anti-ZnT8, Sc170, and LaSSB levels to start with at 0 weeks.
Alefacept treatment results in decrease in anti-ZnT8 IgA, IgG, and IgM levels.
Alefacept treatment effects on anti-Sc170 IgG, anti-beta2glycoprotein IgG, and LaSSB IgG suggest decreased levels.

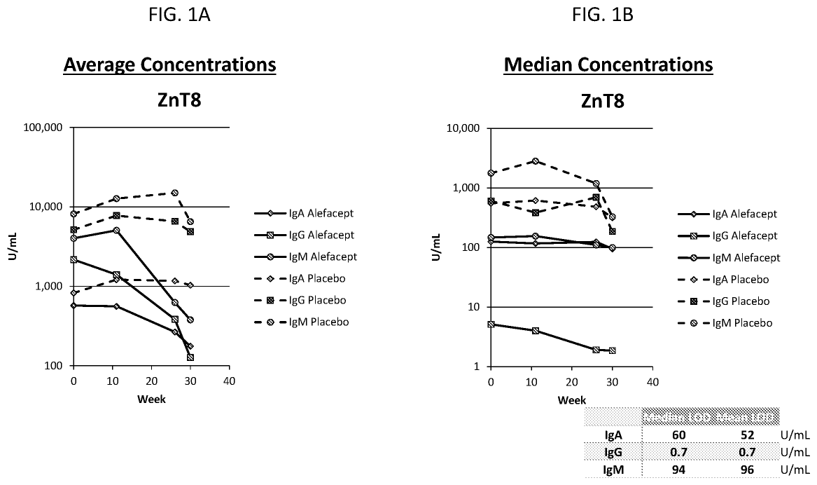

Results obtained for anti-ZnT8 are shown in FIG. 1, with FIG. 1A plotting average anti-ZnT8 concentrations and FIG. 1B plotting median anti-ZnT8 concentrations. As can be seen from the results: 1) IgA levels are higher in placebo group at all time points, and may be decreasing with time in treatment group. 2) IgG levels are higher in placebo group at all time points. Treatment group shows decrease in levels with time. 3) IgM levels are higher in placebo group at all time points, and may be decreasing after 11 weeks especially in the treatment group.

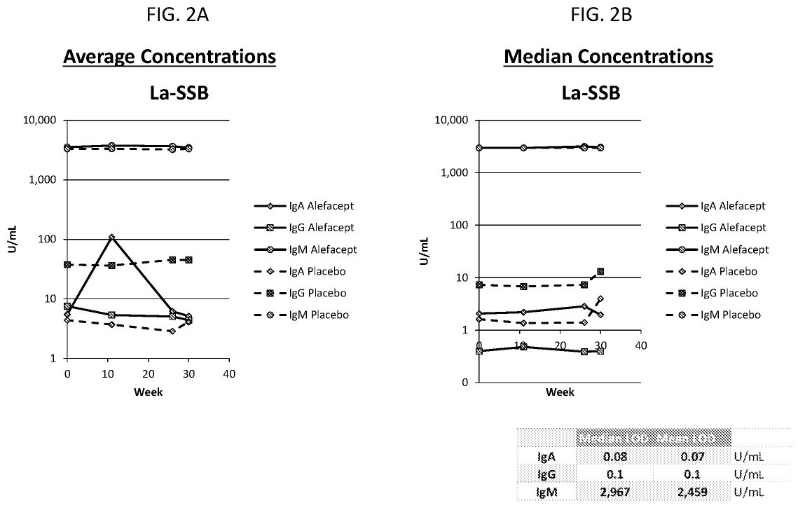

Results obtained for LaSSB autoantibodies are shown in FIG. 2, with FIG. 2A plotting average LaSSB autoantibody concentrations and FIG. 2B plotting median LaSSB autoantibody concentrations. As can be seen from the results: 1) IgA levels may be higher at 11 and 26 week time points in treatment group. 2) Higher IgG levels seen in placebo group versus treatment group.

### Example 3. Analysis of Only Treated Patients for Positive Vs. Negative Outcomes

Autoimmune biomarkers from treated patients were analyzed for positive and negative outcomes with alefacept treatment. Samples were analyzed at 0 weeks (at the beginning of treatment), 11 weeks, 26 weeks and 30 weeks. Biomarkers with a Mann-Whitney test value below a threshold of 0.05 were considered significant (shown in gray in the tables below). As the final ROC analysis contained 810 tests (81 biomarkers/table×10 tables). A p-value of 6.2×10⁻⁵ will remain significant at the level of 0.05 after applying a Bonferroni adjustment as a conservative correction for multiple comparisons. Thus, a threshold of 0.05 was chosen.

A summary of the biomarker significance at all timepoints combined is provided in Table 9.

TABLE 9

| | Mann- | | | | positive | negative | geometric |
|---|---|---|---|---|---|---|---|
| | Whitney | | positive | negative | geometric | geometric | mean |
| Biomarker | Test | AUC | count | count | mean | mean | ratio |
| beta2glycoprotein.IgA | 0.04938 | 0.743 | 9 | 16 | 6120 | 770 | 7.95 |
| IA2.IgM | 0.05260 | 0.754 | 9 | 19 | 681 | 231 | 2.95 |
| IA2.IgG | 0.05502 | 0.731 | 9 | 19 | 187 | 12 | 15.20 |
| MPO.IgA | 0.09267 | 0.708 | 9 | 16 | 542 | 173 | 3.13 |
| DGP.IgG | 0.10764 | 0.701 | 9 | 16 | 308 | 54 | 5.69 |
| TGM2.IgG | 0.10825 | 0.583 | 9 | 16 | 6.2 | 8.1 | 0.76 |
| ProIAA.IgG | 0.11220 | 0.421 | 9 | 19 | 25 | 49 | 0.51 |
| aNCA.PR3.IgG | 0.12075 | 0.938 | 9 | 16 | 104 | 39 | 2.67 |
| U1RNPC.IgG | 0.12104 | 0.306 | 9 | 16 | 178 | 116 | 1.53 |
| MPO.IgM | 0.13355 | 0.444 | 9 | 16 | 841 | 381 | 2.21 |
| Sm.IgG | 0.13564 | 0.313 | 9 | 16 | 23 | 20 | 1.17 |
| Jo.1.IgA | 0.15643 | 0.673 | 9 | 19 | 100 | 83 | 1.20 |
| Thyroglobulin.IgG | 0.18011 | 0.771 | 9 | 16 | 1280 | 307 | 4.17 |
| IA2.IgA | 0.18388 | 0.661 | 9 | 19 | 341 | 249 | 1.37 |
| IAA.IgG | 0.19967 | 0.363 | 9 | 19 | 21 | 53 | 0.39 |
| RNP68.70.IgG | 0.20715 | 0.340 | 9 | 16 | 5.9 | 7.2 | 0.83 |
| U1.RNPA.IgM | 0.21130 | 1.000 | 9 | 16 | 1450 | 453 | 3.20 |
| Jo.1.IgM | 2.52E−01 | 0.766 | 9 | 19 | 44 | 34 | 1.28 |
| IF.IgA | 0.26607 | 0.392 | 9 | 19 | 48 | 66 | 0.73 |
| RoSSA60.IgG | 0.29307 | 0.389 | 9 | 16 | 0.3 | 0.4 | 0.66 |
| RNP68.70.IgA | 0.29400 | 0.382 | 9 | 16 | 0.0000 | 0.0004 | 0.00 |
| Thyroglobulin.IgA | 3.07E−01 | 0.875 | 9 | 16 | 3050 | 898 | 3.40 |
| IAA.IgM | 0.32287 | 0.404 | 9 | 19 | 181 | 261 | 0.69 |
| ZnT8.IgM | 0.32627 | 0.456 | 9 | 19 | 260 | 395 | 0.66 |
| CCP.IgA | 0.33574 | 0.382 | 9 | 16 | 55 | 42 | 1.32 |
| RoSSA60.IgM | 0.37605 | 0.729 | 9 | 16 | 1820 | 476 | 3.82 |
| U1.RNPA.IgA | 0.39293 | 0.799 | 9 | 16 | 227 | 91 | 2.49 |
| La.SSb.IgG | 0.41134 | 0.611 | 9 | 16 | 1.3 | 0.8 | 1.63 |
| CENP.B.IgA | 0.41924 | 0.604 | 9 | 16 | 35 | 18 | 1.98 |
| La.SSb.IgA | 4.19E−01 | 0.604 | 9 | 16 | 3.0 | 1.6 | 1.86 |
| MPO.IgG | 0.41924 | 0.604 | 9 | 16 | 146 | 57 | 2.58 |
| IF.IgM | 0.42055 | 0.643 | 9 | 19 | 51 | 32 | 1.63 |
| ZnT8.IgG | 0.42768 | 0.439 | 9 | 19 | 8.4 | 27 | 0.31 |
| ProIAA.IgM | 0.43034 | 0.550 | 9 | 19 | 595 | 692 | 0.86 |
| Thyroglobulin.IgM | 0.43188 | 0.660 | 9 | 16 | 3110 | 917 | 3.39 |
| Jo.1.IgG | 0.43850 | 0.596 | 9 | 19 | 13 | 10 | 1.39 |
| MPO.IgG | 0.44294 | 0.611 | 9 | 16 | 38 | 19 | 2.03 |
| CCP.IgG | 0.45230 | 0.403 | 9 | 16 | 26 | 19 | 1.37 |
| Sm.IgA | 0.45230 | 0.403 | 9 | 16 | 132 | 85 | 1.56 |
| TPO.IgA | 0.46870 | 0.639 | 9 | 16 | 540 | 204 | 2.65 |
| TGM2.IgM | 0.47473 | 0.694 | 9 | 16 | 1190 | 457 | 2.60 |
| Scl.70.IgM | 0.47830 | 0.424 | 9 | 16 | 2240 | 799 | 2.80 |
| aNCA.PR3.IgA | 0.48981 | 0.611 | 9 | 16 | 734 | 269 | 2.73 |
| RNP68.70.IgM | 0.49012 | 0.694 | 9 | 16 | 1230 | 369 | 3.33 |
| Ro.SSA52.IgA | 0.50499 | 0.938 | 9 | 16 | 12 | 8.0 | 1.43 |
| DGP.IgA | 0.52246 | 0.583 | 9 | 16 | 154 | 66 | 2.32 |
| U1RNPC.IgA | 0.57056 | 0.583 | 9 | 16 | 231 | 74 | 3.14 |
| Smith.IgM | 0.57549 | 0.646 | 9 | 16 | 1060 | 379 | 2.80 |
| La.SSb.IgM | 0.58913 | 0.556 | 9 | 16 | 3370 | 956 | 3.53 |
| DGP.IgM | 0.59041 | 0.438 | 9 | 16 | 1010 | 383 | 2.64 |
| MPO.IgM | 0.60999 | 0.549 | 9 | 16 | 1030 | 403 | 2.56 |
| ZnT8.IgA | 6.23E−01 | 0.439 | 9 | 19 | 146 | 217 | 0.67 |
| GAD65.IgM | 0.65049 | 0.602 | 9 | 19 | 54 | 49 | 1.10 |
| beta2glycoprotein.IgM | 0.66261 | 0.708 | 9 | 16 | 14300 | 2860 | 5.00 |
| IAA.IgA | 0.67539 | 0.456 | 9 | 19 | 162 | 191 | 0.85 |
| TGM2.IgA | 0.68990 | 0.875 | 9 | 16 | 3.1 | 4.5 | 0.68 |
| CENP.B.IgM | 0.70052 | 0.646 | 9 | 16 | 3030 | 756 | 4.01 |
| TPO.IgG | 0.70052 | 0.646 | 9 | 16 | 4.9 | 2.0 | 2.48 |
| Scl.70.IgG | 0.71823 | 0.451 | 9 | 16 | 39 | 22 | 1.73 |
| U1RNPC.IgM | 0.74136 | 0.653 | 9 | 16 | 1470 | 447 | 3.29 |
| Ro.SSA52.IgG | 0.76364 | 0.938 | 9 | 16 | 51 | 27 | 1.89 |
| TPO.IgM | 0.76364 | 0.938 | 9 | 16 | 1900 | 595 | 3.19 |
| ProIAA.IgA | 0.78924 | 0.614 | 9 | 19 | 366 | 405 | 0.90 |
| Sm.IgM | 0.79236 | 0.618 | 9 | 16 | 2520 | 682 | 3.70 |
| IF.IgG | 0.79587 | 0.760 | 9 | 19 | 0.33 | 0.39 | 0.85 |
| Smith.IgA | 0.85019 | 0.771 | 9 | 16 | 142 | 68 | 2.09 |
| MPO.IgA | 0.87130 | 0.833 | 9 | 16 | 906 | 290 | 3.12 |
| U1.RNPA.IgG | 0.87130 | 0.833 | 9 | 16 | 1.9 | 1.6 | 1.19 |
| GAD65.IgA | 0.88493 | 0.520 | 9 | 19 | 34 | 30 | 1.13 |
| GAD65.IgG | 0.88493 | 0.520 | 9 | 19 | 4.7 | 4.8 | 0.96 |
| aNCA.PR3.IgM | 0.96002 | 0.882 | 9 | 16 | 1590 | 509 | 3.12 |
| Smith.IgG | 0.97274 | 0.736 | 9 | 16 | 110 | 50 | 2.20 |
| beta2glycoprotein.IgG | 0.97787 | 0.493 | 9 | 16 | 59 | 34 | 1.76 |
| CENP.B.IgG | 0.97787 | 0.507 | 9 | 16 | 13 | 7.6 | 1.75 |
| RoSSA60.IgA | 9.78E−01 | 0.493 | 9 | 16 | 3.1 | 2.7 | 1.14 |
| CCP.IgM | 1.00000 | 0.597 | 9 | 16 | 3450 | 890 | 3.88 |
| Scl.70.IgA | 1.00000 | 0.500 | 9 | 16 | 38 | 20 | 1.94 |
| Ro.SSA52.IgM | NA | 1.000 | 9 | 16 | 1810 | 555 | 3.26 |

A summary of the biomarker significance 0 weeks after the start of treatment with alefacept is provided in Table 10.

TABLE 10

| | Mann- | | | | positive | negative | geometric |
|---|---|---|---|---|---|---|---|
| | Whitney | | positive | negative | geometric | geometric | mean |
| Biomarker | Test | AUC | count | count | mean | mean | ratio |
| beta2glycoprotein.IgA | 0.04938 | 0.743 | 9 | 16 | 6120 | 770 | 7.95 |
| IA2.IgM | 0.05260 | 0.754 | 9 | 19 | 681 | 231 | 2.95 |
| IA2.IgG | 0.05502 | 0.731 | 9 | 19 | 187 | 12 | 15.20 |
| MPO.IgA | 0.09267 | 0.708 | 9 | 16 | 542 | 173 | 3.13 |
| DGP.IgG | 0.10764 | 0.701 | 9 | 16 | 308 | 54 | 5.69 |
| TGM2.IgG | 0.10825 | 0.583 | 9 | 16 | 6.2 | 8.1 | 0.76 |
| ProIAA.IgG | 0.11220 | 0.421 | 9 | 19 | 25 | 49 | 0.51 |
| aNCA.PR3.IgG | 0.12075 | 0.938 | 9 | 16 | 104 | 39 | 2.67 |
| U1RNPC.IgG | 0.12104 | 0.306 | 9 | 16 | 178 | 116 | 1.53 |
| MPO.IgM | 0.13355 | 0.444 | 9 | 16 | 841 | 381 | 2.21 |
| Sm.IgG | 0.13564 | 0.313 | 9 | 16 | 23 | 20 | 1.17 |
| Jo.1.IgA | 0.15643 | 0.673 | 9 | 19 | 100 | 83 | 1.20 |
| Thyroglobulin.IgG | 0.18011 | 0.771 | 9 | 16 | 1280 | 307 | 4.17 |
| IA2.IgA | 0.18388 | 0.661 | 9 | 19 | 341 | 249 | 1.37 |
| IAA.IgG | 0.19967 | 0.363 | 9 | 19 | 21 | 53 | 0.39 |
| RNP68.70.IgG | 0.20715 | 0.340 | 9 | 16 | 5.9 | 7.2 | 0.83 |
| U1.RNPA.IgM | 0.21130 | 1.000 | 9 | 16 | 1450 | 453 | 3.20 |
| Jo.1.IgM | 2.52E−01 | 0.766 | 9 | 19 | 44 | 34 | 1.28 |
| IF.IgA | 0.26607 | 0.392 | 9 | 19 | 48 | 66 | 0.73 |
| RoSSA60.IgG | 0.29307 | 0.389 | 9 | 16 | 0.3 | 0.4 | 0.66 |
| RNP68.70.IgA | 0.29400 | 0.382 | 9 | 16 | 0.0000 | 0.0004 | 0.00 |
| Thyroglobulin.IgA | 3.07E−01 | 0.875 | 9 | 16 | 3050 | 898 | 3.40 |
| IAA.IgM | 0.32287 | 0.404 | 9 | 19 | 181 | 261 | 0.69 |
| ZnT8.IgM | 0.32627 | 0.456 | 9 | 19 | 260 | 395 | 0.66 |
| CCP.IgA | 0.33574 | 0.382 | 9 | 16 | 55 | 42 | 1.32 |
| RoSSA60.IgM | 0.37605 | 0.729 | 9 | 16 | 1820 | 476 | 3.82 |
| U1.RNPA.IgA | 0.39293 | 0.799 | 9 | 16 | 227 | 91 | 2.49 |
| La.SSb.IgG | 0.41134 | 0.611 | 9 | 16 | 1.3 | 0.8 | 1.63 |
| CENP.B.IgA | 0.41924 | 0.604 | 9 | 16 | 35 | 18 | 1.98 |
| La.SSb.IgA | 4.19E−01 | 0.604 | 9 | 16 | 3.0 | 1.6 | 1.86 |
| MPO.IgG | 0.41924 | 0.604 | 9 | 16 | 146 | 57 | 2.58 |
| IF.IgM | 0.42055 | 0.643 | 9 | 19 | 51 | 32 | 1.63 |
| ZnT8.IgG | 0.42768 | 0.439 | 9 | 19 | 8.4 | 27 | 0.31 |
| ProIAA.IgM | 0.43034 | 0.550 | 9 | 19 | 595 | 692 | 0.86 |
| Thyroglobulin.IgM | 0.43188 | 0.660 | 9 | 16 | 3110 | 917 | 3.39 |
| Jo.1.IgG | 0.43850 | 0.596 | 9 | 19 | 13 | 10 | 1.39 |
| MPO.IgG | 0.44294 | 0.611 | 9 | 16 | 38 | 19 | 2.03 |
| CCP.IgG | 0.45230 | 0.403 | 9 | 16 | 26 | 19 | 1.37 |
| Sm.IgA | 0.45230 | 0.403 | 9 | 16 | 132 | 85 | 1.56 |
| TPO.IgA | 0.46870 | 0.639 | 9 | 16 | 540 | 204 | 2.65 |
| TGM2.IgM | 0.47473 | 0.694 | 9 | 16 | 1190 | 457 | 2.60 |
| Scl.70.IgM | 0.47830 | 0.424 | 9 | 16 | 2240 | 799 | 2.80 |
| aNCA.PR3.IgA | 0.48981 | 0.611 | 9 | 16 | 734 | 269 | 2.73 |
| RNP68.70.IgM | 0.49012 | 0.694 | 9 | 16 | 1230 | 369 | 3.33 |
| Ro.SSA52.IgA | 0.50499 | 0.938 | 9 | 16 | 12 | 8.0 | 1.43 |
| DGP.IgA | 0.52246 | 0.583 | 9 | 16 | 154 | 66 | 2.32 |
| U1RNPC.IgA | 0.57056 | 0.583 | 9 | 16 | 231 | 74 | 3.14 |
| Smith.IgM | 0.57549 | 0.646 | 9 | 16 | 1060 | 379 | 2.80 |
| La.SSb.IgM | 0.58913 | 0.556 | 9 | 16 | 3370 | 956 | 3.53 |
| DGP.IgM | 0.59041 | 0.438 | 9 | 16 | 1010 | 383 | 2.64 |
| MPO.IgM | 0.60999 | 0.549 | 9 | 16 | 1030 | 403 | 2.56 |
| ZnT8.IgA | 6.23E−01 | 0.439 | 9 | 19 | 146 | 217 | 0.67 |
| GAD65.IgM | 0.65049 | 0.602 | 9 | 19 | 54 | 49 | 1.10 |
| beta2glycoprotein.IgM | 0.66261 | 0.708 | 9 | 16 | 14300 | 2860 | 5.00 |
| IAA.IgA | 0.67539 | 0.456 | 9 | 19 | 162 | 191 | 0.85 |
| TGM2.IgA | 0.68990 | 0.875 | 9 | 16 | 3.1 | 4.5 | 0.68 |
| CENP.B.IgM | 0.70052 | 0.646 | 9 | 16 | 3030 | 756 | 4.01 |
| TPO.IgG | 0.70052 | 0.646 | 9 | 16 | 4.9 | 2.0 | 2.48 |
| Scl.70.IgG | 0.71823 | 0.451 | 9 | 16 | 39 | 22 | 1.73 |
| U1RNPC.IgM | 0.74136 | 0.653 | 9 | 16 | 1470 | 447 | 3.29 |
| Ro.SSA52.IgG | 0.76364 | 0.938 | 9 | 16 | 51 | 27 | 1.89 |
| TPO.IgM | 0.76364 | 0.938 | 9 | 16 | 1900 | 595 | 3.19 |
| ProIAA.IgA | 0.78924 | 0.614 | 9 | 19 | 366 | 405 | 0.90 |
| Sm.IgM | 0.79236 | 0.618 | 9 | 16 | 2520 | 682 | 3.70 |
| IF.IgG | 0.79587 | 0.760 | 9 | 19 | 0.33 | 0.39 | 0.85 |
| Smith.IgA | 0.85019 | 0.771 | 9 | 16 | 142 | 68 | 2.09 |
| MPO.IgA | 0.87130 | 0.833 | 9 | 16 | 906 | 290 | 3.12 |
| U1.RNPA.IgG | 0.87130 | 0.833 | 9 | 16 | 1.9 | 1.6 | 1.19 |
| GAD65.IgA | 0.88493 | 0.520 | 9 | 19 | 34 | 30 | 1.13 |
| GAD65.IgG | 0.88493 | 0.520 | 9 | 19 | 4.7 | 4.8 | 0.96 |
| aNCA.PR3.IgM | 0.96002 | 0.882 | 9 | 16 | 1590 | 509 | 3.12 |
| Smith.IgG | 0.97274 | 0.736 | 9 | 16 | 110 | 50 | 2.20 |
| beta2glycoprotein.IgG | 0.97787 | 0.493 | 9 | 16 | 59 | 34 | 1.76 |
| CENP.B.IgG | 0.97787 | 0.507 | 9 | 16 | 13 | 7.6 | 1.75 |
| RoSSA60.IgA | 9.78E−01 | 0.493 | 9 | 16 | 3.1 | 2.7 | 1.14 |
| CCP.IgM | 1.00000 | 0.597 | 9 | 16 | 3450 | 890 | 3.88 |
| Scl.70.IgA | 1.00000 | 0.500 | 9 | 16 | 38 | 20 | 1.94 |
| Ro.SSA52.IgM | NA | 1.000 | 9 | 16 | 1810 | 555 | 3.26 |

A summary of the biomarker significance 11 weeks after the start of treatment with alefacept is provided in Table 11.

TABLE 11

| | Mann- | | | | positive | negative | geometric |
|---|---|---|---|---|---|---|---|
| | Whitney | | positive | negative | geometric | geometric | mean |
| Biomarker | Test | AUC | count | count | mean | mean | ratio |
| IA2.IgG | 0.03410 | 0.750 | 9 | 20 | 126 | 10 | 12.12 |
| ProIAA.IgG | 0.03608 | 0.294 | 9 | 20 | 29 | 67 | 0.43 |
| TGM2.IgG | 0.06202 | 0.361 | 9 | 20 | 7 | 13 | 0.52 |
| IA2.IgM | 0.08070 | 0.728 | 9 | 20 | 548 | 232 | 2.36 |
| DGP.IgG | 0.08540 | 0.706 | 9 | 20 | 263 | 76 | 3.48 |
| IF.IgM | 0.09875 | 0.733 | 9 | 20 | 79 | 31 | 2.59 |
| MPO.IgM | 0.10134 | 0.606 | 9 | 20 | 846 | 1180 | 0.72 |
| Smith.IgG | 0.11522 | 0.750 | 9 | 20 | 86 | 101 | 0.85 |
| beta2glycoprotein.IgA | 0.11554 | 0.689 | 9 | 20 | 5730.0 | 3190.0 | 1.80 |
| Sm.IgG | 0.11554 | 0.311 | 9 | 20 | 23 | 38 | 0.60 |
| RNP68.70.IgG | 0.12719 | 0.317 | 9 | 20 | 6 | 12 | 0.51 |
| IAA.IgG | 0.13969 | 0.322 | 9 | 20 | 36.3 | 107.0 | 0.34 |
| RNP68.70.IgA | 0.15045 | 0.328 | 9 | 20 | 0 | 0 | 0.00 |
| U1.RNPA.IgM | 0.15672 | 1.000 | 9 | 20 | 1440 | 1430 | 1.01 |
| Sm.IgA | 1.83E−01 | 0.339 | 9 | 20 | 155 | 359 | 0.43 |
| CCP.IgA | 0.19882 | 0.344 | 9 | 20 | 65 | 154 | 0.42 |
| Thyroglobulin.IgG | 0.21105 | 0.794 | 9 | 20 | 1100 | 856 | 1.29 |
| MPO.IgG | 2.16E−01 | 0.650 | 9 | 20 | 160 | 138 | 1.16 |
| IA2.IgA | 0.23853 | 0.644 | 9 | 20 | 308 | 237 | 1.30 |
| MPO.IgM | 0.25330 | 0.522 | 9 | 20 | 1030 | 1260 | 0.82 |
| RoSSA60.IgA | 0.27360 | 0.367 | 9 | 20 | 2.8 | 5.0 | 0.56 |
| Jo.1.IgM | 0.27840 | 0.806 | 9 | 20 | 37 | 31 | 1.22 |
| IAA.IgM | 0.32160 | 0.389 | 9 | 20 | 240 | 366 | 0.66 |
| U1.RNPA.IgA | 0.35768 | 0.800 | 9 | 20 | 227 | 234 | 0.97 |
| ZnT8.IgM | 0.36070 | 0.506 | 9 | 20 | 218 | 415 | 0.53 |
| Ro.SSA52.IgA | 0.36185 | 0.900 | 9 | 20 | 12 | 13 | 0.86 |
| Thyroglobulin.IgM | 0.36265 | 0.728 | 9 | 20 | 3090 | 3240 | 0.95 |
| La.SSb.IgA | 0.36480 | 0.611 | 9 | 20 | 2.8 | 1.9 | 1.45 |
| U1RNPC.IgG | 0.36480 | 0.389 | 9 | 20 | 238 | 311 | 0.77 |
| MPO.IgA | 0.37985 | 0.622 | 9 | 20 | 564 | 469 | 1.20 |
| Scl.70.IgG | 0.39015 | 0.394 | 9 | 20 | 42.0000 | 51.1000 | 0.82 |
| RoSSA60.IgG | 0.43645 | 0.411 | 9 | 20 | 0 | 0 | 0.79 |
| aNCA.PR3.IgG | 0.45518 | 0.906 | 9 | 20 | 104 | 89 | 1.17 |
| La.SSb.IgG | 0.46476 | 0.594 | 9 | 20 | 1 | 1 | 1.78 |
| GAD65.IgG | 0.47216 | 0.589 | 9 | 20 | 3 | 3 | 1.13 |
| CCP.IgM | 0.48777 | 0.606 | 9 | 20 | 3650 | 3480 | 1.05 |
| MPO.IgA | 0.50976 | 0.828 | 9 | 20 | 874 | 795 | 1.10 |
| beta2glycoprotein.IgG | 0.53152 | 0.422 | 9 | 20 | 48.4 | 63 | 0.76 |
| ZnT8.IgA | 0.53152 | 0.422 | 9 | 20 | 138 | 215 | 0.64 |
| IAA.IgA | 0.53949 | 0.433 | 9 | 20 | 201 | 286 | 0.70 |
| TGM2.IgA | 0.54280 | 0.806 | 9 | 20 | 3 | 4 | 0.72 |
| La.SSb.IgM | 0.58748 | 0.606 | 9 | 20 | 3660 | 3500 | 1.05 |
| GAD65.IgA | 0.59429 | 0.567 | 9 | 20 | 27 | 27 | 1.00 |
| aNCA.PR3.IgM | 0.62928 | 0.950 | 9 | 20 | 1550 | 1610 | 0.96 |
| TPO.IgM | 0.62928 | 0.950 | 9 | 20 | 1790 | 1910 | 0.94 |
| Scl.70.IgM | 0.63694 | 0.450 | 9 | 20 | 2550.0 | 3010.0 | 0.85 |
| IF.IgA | 0.65287 | 0.467 | 9 | 20 | 59 | 71 | 0.82 |
| IF.IgG | 0.65360 | 0.794 | 9 | 20 | 0 | 0 | 0.93 |
| CCP.IgG | 0.66012 | 0.444 | 9 | 20 | 29 | 37 | 0.76 |
| U1RNPC.IgA | 0.72354 | 0.550 | 9 | 20 | 260 | 212 | 1.23 |
| Smith.IgA | 0.76636 | 0.667 | 9 | 20 | 149 | 173 | 0.86 |
| U1.RNPA.IgG | 0.77458 | 0.856 | 9 | 20 | 1.8 | 2.0 | 0.91 |
| GAD65.IgM | 0.78582 | 0.617 | 9 | 20 | 45 | 43 | 1.04 |
| TPO.IgG | 0.78799 | 0.622 | 9 | 20 | 5 | 3 | 1.67 |
| ProIAA.IgM | 0.78978 | 0.544 | 9 | 20 | 692 | 713 | 0.97 |
| ZnT8.IgG | 7.94E−01 | 0.494 | 9 | 20 | 9 | 18 | 0.53 |
| aNCA.PR3.IgA | 0.82405 | 0.572 | 9 | 20 | 697 | 805 | 0.87 |
| RoSSA60.IgM | 0.82405 | 0.628 | 9 | 20 | 1790 | 1610 | 1.11 |
| CENP.B.IgM | 0.82733 | 0.594 | 9 | 20 | 3130.0 | 3050.0 | 1.03 |
| Jo.1.IgG | 0.83515 | 0.528 | 9 | 20 | 9 | 8 | 1.17 |
| Smith.IgM | 0.83627 | 0.628 | 9 | 20 | 1120 | 1200 | 0.93 |
| TGM2.IgM | 0.85650 | 0.656 | 9 | 20 | 1390 | 1450 | 0.96 |
| RNP68.70.IgM | 0.86271 | 0.622 | 9 | 20 | 1160 | 1150 | 1.01 |
| MPO.IgG | 8.69E−01 | 0.483 | 9 | 20 | 29 | 29 | 1.01 |
| CENP.B.IgG | 0.87143 | 0.478 | 9 | 20 | 14 | 13 | 1.14 |
| DGP.IgA | 0.87143 | 0.522 | 9 | 20 | 140 | 145 | 0.97 |
| TPO.IgA | 0.88070 | 0.611 | 9 | 20 | 444 | 581 | 0.76 |
| CENP.B.IgA | 0.90797 | 0.483 | 9 | 20 | 45 | 59 | 0.76 |
| Scl.70.IgA | 0.90797 | 0.517 | 9 | 20 | 46.6 | 45.0 | 1.04 |
| beta2glycoprotein.IgM | 0.91770 | 0.667 | 9 | 20 | 14000.0 | 13000.0 | 1.08 |
| Thyroglobulin.IgA | 0.92046 | 0.822 | 9 | 20 | 3310 | 3530 | 0.94 |
| U1RNPC.IgM | 0.94018 | 0.622 | 9 | 20 | 1480 | 1450 | 1.02 |
| ProIAA.IgA | 0.95880 | 0.661 | 9 | 20 | 377 | 440 | 0.86 |
| Ro.SSA52.IgG | 0.96444 | 0.906 | 9 | 20 | 51 | 51 | 1.00 |
| DGP.IgM | 0.98115 | 0.522 | 9 | 20 | 1150 | 1150 | 1.00 |
| Jo.1.IgA | 0.98156 | 0.506 | 9 | 20 | 91 | 84 | 1.08 |
| Sm.IgM | 1.00E+00 | 0.556 | 9 | 20 | 2510 | 2540 | 0.99 |
| Ro.SSA52.IgM | NA | 1.000 | 9 | 20 | 1810 | 1810 | 1.00 |

A summary of the biomarker significance 26 weeks after the start of treatment with alefacept is provided in Table 12.

TABLE 12

| | Mann- | | | | positive | negative | geometric |
|---|---|---|---|---|---|---|---|
| | Whitney | | positive | negative | geometric | geometric | mean |
| Biomarker | Test | AUC | count | count | mean | mean | ratio |
| IA2.IgG | 0.04502 | 0.739 | 9 | 20 | 79 | 6.8 | 11.72 |
| MPO.IgM | 0.05410 | 0.417 | 9 | 20 | 836 | 1180 | 0.71 |
| beta2glycoprotein.IgA | 0.06174 | 0.722 | 9 | 20 | 5430 | 2560 | 2.12 |
| Smith.IgG | 0.10134 | 0.606 | 9 | 20 | 90 | 118 | 0.76 |
| IA2.IgA | 0.13969 | 0.678 | 9 | 20 | 285 | 211 | 1.35 |
| ZnT8.IgM | 1.45E−01 | 0.450 | 9 | 20 | 159 | 315 | 0.50 |
| RNP68.70.IgG | 0.15308 | 0.328 | 9 | 20 | 6.1 | 11 | 0.54 |
| U1.RNPA.IgM | 0.15672 | 1.000 | 9 | 20 | 1500 | 1430 | 1.05 |
| La.SSb.IgG | 0.19463 | 0.661 | 9 | 20 | 1.4 | 0.6 | 2.54 |
| RoSSA60.IgG | 0.19463 | 0.344 | 9 | 20 | 0.3 | 0.3 | 0.95 |
| IF.IgG | 0.22086 | 0.700 | 9 | 20 | 0.2 | 0.3 | 0.74 |
| Scl.70.IgM | 2.39E−01 | 0.361 | 9 | 20 | 2570 | 3300 | 0.78 |
| DGP.IgG | 0.25337 | 0.639 | 9 | 20 | 219 | 127 | 1.72 |
| TGM2.IgG | 0.26078 | 0.422 | 9 | 20 | 9.5 | 14 | 0.69 |
| CCP.IgA | 2.95E−01 | 0.372 | 9 | 20 | 77 | 128 | 0.60 |
| La.SSb.IgA | 0.29484 | 0.628 | 9 | 20 | 2.5 | 1.5 | 1.69 |
| IA2.IgM | 0.29734 | 0.689 | 9 | 20 | 412 | 217 | 1.90 |
| MPO.IgG | 0.31713 | 0.622 | 9 | 20 | 139 | 117 | 1.19 |
| Thyroglobulin.IgG | 0.32842 | 0.806 | 9 | 20 | 1150 | 755 | 1.52 |
| Sm.IgA | 0.34572 | 0.389 | 9 | 20 | 185 | 296 | 0.63 |
| Thyroglobulin.IgA | 0.36185 | 0.900 | 9 | 20 | 3050 | 3110 | 0.98 |
| Thyroglobulin.IgM | 0.36265 | 0.728 | 9 | 20 | 3080 | 3170 | 0.97 |
| IAA.IgA | 0.38292 | 0.394 | 9 | 20 | 188 | 257 | 0.73 |
| IAA.IgM | 0.38310 | 0.394 | 9 | 20 | 281 | 386 | 0.73 |
| MPO.IgA | 0.38892 | 0.783 | 9 | 20 | 1050 | 832 | 1.26 |
| Smith.IgA | 0.38892 | 0.644 | 9 | 20 | 143 | 164 | 0.87 |
| Sm.IgG | 0.39015 | 0.394 | 9 | 20 | 26 | 35 | 0.73 |
| TGM2.IgM | 0.39395 | 0.572 | 9 | 20 | 1320 | 1560 | 0.85 |
| aNCA.PR3.IgG | 0.40914 | 0.911 | 9 | 20 | 130 | 72 | 1.80 |
| IAA.IgG | 0.40916 | 0.400 | 9 | 20 | 40 | 94 | 0.43 |
| RNP68.70.IgA | 0.40916 | 0.406 | 9 | 20 | 0.0001 | 0.0002 | 0.50 |
| ZnT8.IgG | 0.42090 | 0.428 | 9 | 20 | 4.1 | 11 | 0.39 |
| CENP.B.IgM | 0.42712 | 0.639 | 9 | 20 | 3320 | 3090 | 1.07 |
| MPO.IgM | 0.44379 | 0.511 | 9 | 20 | 1050 | 1340 | 0.78 |
| Jo.1.IgA | 0.44386 | 0.594 | 9 | 20 | 94 | 85 | 1.11 |
| U1RNPC.IgA | 0.46476 | 0.594 | 9 | 20 | 261 | 187 | 1.40 |
| IF.IgA | 0.47490 | 0.456 | 9 | 20 | 49 | 61 | 0.80 |
| Scl.70.IgA | 0.50139 | 0.583 | 9 | 20 | 51 | 35 | 1.45 |
| U1.RNPA.IgA | 0.52267 | 0.761 | 9 | 20 | 222 | 218 | 1.02 |
| ProIAA.IgM | 0.52502 | 0.522 | 9 | 20 | 603 | 671 | 0.90 |
| La.SSb.IgM | 0.53685 | 0.539 | 9 | 20 | 3500 | 3640 | 0.96 |
| Ro.SSA52.IgA | 0.55098 | 0.950 | 9 | 20 | 12 | 12 | 0.97 |
| RoSSA60.IgA | 0.58756 | 0.439 | 9 | 20 | 2.9 | 4.2 | 0.69 |
| DGP.IgA | 0.59429 | 0.567 | 9 | 20 | 172 | 167 | 1.03 |
| RNP68.70.IgM | 0.60768 | 0.528 | 9 | 20 | 1160 | 1240 | 0.94 |
| U1.RNPA.IgG | 0.61103 | 0.800 | 9 | 20 | 2.0 | 1.7 | 1.20 |
| beta2glycoprotein.IgG | 0.62684 | 0.561 | 9 | 20 | 56 | 56 | 1.01 |
| U1RNPC.IgG | 0.62684 | 0.439 | 9 | 20 | 219 | 256 | 0.86 |
| Ro.SSA52.IgG | 0.62928 | 0.950 | 9 | 20 | 53 | 50 | 1.07 |
| TPO.IgM | 0.62928 | 0.950 | 9 | 20 | 1900 | 1890 | 1.01 |
| TPO.IgG | 0.62957 | 0.678 | 9 | 20 | 5.1 | 1.7 | 3.04 |
| DGP.IgM | 0.68828 | 0.461 | 9 | 20 | 1110 | 1240 | 0.90 |
| GAD65.IgG | 0.69405 | 0.550 | 9 | 20 | 2.9 | 3.1 | 0.94 |
| MPO.IgA | 0.70487 | 0.561 | 9 | 20 | 559 | 519 | 1.08 |
| ProIAA.IgA | 7.09E−01 | 0.772 | 9 | 20 | 408 | 361 | 1.13 |
| ProIAA.IgG | 0.73619 | 0.517 | 9 | 20 | 32 | 51 | 0.63 |
| MPO.IgG | 7.39E−01 | 0.583 | 9 | 20 | 31 | 27 | 1.13 |
| CCP.IgG | 0.76365 | 0.461 | 9 | 20 | 31 | 34 | 0.90 |
| CENP.B.IgA | 0.76365 | 0.461 | 9 | 20 | 39 | 45 | 0.85 |
| aNCA.PR3.IgA | 0.78006 | 0.656 | 9 | 20 | 736 | 716 | 1.03 |
| TGM2.IgA | 0.78321 | 0.856 | 9 | 20 | 2.9 | 5.3 | 0.56 |
| beta2glycoprotein.IgM | 0.78978 | 0.544 | 9 | 20 | 14100 | 14600 | 0.97 |
| ZnT8.IgA | 0.79533 | 0.467 | 9 | 20 | 123 | 169 | 0.73 |
| RoSSA60.IgM | 0.82733 | 0.606 | 9 | 20 | 1850 | 1680 | 1.10 |
| TPO.IgA | 0.82733 | 0.594 | 9 | 20 | 480 | 494 | 0.97 |
| IF.IgM | 8.31E−01 | 0.500 | 9 | 20 | 54 | 47 | 1.16 |
| CENP.B.IgG | 0.83515 | 0.528 | 9 | 20 | 15 | 11 | 1.31 |
| GAD65.IgA | 0.87143 | 0.522 | 9 | 20 | 25 | 25 | 0.96 |
| Jo.1.IgM | 0.88948 | 0.700 | 9 | 20 | 32 | 34 | 0.96 |
| U1RNPC.IgM | 0.92332 | 0.544 | 9 | 20 | 1500 | 1560 | 0.96 |
| Sm.IgM | 0.94278 | 0.539 | 9 | 20 | 2590 | 2610 | 0.99 |
| Scl.70.IgG | 0.94471 | 0.511 | 9 | 20 | 41 | 40 | 1.02 |
| Smith.IgM | 0.95880 | 0.644 | 9 | 20 | 1170 | 1200 | 0.98 |
| GAD65.IgM | 0.96100 | 0.567 | 9 | 20 | 39 | 50 | 0.78 |
| aNCA.PR3.IgM | 0.96444 | 0.906 | 9 | 20 | 1580 | 1610 | 0.98 |
| CCP.IgM | 1.00000 | 0.544 | 9 | 20 | 3860 | 3770 | 1.02 |
| Jo.1.IgG | 1.00000 | 0.500 | 9 | 20 | 7.7 | 6.9 | 1.11 |
| Ro.SSA52.IgM | NA | 1.000 | 9 | 20 | 1810 | 1810 | 1.00 |

A summary of the biomarker significance 30 weeks after the start of treatment with alefacept is provided in Table 13.

TABLE 13

| | Mann- | | | | positive | negative | geometric |
|---|---|---|---|---|---|---|---|
| | Whitney | | positive | negative | geometric | geometric | mean |
| Biomarker | Test | AUC | count | count | mean | mean | ratio |
| ZnT8.IgM | 0.01153 | 0.350 | 9 | 20 | 109 | 264 | 0.41 |
| beta2glycoprotein.IgA | 0.02307 | 0.767 | 9 | 20 | 5790 | 2640 | 2.19 |
| IA2.IgG | 0.06896 | 0.717 | 9 | 20 | 31 | 5.4 | 5.69 |
| U1.RNPA.IgA | 7.08E−02 | 0.850 | 9 | 20 | 307 | 217 | 1.41 |
| MPO.IgG | 0.08540 | 0.706 | 9 | 20 | 166 | 131 | 1.27 |
| TPO.IgA | 0.13172 | 0.700 | 9 | 20 | 620 | 501 | 1.24 |
| aNCA.PR3.IgG | 0.14217 | 0.917 | 9 | 20 | 191 | 73 | 2.62 |
| TGM2.IgG | 0.15538 | 0.511 | 9 | 20 | 6.6 | 13 | 0.51 |
| U1.RNPA.IgG | 0.15969 | 0.856 | 9 | 20 | 2.7 | 1.7 | 1.58 |
| aNCA.PR3.IgA | 0.17424 | 0.717 | 9 | 20 | 863 | 717 | 1.20 |
| Ro.SSA52.IgG | 0.21195 | 0.950 | 9 | 20 | 57 | 50 | 1.14 |
| TPO.IgM | 0.21195 | 0.950 | 9 | 20 | 2110 | 1940 | 1.09 |
| RNP68.70.IgG | 0.21600 | 0.350 | 9 | 20 | 6.6 | 11 | 0.62 |
| U1RNPC.IgA | 0.25784 | 0.639 | 9 | 20 | 274 | 184 | 1.49 |
| IA2.IgM | 0.27128 | 0.683 | 9 | 20 | 327 | 205 | 1.60 |
| Smith.IgG | 0.27191 | 0.711 | 9 | 20 | 90 | 111 | 0.81 |
| La.SSb.IgG | 0.28861 | 0.633 | 9 | 20 | 1.3 | 0.6 | 2.26 |
| Thyroglobulin.IgG | 0.30077 | 0.811 | 9 | 20 | 1530 | 895 | 1.71 |
| RNP68.70.IgM | 0.33533 | 0.500 | 9 | 20 | 1120 | 1200 | 0.93 |
| DGP.IgG | 0.34045 | 0.617 | 9 | 20 | 170 | 113 | 1.50 |
| La.SSb.IgA | 0.34045 | 0.617 | 9 | 20 | 2.1 | 1.3 | 1.61 |
| MPO.IgG | 0.34572 | 0.617 | 9 | 20 | 38 | 33 | 1.14 |
| RoSSA60.IgG | 0.35738 | 0.400 | 9 | 20 | 0.28 | 0.30 | 0.91 |
| MPO.IgA | 3.97E−01 | 0.844 | 9 | 20 | 972 | 812 | 1.20 |
| IF.IgM | 0.40449 | 0.633 | 9 | 20 | 49 | 32 | 1.55 |
| Scl.70.IgM | 0.40824 | 0.417 | 9 | 20 | 2330 | 2910 | 0.80 |
| MPO.IgM | 0.41369 | 0.594 | 9 | 20 | 882 | 1090 | 0.81 |
| Sm.IgG | 0.41652 | 0.400 | 9 | 20 | 26 | 38 | 0.70 |
| ProIAA.IgA | 4.18E−01 | 0.767 | 9 | 20 | 532 | 377 | 1.41 |
| MPO.IgM | 0.43835 | 0.567 | 9 | 20 | 995 | 1230 | 0.81 |
| IA2.IgA | 0.44386 | 0.594 | 9 | 20 | 241 | 212 | 1.14 |
| Scl.70.IgA | 0.44386 | 0.594 | 9 | 20 | 45 | 29 | 1.57 |
| IAA.IgM | 0.46387 | 0.428 | 9 | 20 | 297 | 383 | 0.78 |
| beta2glycoprotein.IgG | 0.47216 | 0.589 | 9 | 20 | 63 | 51 | 1.25 |
| Smith.IgA | 0.48717 | 0.767 | 9 | 20 | 182 | 159 | 1.14 |
| TPO.IgG | 0.49365 | 0.656 | 9 | 20 | 11 | 1.9 | 5.63 |
| ProIAA.IgM | 0.49944 | 0.556 | 9 | 20 | 698 | 700 | 1.00 |
| U1RNPC.IgM | 0.51586 | 0.489 | 9 | 20 | 1460 | 1570 | 0.93 |
| TGM2.IgA | 0.54280 | 0.806 | 9 | 20 | 2.9 | 5.3 | 0.54 |
| Thyroglobulin.IgA | 0.55098 | 0.950 | 9 | 20 | 3050 | 3190 | 0.96 |
| Jo.1.IgG | 0.56249 | 0.572 | 9 | 20 | 6.9 | 5.7 | 1.23 |
| Scl.70.IgG | 0.56249 | 0.572 | 9 | 20 | 41 | 35 | 1.19 |
| La.SSb.IgM | 0.56991 | 0.544 | 9 | 20 | 3390 | 3450 | 0.98 |
| ZnT8.IgG | 5.85E−01 | 0.461 | 9 | 20 | 2.4 | 6.8 | 0.36 |
| RNP68.70.IgA | 0.58682 | 0.450 | 9 | 20 | 0.0001 | 0.0001 | 1.00 |
| MPO.IgA | 0.61910 | 0.583 | 9 | 20 | 526 | 463 | 1.14 |
| IAA.IgG | 0.62045 | 0.439 | 9 | 20 | 90 | 132 | 0.68 |
| CCP.IgG | 6.37E−01 | 0.444 | 9 | 20 | 30 | 34 | 0.89 |
| Thyroglobulin.IgM | 0.64165 | 0.817 | 9 | 20 | 3070 | 3030 | 1.01 |
| CCP.IgA | 0.66012 | 0.444 | 9 | 20 | 87 | 120 | 0.72 |
| RoSSA60.IgA | 0.67134 | 0.450 | 9 | 20 | 3.2 | 4.7 | 0.69 |
| Sm.IgM | 0.68247 | 0.517 | 9 | 20 | 2560 | 2660 | 0.96 |
| ProIAA.IgG | 0.68419 | 0.506 | 9 | 20 | 74 | 73 | 1.01 |
| IAA.IgA | 0.68861 | 0.550 | 9 | 20 | 261 | 258 | 1.01 |
| CENP.B.IgG | 0.69405 | 0.550 | 9 | 20 | 13 | 10 | 1.34 |
| Jo.1.IgA | 0.69405 | 0.550 | 9 | 20 | 88 | 84 | 1.05 |
| CENP.B.IgM | 0.71384 | 0.544 | 9 | 20 | 2930 | 3040 | 0.96 |
| GAD65.IgG | 0.76365 | 0.539 | 9 | 20 | 1.9 | 2.3 | 0.85 |
| GAD65.IgA | 0.77727 | 0.539 | 9 | 20 | 23 | 23 | 0.98 |
| DGP.IgM | 0.79518 | 0.478 | 9 | 20 | 996 | 1090 | 0.91 |
| TGM2.IgM | 0.80248 | 0.689 | 9 | 20 | 1300 | 1410 | 0.92 |
| ZnT8.IgA | 0.83196 | 0.472 | 9 | 20 | 117 | 138 | 0.85 |
| CENP.B.IgA | 0.83515 | 0.528 | 9 | 20 | 45 | 44 | 1.03 |
| DGP.IgA | 8.35E−01 | 0.528 | 9 | 20 | 131 | 147 | 0.89 |
| Smith.IgM | 0.85356 | 0.644 | 9 | 20 | 1130 | 1220 | 0.93 |
| U1RNPC.IgG | 0.87143 | 0.478 | 9 | 20 | 238 | 276 | 0.86 |
| IF.IgA | 0.88705 | 0.533 | 9 | 20 | 59 | 62 | 0.95 |
| CCP.IgM | 0.90355 | 0.561 | 9 | 20 | 3660 | 3830 | 0.96 |
| GAD65.IgM | 0.90423 | 0.561 | 9 | 20 | 33 | 41 | 0.80 |
| beta2glycoprotein.IgM | 0.94092 | 0.600 | 9 | 20 | 14500 | 14000 | 1.04 |
| Jo.1.IgM | 0.97896 | 0.672 | 9 | 20 | 34 | 34 | 0.98 |
| RoSSA60.IgM | 0.98029 | 0.594 | 9 | 20 | 1720 | 1720 | 1.00 |
| Sm.IgA | 0.98156 | 0.506 | 9 | 20 | 305 | 275 | 1.11 |
| aNCA.PR3.IgM | 1.00000 | 0.900 | 9 | 20 | 1630 | 1660 | 0.98 |
| IF.IgG | 1.00000 | 0.900 | 9 | 20 | 0.2 | 0.3 | 0.83 |
| Ro.SSA52.IgA | NA | 1.000 | 9 | 20 | 12 | 12 | 1.00 |
| Ro.SSA52.IgM | NA | 1.000 | 9 | 20 | 1810 | 1810 | 1.00 |
| U1.RNPA.IgM | NA | 1.000 | 9 | 20 | 1430 | 1430 | 1.00 |

Key results of the comparison of treated subjects for positive vs. negative outcomes are summarized as follows:
Positive outcome of Alefacept treatment correlates with higher levels of anti-IA2 IgG and anti-beta2glycoprotein IgA levels at 0 weeks and other time points.
Higher anti-DGP IgG, anti-IA2 IgM, and anti-MPO IgA at 0 weeks correlates with positive outcome of Alefacept treatment.
Lower proinsulin IgG at 11 weeks, anti-MPO IgM at 26 weeks, anti-ZnT8 IgM at 30 weeks correlate with better outcome.

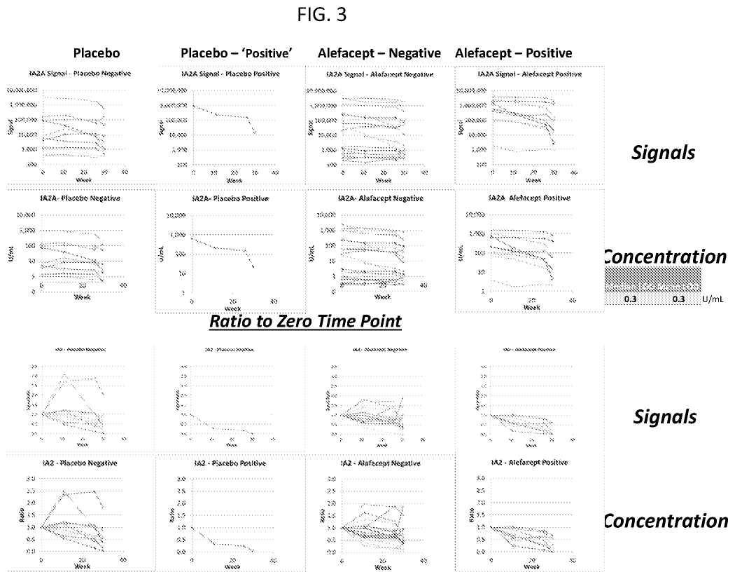

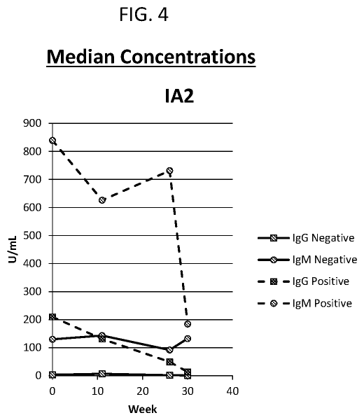

Results obtained for anti-IA2 IgG are shown in FIG. 3. Each plotted line represents an individual patient at all available time points. The graphs are sorted based on treatment group (Placebo or Alefacept), and treatment outcome (Positive or Negative). The data are shown in terms of sample signals and calculated concentrations (top half). The ratios of signals or concentrations to each patient's 0 time point sample values are shown as well (bottom half). As can be seen from the results: 1) The treatment response group shows less elevation of autoimmunity at earlier time points relative to treatment negative and placebo groups. 2) There are overall higher signals in the positive response group. Median values for all treated patients in the positive and negative response groups are summarized in FIG. 4: higher levels of anti-IA2 IgG are seen in the positive response group relative to negative response group at earlier time points.

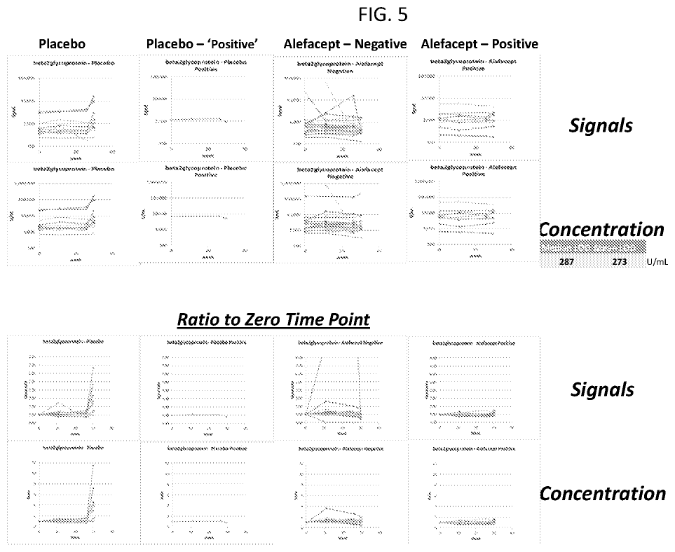

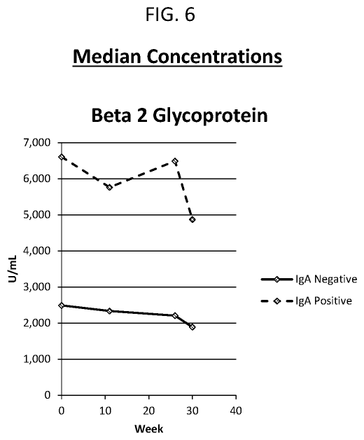

Results obtained for anti-beta2glycoprotein IgA are shown in FIG. 5. Each plotted line represents an individual patient at all available time points. The graphs are sorted based on treatment group (Placebo or Alefacept), and treatment outcome (Positive or Negative). The data are shown in terms of sample signals and calculated concentrations (top half). The ratios of signals or concentrations to each patient's 0 time point sample values are shown as well (bottom half). As can be seen from the results: 1) Alefacept treatment prevents elevation of reactivity at later time points. 2) The treatment response group tends to have higher levels of reactivity the negative response group (See median levels in FIG. 6)) More changes are seen in treatment negative group.

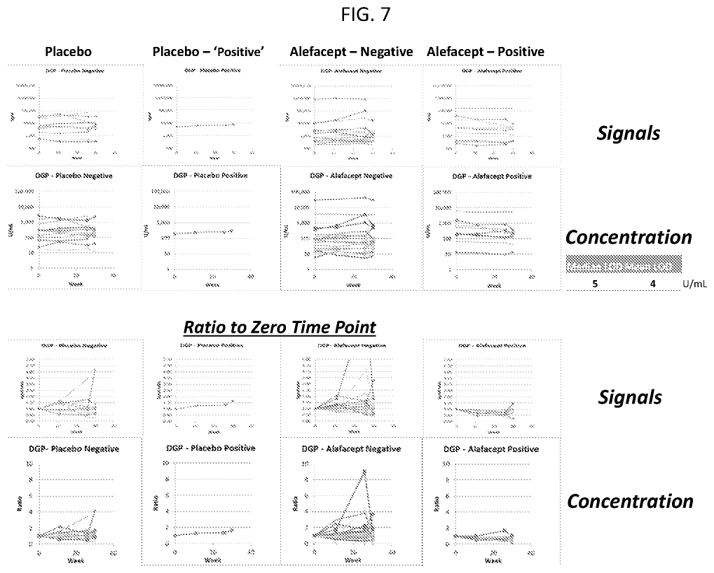

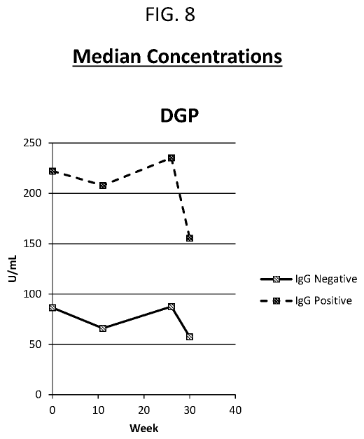

Results obtained for anti-DGP IgG are shown in FIG. 7 and FIG. 8. In FIG. 7, each plotted line represents an individual patient at all available time points. The graphs are sorted based on treatment group (Placebo or Alefacept), and treatment outcome (Positive or Negative). The data are shown in terms of sample signals and calculated concentrations (top half). The ratios of signals or concentrations to each patient's 0 time point sample values are shown as well (bottom half). FIG. 8 shows median concentration values at each time point for the positive and negative response groups. As can be seen from the results: 1) Alefacept treatment positive response group tends to have higher concentrations than the treatment negative response group.

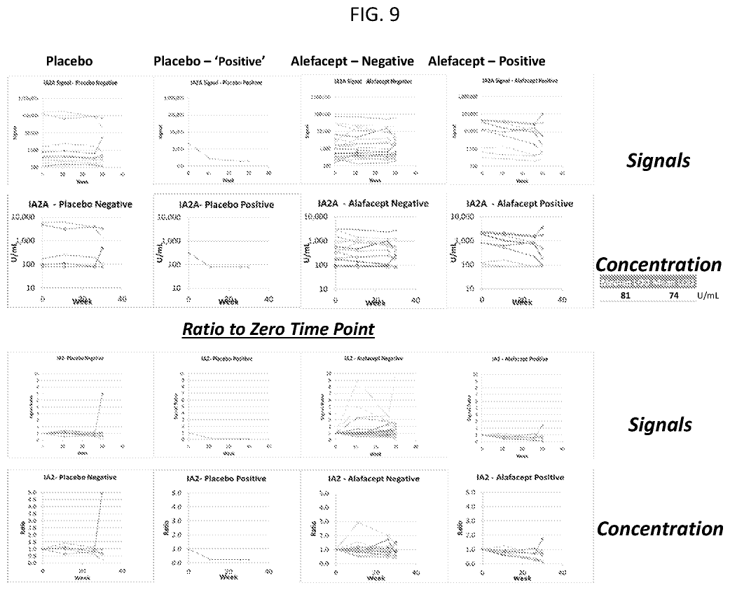

Results obtained for anti-IA2 IgM are shown in FIG. 9 and FIG. 4. In FIG. 9, each plotted line represents an individual patient at all available time points. The graphs are sorted based on treatment group (Placebo or Alefacept), and treatment outcome (Positive or Negative). The data are shown in terms of sample signals and calculated concentrations (top half). The ratios of signals or concentrations to each patient's 0 time point sample values are shown as well (bottom half). FIG. 4 shows median concentration values at each time point for the positive and negative response groups. As can be seen from the results: 1) The treatment response group shows less elevation of autoimmunity at earlier time points relative to treatment negative and placebo groups. 2) There are overall higher signals in positive response group.

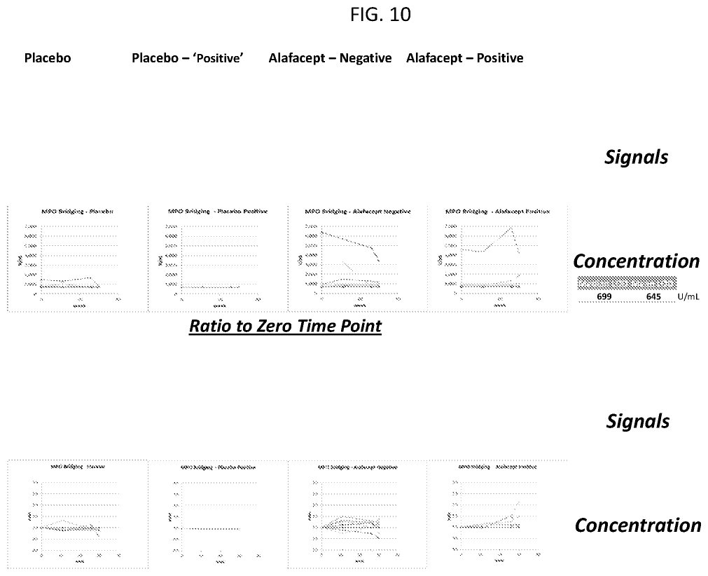

Results obtained for anti-MPO IgA are shown in FIG. 10. As can be seen from the results: Less changes were seen at the 11 week time point in positive response group as compared to placebo and negative response groups.

### Example 4. Analysis of Systemic Lupus Erythematosus Samples

For the systemic lupus erythematosus (SLE) cohort, samples were tested from patients with SLE, with and without flare of disease as defined by SLEDAI scores (described above), for autoantibodies related to SLE and for other autoimmune diseases. This demonstrates the use of the bridging serology format for a number of SLE and other connective tissue disorder-related autoimmune reactivities in such a cohort.

Samples are from 15 Lupus patients and 15 control patients. The patients (HIGH for Lupus patients during flare, and LOW for lupus patients not during flare), along with the visit at which the sample was collected and the patient's SLEDAI score, are shown in Table 14.

TABLE 14

| | | Visit | SLEDI |
|---|---|---|---|
| HIGH | JHP005 | 4 | 10 |
| | JHP008 | 7 | 11 |
| | JHP024 | 1 | 10 |
| | JHP028 | 4 | 16 |
| | JHP033 | 3 | 12 |
| | JHP047 | 1 | 16 |
| | JHP048 | 4 | 17 |
| | JHP057 | 1 | 14 |
| | JHP069 | 2 | 15 |
| | JHP075 | 3 | 11 |
| | JHP100 | 3 | 12 |
| | JHP115 | 4 | 14 |
| | JHP212 | 2 | 12 |
| | JHP251 | 1 | 18 |
| | JHP269 | 6 | 16 |
| LOW | JHP005 | 2 | 4 |
| | JHP008 | 2 | 2 |
| | JHP024 | 4 | 0 |
| | JHP028 | 1 | 4 |
| | JHP033 | 4 | 0 |
| | JHP047 | 3 | 4 |
| | JHP048 | 3 | 0 |
| | JHP057 | 5 | 4 |
| | JHP069 | 4 | 8 |
| | JHP075 | 2 | 1 |
| | JHP100 | 1 | 4 |
| | JHP115 | 1 | 6 |
| | JHP212 | 4 | 2 |
| | JHP251 | 6 | 2 |
| | JHP269 | 2 | 2 |

Two samples are provided for each patient: one sample corresponding to a flare-up in symptoms and the second sample collected when symptoms subsided. Samples from flare-up may be collected before or after the low-symptom sample. Eight non-flare samples were collected pre-flare, and seven non-flare samples were collected post-flare. The period between sample collections varied.

The objectives of this study were to:
Evaluate the performance of SLE-related markers in the classical serology and bridging formats;
Identify potential novel SLE markers; and
Identify potential markers of SLE flare development.

The samples were analyzed on the biomarker panels described in Example 1 using the statistical analysis described there.

For analysis of disease samples versus non-diseased controls, flare and non-flare samples were combined into the disease sample group and compared against controls. Top predictors as ranked by the Mann-Whitney-Wilcoxon test are shown in Table 15. All listed biomarkers are significant after Bonferroni correction for multiple comparisons. (Threshold=4.2e-04)

TABLE 15

| | Mann-Whitney-Wilcoson |
|---|---|
| Biomarker | Test p-value |
| Smith.IgG | 2.48E−10 |
| RoSSA60.IgG | 2.85E−09 |
| U1.RNPA.IgG | 1.34E−08 |
| IAA.IgM | 3.08E−08 |
| Ro.SSA52.IgG | 7.66E−08 |
| GAD65.IgM | 2.83E−07 |
| ZnT8.IgG | 5.04E−07 |
| RoSSA60.IgA | 5.33E−07 |
| Ro.SSA52.IgM | 2.40E−06 |
| aNCA.PR3.IgG | 3.02E−06 |
| Jo.1.IgA | 1.76E−05 |
| Smith. IgA | 2.85E−05 |
| U1.RNPA.IgM | 3.10E−05 |
| MPO.IgA | 3.69E−05 |
| U1RNPC.IgG | 4.16E−05 |
| GAD65.IgG | 4.63E−05 |
| La.SSb.IgA | 5.05E−05 |
| TPO.IgG | 5.30E−05 |
| MPO.IgG | 5.59E−05 |
| IAA.IgA | 6.95E−05 |
| TPO.IgM | 6.99E−05 |
| ZnT8.IgM | 8.85E−05 |
| IF.IgG | 1.26E−04 |
| Sm.IgG | 1.32E−04 |
| La.SSb.IgG | 2.08E−04 |

For analysis of SLE flare, flare samples were compared to non-flare samples. Top predictors of flare as ranked by the Mann-Whitney-Wilcoxon test are shown in Table 16.

TABLE 16

| | Mann-Whitney- Wilcoxon |
|---|---|
| Biomarker | signed-rank test p-value |
| IAA.IgM | 0.000 |
| MPO.IgA | 0.001 |
| Jo.1.IgA | 0.002 |
| ZnT8.IgM | 0.003 |
| GAD65.IgG | 0.005 |
| Smith.IgA | 0.006 |
| IA2.IgA | 0.007 |
| GAD65.IgM | 0.009 |
| Jo.1.IgG | 0.010 |
| beta2glycoprotein.IgA | 0.011 |
| ZnT8.IgG | 0.012 |
| IAA.IgA | 0.020 |

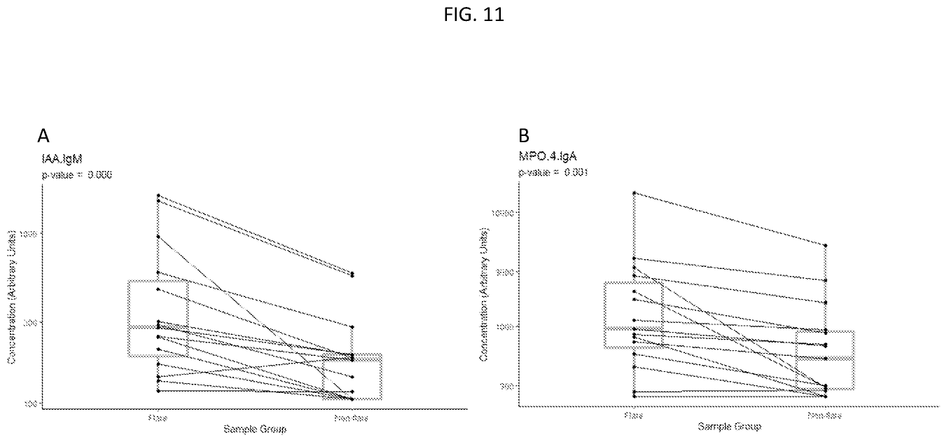

Whisker box plots of the concentrations detected for each sample in the flare and non-flare groups are shown in FIGS. 11A (IAA.IgM) and 11B (MPO.IgA).

### Example 5. Analysis of Systemic Lupus Erythematosus Samples with Other Biomarker Panels

The SLE samples described in Example 4 were analyzed to determine potential new panels for analysis of SLE.

For a comparison of diseased versus control samples, flare and non-flare samples were combined. Mann-Whitney-Wilcoxon test p-values were computed for the biomarkers show in Table 17 below. Tests whether two independent samples were selected from populations with the same distribution.

TABLE 17

| | | MWW test |
|---|---|---|
| | Biomarker | p-value |
| 1* | Smith.IgG | 2.48e−10 |
| 2* | RoSSA60.IgG | 2.85e−09 |
| 3* | U1.RNPA.IgG | 1.34e−08 |
| 4* | IAA.IgM | 3.08e−08 |
| 5* | Ro.SSA52.IgG | 7.66e−08 |
| 6* | GAD65.IgM | 2.83e−07 |
| 7* | TNFa | 3.64e−07 |
| 8* | ZnT8.IgG | 5.04e−07 |
| 9* | RoSSA60.IgA | 5.33e−07 |
| 10* | IL.15 | 8.88e−07 |
| 11* | MIP.1a | 1.01e−06 |
| 12* | IL.10 | 1.12e−06 |
| 13* | Ro.SSA52.IgM | 2.40e−06 |
| 14* | NFL | 2.95e−06 |
| 15* | IL.8 | 2.95e−06 |
| 16* | aNCA.PR3.IgG | 3.02e−06 |
| 17* | IL.6 | 7.79e−06 |
| 18* | Jo.1.IgA | 1.76e−05 |
| 19* | Smith.IgA | 2.85e−05 |
| 20* | U1.RNPA.IgM | 3.10e−05 |
| 21* | MPO.IgA | 3.69e−05 |
| 22* | IL.2 | 3.71e−05 |
| 23* | U1RNPC.IgG | 4.16e−05 |
| 24* | GAD65.IgG | 4.63e−05 |
| 25* | La.SSb.IgA | 5.05e−05 |
| 26* | TPO.IgG | 5.30e−05 |
| 27* | MPO.IgG | 5.59e−05 |
| 28* | IAA.IgA | 6.95e−05 |
| 29* | TPO.IgM | 6.99e−05 |
| 30* | ZnT8.IgM | 8.85e−05 |
| 31* | IF.IgG | 1.26e−04 |
| 32* | Sm.IgG | 1.32e−04 |
| 33* | La.SSb.IgG | 2.08e−04 |
| 34* | IL.21 | 2.57e−04 |
| 35* | IL.23 | 2.61e−04 |
| 36 | IP.10 | 5.08e−04 |
| 37 | Jo.1.IgM | 9.62e−04 |
| 38 | aNCA.PR3.IgA | 1.31e−03 |
| 39 | RNP68.70.IgG | 1.32e−03 |
| 40 | Thyroglobulin.IgA | 1.58e−03 |
| 41 | aNCA.PR3.IgM | 1.74e−03 |
| 42 | Smith.IgM | 2.09e−03 |
| 43 | RoSSA60.IgM | 3.26e−03 |
| 44 | IL.1b | 3.35e−O3 |
| 45 | GAD65.IgA | 3.40e−03 |
| 46 | VEGF | 4.16e−03 |
| 47 | IAA.IgG | 4.22e−03 |
| 48 | IL.12.23p40 | 4.52e−03 |
| 49 | TGM2.IgM | 5.51e−03 |
| 50 | U1.RNPA.IgA | 5.72e−03 |
| 51 | MCP.4 | 6.75e−03 |
| 52 | RNP68.70.IgM | 6.93e−03 |
| 53 | U1RNPC.IgM | 6.93e−03 |
| 54 | DGP.IgG | 7.23e−03 |
| 55 | IL.17a | 7.24e−03 |
| 56 | Ro.SSA52.IgA | 7.32e−03 |
| 57 | Eotaxin | 8.62e−03 |
| 58 | B2M | 9.43e−03 |
| 59 | beta2glycoprotein.IgA | 1.03e−02 |
| 60 | U1RNPC.IgA | 1.08e−02 |
| 61 | IFNg | 1.31e−02 |
| 62 | CCP.IgM | 1.46e−02 |
| 63 | Scl.70.IgM | 1.46e−02 |
| 64 | Sm.IgM | 1.46e−02 |
| 65 | Sm.IgA | 1.47e−02 |
| 66 | ProIAA.IgM | 1.49e−02 |
| 67 | IA2.IgA | 1.74e−02 |
| 68 | beta2glycoprotein.IgG | 1.83e−02 |
| 69 | Thyroglobulin.IgG | 1.87e−02 |
| 70 | ZnT8.IgA | 2.69e−02 |
| 71 | MPO.IgM | 2.70e−02 |
| 72 | NGAL | 2.98e−02 |
| 73 | CENP.B.IgM | 3.04e−02 |
| 74 | MCP.1 | 3.21e−02 |
| 75 | MDC | 3.21e−02 |
| 76 | CENP.B.IgA | 3.40e−02 |
| 77 | RNP68.70.IgA | 3.85e−02 |
| 78 | CCP.IgG | 4.49e−02 |
| 79 | IF.IgA | 5.19e−02 |
| 80 | IL.7 | 5.82e−02 |
| 81 | La.SSb.IgM | 6.36e−02 |
| 82 | MPO.IgM | 6.36e−02 |
| 83 | OPN | 6.42e−02 |
| 84 | TARC | 6.73e−02 |
| 85 | IL.1a | 6.98e−02 |
| 86 | CENP.B.IgG | 8.17e−02 |
| 87 | ProIAA.IgG | 8.82e−02 |
| 88 | IL12p70 | 8.92e−02 |
| 89 | IL.16 | 8.98e−02 |
| 90 | TSLP | 9.24e−02 |
| 91 | IA2.IgM | 9.96e−02 |
| 92 | MPO.IgA | 1.07e−01 |
| 93 | CCP.IgA | 1.23e−01 |
| 94 | beta2glycoprotein.IgM | 1.35e−01 |
| 95 | TGM2.IgG | 1.70e−01 |
| 96 | TPO.IgA | 1.70e−01 |
| 97 | DGP.IgA | 1.90e−01 |
| 98 | Cystatin.C | 1.93e−01 |
| 99 | MPO.IgG | 2.07e−01 |
| 100 | Thyroglobulin.IgM | 2.93e−01 |
| 101 | IF.IgM | 3.48e−01 |
| 102 | MIP.1b | 3.49e−01 |
| 103 | TNFb | 3.95e−01 |
| 104 | IL.5 | 3.99e−01 |
| 105 | IL.22 | 4.01e−01 |
| 106 | EGF | 4.12e−01 |
| 107 | Scl.70.IgA | 4.16e−01 |
| 108 | UMOD | 4.56e−01 |
| 109 | Scl.70.IgG | 5.05e−01 |
| 110 | Eotaxin.3 | 5.25e−01 |
| 111 | Jo.1.IgG | 5.93e−01 |
| 112 | ProIAA.IgA | 6.21e−01 |
| 113 | IA2.IgG | 6.36e−01 |
| 114 | ILA | 8.19e−01 |
| 115 | NFH | 8.21e−01 |
| 116 | TGM2.IgA | 8.60e−01 |
| 117 | GM.CSF | 8.88e−01 |
| 118 | DGP.IgM | 8.90e−01 |
| 119 | IL.13 | 9.49e−01 |

A * in Table 16 denotes significance at the 5 percent level. As can be seen in Table 17, 35 biomarkers are significant after Bonferroni correction for multiple comparisons. (Threshold=4.2e-04).

For a comparison of flare versus non-flare samples, Wilcoxon signed-rank test p-values were computed for the biomarkers show in Table 18 below. Tests whether two dependent, matched samples were selected from populations with the same distribution.

TABLE 18

| | | Wilcoxon |
|---|---|---|
| | | signed-rank |
| | Biomarker | test p-vilue |
| 1* | IAA.IgM | 0.000 |
| 2 | MPO.IgA | 0.001 |
| 3 | TARC | 0.001 |
| 4 | Jo.1.IgA | 0.002 |
| 5 | ZnT8.IgM | 0.003 |
| 6 | GAD65.IgG | 0.005 |
| 7 | Smith.IgA | 0.006 |
| 8 | IA2.IgA | 0.007 |
| 9 | MIP-1b | 0.007 |
| 10 | GAD65.IgM | 0.009 |
| 11 | Jo.1.IgG | 0.010 |
| 12 | IL-10 | 0.010 |
| 13 | beta2glycoprotein.IgA | 0.011 |
| 14 | ZnT8.IgG | 0.012 |
| 15 | MCP-4 | 0.015 |
| 16 | IAA.IgA | 0.020 |
| 17 | IL-6 | 0.022 |
| 18 | MIP-1a | 0.026 |
| 19 | DGP.IgG | 0.028 |
| 20 | IL-17a | 0.030 |
| 21 | La.SSb.IgG | 0.031 |
| 22 | IAA.IgG | 0.035 |
| 23 | TNFa | 0.035 |
| 24 | IL-7 | 0.041 |
| 25 | Eotaxin | 0.041 |
| 26 | MPO.IgM | 0.045 |
| 27 | IL-21 | 0.050 |
| 28 | beta2glycoprotein.IgG | 0.055 |
| 29 | Smith.IgM | 0.056 |
| 30 | CCP.IgM | 0.059 |
| 31 | GAD65.IgA | 0.060 |
| 32 | IL-1b | 0.060 |
| 33 | IL-12/23p40 | 0.064 |
| 34 | IA2.IgG | 0.073 |
| 35 | IFNg | 0.073 |
| 36 | Cystatin C | 0.073 |
| 37 | IL-23 | 0.081 |
| 38 | MCP-1 | 0.083 |
| 39 | CENP.B.IgM | 0.100 |
| 40 | ProIAA.IgA | 0.100 |
| 41 | Ro.SSA52.IgA | 0.100 |
| 42 | ProIAA.IgM | 0.106 |
| 43 | Scl.70.IgM | 0.106 |
| 44 | Eotaxin 3 | 0.107 |
| 45 | IP-10 | 0.107 |
| 46 | Jo.1.IgM | 0.108 |
| 47 | DGP.IgA | 0.121 |
| 48 | IL-15 | 0.121 |
| 49 | IF.IgG | 0.126 |
| 50 | IL-5 | 0.135 |
| 51 | Smith.IgG | 0.149 |
| 52 | RoSSA60.IgM | 0.151 |
| 53 | MPO.IgG | 0.169 |
| 54 | NFL | 0.169 |
| 55 | DGP.IgM | 0.170 |
| 56 | TGM2.IgG | 0.178 |
| 57 | IL-1a | 0.178 |
| 58 | MPO.IgM | 0.181 |
| 59 | IL-4 | 0.188 |
| 60 | U1RNPC.IgM | 0.208 |
| 61 | OPN | 0.208 |
| 62 | IL-2 | 0.229 |
| 63 | La.SSb.IgA | 0.230 |
| 64 | TSLP | 0.252 |
| 65 | B2M | 0.252 |
| 66 | IA2.IgM | 0.266 |
| 67 | IL-22 | 0.277 |
| 68 | Sm.IgM | 0.281 |
| 69 | RNP68.70.IgM | 0.295 |
| 70 | GM-CSF | 0.295 |
| 71 | ZnT8.IgA | 0.330 |
| 72 | MDC | 0.330 |
| 73 | TGM2.IgM | 0.353 |
| 74 | UMOD | 0.359 |
| 75 | beta2glycoprotein.IgM | 0.371 |
| 76 | CCP.IgA | 0.414 |
| 77 | U1.RNPA.IgA | 0.415 |
| 78 | TNFb | 0.415 |
| 79 | Thyroglobulin.IgG | 0.418 |
| 80 | TPO.IgG | 0.441 |
| 81 | Thyroglobulin.IgA | 0.477 |
| 82 | VEGF | 0.489 |
| 83 | TPO.IgM | 0.490 |
| 84 | Ro.SSA52.IgM | 0.505 |
| 85 | NFH | 0.524 |
| 86 | NGAL | 0.524 |
| 87 | aNCA.PR3.IgG | 0.576 |
| 88 | IL-13 | 0.584 |
| 89 | RoSSA60.IgA | 0.616 |
| 90 | U1.RNPA.IgM | 0.639 |
| 91 | IL-8 | 0.639 |
| 92 | CENP.B.IgA | 0.675 |
| 93 | IL-16 | 0.679 |
| 94 | aNCA.PR3.IgA | 0.756 |
| 95 | Sm.IgA | 0.780 |
| 96 | IPO.IgA | 0.780 |
| 97 | RNP68.70.IgG | 0.784 |
| 98 | La.SSb.IgM | 0.789 |
| 99 | MPO.IgA | 0.834 |
| 100 | IL12p70 | 0.834 |
| 101 | Ro.SSA52.IgG | 0.845 |
| 102 | aNCA.PR3.IgM | 0.847 |
| 103 | U1.RNPA.IgG | 0.847 |
| 104 | RNP68.70.IgA | 0.889 |
| 105 | Scl.70.IgA | 0.889 |
| 106 | MPO.IgG | 0.890 |
| 107 | IF.IgM | 0.906 |
| 108 | U1RNPC.IgA | 0.906 |
| 109 | U1RNPC.IgG | 0.906 |
| 110 | IF.IgA | 0.950 |
| 111 | EGF | 0.950 |
| 112 | CCP.IgG | 0.965 |
| 113 | CENP.B.IgG | 0.965 |
| 114 | Sm.IgG | 0.969 |
| 115 | Scl.70.IgG | 0.978 |
| 116 | ProIAA.IgG | 1.000 |
| 117 | RoSSA60.IgG | 1.000 |
| 118 | TGM2.IgA | 1.000 |
| 119 | Thyroglobulin.IgM | 1.000 |

A * in Table 18 denotes significance at the 5 percent level.

Samples were assayed for a correlation between biomarkers and SLEDAI scores. Spearman rank correlation was tested in order to assess the correlation between the rank order of sample concentrations with SLEDAI score. Correlation coefficients and p-values were computed for the biomarkers shown in Table 19.

TABLE 19

| | | Spearman | |
|---|---|---|---|
| | | Correlation | p- |
| | Biomarker | Coefficient | value |
| 1 | Jo.1.IgA | 0.512 | 0.004 |
| 2 | IP-10 | 0.498 | 0.005 |
| 3 | IL-6 | 0.477 | 0.008 |
| 4 | beta2glycoprotein.IgA | 0.471 | 0.009 |
| 5 | TNFa | 0.433 | 0.017 |
| 6 | MIP-1a | 0.428 | 0.018 |
| 7 | Jo.1.IgG | 0.412 | 0.024 |
| 8 | CENP.B.IgM | −0.409 | 0.025 |
| 9 | B2M | 0.394 | 0.031 |
| 10 | ZnT8.IgG | 0.387 | 0.035 |
| 11 | IAA.IgM | 0.377 | 0.04 |
| 12 | Eotaxin 3 | 0.375 | 0.041 |
| 13 | CCP.IgM | −0.374 | 0.042 |
| 14 | TARC | −0.373 | 0.042 |
| 15 | IL-1b | 0.369 | 0.045 |
| 16 | IFNg | 0.361 | 0.05 |
| 17 | OPN | 0.357 | 0.053 |
| 18 | MPO.IgM | −0.343 | 0.063 |
| 19 | MIP-1b | 0.342 | 0.064 |
| 20 | IL-2 | 0.333 | 0.072 |
| 21 | Cystatin C | 0.327 | 0.078 |
| 22 | RoSSA60.IgM | −0.323 | 0.082 |
| 23 | Sm.IgM | −0.323 | 0.082 |
| 24 | IA2.IgG | 0.32 | 0.085 |
| 25 | MCP-1 | 0.32 | 0.085 |
| 26 | ProIAA.IgA | 0.317 | 0.088 |
| 27 | ZnT8.IgM | 0.315 | 0.09 |
| 28 | IA2.IgA | 0.312 | 0.093 |
| 29 | IL-17a | 0.3 | 0.107 |
| 30 | MPO.IgG | 0.299 | 0.109 |
| 31 | IL-12/23p40 | 0.29 | 0.12 |
| 32 | IL-5 | 0.288 | 0.122 |
| 33 | MPO.IgA | 0.287 | 0.124 |
| 34 | NFL | 0.286 | 0.125 |
| 35 | IL-10 | 0.283 | 0.13 |
| 36 | ProIAA.IgM | 0.282 | 0.131 |
| 37 | IL-8 | 0.276 | 0.139 |
| 38 | beta2glycoprotein.IgG | 0.273 | 0.144 |
| 39 | GAD65.IgG | 0.273 | 0.144 |
| 40 | ZnT8.IgA | 0.273 | 0.144 |
| 41 | Thyroglobulin.IgA | 0.269 | 0.15 |
| 42 | U1RNPC.IgM | −0.262 | 0.161 |
| 43 | RNP68.70.IgM | −0.261 | 0.163 |
| 44 | Scl.70.IgM | −0.244 | 0.193 |
| 45 | TGM2.IgG | 0.242 | 0.197 |
| 46 | TSLP | 0.229 | 0.225 |
| 47 | La.SSb.IgA | 0.227 | 0.228 |
| 48 | IF.IgG | 0.224 | 0.233 |
| 49 | TGM2.IgM | 0.224 | 0.235 |
| 50 | IAA.IgA | 0.206 | 0.275 |
| 51 | Smith.IgA | 0.205 | 0.277 |
| 52 | IL-1a | 0.202 | 0.285 |
| 53 | beta2glycoprotein.IgM | 0.199 | 0.291 |
| 54 | UMOD | −0.197 | 0.297 |
| 55 | Jo.1.IgM | 0.195 | 0.302 |
| 56 | CENP.B.IgG | −0.192 | 0.31 |
| 57 | NFH | 0.184 | 0.33 |
| 58 | TNFb | 0.178 | 0.347 |
| 59 | IL-21 | 0.176 | 0.352 |
| 60 | GAD65.IgA | 0.175 | 0.355 |
| 61 | MCP-4 | −0.168 | 0.375 |
| 62 | GM-CSF | 0.164 | 0.386 |
| 63 | Ro.SSA52.IgM | 0.163 | 0.389 |
| 64 | IL-23 | 0.159 | 0.4 |
| 65 | MPO.IgM | 0.155 | 0.414 |
| 66 | Thyroglobulin.IgG | 0.155 | 0.415 |
| 67 | IF.IgA | 0.153 | 0.418 |
| 68 | Sm.IgG | −0.145 | 0.445 |
| 69 | IL-15 | 0.145 | 0.444 |
| 70 | GAD65.IgM | 0.144 | 0.446 |
| 71 | IL-7 | −0.144 | 0.448 |
| 72 | Scl.70.IgG | −0.14 | 0.46 |
| 73 | Smith.IgM | 0.134 | 0.479 |
| 74 | U1RNPC.IgG | −0.133 | 0.484 |
| 75 | RoSSA60.IgA | −0.132 | 0.488 |
| 76 | CCP.IgG | −0.13 | 0.492 |
| 77 | RNP68.70.IgA | −0.13 | 0.494 |
| 78 | NGAL | 0.13 | 0.495 |
| 79 | IAA.IgG | 0.126 | 0.506 |
| 80 | La.SSb.IgM | 0.122 | 0.519 |
| 81 | Scl.70.IgA | −0.12 | 0.529 |
| 82 | IF.IgM | 0.119 | 0.53 |
| 83 | ProIAA.IgG | 0.117 | 0.539 |
| 84 | Ro.SSA52.IgG | −0.116 | 0.542 |
| 85 | IL-22 | 0.113 | 0.553 |
| 86 | aNCA.PR3.IgG | 0.112 | 0.554 |
| 87 | IL-4 | 0.112 | 0.556 |
| 88 | IL-16 | 0.106 | 0.578 |
| 89 | IL-13 | 0.104 | 0.585 |
| 90 | La.SSb.IgG | 0.099 | 0.603 |
| 91 | Ro.SSA52.IgA | 0.099 | 0.604 |
| 92 | VEGF | 0.098 | 0.606 |
| 93 | aNCA.PR3.IgM | 0.095 | 0.617 |
| 94 | U1.RNPA.IgM | 0.091 | 0.631 |
| 95 | TPO.IgA | 0.09 | 0.638 |
| 96 | IA2.IgM | −0.088 | 0.644 |
| 97 | Eotaxin | 0.077 | 0.684 |
| 98 | Thyroglobulin.IgM | 0.074 | 0.699 |
| 99 | DGP.IgG | 0.069 | 0.718 |
| 100 | U1.RNPA.IgA | 0.069 | 0.719 |
| 101 | aNCA.PR3.IgA | 0.066 | 0.727 |
| 102 | RNP68.70.IgG | −0.061 | 0.75 |
| 103 | TGM2.IgA | 0.061 | 0.75 |
| 104 | IL12p70 | 0.056 | 0.767 |
| 105 | CCP.IgA | −0.05 | 0.791 |
| 106 | MDC | −0.05 | 0.792 |
| 107 | MPO.IgA | −0.049 | 0.797 |
| 108 | Sm.IgA | −0.048 | 0.802 |
| 109 | U1.RNPA.IgG | −0.048 | 0.803 |
| 110 | EGF | 0.04 | 0.835 |
| 111 | U1RNPC.IgA | 0.037 | 0.846 |
| 112 | RoSSA60.IgG | −0.036 | 0.852 |
| 113 | TPO.IgG | 0.026 | 0.891 |
| 114 | CENP.B.IgA | −0.022 | 0.907 |
| 115 | Smith.IgG | 0.016 | 0.935 |
| 116 | DGP.IgM | −0.014 | 0.943 |
| 117 | MPO.IgG | −0.014 | 0.942 |
| 118 | TPO.IgM | 0.014 | 0.942 |
| 119 | DGP.IgA | 0.012 | 0.949 |

Non-flare samples collected pre-flare were compared with flare samples. Wilcoxon signed-rank test p-values were computed for the biomarkers show in Table 20 below. Tests whether two dependent, matched samples were selected from populations with the same distribution.

TABLE 20

| | | Wilcoxon |
|---|---|---|
| | | signed-rank |
| | Biomarker | test p-value |
| 1 | MCP-1 | 0.008 |
| 2 | Cystatin C | 0.008 |
| 3 | IAA.IgM | 0.016 |
| 4 | IL-5 | 0.016 |
| 5 | IL-7 | 0.016 |
| 6 | IL-1b | 0.023 |
| 7 | aNCA.PR3.IgG | 0.035 |
| 8 | MPO.IgA | 0.035 |
| 9 | ZnT8.IgM | 0.036 |
| 10 | Jo.1.IgG | 0.039 |
| 11 | IL-21 | 0.039 |
| 12 | IL-6 | 0.039 |
| 13 | MIP-1b | 0.039 |
| 14 | TARC | 0.039 |
| 15 | MCP-4 | 0.039 |
| 16 | IAA.IgA | 0.052 |
| 17 | Jo.1.IgM | 0.052 |
| 18 | IL-10 | 0.055 |
| 19 | MIP-1a | 0.055 |
| 20 | Smith.IgA | 0.059 |
| 21 | GAD65.IgM | 0.076 |
| 22 | Jo.1.IgA | 0.078 |
| 21 | NGAL | 0.078 |
| 24 | IL-1a | 0.100 |
| 25 | beta2glycoprotein.IgA | 0.106 |
| 26 | TPO.IgG | 0.106 |
| 27 | GAD65.IgG | 0.109 |
| 28 | IA2.IgG | 0.109 |
| 29 | IFNg | 0.109 |
| 30 | B2M | 0.109 |
| 31 | IL-22 | 0.148 |
| 32 | IP-10 | 0.148 |
| 33 | ZnT8.IgG | 0.151 |
| 34 | OPN | 0.181 |
| 35 | IL-15 | 0.195 |
| 36 | IL-16 | 0.195 |
| 37 | Eotaxin 3 | 0.195 |
| 38 | IAA.IgG | 0.201 |
| 39 | ProIAA.IgM | 0.201 |
| 40 | CCP.IgA | 0.205 |
| 41 | IA2.IgA | 0.250 |
| 42 | TSLP | 0.250 |
| 43 | IL-8 | 0.250 |
| 44 | Eotaxin | 0.250 |
| 45 | GAD65.IgA | 0.272 |
| 46 | aNCA.PR3.IgA | 0.281 |
| 47 | beta2glycoprotein.IgG | 0.281 |
| 48 | IF.IgG | 0.295 |
| 49 | La.SSb.IgG | 0.295 |
| 50 | Smith.IgM | 0.295 |
| 51 | IL-2 | 0.313 |
| 52 | IL-12/23p40 | 0.313 |
| 53 | CENP.B.IgA | 0.353 |
| 54 | IF.IgM | 0.353 |
| 55 | CCP.IgM | 0.371 |
| 56 | CENP.B.IgM | 0.371 |
| 57 | ProIAA.IgA | 0.371 |
| 58 | Ro.SSA52.IgA | 0.371 |
| 59 | Scl.70.IgM | 0.371 |
| 60 | Thyroglobulin.IgG | 0.371 |
| 61 | MPO.IgG | 0.383 |
| 62 | U1.RNPA.IgM | 0.383 |
| 63 | GM-CSF | 0.383 |
| 64 | IL-23 | 0.383 |
| 65 | NFL | 0.383 |
| 66 | TNFa | 0.383 |
| 67 | TNFb | 0.402 |
| 68 | RoSSA60.IgM | 0.423 |
| 69 | IL-13 | 0.423 |
| 70 | Scl.70.IgA | 0.447 |
| 71 | IL-17a | 0.447 |
| 72 | EGF | 0.447 |
| 73 | U1.RNPA.IgG | 0.547 |
| 74 | IA2.IgM | 0.554 |
| 75 | IF.IgA | 0.554 |
| 76 | Smith.IgG | 0.554 |
| 77 | RNP68.70.IgM | 0.584 |
| 78 | U1RNPC.IgM | 0.584 |
| 79 | IL-4 | 0.641 |
| 80 | DGP.IgG | 0.673 |
| 81 | DGP.IgM | 0.673 |
| 82 | RNP68.70.IgG | 0.673 |
| 83 | Sm.IgA | 0.673 |
| 84 | TPO.IgA | 0.673 |
| 85 | U1.RNPA.IgA | 0.675 |
| 86 | Ro.SSA52.IgG | 0.742 |
| 87 | NFH | 0.742 |
| 88 | MDC | 0.742 |
| 89 | MPO.IgM | 0.787 |
| 90 | U1RNPC.IgA | 0.834 |
| 91 | aNCA.PR3.IgM | 0.844 |
| 92 | DGP.IgA | 0.844 |
| 93 | VEGF | 0.844 |
| 94 | UMOD | 0.844 |
| 95 | CENP.B.IgG | 0.933 |
| 96 | MPO.IgA | 0.933 |
| 97 | RNP68.70.IgA | 0.933 |
| 98 | RoSSA60.IgA | 0.933 |
| 99 | TPO.IgM | 0.933 |
| 100 | U1RNPC.IgG | 0.933 |
| 101 | IL12p70 | 0.933 |
| 102 | MPO.IgG | 0.945 |
| 103 | CCP.IgG | 1.000 |
| 104 | beta2glycoprotein.IgM | 1.000 |
| 105 | La.SSb.IgA | 1.000 |
| 106 | La.SSb.IgM | 1.000 |
| 107 | MPO.IgM | 1.000 |
| 108 | ProIAA.IgG | 1.000 |
| 109 | Ro.SSA52.IgM | 1.000 |
| 110 | RoSSA60.IgG | 1.000 |
| 111 | Scl.70.IgG | 1.000 |
| 112 | Sm.IgG | 1.000 |
| 113 | Sm.IgM | 1.000 |
| 114 | TGM2.IgG | 1.000 |
| 115 | TGM2.IgM | 1.000 |
| 116 | Thyroglobulin.IgA | 1.000 |
| 117 | Thyroglobulin.IgM | 1.000 |
| 118 | ZnT8.IgA | 1.000 |
| 119 | TGM2.IgA | |

### Panel Selection Using LASSO

Least absolute shrinkage and selection operator (LASSO) panel selection was used to select panels to best differentiate between disease and control samples and to elect panels to best differentiate between flare and non-flare samples.

Regularized logistic regression was used in order to predict the outcome. Outcome probability μ is estimated as a function of measured parameters:

μ=1/(1+exp(−(β₀+β₁X₁+β₂X₂+ . . . +β_{N}X_{N})))
where X values are the predictors, (3 values are the coefficients for each predictor, (30 is the intercept, and N is the number of predictors.

The Cost function is calculated as:

$$Cost ( µ , y ) = [ \frac{1}{M} {\sum }_{i = 1}^{M} - {y}_{i} ln {µ}_{i} - ( 1 - {y}_{i} ) ln ( 1 - {µ}_{i} ) ] + λ {\sum }_{j = 1}^{N} \vert {β}_{j} \vert$$

where μ is the outcome probability, y is the outcome, M is the number of samples, β values are the coefficients for each predictor, N is the number of predictors, and λ, is the regularization parameter.

The Regularization parameter λ, prevents overfitting by increasing the cost for large coefficients. LASSO regularization selects panels by scaling correlated predictors to 0. Panels that resulted in a cost within one standard error of the minimum were selected. All data were log transformed. Missing values were imputed with the k-nearest neighbor algorithm. Panels were selected to best predict disease vs. control samples and flare vs. non-flare samples using the lassoglm( ) function in MATLAB.

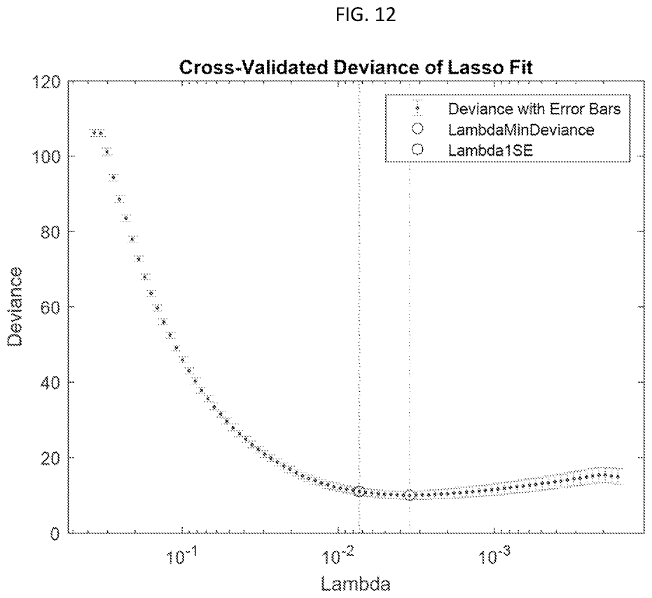

Individual predictors that were significant at the 5 percent level after correction for multiple comparisons (n=35) were considered as candidate predictors for panel selection. FIG. 12 shows a plot displaying cross validated deviance, as determined by the cost function, for different lambda values. Hold-out cross validation was repeated 150 times to determine coefficients. Seventy samples were randomly chosen to train the model, the remaining 9 used to test. Two panels resulted in deviance calculations within 1 standard error of the minimum.

Selected panels:
Panel of 8: anti-Smith-IgG, anti-RoSSA60-IgG, anti-RoSSA60-IgA, IL-15, MIP-1a, NFL, IL-2, IL-21
Panel of 9: anti-Smith-IgG, anti-RoSSA60-IgG, anti-RoSSA60-IgA, IL-15, MIP-1a, IL-10, NFL, IL-2, IL-21

Coefficients were input into the outcome probability equation for prediction.

The panel of 8 equation:

μ=1/(1+exp(−(−34.8493+2.1650 log[SmithIgG]+1.0656 log[RoSSA60IgG]+0.3893 log[RoSSA60IgA]+0.9297 log[IL15]+2.3058 log[MIP1a]+4.7343 log[NFL]+0.1642 log[IL2]+1.2715 log[IL21])))

The panel of 9 equation:

μ=1/(1+exp(−(−41.7623+2.5928 log[SmithIgG]+1.2996 log[RoSSA60IgG]+0.4162 log[RoSSA60IgA]+0.9938 log[IL15]+2.6522 log[MIP1a]+0.0290 log[IL10]+5.8344 log[NFL]+0.1529 log[IL2]+1.5518 log[IL21])))

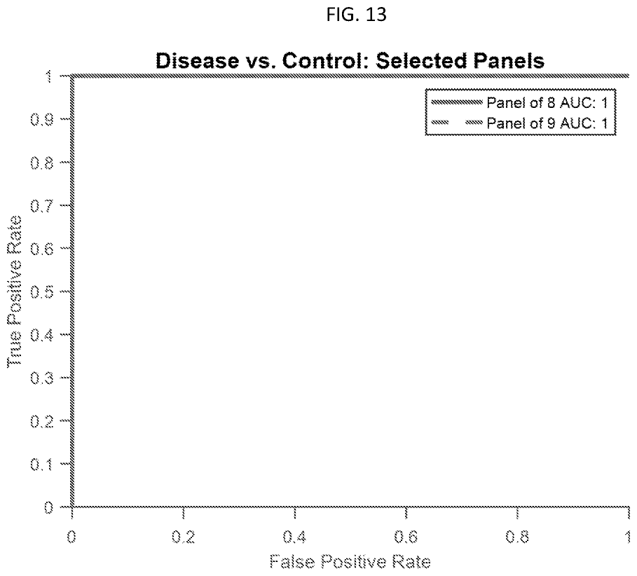

Outcome probability equations were applied to the original dataset, and ROC curves were generated as shown in FIG. 13. Both equations resulted in AUC values of 1. For further validation, the equations will be applied to an independently generated dataset to assess predictive efficacy. This will evaluate the degree of overfitting.

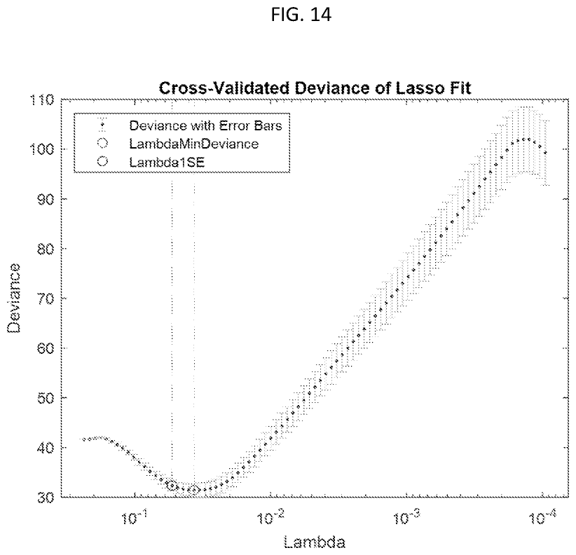

For flare versus non-flare panel prediction, as only one biomarker was significant after correction for multiple comparisons, significant predictors before correction (n=27) were considered as candidate predictors for panel generation. The plot in FIG. 14 displays cross validated deviance, as determined by the cost function, for different lambda values. Stratified hold-out cross validation were repeated 150 times to determine coefficients.

Twelve flare and twelve non-flare samples randomly chosen to train model, the remaining six samples (3 flare, 3 non-flare) were used to test the model. Three panels resulted in deviance calculations within 1 standard error of the minimum.

Selected panels:
Panel of 7: IAA-IgM, MPO-IgA, TARC, Jo-1-IgA, GAD65-IgM, MIP-1a, IL-7
Panel of 8: IAA-IgM, MPO-IgA, TARC, Jo-1-IgA, GAD65-IgM, MIP-1a, IL-7, Eotaxin
Panel of 9: IAA-IgM, MPO-IgA, TARC, Jo-1-IgA, GAD65-IgM, MIP-1a, IL-7, Eotaxin, MPO-IgM

Coefficients were input into the outcome probability equation for prediction.

The panel of 7 equation:

μ=1/(1+exp(−(4.7788+1.7941 log[IAAIgM]+1.0626 log[MPO4IgA]−3.0951 log[TARC]+0.3676 log[Jo1IgA]+0.4523 log[GADIgM]+1.5329 log[MIP1a]−2.0244 log[IL7])))

The panel of 8 equation:

μ=1/(1+exp(−(6.7944+1.9339 log[IAAIgM]+1.1880 log[MPO4IgA]−3.4720 log[TARC]+0.4990 log[Jo1IgA]+0.6425 log[GADIgM]+1.8593 log[MIP1a]−2.5075 log[IL7]−0.3740 log[Eotaxin])))

The panel of 9 equation:

μ=1/(1+exp(—(8.2169+2.0902 log[IAAIgM]+1.1806 log[MPO4IgA]−3.9119 log[TARC]+0.7690 log[Jo1/gA]+0.8765 log[GADIgM]+2.2014 log[MIP1a]−2.9674 log[IL7]−0.8468 log[Eotaxin]+0.2702 log[MP04IgM])))

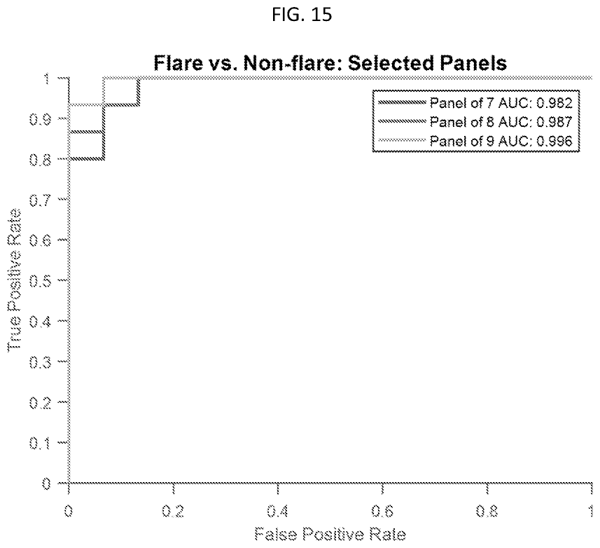

Outcome probability equations were applied to the original dataset, and ROC curves were generated as shown in FIG. 15. All three equations resulted in AUC values of above 0.98. For further validation, the equations will be applied to an independently generated dataset to assess predictive efficacy. This will evaluate the degree of overfitting.

As shown in this example, many individual biomarkers are successfully able to differentiate between disease vs. control samples. Only Jo-1-IgA ranked in the top 10 biomarkers for both Wilcoxon signed-rank and Spearman rank correlation tests. Differences stem from binary vs. ordinal groupings. In addition, the Spearman rank correlation test did not take into account the matched nature of the samples. Some biomarkers displayed substantially different rankings between flare vs. non-flare and flare vs. pre-flare tests. There may be a temporal offset between certain biomarker fluctuations and observable flare symptoms, leading to this discrepancy.

### Example 6. Analysis of Celiac Disease Samples

The Celiac disease samples described in Example 1, were assayed using the biomarker panels described in Example 1.

Three types of samples were tested:
1) Newly diagnosed, untreated celiac disease (untreated; N=20). The clinical characteristics of the patients from which the samples were obtained include: a) Suspicion for celiac disease leading to clinically indicated celiac serology request/sample collection; b) Confirmed positive IgA-TGM2 serology; c) Small bowel biopsy demonstrating characteristic changes of celiac disease histopathology with villus atrophy (Marsh III lesion) and d) Gluten free diet (GFD) not yet initiated or initiated no more than 4 weeks prior to serum collection
2) Treated celiac disease (treated; N=20). The clinical characteristics of the patients from which the samples were obtained include: a) Patient with known celiac disease at follow up visit. Clinically indicated celiac serology request/sample collection for monitoring serologic response to GFD; b) Gluten free diet initiated at least 12 months prior to serum collection; c) Confirmed celiac disease with small bowel biopsy demonstrating characteristic changes of celiac disease histopathology with villus atrophy (Marsh III lesion) at the time of initial diagnosis; and d) Positive IgA-TGM2 serology prior to/at diagnosis.
3) Non-celiac controls (NC; N=20). The clinical characteristics of the patients from which the samples were obtained include: a) Celiac disease excluded clinically and by negative celiac serology; b) Gastro-intestinal symptoms caused by confirmed gastrointestinal disorders: i) Irritable bowel syndrome (IBS) or ii) Gastroesophageal reflux disease (GERD); and c) Ingesting a normal, gluten-containing diet prior to serum collection.

Individual biomarkers were assessed for their ability to distinguish between: 1) the disease (combined untreated and treated groups) and control groups, 2) the untreated and control groups and 3) the treated and untreated groups.

Individual biomarkers were ranked by receiving operating characteristics (ROC) and area under the curve (AUC) values as is known in the art.

Samples from disease versus control subjects were compared. Top predictors as ranked by ROC and AUC are shown in Table 21.

TABLE 21

| | Biomarker | AUC | 95% CI |
|---|---|---|---|
| 1 | TGM2.IgA | 0.98 | 0.956-1 |
| 2 | DGP.IgA | 0.93 | 0.869-0.998 |
| 3 | TGM2.IgG | 0.89 | 0.82-0.967 |
| 4 | DGP.IgG | 0.86 | 0.773-0.957 |
| 5 | Jo.1.IgA | 0.8 | 0.684-0.918 |
| 6 | beta2glycoprotein.IgG | 0.78 | 0.652-0.903 |
| 7 | TGM2.IgM | 0.76 | 0.643-0.885 |
| 8 | Scl.70.IgA | 0.74 | 0.607-0.868 |
| 9 | Smith.IgG | 0.71 | 0.593-0.828 |
| 10 | RoSSA60.IgG | 0.7 | 0.548-0.844 |
| 11 | GAD65.IgA | 0.69 | 0.555-0.834 |
| 12 | ZnT8.IgG | 0.69 | 0.554-0.825 |
| 13 | IF.IgA | 0.68 | 0.523-0.832 |
| 14 | Smith.IgA | 0.68 | 0.6-0.75 |
| 15 | ZnT8.IgA | 0.68 | 0.536-0.824 |
| 16 | La.SSb.IgA | 0.67 | 0.528-0.822 |
| 17 | CCP.IgA | 0.66 | 0.515-0.811 |
| 18 | CENP.B.IgA | 0.66 | 0.502-0.815 |
| 19 | RoSSA60.IgA | 0.66 | 0.521-0.801 |
| 20 | Sm.IgA | 0.66 | 0.508-0.802 |
| 21 | aNCA.PR3.IgA | 0.65 | 0.526-0.774 |
| 22 | CENP.B.IgG | 0.65 | 0.499-0.801 |
| 23 | RNP68.70.IgA | 0.64 | 0.492-0.797 |
| 24 | Scl.70.IgG | 0.64 | 0.491-0.789 |
| 25 | DGP.IgM | 0.63 | 0.485-0.772 |
| 26 | MPO.4.IgA | 0.62 | 0.478-0.762 |
| 27 | MPO.4.IgG | 0.62 | 0.464-0.783 |
| 28 | MPO.IgA | 0.62 | 0.462-0.768 |
| 29 | MPO.IgG | 0.62 | 0.467-0.775 |
| 30 | Sm.IgG | 0.62 | 0.461-0.772 |
| 31 | TPO.IgA | 0.62 | 0.474-0.764 |
| 32 | aNCA.PR3.IgG | 0.61 | 0.498-0.723 |
| 33 | ProIAA.IgA | 0.61 | 0.547-0.678 |
| 34 | Ro.SSA52.IgG | 0.61 | 0.511-0.702 |
| 35 | U1.RNPA.IgG | 0.61 | 0.499-0.725 |
| 36 | CCP.IgG | 0.6 | 0.446-0.761 |
| 37 | beta2glycoprotein.IgA | 0.6 | 0.446-0.754 |
| 38 | GAD65.IgG | 0.6 | 0.446-0.746 |
| 39 | Jo.1.IgG | 0.6 | 0.442-0.76 |
| 40 | RNP68.70.IgG | 0.6 | 0.444-0.753 |
| 41 | U1RNPC.IgA | 0.6 | 0.451-0.746 |
| 42 | IA2.IgM | 0.59 | 0.433-0.747 |
| 43 | IAA.IgA | 0.59 | 0.444-0.733 |
| 44 | TPO.IgG | 0.59 | 0.443-0.732 |
| 45 | IA2.IgA | 0.58 | 0.432-0.737 |
| 46 | IAA.IgG | 0.58 | 0.519-0.631 |
| 47 | IF.IgG | 0.58 | 0.472-0.686 |
| 48 | Jo.1.IgM | 0.58 | 0.434-0.717 |
| 49 | La.SSb.IgG | 0.58 | 0.417-0.735 |
| 50 | Sm.IgM | 0.58 | 0.43-0.725 |
| 51 | Thyroglobulin.IgG | 0.58 | 0.436-0.722 |
| 52 | Thyroglobulin.IgM | 0.58 | 0.486-0.684 |
| 53 | U1.RNPA.IgA | 0.58 | 0.461-0.689 |
| 54 | beta2glycoprotein.IgM | 0.57 | 0.417-0.728 |
| 55 | IA2.IgG | 0.57 | 0.415-0.727 |
| 56 | U1RNPC.IgM | 0.57 | 0.426-0.711 |
| 57 | IF.IgM | 0.56 | 0.391-0.721 |
| 58 | MPO.4.IgM | 0.56 | 0.405-0.724 |
| 59 | CENP.B.IgM | 0.55 | 0.403-0.699 |
| 60 | ProIAA.IgM | 0.55 | 0.462-0.644 |
| 61 | RNP68.70.IgM | 0.55 | 0.394-0.703 |
| 62 | Scl.70.IgM | 0.55 | 0.397-0.7 |
| 63 | ZnT8.IgM | 0.54 | 0.398-0.682 |
| 64 | MPO.IgM | 0.52 | 0.374-0.665 |
| 65 | Thyroglobulin.IgA | 0.52 | 0.42-0.616 |
| 66 | Ro.SSA52.IgA | 0.51 | 0.449-0.577 |
| 67 | U1RNPC.IgG | 0.51 | 0.353-0.672 |
| 68 | ProIAA.IgG | 0.5 | 0.442-0.56 |
| 69 | RoSSA60.IgM | 0.5 | 0.353-0.643 |
| 70 | CCP.IgM | 0.49 | 0.341-0.647 |
| 71 | La.SSb.IgM | 0.49 | 0.346-0.637 |
| 72 | Smith.IgM | 0.49 | 0.345-0.64 |
| 73 | GAD65.IgM | 0.48 | 0.342-0.623 |
| 74 | U1.RNPA.IgM | 0.48 | 0.367-0.591 |
| 75 | IAA.IgM | 0.46 | 0.334-0.595 |
| 76 | Ro.SSA52.IgM | 0.46 | 0.389-0.534 |
| 77 | aNCA.PR3.IgM | 0.45 | 0.328-0.568 |
| 78 | TPO.IgM | 0.45 | 0.353-0.554 |

Samples from untreated and control subjects were compared. Top predictors as ranked by ROC and AUC are shown in Table 22.

TABLE 22

| | Biomarker | AUC | 95% CI |
|---|---|---|---|
| 1 | TGM2.IgA | 1 | 1-1 |
| 2 | DGP.IgA | 0.99 | 0.971-1 |
| 3 | DGP.IgG | 0.99 | 0.976-1 |
| 4 | TGM2.IgG | 0.96 | 0.909-1 |
| 5 | TGM2.IgM | 0.87 | 0.767-0.981 |
| 6 | Smith.IgG | 0.81 | 0.675-0.937 |
| 7 | Jo.1.IgA | 0.8 | 0.657-0.938 |
| 8 | DGP.IgM | 0.78 | 0.635-0.928 |
| 9 | beta2glycoprotein.IgG | 0.76 | 0.602-0.928 |
| 10 | Scl.70.IgA | 0.74 | 0.588-0.902 |
| 11 | Smith.IgA | 0.72 | 0.613-0.837 |
| 12 | aNCA.PR3.IgA | 0.7 | 0.552-0.848 |
| 13 | RoSSA60.IgA | 0.7 | 0.524-0.866 |
| 14 | CCP.IgA | 0.68 | 0.503-0.849 |
| 15 | IF.IgA | 0.68 | 0.511-0.849 |
| 16 | RNP68.70.IgA | 0.67 | 0.495-0.845 |
| 17 | ZnT8.IgA | 0.67 | 0.498-0.837 |
| 18 | CENP.B.IgA | 0.66 | 0.481-0.832 |
| 19 | GAD65.IgA | 0.66 | 0.492-0.833 |
| 20 | Sm.IgA | 0.66 | 0.48-0.835 |
| 21 | La.SSb.IgA | 0.65 | 0.467-0.833 |
| 22 | ProIAA.IgA | 0.65 | 0.547-0.753 |
| 23 | RoSSA60.IgG | 0.64 | 0.457-0.813 |
| 24 | Thyroglobulin.IgG | 0.63 | 0.466-0.794 |
| 25 | ZnT8.IgG | 0.63 | 0.447-0.815 |
| 26 | beta2glycoprotein.IgA | 0.62 | 0.439-0.796 |
| 27 | MPO.4.IgA | 0.62 | 0.457-0.781 |
| 28 | MPO.4.IgG | 0.62 | 0.434-0.799 |
| 29 | MPO.IgA | 0.62 | 0.438-0.797 |
| 30 | TPO.IgA | 0.62 | 0.456-0.789 |
| 31 | ZnT8.IgM | 0.62 | 0.454-0.791 |
| 32 | IAA.IgA | 0.61 | 0.436-0.784 |
| 33 | U1.RNPA.IgA | 0.61 | 0.463-0.752 |
| 34 | U1RNPC.IgA | 0.61 | 0.424-0.789 |
| 35 | Scl.70.IgG | 0.6 | 0.417-0.783 |
| 36 | Thyroglobulin.IgM | 0.6 | 0.476-0.724 |
| 37 | aNCA.PR3.IgG | 0.59 | 0.457-0.718 |
| 38 | GAD65.IgG | 0.59 | 0.409-0.771 |
| 39 | CCP.IgG | 0.58 | 0.39-0.763 |
| 40 | IAA.IgG | 0.58 | 0.495-0.655 |
| 41 | IF.IgG | 0.58 | 0.448-0.712 |
| 42 | IF.IgM | 0.58 | 0.401-0.767 |
| 43 | ProIAA.IgM | 0.58 | 0.464-0.701 |
| 44 | Ro.SSA52.IgG | 0.58 | 0.467-0.703 |
| 45 | U1.RNPA.IgG | 0.58 | 0.443-0.707 |
| 46 | CENP.B.IgG | 0.57 | 0.389-0.756 |
| 47 | IA2.IgM | 0.57 | 0.391-0.749 |
| 48 | RNP68.70.IgG | 0.57 | 0.385-0.75 |
| 49 | MPO.4.IgM | 0.56 | 0.369-0.744 |
| 50 | TPO.IgG | 0.56 | 0.391-0.724 |
| 51 | beta2glycoprotein.IgM | 0.55 | 0.367-0.733 |
| 52 | CENP.B.IgM | 0.55 | 0.37-0.735 |
| 53 | IA2.IgA | 0.55 | 0.362-0.73 |
| 54 | Jo.1.IgM | 0.55 | 0.369-0.729 |
| 55 | Sm.IgG | 0.55 | 0.355-0.74 |
| 56 | Thyroglobulin.IgA | 0.55 | 0.427-0.681 |
| 57 | IA2.IgG | 0.54 | 0.368-0.722 |
| 58 | RNP68.70.IgM | 0.54 | 0.37-0.72 |
| 59 | RoSSA60.IgM | 0.54 | 0.357-0.725 |
| 60 | Sm.IgM | 0.54 | 0.357-0.718 |
| 61 | Smith.IgM | 0.54 | 0.369-0.716 |
| 62 | Jo.1.IgG | 0.53 | 0.344-0.716 |
| 63 | MPO.IgG | 0.53 | 0.344-0.716 |
| 64 | MPO.IgM | 0.53 | 0.353-0.702 |
| 65 | ProIAA.IgG | 0.53 | 0.444-0.611 |
| 66 | U1RNPC.IgM | 0.53 | 0.349-0.716 |
| 67 | La.SSb.IgG | 0.52 | 0.335-0.71 |
| 68 | Ro.SSA52.IgA | 0.52 | 0.442-0.608 |
| 69 | U1RNPC.IgG | 0.52 | 0.34-0.71 |
| 70 | Scl.70.IgM | 0.51 | 0.322-0.693 |
| 71 | U1.RNPA.IgM | 0.49 | 0.361-0.619 |
| 72 | IAA.IgM | 0.48 | 0.326-0.634 |
| 73 | CCP.IgM | 0.46 | 0.272-0.638 |
| 74 | aNCA.PR3.IgM | 0.46 | 0.323-0.592 |
| 75 | GAD65.IgM | 0.46 | 0.307-0.613 |
| 76 | TPO.IgM | 0.46 | 0.344-0.571 |
| 77 | La.SSb.IgM | 0.45 | 0.273-0.625 |
| 78 | Ro.SSA52.IgM | 0.45 | 0.383-0.517 |

Samples from treated and untreated subjects were compared. Top predictors as ranked by ROC and AUC values are shown in Table 23.

TABLE 23

| | Biomarker | AUC | 95% CI |
|---|---|---|---|
| 1 | DGP.IgG | 0.94 | 0.872-1 |
| 2 | DGP.IgA | 0.92 | 0.837-1 |
| 3 | TGM2.IgA | 0.92 | 0.829-1 |
| 4 | TGM2.IgG | 0.86 | 0.74-0.977 |
| 5 | MPO.IgG | 0.82 | 0.691-0.949 |
| 6 | DGP.IgM | 0.78 | 0.633-0.925 |
| 7 | TGM2.IgM | 0.78 | 0.636-0.929 |
| 8 | Smith.IgG | 0.74 | 0.591-0.894 |
| 9 | CENP.B.IgG | 0.71 | 0.54-0.875 |
| 10 | Jo.1.IgG | 0.66 | 0.486-0.834 |
| 11 | ZnT8.IgM | 0.66 | 0.496-0.819 |
| 12 | RoSSA60.IgG | 0.64 | 0.469-0.821 |
| 13 | Thyroglobulin.IgG | 0.63 | 0.464-0.796 |
| 14 | La.SSb.IgG | 0.62 | 0.44-0.805 |
| 15 | Scl.70.IgG | 0.62 | 0.435-0.8 |
| 16 | Sm.IgG | 0.62 | 0.443-0.802 |
| 17 | aNCA.PR3.IgA | 0.61 | 0.44-0.775 |
| 18 | La.SSb.IgM | 0.6 | 0.429-0.769 |
| 19 | Scl.70.IgM | 0.59 | 0.405-0.77 |
| 20 | MPO.4.IgM | 0.58 | 0.387-0.763 |
| 21 | RoSSA60.IgM | 0.58 | 0.411-0.759 |
| 22 | U1.RNPA.IgG | 0.58 | 0.423-0.737 |
| 23 | RNP68.70.IgG | 0.57 | 0.39-0.755 |
| 24 | Sm.IgM | 0.57 | 0.403-0.744 |
| 25 | Smith.IgM | 0.57 | 0.396-0.736 |
| 26 | ZnT8.IgG | 0.57 | 0.383-0.752 |
| 27 | CCP.IgA | 0.56 | 0.37-0.74 |
| 28 | CCP.IgG | 0.56 | 0.376-0.744 |
| 29 | CCP.IgM | 0.56 | 0.386-0.744 |
| 30 | IA2.IgM | 0.56 | 0.378-0.737 |
| 31 | U1RNPC.IgM | 0.56 | 0.39-0.72 |
| 32 | IA2.IgA | 0.55 | 0.367-0.736 |
| 33 | IF.IgM | 0.55 | 0.369-0.736 |
| 34 | Sm.IgA | 0.55 | 0.368-0.74 |
| 35 | TPO.IgG | 0.55 | 0.373-0.732 |
| 36 | aNCA.PR3.IgG | 0.54 | 0.377-0.703 |
| 37 | GAD65.IgA | 0.54 | 0.361-0.729 |
| 38 | GAD65.IgM | 0.54 | 0.383-0.697 |
| 39 | IA2.IgG | 0.54 | 0.352-0.718 |
| 40 | Jo.1.IgM | 0.54 | 0.359-0.721 |
| 41 | Ro.SSA52.IgG | 0.54 | 0.395-0.693 |
| 42 | beta2glycoprotein.IgM | 0.53 | 0.35-0.715 |
| 43 | CENP.B.IgA | 0.53 | 0.343-0.717 |
| 44 | Jo.1.IgA | 0.53 | 0.337-0.718 |
| 45 | TPO.IgA | 0.53 | 0.347-0.708 |
| 46 | beta2glycoprotein.IgA | 0.52 | 0.335-0.705 |
| 47 | MPO.4.IgG | 0.52 | 0.326-0.709 |
| 48 | Ro.SSA52.IgM | 0.52 | 0.476-0.574 |
| 49 | Scl.70.IgA | 0.52 | 0.338-0.707 |
| 50 | U1RNPC.IgA | 0.52 | 0.34-0.71 |
| 51 | U1RNPC.IgG | 0.52 | 0.331-0.704 |
| 52 | ZnT8.IgA | 0.52 | 0.334-0.706 |
| 53 | GAD65.IgG | 0.51 | 0.327-0.698 |
| 54 | IF.IgG | 0.5 | 0.353-0.652 |
| 55 | La.SSb.IgA | 0.5 | 0.31-0.685 |
| 56 | MPO.IgA | 0.5 | 0.318-0.687 |
| 57 | RNP68.70.IgM | 0.5 | 0.326-0.674 |
| 58 | TPO.IgM | 0.5 | 0.402-0.593 |
| 59 | CENP.B.IgM | 0.49 | 0.315-0.66 |
| 60 | IAA.IgG | 0.49 | 0.374-0.604 |
| 61 | aNCA.PR3.IgM | 0.48 | 0.361-0.604 |
| 62 | IF.IgA | 0.48 | 0.295-0.668 |
| 63 | MPO.IgM | 0.48 | 0.311-0.654 |
| 64 | Ro.SSA52.IgA | 0.48 | 0.394-0.561 |
| 65 | U1.RNPA.IgM | 0.48 | 0.354-0.596 |
| 66 | beta2glycoprotein.IgG | 0.47 | 0.276-0.654 |
| 67 | IAA.IgM | 0.47 | 0.328-0.617 |
| 68 | MPO.4.IgA | 0.47 | 0.291-0.657 |
| 69 | Thyroglobulin.IgM | 0.47 | 0.321-0.609 |
| 70 | RoSSA60.IgA | 0.46 | 0.278-0.647 |
| 71 | IAA.IgA | 0.45 | 0.267-0.628 |
| 72 | ProIAA.IgG | 0.45 | 0.383-0.517 |
| 73 | U1.RNPA.IgA | 0.45 | 0.298-0.607 |
| 74 | ProIAA.IgM | 0.44 | 0.312-0.566 |
| 75 | RNP68.70.IgA | 0.44 | 0.256-0.629 |
| 76 | Thyroglobulin.IgA | 0.43 | 0.312-0.553 |
| 77 | ProIAA.IgA | 0.42 | 0.285-0.55 |
| 78 | Smith.IgA | 0.37 | 0.217-0.516 |

Panel Selection Using LASSO

LASSO panel selection was used to select panels to best differentiate between disease and control samples and to elect panels to best differentiate between flare and non-flare samples.

LASSO values were calculated as described, and using the equations presented in, Example 5. The optimal λ, value was determined using 10-fold cross validation. Panels generated using λ values that both minimize cost and result in cost estimates that are 1-standard error above the minimum are reported.

For the disease (treated and untreated) vs control data, the λ, value at minimum binomial deviance (λmin) equaled 0.004602123. The λ, value at 1 standard error above minimum binomial deviance (λ1se) equaled 0.09034155.

As shown in Table 24, using min in the cost function resulted in a panel of 12 biomarkers.

TABLE 24

| Predictor | Coefficient |
|---|---|
| Intercept | −42.6240503 |
| ACPA.IgG | −0.3052416 |
| beta2glycoprotein.IgG | 0.4496318 |
| CENP.B.IgG | 0.3785243 |
| GAD.IgA | 3.062002 |
| GAD.IgG | −2.3604885 |
| IA2.IgM | −0.9187905 |
| Jo.1.IgA | 4.6118991 |
| ProIAA.IgA | 3.8621275 |
| ProIAA.IgM | 2.5729241 |
| TGM2.IgA | 2.7352236 |
| U1.RNPA.IgA | 0.6389926 |
| ZnT8.IgA | 3.2659354 |

As shown in Table 25, using λ1 se in the cost function resulted in a panel of 1 biomarker.

TABLE 25

| Predictor | Coefficient |
|---|---|
| Intercept | −1.33705 |
| TGM2.IgA | 1.07991 |

For the untreated vs control data, the k value at minimum binomial deviance (λmin) equaled 0.004786186. The k value at 1 standard error above minimum binomial deviance (λ1se) equaled 0.03537337.

As shown in Table 26, using λmin in the cost function resulted in a panel of 6 biomarkers.

TABLE 26

| Predictor | Coefficient |
|---|---|
| Intercept | −12.5001886 |
| CENP.B.IgG | −0.45593214 |
| DGP.IgA | 0.7612091 |
| DGP.IgG | 1.61519735 |
| IA2.IgM | −0.08484583 |
| Jo.1.IgA | 1.31128797 |
| TGM2.IgA | 1.60413163 |

As shown in Table 27, using λ1se in the cost function resulted in a panel of 3 biomarkers.

TABLE 27

| Predictor | Coefficient |
|---|---|
| Intercept | −5.0558433 |
| DGP.IgA | 0.1115868 |
| DGP.IgG | 0.7320947 |
| TGM2.IgA | 1.1710355 |

For the treated vs untreated data, the k value at minimum binomial deviance (λmin) equaled 0.05959323. The k value at 1 standard error above minimum binomial deviance (λ1se) equaled 0.1314109.

As shown in Table 28, using min in the cost function resulted in a panel of 6 biomarkers.

TABLE 28

| Predictor | Coefficient |
|---|---|
| Intercept | 1.0327205 |
| DGP.IgA | −0.5825658 |
| DGP.IgG | −0.9898621 |
| Jo.1.IgM | 0.3547266 |
| MPO.IgG | 2.5588341 |
| TGM2.IgA | −0.3581559 |
| TGM2.IgG | −0.3419483 |

As shown in Table 29, using λ1se in the cost function resulted in a panel of 5 biomarkers.

TABLE 29

| Predictor | Coefficient |
|---|---|
| Intercept | 1.83160384 |
| DGP.IgA | −0.40811861 |
| DGP.IgG | −0.6497114 |
| MPO.IgG | 1.07665597 |
| TGM2.IgA | −0.13997709 |
| TGM2.IgG | −0.04754967 |

Anti-TGM2 and anti-DGP antibodies ranked highly as individual biomarkers for all three comparisons above. These markers were present in the selected panels, sometimes for multiple isotypes. Using min in the cost function: 1) TGM2.IgA was present in the disease vs. control panel; 2) TGM2 IgA, DGP IgA, and DGP IgG were present in the untreated Celiac vs. control panel; and 3) TGM2 IgA, TGM2 IgG, DGP IgA, and DGP IgG were present in the treated vs untreated Celiac panel. Studies were performed to gauge the extent to which anti-TGM2 and anti-DGP antibodies affect panel selection, and to determine whether panels can be successfully generated excluding these two markers.

Panels to best predict the same three outcomes as discussed above were generated using logistic LASSO regression excluding the classical Celiac biomarkers as discussed.

For the disease (treated and untreated) vs control data excluding DGP and TGM2, the λ, value at minimum binomial deviance (λmin) equaled 0.02540352. The λ, value at 1 standard error above minimum binomial deviance (λ1se) equaled 0.1558736.

As shown in Table 30, using λmin in the cost function resulted in a panel of 18 biomarkers.

TABLE 30

| Predictor | Coefficient |
|---|---|
| (Intercept) | −19.0488884 |
| aNCA.PR3.IgG | 0.330016669 |
| beta2glycoprotien.IgA | −0.00454479 |
| beta2glycoprotien.IgG | 1.61100838 |
| CENP.B.IgG | 0.106696816 |
| GAD.IgA | 1.655111117 |
| GAD.IgG | −2.66494473 |
| IA2.IgG | 2.233493239 |
| IA2.IgM | −1.87515524 |
| Jo.1.IgA | 6.688463434 |
| MPO.4.IgA | 0.145124869 |
| MPO.4.IgM | −1.32379585 |
| ProIAA.IgM | 0.157402269 |
| RNP68.70.IgM | −0.16153643 |
| Ro.SSA52.IgG | 0.399711208 |
| Ro.SSA52.IgM | −0.62180489 |
| RoSSA60.IgM | 2.382378472 |
| Smith.IgG | 0.198628675 |
| U1RNPC.IgA | −0.14093777 |

As shown in Table 31, using λ1se in the cost function resulted in a panel of 2 biomarkers.

TABLE 31

| Predictor | Coefficient |
|---|---|
| (Intercept) | −3.0818283 |
| Beta2glycoprotein.IgG | 0.06957892 |
| Jo.1.IgA | 1.77735523 |

For the untreated vs control data excluding DGP and TGM2, the λ, value at minimum binomial deviance (λmin) equaled 0.08019048. The λ, value at 1 standard error above minimum binomial deviance (λ1se) equaled 0.1110547.

As shown in Table 32, using λmin in the cost function resulted in a panel of 5 biomarkers.

TABLE 32

| Predictor | Coefficient |
|---|---|
| (Intercept) | −13.5661192 |
| beta2glycoprotien.IgG | 0.5327887 |
| IA2.IgM | −0.5353241 |
| Jo.1.IgA | 4.1043195 |
| Ro.SSA52.IgG | 1.62921477 |
| Smith.IgG | 0.9569802 |

As shown in Table 33, using λ1se in the cost function resulted in a panel of 5 biomarkers.

TABLE 33

| Predictor | Coefficient |
|---|---|
| (Intercept) | −9.4626352 |
| beta2glycoprotien.IgG | 0.3185424 |
| IA2.IgM | −0.1568486 |
| Jo.1.IgA | 2.9634465 |
| Ro.SSA52.IgG | 0.8422856 |
| Smith.IgG | 0.6962791 |

For the treated vs untreated data excluding DGP and TGM2, the k value at minimum binomial deviance (λmin) equaled 0.1275742. The k value at 1 standard error above minimum binomial deviance (λ1se) equaled 0.2128065.

As shown in Table 34, using λmin in the cost function resulted in a panel of 3 biomarkers.

TABLE 34

| Predictor | Coefficient |
|---|---|
| (Intercept) | −4.3247419 |
| CENP.B.IgG | 0.1493236 |
| MPO.IgG | 2.5146587 |
| Smith.IgG | 0.6358772 |

As shown in Table 35, using λ1se in the cost function resulted in a panel of 1 biomarker.

TABLE 35

| Predictor | Coefficient |
|---|---|
| (Intercept) | −2.327985 |
| MPO.IgG | 1.027876 |

### Example 7. Multiplexed, Isotype-Specific Research-Use Serology Assays for Detection of Autoimmune Reactivities

### Summary of Assays

Background: Autoimmune diseases affect over 50 million Americans. The presence of specific autoantibodies can predict disease onset in at-risk individuals (e.g., Type 1 diabetes, systemic lupus erythematosus, and celiac disease), and assist in distinguishing disorders with similar clinical features (e.g., Type 1 versus Type 2 diabetes). Multiplexed serology panels were developed for profiling IgG, IgA, and IgM autoantibody responses against 24 different autoantigens associated with important autoimmune diseases or connective tissue disorders (72 assays in total). These research-use-only panels were developed on the sensitive Meso-Scale Diagnostics® (MSD®) MULTI ARRAY technology platform and included measurements using bridging and/or classical serology assay formats. Serum-derived calibrators were used for quantitative measurement of each reactivity and as positive/negative controls for assay performance tracking. These panels were applied to three sample sets as described in Example 1: (1) samples from a drug trial (T1DAL) for Type 1 diabetes (ITN), (2) samples from a clinical study on gluten-free diets for celiac disease (Harvard University), and (3) matched lupus disease samples from individuals at or without flare (University of Minnesota).

Results: Assay performance data for calibrators, controls, and test samples are presented to demonstrate the reproducibility and robustness of the assay methods. Selected data are shown for markers that distinguish subgroups within a study set.

Conclusion: The multiplexed isotype-specific autoantibody assays provided reliable, quantitative, and sensitive measurement of 72 specific reactivities while requiring less than 200 μL of serum/plasma per sample. This platform provides a new tool that can be used in autoimmune disease research to broadly profile autoimmune reactivities in each sample.

### Methods

Assay panels were formatted for use in Bridging Simultaneous, Bridging Sequential, or Classical Serology assays, all using MSD®'s U-PLEX technology. The approach combines the performance advantages of serology assays with the sample-sparing advantages of multiplexed assays. Simultaneous detection of multiple autoantigen reactivities minimizes the amount of sample needed (<25 μL of diluted sample to detect all reactivities per panel, in duplicate). Samples are tested along with human serum-derived positive and negative controls and calibrators, to quantitate the autoimmune responses for each analyte and assess assay reproducibility.

Detection was performed using MSD®'s electrochemiluminescence (ECL) detection technology using SULFO-TAG™ labels that emit light upon electrochemical stimulation initiated at the electrode surfaces of MULTI-ARRAY® and MULTI-SPOT® microplates.

The samples were tested on multiple panels to measure reactivity to 24 autoantigens listed in Table 36, detecting IgA, IgG and IgM isotypes of each autoimmune reactivity, using the assay formats indicated in the table. Smith and MPO reactivities were measured in both bridging and classical serology formats. Thirty-eight samples were tested in duplicate on each assay plate along with MSD calibrator, and MSD positive and negative control samples. Samples for the bridging simultaneous assays were acid treated. Sample dilutions used ranged from 6 to 30-fold.

TABLE 36

| Autoantigen | Relevant Disease | Assay Format |
|---|---|---|
| Zinc Transported 8 protein (ZnT8) | Type 1 Diabetes | Bridging, Simultaneous |
| Insulinoma-2 (IA2) | Type 1 Diabetes | Bridging, Simultaneous |
| Insulin | Type 1 Diabetes | Bridging, Simultaneous |
| Proinsulin | Type 1 Diabetes | Bridging, Simultaneous |
| Glutamic acid decarboxylase | Type 1 Diabetes | Bridging, Simultaneous |
| (GAD or GAD65) | | |
| Intrinsic factor | Pernicious Anemia | Bridging, Simultaneous |
| Jo-1 | Polymyositis | Bridging, Simultaneous |
| Transglutaminase (tTG or TGM2) | Celiac disease | Bridging, Sequential |
| Deamidated forms of gliadin peptides | Celiac disease | Bridging, Sequential |
| (DGP) | | |
| Thyroid peroxidase (TPO) | Hashimoto's thyroiditis | Bridging, Sequential |
| Thyroglobulin | Hashimoto's thyroiditis | Bridging, Sequential |
| U1 RNP A | MCTD, SLE | Bridging, Sequential |
| Ro/SSA-52 | MCTD, SLE, Sjogren's | Bridging, Sequential |
| | syndrome | |
| aNCA-PR3 | Vasculitis | Bridging, Sequential |
| MPO | Vasculitis | Bridging and Classical |
| | | Serology |
| Smith (enriched for SmD) | MCTD, SLE | Bridging and Classical |
| | | Serology |
| CENP B | Scleroderma, SLE | Classical Serology |
| Ro/SSA 60 | MCTD, SLE, Sjogren's | Classical Serology |
| | syndrome | |
| Scl 70 (topoisomerase I) | Scleroderma, MCTD | Classical Serology |
| La/SSB | Sjogren's Syndrome, SLE | Classical Serology |
| beta2glycoprotein | APS | Classical Serology |
| ACPA (anti-CCP) (2 peptides) | Rheumatoid Arthritis | Classical Serology |
| U1 RNP68/70 | MCTD, SLE | Classical Serology |
| U1 RNP C | MCTD, SLE | Classical Serology |

Calibrators and controls were prepared from screened human serum/plasma samples. Multiple individual patient samples were sourced and tested for reactivity to each antigen, to identify ones with high levels of autoantibodies. In most cases, each isotype (IgA, IgG, and IgM) required unique samples. The sample signals had to be high enough such that, following pooling of samples for individual reactants in a panel of assays, sufficient dynamic range remained for calibrator materials. No recombinant material is available for use as calibrator or control.

Positive and negative controls were run in duplicate per plate, over 6 plates per panel, run across 6 days, by 3 analysts. The summarized positive controls signals and calculated concentrations demonstrate robust performance for most assays. Results are shown in Table 37.

TABLE 37

| | | IgA Assay | | IgG Assay | | IgM Assay | |
|---|---|---|---|---|---|---|---|
| Antigen | | Average | CV | Average | CV | Average | CV |
| ACPA | Signal | 8,764 | 29% | 8,911 | 18% | 91 | 14% |
| | Concentration (U/mL | 75 | 17% | 19 | 4% | 54 | 23% |
| aNCA PR3 | Signal | 6,363 | 44% | 428,117 | 6% | 5,071 | 88% |
| | Concentration (U/mL | 102 | 5% | 345 | 8% | 157 | 32% |
| Beta2glycoprotien | Signal | 24,589 | 4% | 158,499 | 8% | 1,581 | 16% |
| | Concentration (U/mL | 9,535 | 11% | 545 | 5% | 780 | 5% |
| CENP B | Signal | 16,031 | 9% | 49,201 | 3% | 125 | 18% |
| | Concentration (U/mL | 75 | 5% | 19 | 2% | 63 | 15% |
| DGP | Signal | 57,818 | 21% | 113,282 | 17% | 17,947 | 21% |
| | Concentration (U/mL | 59 | 15% | 129 | 12% | 116 | 8% |
| GAD65 | Signal | 43,544 | 7% | 7,739 | 15% | 46,137 | 17% |
| | Concentration (U/mL | 184 | 9% | 5 | 8% | 378 | 6% |
| IA2 | Signal | 5,196 | 18% | 2,151 | 41% | 2,564 | 22% |
| | Concentration (U/mL | 342 | 6% | 3 | 53% | 298 | 14% |
| Insulin | Signal | 4,187 | 29% | 10,005 | 35% | 1,821 | 25% |
| | Concentration (U/mL | 645 | 9% | 738 | 28% | 665 | 19% |
| IF | Signal | 1,832 | 11% | 61,493 | 15% | 48,916 | 18% |
| | Concentration (U/mL | 171 | 11% | 54 | 15% | 1,212 | 15% |
| Jo-1 | Signal | 30,585 | 36% | 224,436 | 20% | 90,723 | 17% |
| | Concentration (U/mL | 359 | 9% | 1,542 | 67% | 462 | 9% |
| La SSb | Signal | 69,293 | 6% | 4,172,910 | 2% | 7,609 | 8% |
| | Concentration (U/mL | 153 | 5% | 784 | 8% | 326 | 3% |
| MPO - Classical | Signal | 10,866 | 13% | 16,917 | 6% | 2,029 | 10% |
| | Concentration (U/mL | 68 | 3% | 17 | 2% | 74 | 4% |
| MPO-Bridging | Signal | 14,448 | 25% | 5,870 | 19% | 318,872 | 10% |
| | Concentration (U/mL | 475 | 13% | 14 | 9% | 730 | 3% |
| Pro-insulin | Signal | 361 | 16% | 1,289 | 39% | 227 | 18% |
| | Concentration (U/mL | 781 | 10% | 497 | 33% | 855 | 11% |
| RNP68 70 | Signal | 298,266 | 3% | 230,331 | 4% | 1,254 | 5% |
| | Concentration (U/mL | 254 | 6% | 62 | 4% | 77 | 6% |
| Ro/SSA52 | Signal | 13,762 | 37% | 6,342 | 20% | 71,466 | 93% |
| | Concentration (U/mL | 8 | 48% | 13 | 13% | 1,645 | 72% |
| RoSSA60 | Signal | 86,364 | 3% | 334,172 | 3% | 369 | 8% |
| | Concentration (U/mL | 139 | 2% | 35 | 5% | 63 | 5% |
| Scl 70 | Signal | 114,469 | 8% | 45,140 | 2% | 541 | 4% |
| | Concentration (U/mL | 88 | 3% | 17 | 2% | 65 | 4% |
| Smith - Classical | Signal | 5,013 | 16% | 9,017 | 4% | 188 | 8% |
| | Concentration (U/mL | 198 | 3% | 19 | 5% | 73 | 18% |
| Smith Bridging | Signal | 4,699 | 27% | 8,187 | 22% | 5,822 | 13% |
| | Concentration (U/mL | 25 | 19% | 156 | 15% | 123 | 10% |
| TGM2 | Signal | 160,568 | 14% | 124,169 | 13% | 13,372 | 24% |
| | Concentration (U/mL | 27 | 23% | 86 | 10% | 219 | 12% |
| Thyroglobulin | Signal | 2,543 | 18% | 32,701 | 16% | 4,360 | 9% |
| | Concentration (U/mL | 683 | 17% | 2,341 | 19% | 681 | 5% |
| TPO | Signal | 7,102 | 17% | 399,102 | 17% | 5,858 | 131% |
| | Concentration (U/mL | 60 | 6% | 39 | 10% | 123 | 52% |
| U1 RNPA | Signal | 11,819 | 22% | 1,779 | 7% | 3,709 | 61% |
| | Concentration (U/mL | 190 | 21% | 1 | 29% | 111 | 43% |
| U2RNPC | Signal | 128,632 | 8% | 6,951 | 16% | 606 | 9% |
| | Concentration (U/mL | 408 | 6% | 75 | 7% | 70 | 5% |
| ZnT8 | Signal | 2,863 | 30% | 14,425 | 29% | 1,902 | 13% |
| | Concentration (U/mL | 510 | 9% | 135 | 34% | 1,280 | 36% |

### Sample Cohorts

As described above, three sample cohorts were used: a T1DAL cohort, a Celiac disease cohort and a Systemic Lupus Erythematosus cohort.

### TIDAL/ITN045AI Cohort—INDUCING REMISSION IN NEW ONSET TIDM WITH ALEFACEPT (Amevive®)

This was a multi-center, prospective, double-blind, placebo-controlled, 50-patient, 2:1 randomized, phase II clinical trial for individuals with recent-onset Type 1 Diabetes, aged 12-35 years. Participants received weekly injections of alefacept (15 mg) or placebo for 12 weeks, followed by a 12-week pause before resuming another 12 weeks of dosing, for a total course of 24 weeks of alefacept or placebo. (Rigby et al., 2013) https://www.ncbi.nlm.nih.gov/pmc/articles/PMC3957186/pdf/nihms543103.pdf). Alefacept is a dimeric fusion protein, consisting of the leukocyte function antigen-3 protein and Fc portion of human IgG1, that blocks the T-cell CD2 receptor, thus preventing T-cell proliferation. It also induces apoptosis of effector memory T cells.

The purpose of assaying this cohort was to measure autoantibodies during Alefacept treatment and stratify with response (change in disease progression) to see if there is any correlation. Samples were provided blinded with respect to treatment group and outcome.

Celiac Disease Cohort

For the celiac disease cohort, there were three types of samples, as described below.

Newly diagnosed, untreated celiac disease (20 samples). Patients were confirmed positive by anti-TGM2 (tTG or TGM2) IgA serology and small bowel biopsy demonstrating characteristic changes of celiac disease histopathology with villus atrophy (Marsh III lesion). A gluten free diet (GFD) had not yet been initiated or was initiated no more than 4 weeks prior to serum collection.

Treated celiac disease (20 samples). These are patients with known celiac disease at a follow up visit with clinically indicated celiac serology request/sample collection for monitoring serologic response to GFD. The gluten free diet had been initiated at least 12 months prior to serum collection.

Non-celiac (NC) controls (20 samples). Celiac disease in these patients was excluded clinically and by negative celiac serology. These patients had gastro-intestinal symptoms caused by confirmed gastrointestinal disorders and were ingesting a normal, gluten-containing diet prior to serum collection.

The purpose of assaying this cohort was to identify markers of celiac disease, and markers of GFD treatment response.

Systemic Lupus Erythematosus (SLE) Cohort

The samples for this cohort included sera from 15 SLE patients. For each patient, samples were from one flare time point (HIGH) and one non-flare time point (LOW). The visit number at which samples were collected and their SLEDAI scores were provided. The HIGH sample may have been collected from a time point that followed or preceded the LOW sample in a given patient. Additionally, 15 control samples from matched normal subjects were provided (single time point for each).

The purpose of assaying this cohort was to evaluate the performance of SLE-related markers in the classical serology and/or bridging formats, identify potential novel SLE markers, and identify potential markers of SLE flare development.

### T1DAL Testing—Sample Reproducibility

All samples were tested in duplicate on all assays. Replicate measurements were highly reproducible along the dynamic ranges of each assay.

### Celiac Disease Study

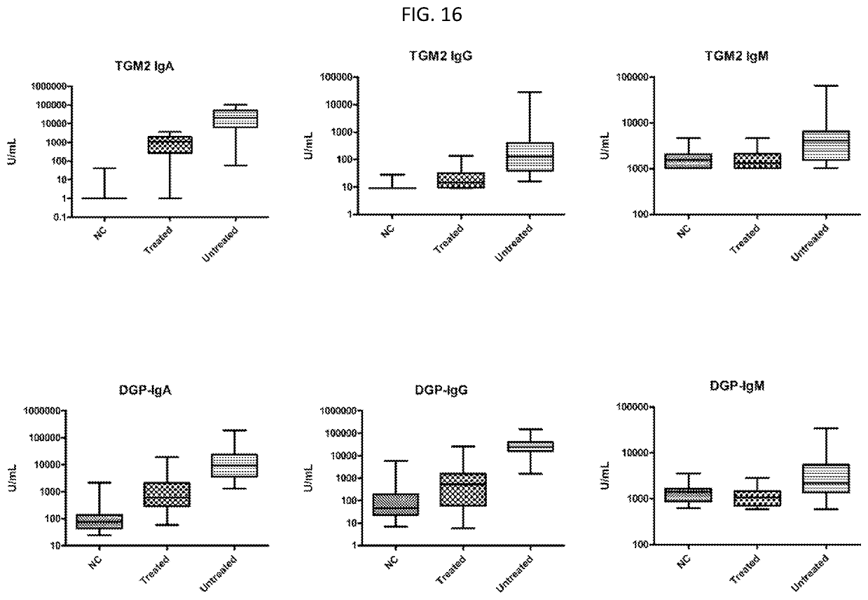

Data are shown in FIG. 16 for two classical celiac disease markers. Anti-TGM2 and anti-DGP antibody levels are highest in Untreated patient samples, and clearly reduced in Treated patient samples. Levels in celiac patient samples are elevated relative to non-celiac (NC) controls regardless of treatment status except for IgM reactivities that were comparable for NC and treated patient samples.

### SLE Study

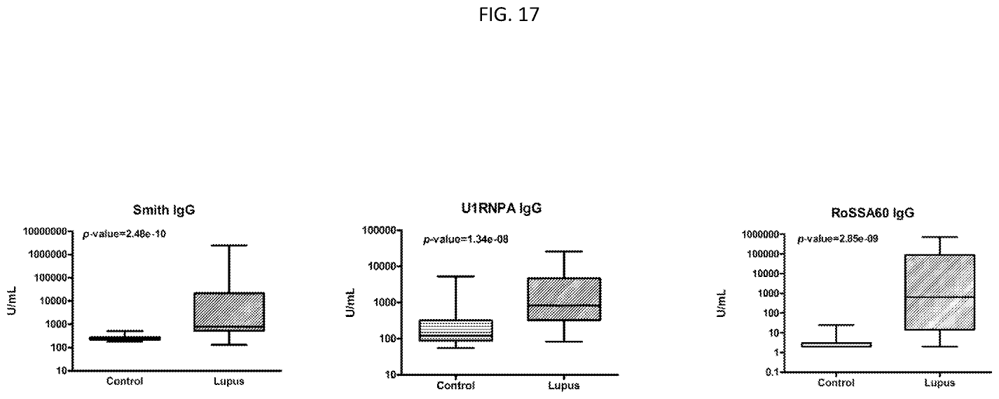

The classical SLE markers were shown to separate SLE from matched control samples very efficiently. Data for the top three performing markers are shown in FIG. 17. This is likely the first demonstration of the use of a bridging serology assay for an SLE marker (Intrinsic Factor, Jo-1, MPO, Smith, U1 RNPA, Ro/SSA52 and aNCA PR3).

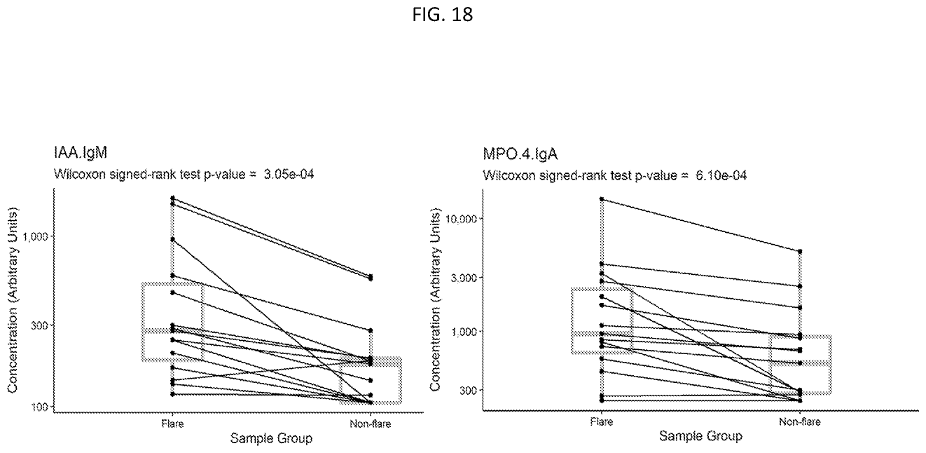

When markers were ranked by the Mann-Whitney-Wilcoxon test for their ability to distinguish SLE flare from non-flare samples, the top predictors were not the classical SLE markers, but were IAA-IgM and MPO-IgA, autoantigens associated with Type 1 diabetes and vasculitis, respectively. Results are shown in FIG. 18. Each line below represents one patient, connecting the flare and non-flare marker concentrations.

### CONCLUSION

Based on experience with the sample testing discussed above, all the listed markers (24 autoantigen reactivities×3 isotypes) can be tested in samples in duplicate using a total of ˜150 μL serum/plasma, yielding quantitative measures for each reactivity. These results demonstrate the sample-sparing capability of the approach described, and the robustness, reproducibility, and versatility of the multiplexed assays described herein. The broad applicability of the bridging serology approach with its inherent advantages, including increased specificity, is also demonstrated for markers that are not normally assessed in this format.

### Example 8. Validation and Testing of a Five Marker Panel With Samples From Type 1 Diabetes Subjects

This Example describes the validation and testing of panel of five biomarkers—anti-TGM2, anti-GAD65, anti-ZnT8, anti-insulin and anti-IA2. After validation, the panel was tested with samples from Type 1 Diabetes subjects.

### Sample Preparation and Reagents

The following protocol was used for measuring autoantibodies against TGM2, GAD65, ZnT8, Insulin and IA-2 from human serum. The reagents used are shown in Table 38. These reagents are available from Meso Scale Diagnostics (MSD) in Rockville, Md., USA.

TABLE 38

| | Storage | |
|---|---|---|
| Required Materials | temp. | |
| U-PLEX ® 5-Assay, 96-Well SECTOR Plate | 2-8° | C. |
| U-PLEX ® Linker 1 | 2-8° | C. |
| U-PLEX ® Linker 2 | 2-8° | C. |
| U-PLEX ® Linker 3 | 2-8° | C. |
| U-PLEX ® Linker 8 | 2-8° | C. |
| U-PLEX ® Linker 10 | 2-8° | C. |
| U-PLEX ® Stop Solution | 2-8° | C. |
| Biotin Hu (Human) TGM2 Protein (25X) | <−70° | C. |
| Biotin Hu (Human) GAD65 Protein (25X) | <−70° | C. |
| Biotin Hu (Human) ZnT8 Protein (25X) | <−70° | C. |
| Biotin Hu (Human) Insulin Protein (25X) | <−70° | C. |
| Biotin Hu (Human) IA2 Protein (25X) | <−70° | C. |
| SULFO-TAG ™ Hu TGM2 Protein (100X) | <−70° | C. |
| SULFO-TAG ™ Hu GAD65 Protein (100X) | <−70° | C. |
| SULFO-TAG ™ Hu ZnT8 Protein (100X) | <−70° | C. |
| SULFO-TAG Hu Insulin Protein (100X) | <−70° | C. |
| SULFO-TAG Hu IA-2 Protein (100X) | <−70° | C. |
| Hu T1D Antibody Panel 1 Calibrator (5X) | <−70° | C. |
| Diluent 55 | <−70° | C. |
| Hu (Human) T1D Antibody Panel 1 Positive Control 1 | <−70° | C. |
| (1X) | | |
| Hu (Human) T1D Antibody Panel 1 Positive Control 2 | <−70° | C. |
| (1X) | | |
| Hu (Human) T1D Antibody Panel 1 Negative Control | <−70° | C. |
| (1X) | | |
| MSD Diluent 100 | 2-8° | C. |

| | |
|---|---|
| MSD Wash Buffer (20X) | RT |
| MSD GOLD ™ Read Buffer | RT |

Other materials and equipment that were used in performing the protocol include: 96-well polypropylene round-bottom dilution plates; adhesive plate seals; tabletop centrifuge or micro-centrifuge; microtiter plate shaker; microcentrifuge tubes for making serial dilutions; automated plate washer or other efficient multi-channel pipetting equipment; and appropriate liquid handling equipment.

The sample preparation protocol was performed in the following steps.

Step 1—Thaw Reagents—Calibrators, controls, and samples were thawed on ice. All reagents and plates were allowed to stand at room temperature (RT) to acclimate for at least 30 minutes. The following general reagents were used in assaying 1 plate: 1 U-PLEX plate; 450 μL of Linker 1; 450 μL of Linker 2; 450 μL of Linker 3; 450 μL of Linker 8; 450 μL of Linker 10; 2 mL Stop Solution; and 5 mL Diluent 100.

Step 2—Preparation of U-PLEX® Linker Coupled Biotin Protein Mix—As Biotin Hu TGM2 protein, Biotin Hu GAD65 protein, Biotin Hu ZnT8 protein, Biotin Hu Insulin protein and Biotin Hu IA-2 protein are provided at 25× of the working concentration, each protein was diluted prior to use. The U-PLEX linker coupled biotin protein mix was prepared for 1 plate. Each biotin protein was diluted in MSD Diluent 100 as follows:
1× Biotin Hu TGM2: 15 μL of 25× Biotin Hu TGM2 Protein+360 μL of Diluent 100
1× Biotin Hu GAD65 Protein: 15 μL of 25× Biotin Hu GAD65 Protein+360 μL of Diluent 100
1× Biotin Hu ZnT8 Protein: 15 μL of 25× Biotin Hu ZnT8 Protein+360 μL of Diluent 100
1× Biotin Hu Insulin Protein: 15 μL of 25× Biotin Hu Insulin Protein+360 μL of Diluent 100
1× Biotin Hu IA-2 Protein: 15 μL of 25× Biotin Hu IA-2 Protein+360 μL of Diluent 100

Individual U-PLEX® Linker-coupled antibody solutions were created in separate microcentrifuge tubes by making the following combinations:
300 μL of 1× Biotin Hu TGM2 Protein+450 μL of Linker 1
300 μL of 1× Biotin Hu GAD65 Protein+450 μL of Linker 2
300 μL of 1× Biotin Hu ZnT8 Protein+450 μL of Linker 3
300 μL of 1× Biotin Hu Insulin Protein+450 μL of Linker 8
300 μL of 1× Biotin Hu IA-2 Protein+450 μL of Linker 10

Individual tubes were mixed by vortexing and incubated at RT for 30 minutes. After incubation, 300 μL of Stop Solution was added to each tube and mixed by vortexing. Tubes were then incubated at RT for an additional 30 mins.

840 μL of each U-PLEX Linker coupled biotin protein was combined together to prepare the U-PLEX Linker coupled biotin protein mix.

Step 3—Prepare Calibrator—The 5× Kit Calibrator was diluted 5-fold in Calibrator Diluent (Diluent 55) to prepare Calibrator 1. Calibrator 1 was then serially diluted by 3-fold in Calibrator Diluent to prepare an 8-point calibration curve as shown in Table 39. Calibrator Diluent alone was used as Calibrator 8.

TABLE 39

| | | Source | Diluent 55 | Total | Final |
|---|---|---|---|---|---|
| | | Volume | Volume | vol | vol |
| Calibrator | Source | (μL) | (μL) | (μL) | (μL) |
| Cal 1 | Hu T1D Antibody | 15 | 60 | 75 | 50 |
| | Panel 1 Calibrator | | | | |
| | (5X) | | | | |
| Cal 2 | Cal 1 | 25 | 50 | 75 | 50 |
| Cal 3 | Cal 2 | 25 | 50 | 75 | 50 |
| Cal 4 | Cal 3 | 25 | 50 | 75 | 50 |
| Cal 5 | Cal4 | 25 | 50 | 75 | 50 |
| Cal 6 | Cal 5 | 25 | 50 | 75 | 50 |
| Cal 7 | Cal 6 | 25 | 50 | 75 | 50 |
| Cal 8 | — | — | 50 | 75 | 50 |

Step 4—Prepare Samples—All controls and samples (as described below) were tested neat—no prior dilution was required.

Step 5—Prepare SULFO-TAG Protein Mix—SULFO-TAG Hu TGM2 Protein, SULFO-TAG Hu GAD65 Protein, SULFO-TAG Hu ZnT8 Protein, SULFO-TAG Hu Insulin and SULFO-TAG Hu IA-2 Protein were provided at 100× stock concentrations and were used at a working concentration of 1×. The SULFO-TAG Protein Mix for 1 plate was prepared by combining:
25 μL of 100× SULFO-TAG Hu TGM2 Protein
25 μL of 100× SULFO-TAG Hu GAD65 Protein
25 μL of 100× SULFO-TAG Hu ZnT8 Protein
25 μL of 100× SULFO-TAG Hu Insulin Protein
25 μL of 100× SULFO-TAG Hu IA-2 Protein
2375 μL of Diluent 100

### Plating and Analysis

The samples and reagent mixtures were combined and assayed according to the following steps.

Step 1-70 μL of the U-PLEX linker coupled biotin protein mix, 40 μL of the SULFO-TAG protein mix and 30 μL of samples or standard were added to each well of a round-bottom 96-well polypropylene plate. The plate was sealed with adhesive plate seal and incubated with shaking for 1 hour at room temperature.

Step 2-50 μL from each of the polypropylene plate was transferred to the U-PLEX plate. The plate was sealed with adhesive plate seal and incubated with shaking for 1 hour at room temperature.

Step 3—The assay plate was washed three times with at least 150 μL/well of MSD Wash Buffer. 150 μL/well of MSD GOLD Read Buffer was added to the assay plate. Assay plates were read on the MSD SECTOR instrument immediately after adding Read Buffer. Care was taken to avoid introducing bubbles when adding Read Buffer.

### Calibration Curve Reproducibility and Assay Limits

Calibration curves were made for each of the five proteins—TGM2, GAD65, ZnT8, Insulin and IA2—using 3-fold serial dilution as described above. Calibration curve signals from 26 plates and 64 measurements collected by 4 different operators were analyzed. Two validation lots were tested—Validation Lot 1 and Validation Lot 2. For each of the five markers for both lots, inter-run signal percent coefficient of variation (% CV) was within 20% at all the non-zero calibration points and intra-run signal % CV was within 10% at all the non-zero calibration points.

The assay Limit of Blank (LoB) was determined for both validation lots. The LoB was calculated as the concentration of the signal that corresponds to the 95th percentile of the signal distribution of blank samples. The blanks tested are Calibrator 08 as described above. Calibrator 08 is made with negative human serum matrix. The LoB signal was calculated as follows: LoB Signal=(Mean Signal of Cal 08+1.645*(Std. Deviation of blank Signal)). LoB (U/mL) refers to the concentration corresponding to the LoB signal. Blanks (Calibrator 08) were assayed across a total of 64 measurements using 26 plates assayed by 4 operators. Values for each assay in each of Validation Lot 1 and Validation Lot 2 are shown in Table 40.

TABLE 40

| | Validation Lot 2 | Validation Lot 1 |
|---|---|---|
| Assay | LoB (U/mL) | LoB (U/mL) |
| Anti-TGM2 | 2.6 | 2.8 |
| Anti-GAD65 | 0.8 | 0.7 |
| Anti-ZnT8 | 1.5 | 1.9 |
| Anti-Insulin | 0.9 | 0.4 |
| Anti-IA-2 | 0.8 | 0.2 |

The lower limit of quantitation (LLOQ) was also determined for both validation lots. The LLOQ was established as the lowest concentration that has a total error of <40%. At least 85% of the total measurements of a LLOQ samples meet the above acceptance criteria. Twenty-eight measurements were performed for each LLOQ sample in 7 runs with 3 operators. Results are shown in Table 41.

TABLE 41

| | Validation Lot 2 | Validation Lot 1 |
|---|---|---|
| Assay | LLOQ (U/mL) | LLOQ (U/mL) |
| Anti-TGM2 | 21.2 | 19.0 |
| Anti-GAD65 | 8.4 | 7.9 |
| Anti-ZnT8 | 6.3 | 6.0 |
| Anti-Insulin | 1.9 | 1.5 |
| Anti-IA-2 | 2.4 | 1.8 |

### Assessing Intra-Assay Precision

A single assay run was performed in order to assess the intra-run precision of the assay. The Human T1D Antibody Panel 1 described above has two matrix-based positive controls: Human T1D Antibody Panel 1 Positive control 1 (PC1) and Human T1D Antibody Panel 1 Positive Control 2 (PC2). Twenty replicates of the two positive controls were tested in a single assay run to determine the intra-run assay precision for each analyte in the panel. The intra-run % concentration coefficient of variation for both positive controls across all five assays is within 10% and in all cases is close to or below 5% as shown in Table 42.

TABLE 42

| | Intra-run Conc. | Intra-run Conc. |
|---|---|---|
| | % CV (PC1) | % CV (PC2) |
| Anti-TGM2 | 2.8 | 4.3 |
| Anti-GAD65 | 2.3 | 2.0 |
| Anti-ZnT8 | 3.2 | 3.1 |
| Anti-Insulin | 3.7 | 5.1 |
| Anti-IA-2 | 3.4 | 3.5 |

### Assessing Inter-Assay Precision and Accuracy

In order to assess the repeatability of results using the panel over multiple runs, precision and accuracy across runs were evaluated by using the two matrix-based positive controls: Positive Control 1 (PC1) and Positive Control 2 (PC2). The concentrations of the positive controls from 26 runs corresponding to a total of 82 measurements were collected from 4 operators. The accuracy of the two positive controls was calculated as: % Accuracy=(Average of 82 measurements/Assigned concentration in QC)×100. The inter-run concentration coefficient of variation for each controls for each assay is within 15%. The accuracy of the two positive controls for all 5 assays is within 80%-120%. These values show that there is good precision and accuracy between runs for the panel.

### Assay Cut-Point Establishment

Clinical cut-points were established for each of the five markers. A clinical cut-point is the signal value above or below which the signal is significant enough to be associated with a positive diagnosis. Clinical cut-points are determined by analyzing “normal” samples, i.e., samples taken from subjects that are not diagnosed with the disease or disorder being tested for. In this example, “normal” subjects are those that do not have Type 1 Diabetes (T1D).

A total of 97 “normal” samples were obtained from a commercial vendor and tested with the Validation Lot 2 kit. The samples may contain zero or one T1D positive antibodies but come from subjects that are asymptomatic. If a sample tests positive for two or more of the five T1D antibodies on the panel, then the sample is removed from the analysis, as it is possible that the sample is from a subject who actually has T1D and thus is not “normal” for the purposes of the T1D cut-point assay. From the 97 samples, there were 41 males and 56 females ranging in age from 20-30 years.

Clinical Cut-points were analyzed by two methods with different percentile distributions:

90th percentile of sample distribution: this distribution is for applications where a higher false positive rate is desired. In the case of a 90th percentile distribution, it may be desirable to follow up with additional confirmatory tests to individuals who tested positive in the initial test. For certain tests and conditions, it may be better to have a false positive than to miss diagnosing an individual who may be at risk for T1D.

98th percentile of sample distribution: this distribution is commonly used in autoantibody measurement in the field along with the 90th percentile and the 95th percentile. The goal of this distribution is to minimize the false positives. The 98th percentile was used to evaluate negative controls and sample measurements.

The 97 samples were analyzed using the methods described above in this example. One sample was found to be an outlier and was removed from the analysis, leaving a total of 96 samples. The cut-points determined for each assay at both the 90th and 98th percentile are shown in Table 43. A plot of the results is shown in FIG. 20, with the top horizontal line in each column representing the 98th percentile cut-point and the bottom horizontal line in each column representing the 90th percentile cut-point.

TABLE 43

| | 90^{th} Percentile distribution | | 98^{th} Percentile distribution | |
|---|---|---|---|---|
| | | # of samples | | # of samples |
| | Cut-point | above Cut- | Cut-point | above Cut- |
| Assay | (U/mL) | point | (U/mL) | point |
| Anti-TGM2 | 5.7 | 10/96 | 8.8 | 2/96 |
| Anti-GAD65 | 6.8 | 11/96 | 13.6 | 2/96 |
| Anti-ZnT8 | 3.3 | 10/96 | 7.5 | 2/96 |
| Anti-Insulin | 0.6 | 14/96 | 1.4 | 2/96 |
| Anti-IA-2 | 0.6 | 10/96 | 2.2 | 2/96 |

### Sample Testing

A total of 172 T1D human serum samples were obtained from commercial vendors. The samples were from individuals ranging in age from 2-30 years. An additional 23 Celiac disease samples were obtained from commercial vendors and tested with the TMG2 assay. However, some of these donors could be on a gluten-free diet and therefore may have diminished anti-TGM2 antibodies. The Celiac disease samples were from 10 Males and 13 Females ranging in age from 4-28 years.

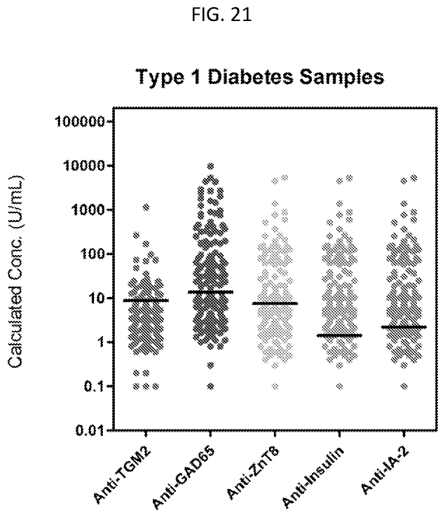

Samples were tested using the Validation Kit Lot 2 with the 98th percentile of normal sample used as the cut-point for each assay. The cut-points for each assay and the number of samples meeting the cut point are shown in Table 44. The data for each assay are plotted in FIG. 21, with the horizontal line representing the 98th percentile cut-point.

TABLE 44

| Analyte | Cut-Point (U/mL) | Number of samples above Cut-Point |
|---|---|---|
| Anti-TGM2 | 8.8 | 39/195 (Celiac + TD samples) |
| Anti-GAD65 | 13.6 | 92/172 (T1D Samples) |
| Anti-ZnT8 | 7.5 | 68/172 (T1D Samples) |
| Anti-Insulin | 1.4 | 61/172 (T1D Samples) |
| Anti-IA-2 | 2.2 | 87/172 (T1D Samples) |

Table 45 shows the number of samples having values above the cut-point for a certain number of assays. As can be seen in the table, 53.5% of samples (92 out of 172) had values above the cut-point for more than one assay.

TABLE 45

| >CP for ‘n’ analytes | Number of samples |
|---|---|
| n = 0 | 25/172 |
| n = 1 | 55/172 |
| n = 2 | 39/172 |
| n = 3 | 37/172 |
| n = 4 | 16/172 |
| n > 1 | 92/172 (53.5%) |

In order to further verify the reproducibility of the five member panel, a total of 107 human serum samples obtained from commercial vendors were tested on three different sets of kits: the original Verification kit, Validation Lot 1, and Validation Lot 2. The sample group contained 102 T1D Samples from 42 males and 60 females ranging in age from 5-30 years. The results showed that sample quantitation is comparable across the three kit lots, with all three kits showing results within ±20% of one another.

The results presented here thus show that the five member panel described has good accuracy and precision and is able to correctly diagnose T1D in samples from patients. The results indicate that the panel can be an important diagnostic that will provide early stage diagnosis of T1D. This early stage diagnosis will allow for early stage treatment that can prevent the significant morbidity associated with the disease.

### Example 9. Determination of Cut-Points For a Five Marker Panel

This Example describes the determination of cut-points for a panel of five biomarkers—anti-TGM2, anti-GAD65, anti-ZnT8, anti-insulin and anti-IA2.

90 “normal” control samples obtained from the Islet Autoantibody Standardization Program (IASP) were tested with assays for anti-GAD65, anti-ZnT8 and anti-IA2 generally as described in Example 8 to determine cut-points for these assays at the 95^{th} percentile. 98 “normal” control samples were obtained from a commercial vendor and tested with the anti-insulin assay generally as described in Example 8 and used to refine the cut-point for anti-insulin at the 95^{th} percentile. 96 “normal” control samples obtained from a commercial vendor were tested across 7 runs with assays for anti-TGM2 generally as described in Example 8 to determine the cut-point for anti-TGM2 at the 98^{th} percentile. The determined cut-points are shown in Table 46.

TABLE 46

| | Cut-point | |
|---|---|---|
| Anti-TGM2 | 11.9 | U/mL |
| Anti-GAD65 | 13.1 | IU/mL |
| Anti-ZnT8 | 7.9 | U/mL |
| Anti-Insulin | 0.65 | U/mL |
| Anti-IA-2 | 1.2 | IU/mL |

What is claimed is:

1. A multiplexed assay method comprising, simultaneously detecting at least two human biomarkers in a biological sample in a multiplexed assay format, wherein the at least two biomarkers comprise (a) anti-IA2 IgG and (b) anti-beta2glycoprotein IgG, wherein the multiplexed assay comprises:
a. combining, in one or more steps:
i. the biological sample;
ii. at least a first and second binding reagent, wherein the first and second binding reagent is a binding partner of anti-IA2 IgG and anti-beta2glycoprotein IgG, respectively;
b. forming at least a first and second binding complex comprising the binding reagents and the biomarkers; and
c. detecting the biomarkers in each of the binding complexes.

2. A multiplexed assay method comprising, simultaneously detecting at least four human biomarkers in a biological sample in a multiplexed assay format, wherein the at least four biomarkers comprise (a) anti-IA2 IgG, (b) anti-beta2glycoprotein IgG, (c) anti-DGP IgG and (d) anti-IA2 IgM, wherein the multiplexed assay comprises:
a. combining, in one or more steps:
i. the biological sample;
ii. at least a first, second, third and fourth binding reagent, wherein the first, second, third and fourth binding reagent is a binding partner of anti-IA2 IgG, anti-beta2glycoprotein IgG, anti-DGP IgG and anti-IA2 IgM, respectively;
b. forming at least a first, second, third and fourth binding complex comprising the binding reagents and the biomarkers; and
c. detecting the biomarkers in each of the binding complexes.

3. A multiplexed assay method comprising, simultaneously detecting at least three human biomarkers in a biological sample in a multiplexed assay format, wherein the at least three biomarkers comprise (a) anti-Smith IgG, (b) anti-RoSSA60 IgG, (c) anti-U1 RNPA IgG, (d) anti-insulin IgM and (e) anti-RoSSA52 IgG, wherein the multiplexed assay comprises:
a. combining, in one or more steps:
i. the biological sample;
ii. at least a first, second and third binding reagent, wherein the first, second and third binding reagent is a binding partner selected from anti-Smith IgG, anti-RoSSA60 IgG, anti-U1 RNPA IgG, anti-insulin IgM and anti-RoSSA52 IgG, respectively;
b. forming at least a first, second and third binding complex comprising the binding reagents and the biomarkers; and
c. detecting the biomarkers in each of the binding complexes.

4. A multiplexed assay method comprising, simultaneously detecting at least five human biomarkers in a biological sample in a multiplexed assay format, wherein the at least five biomarkers are selected from: (a) anti-insulin IgM, (b) anti-MPO IgA, (c) anti-Jo1 IgA, (d) anti-ZnT8 IgM, (e) anti-GAD65 IgG, (f) anti-Smith IgA, (g) anti-IA2 IgA, (h) anti-GAD65 IgM, (i) anti-Jo1 IgG, (j) anti-beta2glycoprotein IgA, (k) anti-ZnT8 IgG, and (1) anti-insulin IgA,
wherein the multiplexed assay comprises:
a. combining, in one or more steps:
i. the biological sample;
ii. at least a first, second, third, fourth and fifth binding reagent, wherein the first, second, third, fourth and fifth binding reagent is a binding partner of a biomarker selected from anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA, respectively;
b. forming at least a first, second, third, fourth and fifth binding complex comprising the binding reagents and the biomarkers; and
c. detecting the biomarkers in each of the binding complexes.

5. A multiplexed assay method comprising, simultaneously detecting at least two human biomarkers in a biological sample in a multiplexed assay format, wherein the at least two biomarkers are selected from: (a) anti-insulin IgM and (b) anti-MPO IgA,
wherein the multiplexed assay comprises:
a. combining, in one or more steps:
i. the biological sample;
ii. at least a first and second binding reagent, wherein the first and second, binding reagent is a binding partner of a biomarker selected from anti-insulin IgM and anti-MPO IgA, respectively;
b. forming at least a first and second binding complex comprising the binding reagents and the biomarkers; and
c. detecting the biomarkers in each of the binding complexes.

6. A multiplexed assay method comprising, simultaneously detecting at least two human biomarkers in a biological sample in a multiplexed assay format, wherein the at least three biomarkers are selected from: (a) anti-DGP IgA, (b) anti-DGP IgG, (c) anti-DGP IgM, (d) anti-TGM2 IgA, (e) anti-TGM2 IgG and (f) anti-TGM2 IgM,
wherein the multiplexed assay comprises:
a. combining, in one or more steps:
i. the biological sample;
ii. at least a first and second binding reagent, wherein the first and second binding reagent is a binding partner of a biomarker selected from anti-DGP IgA, anti-DGP IgG, anti-DGP IgM, anti-TGM2 IgA, anti-TGM2 IgG and anti-TGM2 IgM, respectively;
b. forming at least a first and second binding complex comprising the binding reagents and the biomarkers; and
c. detecting the biomarkers in each of the binding complexes.

7. A multiplexed assay method comprising, simultaneously detecting at least three human biomarkers in a biological sample in a multiplexed assay format, wherein the at least three biomarkers are selected from: (a) anti-DGP IgA, (b) anti-DGP IgG, (c) anti-TGM2 IgA, (d) anti-TGM2 IgG, (e) anti-Jo1 IgA, (f) anti-beta2glycoprotein IgG, (g) anti-CCP IgG, (h) anti-CENP B IgG, (i) anti-GAD65 IgA, (j) anti-GAD65 IgG, (k) anti-IA2 IgM, (1) anti-proinsulin IgA, (m) anti-proinsulin IgM, (n) anti-U1RNPA IgA, (o) anti-ZnT8 IgA, (p) anti-Sc170 IgA, (q) anti-Smith IgA, and (r) anti-RoSSA60 IgG,
wherein the multiplexed assay comprises:
a. combining, in one or more steps:
i. the biological sample;
ii. at least a first, second and third binding reagent, wherein the first, second and third binding reagent is a binding partner of anti-DGP IgA, anti-DGP IgG, anti-TGM2 IgA, anti-TGM2 IgG, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, or anti-RoSSA60 IgG, respectively;
b. forming at least a first, second and third binding complex comprising the binding reagents and the biomarkers; and
c. detecting the biomarkers in each of the binding complexes.

8. The method of claim 2, wherein the first, second, third and fourth binding reagents are immobilized on associated first, second, third and fourth binding domains, and the detecting step comprises measuring the complexes in each of the binding domains.

9. The method of claim 2 or 8, wherein the components combined in step (a) further comprise at least a first, second, third and fourth detection reagent that each bind a biomarker, and the binding complexes formed in step (b) further comprise the at least first, second, third and fourth detection reagents.

10. The method of claim 4, wherein the first, second, third, fourth and fifth binding reagents are immobilized on associated first, second, third, fourth and fifth binding domains, and the detecting step comprises measuring the complexes in each of the binding domains.

11. The method of claim 4 or 10, wherein the components combined in step (a) further comprise at least a first, second, third, fourth and fifth detection reagent that each bind a biomarker, and the binding complexes formed in step (b) further comprise the at least first, second, third, fourth and fifth detection reagents.

12. The method of claim 3, wherein the first, second and third binding reagents are immobilized on associated first, second and third binding domains, and the detecting step comprises measuring the complexes in each of the binding domains.

13. The method of claim 3 or 12, wherein the components combined in step (a) further comprise at least a first, second and third detection reagent that each bind a biomarker, and the binding complexes formed in step (b) further comprise the at least first, second and third detection reagents.

14. The method of claim 7, wherein the first, second, third, fourth, fifth and sixth binding reagents are immobilized on associated first, second, third, fourth, fifth and sixth binding domains, and the detecting step comprises measuring the complexes in each of the binding domains.

15. The method of claim 7 or 14, wherein the components combined in step (a) further comprise at least a first, second, third, fourth, fifth and sixth detection reagent that each bind a biomarker, and the binding complexes formed in step (b) further comprise the at least first, second, third, fourth, fifth and sixth detection reagents.

16. The method of claim 1, 5 or 6, wherein the first and second binding reagents are immobilized on associated first and second binding domains, and the detecting step comprises measuring the complexes in each of the binding domains.

17. The method of claim 2, wherein the first, second, third and fourth binding reagents are immobilized on associated first, second, third and fourth binding domains, and the detecting step comprises measuring the complexes in each of the binding domains.

18. The method of claim 4, wherein the first, second, third, fourth and fifth binding reagents are immobilized on associated first, second, third, fourth and fifth binding domains, and the detecting step comprises measuring the complexes in each of the binding domains.

19. The method of claim 3, wherein the first, second and third binding reagents are immobilized on associated first, second and third binding domains, and the detecting step comprises measuring the complexes in each of the binding domains.

20. The method of claim 7, wherein the first, second, third, fourth, fifth and sixth binding reagents are immobilized on associated first, second, third, fourth, fifth and sixth binding domains, and the detecting step comprises measuring the complexes in each of the binding domains.

21. The method of claim 1, 5 or 6, wherein the components combined in step (a) further comprise at least a first and second detection reagent that each bind a biomarker, and the binding complexes formed in step (b) further comprise the at least first and second detection reagents.

22. The method of claim 2, wherein the components combined in step (a) further comprise at least a first, second, third and fourth detection reagent that each bind a biomarker, and the binding complexes formed in step (b) further comprise the at least first, second, third and fourth detection reagents.

23. The method of claim 4, wherein the components combined in step (a) further comprise at least a first, second, third, fourth and fifth detection reagent that each bind a biomarker, and the binding complexes formed in step (b) further comprise the at least first, second, third, fourth and fifth detection reagents.

24. The method of claim 3, wherein the components combined in step (a) further comprise at least a first, second and third detection reagent that each bind a biomarker, and the binding complexes formed in step (b) further comprise the at least first, second and third detection reagents.

25. The method of claim 7, wherein the components combined in step (a) further comprise at least a first, second, third, fourth, fifth and sixth detection reagent that each bind a biomarker, and the binding complexes formed in step (b) further comprise the at least first, second, third, fourth, fifth and sixth detection reagents.

26. The method of any of claims 21 to 25, wherein the detection reagents each comprises a detectable label.

27. The method of any one of claims 1 to 26, wherein the biomarkers are antibodies and the binding reagents and detection reagents are antigens to the antibodies.

28. The method of any one of claims 1 to 26, wherein the biomarkers are antibodies, the binding reagents are antigens to the biomarker antibodies, and the detection reagents are detection antibodies that bind the biomarker antibodies.

29. The method of any of claims 20 to 28, wherein the binding reagents and the detection reagents are antibodies, antigens or a combination thereof.

30. The method of any of claims 25 to 29, wherein the measuring the concentration comprises measuring the presence of the detectable labels by electrochemiluminescence.

31. The method of any of claims 1 to 30, wherein each of the binding domains is an element of an array of binding domains.

32. The method of claim 31, wherein the array is located within a well of a multi-well plate.

33. The method of any of claims 1 to 32, wherein each of the binding domains are positioned on a surface of one or more particles.

34. The method of any of claims 26 to 31, wherein the detectable label is an electrochemiluminescence label, and the measuring of the detectable label comprises measuring an ECL signal.

35. The method of any of claims 1 to 34, wherein the biological sample is whole blood, serum, plasma, cerebrospinal fluid, urine, saliva, or an extraction or purification therefrom, or dilution thereof.

36. The method of claim 35, wherein the biological sample is serum or plasma.

37. The method of claim 1 or 2, further comprising detecting, in the multiplexed assay format, at least one additional biomarker in the biological sample, wherein the at least one additional biomarker is anti-proinsulin IgG, anti-MPO IgM, anti-ZnT8 IgM or a combination thereof.

38. The method of claim 3 or 4, further comprising detecting, the in multiplexed assay format, at least one additional biomarker in the biological sample, wherein the at least one additional biomarker is anti-IAA IgM, anti-RoSSA52 IgG, anti-GAD65 IgM, anti-ZnT8 IgG, anti-RoSSA60 IgA, anti-RoSSA52 IgM, anti-aNCA PR3 IgG, anti-Jo1 IgA, anti-Smith IgA, anti-U1 RNPA IgM, anti-MPO IgA, anti-U1RNPC IgG, anti-GAD65 IgG, anti-LaSSb IgA, anti-TPO IgG, anti-MPO IgG, anti-insulin IgA, anti-TPO IgM, anti-ZnT8 IgM, anti-IF IgG, anti-Smth IgG, TNF-alpha, IL-15, MIP1a, IL-10, NFL, IL-8, IL-6, IL-2, IL-21,r IL-23 or a combination thereof.

39. The method of claim 7, further comprising detecting, the in multiplexed assay format, at least one additional biomarker in the biological sample, wherein the at least one additional biomarker is anti-beta2glycoprotein IgG, anti-IA2 IgG, anti-MPO IgA, anti-MPO IgM, anti-RNP68/70 IgM, anti-RoSSA52 IgG, anti-RoSSA52 IgM, anti-RoSSA60 IgM or a combination thereof.

40. The method of any of claims 1 to 38, wherein the biological sample is obtained from a subject at risk for Type 1 diabetes.

41. The method of any of claims 1 to 38, wherein the biological sample is obtained from a subject diagnosed with Type 1 diabetes.

42. The method of any of claims 1 to 38, wherein the biological sample is obtained from a subject diagnosed with systemic lupus erythematosus.

43. The method of any of claims 1 to 38, wherein the biological sample is obtained from a having systemic lupus erythematosus flare.

44. The method of any of claims 1 to 38, wherein the biological sample is obtained from a subject diagnosed with celiac disease.

45. An assay method comprising quantifying the amount of a human biomarker in a biological sample, wherein the biomarker is selected from anti-IA2 IgG, anti-beta2glycoprotein IgG, anti-DGP IgG, anti-IA2 IgM, anti-proinsulin IgG, anti-MPO IgM, anti-ZnT8 IgM, or combinations thereof, wherein the quantifying comprises
(a) contacting the biological sample with
(i) a capture reagent that specifically binds to the biomarker in a binding domain on a surface, wherein the binding domain further comprises an anchoring reagent;
(ii) a first detection reagent that specifically binds to the biomarker and is linked to a first nucleic acid probe; and
(iii) a second detection reagent that specifically binds to the biomarker and is linked to a second nucleic acid probe,
thereby forming a complex on the surface comprising the capture reagent, the first biomarker, and the first and second detection reagents;
(b) using an extension process that requires the first and second probes to be in proximity, extending the second probe to form an extended sequence comprising an anchoring region that binds the anchoring reagent;
(c) binding the extended sequence to the anchoring reagent; and
(d) measuring the amount of extended sequence bound to the binding domain, thereby quantifying the amount of the biomarker.

46. The method of claim 44, wherein the biomarker is selected from anti-IA2 IgG, anti-beta2glycoprotein IgG, anti-DGP IgG and anti-IA2 IgM.

47. The method of claim 44, wherein the biomarker is selected from anti-IA2 IgG and anti-beta2glycoprotein IgG.

48. An assay method comprising quantifying the amount of a human biomarker in a biological sample, wherein the biomarker is selected from anti-Smith IgG, anti-RoSSA60 IgG, anti-U1 RNPA IgG, anti-insulin IgM, anti-RoSSA52 IgG, anti-GAD65 IgM, anti-ZnT8 IgG, anti-RoSSA60 IgA, anti-RoSSA52 IgM, anti-aNCA PR3 IgG, anti-Jo1 IgA, anti-Smith IgA or combinations thereof, wherein the quantifying comprises
(a) contacting the biological sample with
(i) a capture reagent that specifically binds to the biomarker in a binding domain on a surface, wherein the binding domain further comprises an anchoring reagent;
(ii) a first detection reagent that specifically binds to the biomarker and is linked to a first nucleic acid probe; and
(iii) a second detection reagent that specifically binds to the biomarker and is linked to a second nucleic acid probe,
thereby forming a complex on the surface comprising the capture reagent, the first biomarker, and the first and second detection reagents;
(b) using an extension process that requires the first and second probes to be in proximity, extending the second probe to form an extended sequence comprising an anchoring region that binds the anchoring reagent;
(c) binding the extended sequence to the anchoring reagent; and
(d) measuring the amount of extended sequence bound to the binding domain, thereby quantifying the amount of the biomarker.

49. An assay method comprising quantifying the amount of a human biomarker in a biological sample, wherein the biomarker is selected from anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA, or combinations thereof, wherein the quantifying comprises
(a) contacting the biological sample with
(i) a capture reagent that specifically binds to the biomarker in a binding domain on a surface, wherein the binding domain further comprises an anchoring reagent;
(ii) a first detection reagent that specifically binds to the biomarker and is linked to a first nucleic acid probe; and
(iii) a second detection reagent that specifically binds to the biomarker and is linked to a second nucleic acid probe,
thereby forming a complex on the surface comprising the capture reagent, the first biomarker, and the first and second detection reagents;
(b) using an extension process that requires the first and second probes to be in proximity, extending the second probe to form an extended sequence comprising an anchoring region that binds the anchoring reagent;
(c) binding the extended sequence to the anchoring reagent; and
(d) measuring the amount of extended sequence bound to the binding domain, thereby quantifying the amount of the biomarker.

50. An assay method comprising quantifying the amount of a human biomarker in a biological sample, wherein the biomarker is selected from anti-DGP IgA, anti-DGP IgG, anti-TGM2 IgA, anti-TGM2 IgG, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, or anti-RoSSA60 IgG, anti-beta2glycoprotein IgA, anti-IA2 IgG, anti-MPO IgA, anti-MPO IgM, anti-RNP68/70 IgM, anti-RoSSA52 IgG, anti-RoSSA52 IgM, anti-RoSSA60 IgM, or combinations thereof, wherein the quantifying comprises
(a) contacting the biological sample with
(i) a capture reagent that specifically binds to the biomarker in a binding domain on a surface, wherein the binding domain further comprises an anchoring reagent;
(ii) a first detection reagent that specifically binds to the biomarker and is linked to a first nucleic acid probe; and
(iii) a second detection reagent that specifically binds to the biomarker and is linked to a second nucleic acid probe,
thereby forming a complex on the surface comprising the capture reagent, the first biomarker, and the first and second detection reagents;
(b) using an extension process that requires the first and second probes to be in proximity, extending the second probe to form an extended sequence comprising an anchoring region that binds the anchoring reagent;
(c) binding the extended sequence to the anchoring reagent; and
(d) measuring the amount of extended sequence bound to the binding domain, thereby quantifying the amount of the biomarker.

51. The method of any one of claims 44 to 49, wherein the biomarkers are antibodies and the binding reagents and detection reagents are antigens to the antibodies.

52. The method of any one of claims 44 to 49, wherein the biomarkers are antibodies, the binding reagents are antigens to the biomarker antibodies, and the detection reagents are detection antibodies that bind the biomarker antibodies.

53. The method of any of claims 44 to 49, wherein the binding reagents and the detection reagents are antibodies, antigens or a combination thereof.

54. The method of any of claims 44 to 52, wherein the extension process comprises PCR.

55. The method of any of claims 44 to 53, wherein the extension process comprises rolling circle amplification.

56. The method of any of claims 44 to 54, wherein binding the extended sequence to the anchoring reagent comprises forming a triple helix between the anchoring reagent and the anchoring region.

57. The method of any of claims 44 to 44, wherein measuring the amount of extended sequence bound to the binding domain comprises contacting the extended sequence with a labeled probe complementary to the detection sequence complement.

58. The method of claim 56, wherein the amount of labeled probe is measured by a measurement of light scattering, optical absorbance, fluorescence, chemiluminescence, electrochemiluminescence, bioluminescence, phosphorescence, radioactivity, magnetic field, or combinations thereof.

59. The method of any of claims 44 to 57, wherein the biological sample is whole blood, serum, plasma, cerebrospinal fluid, urine, saliva, or an extraction or purification therefrom, or dilution thereof.

60. The method of claim 58, wherein the biological sample is serum or plasma.

61. The method of any of claims 44 to 59, wherein the biological sample is obtained from a subject at risk for Type 1 diabetes.

62. The method of any of claims 44 to 59, wherein the biological sample is obtained from a subject diagnosed with Type 1 diabetes.

63. The method of any one of claims 1 to 61, wherein the biological sample is obtained from a subject diagnosed with Type 1 diabetes who is a candidate for treatment with alefacept.

64. The method of claim 62, wherein the subject is subsequently treated with alefacept.

65. The method of any one of claims 1 to 61, wherein the biological sample is obtained from a subject diagnosed with Type 1 diabetes who is being treated with alefacept.

66. The method of any of claims 44 to 59, wherein the biological sample is obtained from a subject diagnosed with systemic lupus erythematosus.

67. The method of any of claims 44 to 59, wherein the biological sample is obtained from a having systemic lupus erythematosus flare.

68. The method of any of claims 44 to 59, wherein the biological sample is obtained from a subject diagnosed with celiac disease.

69. A method of determining if treatment of a human subject having Type 1 diabetes with alefacept is effective, comprising
a. conducting the assay of any of claim 1, 2 or 44 on a biological sample of the human taken at a timepoint following the beginning of treatment with alefacept;
b. detecting the concentration of at least one of anti-IA2 IgG, anti-beta2glycoprotein IgG, anti-DGP IgG, anti-IA2 IgM, anti-MPO IgA, anti-proinsulin IgG, anti-MPO IgM, anti-ZnT8 IgM or a combination thereof; and
c. determining:
if the concentration of at least one of anti-IA2 IgG, anti-beta2glycoprotein IgG, anti-DGP IgG and anti-IA2 IgM is higher compared to a control, wherein the control is a human subject that is not a candidate for treatment with alefacept or a human subject that does not have Type 1 diabetes; or
i. if the concentration of at least one of anti-proinsulin IgG, anti-MPO IgM, anti-ZnT8 IgM is lower compared to a control, wherein the control is a human subject that has Type 1 diabetes;
wherein: (i) if the concentration of at least one of anti-IA2 IgG, anti-beta2glycoprotein IgG, anti-DGP IgG and anti-IA2 IgM is higher than the control, or (ii) if the concentration of at least one of anti-proinsulin IgG, anti-MPO IgM, anti-ZnT8 IgM is lower than the control, reporting that the treatment with alefacept is effective.

70. The method of claim 68, wherein if the concentration of at least two of anti-IA2 IgG, anti-beta2glycoprotein IgG, anti-DGP IgG, anti-IA2 IgM, anti-proinsulin IgG, anti-MPO IgM and anti-ZnT8 IgM is determined compared to the control.

71. The method of claim 68, wherein if the concentration of both anti-IA2 IgG and anti-beta2glycoprotein IgG are higher than the control, reporting that the treatment with alefacept is effective.

72. The method of any of claims 68 to 70, wherein the biological sample is taken at a timepoint 11 weeks, 26 weeks or 30 weeks following the beginning of treatment with alefacept.

73. A method of determining if a human subject having Type 1 diabetes is a candidate for treatment with alefacept, comprising
a. conducting the assay of any of claim 1, 2 or 44 on a biological sample of the human;
b. detecting the concentration of at least one of anti-IA2 IgG, anti-beta2glycoprotein IgG, anti-DGP IgG, anti-IA2 IgM or a combination thereof; and
c. determining if the concentration of at least one of anti-IA2 IgG, anti-beta2glycoprotein IgG, anti-DGP IgG and anti-IA2 IgM is higher compared to a control, wherein the control is a human subject that is not a candidate for treatment with alefacept or a human subject that does not have Type 1 diabetes;
wherein if the concentration of at least one of anti-IA2 IgG, anti-beta2glycoprotein IgG, anti-DGP IgG, anti-IA2 IgM and anti-MPO IgA is higher than the control, reporting that the human subject is a candidate for treatment with alefacept.

74. The method of claim 72, wherein if the concentration of both anti-IA2 IgG and anti-beta2glycoprotein IgG are higher than the control, reporting that the human is a candidate for treatment with alefacept.

75. The method of claim 72 or 73, further comprising administering alefacept to a human reported to be a candidate for treatment with alefacept.

76. A method of determining if a human subject has systemic lupus erythematosus, comprising
a. conducting the assay of claim 3 or 47 on a biological sample of the human;
b. detecting the concentration of at least one of anti-Smith IgG, anti-RoSSA60 IgG, anti-U1 RNPA IgG, anti-insulin IgM, anti-RoSSA52 IgG, anti-GAD65 IgM, anti-ZnT8 IgG, anti-RoSSA60 IgA, anti-RoSSA52 IgM, anti-aNCA PR3 IgG, anti-Jo1 IgA, anti-Smith IgA or a combination thereof; and
c. determining if the concentration of at least one of anti-Smith IgG, anti-RoSSA60 IgG, anti-U1 RNPA IgG, anti-insulin IgM, anti-RoSSA52 IgG, anti-GAD65 IgM, anti-ZnT8 IgG, anti-RoSSA60 IgA, anti-RoSSA52 IgM, anti-aNCA PR3 IgG, anti-Jo1 IgA, and anti-Smith IgA is higher compared to a control, wherein the control is a human subject that does not have systemic lupus erythematosus;
wherein if the concentration of at least one of anti-Smith IgG, anti-RoSSA60 IgG, anti-U1 RNPA IgG, anti-insulin IgM, anti-RoSSA52 IgG, anti-GAD65 IgM, anti-ZnT8 IgG, anti-RoSSA60 IgA, anti-RoSSA52 IgM, anti-aNCA PR3 IgG, anti-Jo1 IgA, and anti-Smith IgA is higher than the control, reporting that the human subject has systemic lupus erythematosus.

77. The method of claim 75, wherein the concentration of at least five biomarkers is determined compared to the control.

78. A method of determining if a human subject is at risk of a systemic lupus erythematosus flare, comprising
a. conducting the assay of claim 4 or 48 on a biological sample of the human;
b. detecting the concentration of at least one of anti-insulin IgM, anti-MPO IgA, anti-Jo 1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, anti-insulin IgA or a combination thereof; and
c. determining if the concentration of at least one of anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA is higher compared to a control, wherein the control is a human subject that does not have systemic lupus erythematosus;
wherein if the concentration of at least one of anti-insulin IgM, anti-MPO IgA, anti-Jo 1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA is higher than the control, reporting that the human subject is at risk of a systemic lupus erythematosus flare.

79. The method of claim 77, wherein the concentration of at least five biomarkers is determined compared to the control.

80. A method of determining if a human subject has celiac disease, comprising
a. conducting the assay of any of claim 6, 7 or 50 on a biological sample of the human;
b. detecting the concentration of at least one of anti-DGP IgA, anti-DGP IgG, anti-DGP IgM, anti-TGM2 IgA, anti-TGM2 IgG, anti-TGM2 IgM, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, anti-RoSSA60 IgG or a combination thereof; and
c. determining if the concentration of at least one of anti-DGP IgA, anti-DGP IgG, anti-DGP IgM, anti-TGM2 IgA, anti-TGM2 IgG, anti-TGM2 IgM, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, and anti-RoSSA60 IgG is higher compared to a control, wherein the control is a human subject that does not have systemic lupus erythematosus;
wherein if the concentration of at least one of anti-DGP IgA, anti-DGP IgG, anti-DGP IgM, anti-TGM2 IgA, anti-TGM2 IgG, anti-TGM2 IgM, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, and anti-RoSSA60 IgG is higher than the control, reporting that the human subject has celiac disease.

81. The method of claim 79, wherein the concentration of at least three biomarkers is determined compared to the control.

82. A kit comprising, in one or more vials, containers, or compartments:
(a) a surface comprising at least a first and second binding reagent immobilized on an associated first and second binding domain, wherein the first and second binding reagent is a binding partner of anti-IA2 IgG and anti-beta2glycoprotein IgG, respectively;
(b) a detection reagent that specifically binds to anti-IA2 IgG; and
(c) a detection reagent that specifically binds to anti-beta2glycoprotein IgG.

83. A kit comprising, in one or more vials, containers, or compartments:
(a) a surface comprising at least a first, second, third and fourth binding reagent immobilized on an associated first, second, third and fourth binding domain, wherein the first, second, third and fourth binding reagent is a binding partner of anti-IA2 IgG, anti-beta2glycoprotein IgG, anti-DGP IgG and anti-IA2 IgM, respectively;
(b) a detection reagent that specifically binds to anti-IA2 IgG;
(c) a detection reagent that specifically binds to anti-beta2glycoprotein IgG;
(d) a detection reagent that specifically binds to anti-DGP IgG; and
(e) a detection reagent that specifically binds to anti-IA2 IgM.

84. A kit comprising, in one or more vials, containers, or compartments:
(a) a surface comprising at least a first, second and third binding reagent immobilized on an associated first, second and third binding domain, wherein the first, second and third binding reagent is a binding partner of anti-Smith IgG, anti-RoSSA60 IgG and anti-U1 RNPA IgG, respectively;
(b) a detection reagent that specifically binds to anti-Smith IgG;
(c) a detection reagent that specifically binds to anti-RoSSA60 IgG; and
(d) a detection reagent that specifically binds to anti-U1 RNPA IgG.

85. A kit comprising, in one or more vials, containers, or compartments:
(a) a surface comprising at least a first, second, third, fourth and fifth binding reagent immobilized on an associated first, second, third, fourth and fifth binding domain, wherein the first, second, third, fourth and fifth binding reagent is a binding partner of anti-insulin IgM, anti-MPO IgA, anti-Jo1 IgA, anti-ZnT8 IgM, anti-GAD65 IgG, respectively;
(b) a detection reagent that specifically binds to anti-insulin IgM;
(c) a detection reagent that specifically binds to anti-MPO IgA;
(d) a detection reagent that specifically binds to anti-Jo1 IgA;
(e) a detection reagent that specifically binds to anti-ZnT8 IgM; and
(f) a detection reagent that specifically binds to anti-GAD65 IgG.

86. A kit comprising, in one or more vials, containers, or compartments:
(a) a surface comprising at least a first and second binding reagent immobilized on an associated first and second binding domain, wherein the first and second binding reagent is a binding partner of anti-insulin IgM and anti-MPO IgA, respectively;
(b) a detection reagent that specifically binds to anti-insulin IgM; and
(c) a detection reagent that specifically binds to anti-MPO IgA.

87. A kit comprising, in one or more vials, containers, or compartments:
(a) a surface comprising at least a first, second, third, fourth, fifth and sixth binding reagent immobilized on an associated first, second, third, fourth, fifth and sixth binding domain, wherein the first, second, third, fourth, fifth and sixth binding reagent is a binding partner of anti-DGP IgA, anti-DGP IgG, anti-TGM2 IgA, anti-TGM2 IgG, anti-Smith IgA, and anti-insulin IgA, respectively;
(b) a detection reagent that specifically binds to anti-DGP IgA;
(c) a detection reagent that specifically binds to anti-DGP IgG;
(d) a detection reagent that specifically binds to anti-TGM2 IgA;
(e) a detection reagent that specifically binds to TGM2 IgG;
(f) a detection reagent that specifically binds to anti-Smith IgA; and
(g) a detection reagent that specifically binds to anti-insulin IgA.

88. A kit comprising, in one or more vials, containers, or compartments:
(a) a surface comprising at least a first, second, third, fourth, fifth and sixth binding reagent immobilized on an associated first, second, third, fourth, fifth and sixth binding domain, wherein the first, second, third, fourth, fifth and sixth binding reagent is a binding partner of a biomarker selected from anti-TGM2 IgA, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, and anti-ZnT8 IgA, respectively; and
(b) detection reagents that specifically binds to six of the biomarkers selected from anti-TGM2 IgA, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, and anti-ZnT8 IgA, respectively.

89. The kit of any of claims 82 to 86, further comprising a calibration reagent, a control reagent, or both.

90. The kit of any of claims 82 to 86, wherein each binding reagents and detection reagents are antigens.

91. The kit of any of claims 82 to 86, wherein the binding reagents are antigens and the detection reagents are antibodies antigens or a combination thereof.

92. The kit of any of claims 82 to 89, wherein each detection reagent comprises a detectable label.

93. The kit of claim 82, further comprising a detection reagent that specifically binds to anti-proinsulin IgG, a detection reagent that specifically binds to anti-MPO IgM, a detection reagent that specifically binds to anti-ZnT8 IgM, or combination thereof.

94. An assay system comprising at least one assay panel selected from:
a) an assay panel comprising anti-insulin, anti-proinsulin, and anti-ZnT8 autoantibodies for IgG, IgA, and IgM isotypes;
b) an assay panel comprising anti-GAD65 and anti-Intrinsic Factor autoantibodies for IgG, IgA, and IgM isotypes;
c) an assay panel comprising anti-IA2 and anti-Jo-1 autoantibodies for IgG, IgA, and IgM isotypes;
d) an assay panel comprising anti-IA2 and anti-Jo-1 autoantibodies for IgG, IgA, and IgM isotypes;
e) an assay panel comprising anti-Smith, anti-Thyroglobulin, anti-MPO, anti-DGP, and anti-TGM2 autoantibodies for IgG, IgA, and IgM isotypes;
f) an assay panel comprising anti-TPO, anti-U1RNPA, anti-RoSSA52, and anti-aNCA PR3 autoantibodies for IgG, IgA, and IgM isotypes;
g) an assay panel comprising anti-CENPB, anti-Sc170, anti-CCP, anti-MPO, anti-RoSSA60, anti-U1RNPC, anti-Smith, and anti-RNP68/70 autoantibodies for IgG, IgA, and IgM isotypes;
h) an assay panel comprising anti-LaSSB and anti-beta 2-glycoprotein autoantibodies for IgG, IgA, and IgM isotypes;
i) an assay panel comprising anti-insulin, anti-MPO, TARC, anti-Jo-1 and anti-GAD65 autoantibodies for IgG, IgA, and IgM isotypes, and MIP-1a, and IL-7;
j) an assay panel comprising anti-insulin IgM, anti-MPO IgA, TARC, anti-Jo-1 IgA and anti-GAD65 IgM autoantibodies for IgG, IgA, and IgM isotypes, and MIP-1a, IL-7 and Eotaxin; or
k) an assay panel comprising anti-insulin IgM, anti-MPO IgA, MPO IgM, TARC, anti-Jo-1 IgA and anti-GAD65 IgM autoantibodies for IgG, IgA, and IgM isotypes, and MIP-1a, IL-7 and Eotaxin.

95. An assay system comprising at least one assay panel selected from:
a) an assay panel comprising anti-IA2 IgG and anti-beta2glycoprotein IgG;
b) an assay panel comprising at least two of anti-IA2 IgG, anti-beta2glycoprotein IgG, anti-DGP IgG, anti-IA2 IgM anti-proinsulin IgG, anti-MPO IgM or anti-ZnT8 IgM;
c) an assay panel comprising at least two of anti-Smith IgG, anti-RoSSA60 IgG, anti-U1 RNPA IgG, anti-insulin IgM and anti-RoSSA52 IgG, anti-GAD65 IgM, anti-ZnT8 IgG, anti-RoSSA60 IgA, anti-RoSSA52 IgM, anti-aNCA PR3 IgG, anti-Jo1 IgA, anti-Smith IgA, anti-U1 RNPA IgM, anti-MPO IgA, anti-U1RNPC IgG, anti-GAD65 IgG, anti-LaSSb IgA, anti-TPO IgG, anti-MPO IgG, anti-insulin IgA, anti-TPO IgM, anti-ZnT8 IgM, anti-IF IgG, anti-Smth IgG, TNF-alpha, IL-15, MIP1a, IL-10, NFL, IL-8, IL-6, IL-2, IL-21 or IL-23;
d) an assay panel comprising at least two of anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG or anti-insulin IgA;
e) as assay panel comprising at anti-insulin IgM and anti-MPO IgA;
f) an assay panel comprising at least two of anti-DGP IgA, anti-DGP IgG, anti-DGP IgM, anti-TGM2 IgA, anti-TGM2 IgG, anti-TGM2 IgM, anti-Smith IgA, or anti-insulin IgA; or
g) an assay panel comprising at least two of anti-DGP IgA, anti-DGP IgG, anti-TGM2 IgA, anti-TGM2 IgG, anti-Jo1 IgA, anti-beta2glycoprotein IgG, anti-CCP IgG, anti-CENP B IgG, anti-GAD65 IgA, anti-GAD65 IgG, anti-IA2 IgM, anti-proinsulin IgA, anti-proinsulin IgM, anti-U1RNPA IgA, anti-ZnT8 IgA, anti-Sc170 IgA, anti-Smith IgA, anti-RoSSA60 IgG, anti-beta2glycoprotein IgA, anti-IA2 IgG, anti-MPO IgA, anti-MPO IgM, anti-RNP68/70 IgM, anti-RoSSA52 IgG, anti-RoSSA52 IgM or anti-RoSSA60 IgM.

96. The assay system of claim 92 or 93, wherein the assays are simultaneous bridging assays, sequential bridging assays, classical serology assays or combinations thereof.

97. The assay system of any of claims 92-94, wherein the assay system comprising at least two, at least three, at least four, at least five, at least six or at least seven of the assay panels.

98. An assay method comprising detecting, quantifying, or both, at least two human biomarkers in a biological sample, wherein the biomarker is (a) anti-IA2 IgG and (b) anti-beta2glycoprotein IgG, wherein the detecting, quantifying, or both, comprises
(a) contacting the biological sample with
(i) a capture reagent that specifically binds to the biomarker in a binding domain on a surface, wherein the binding domain further comprises an anchoring reagent;
(ii) a first detection reagent that specifically binds to the biomarker and is linked to a first nucleic acid probe; and
(iii) a second detection reagent that specifically binds to the biomarker and is linked to a second nucleic acid probe,
thereby forming a complex on the surface comprising the capture reagent, the first biomarker, and the first and second detection reagents;
(b) using an extension process that requires the first and second probes to be in proximity, extending the second probe to form an extended sequence comprising an anchoring region that binds the anchoring reagent;
(c) binding the extended sequence to the anchoring reagent; and
(d) measuring the extended sequence bound to the binding domain, thereby detecting, quantifying, or both, the biomarkers.

99. An assay method comprising detecting, quantifying, or both, at least four human biomarkers in a biological sample, wherein at least four biomarkers comprise (a) anti-IA2 IgG, (b) anti-beta2glycoprotein IgG, (c) anti-DGP IgG and (d) anti-IA2 IgM, wherein the quantifying, detecting, or both, comprises
(a) contacting the biological sample with
(i) a capture reagent that specifically binds to the biomarker in a binding domain on a surface, wherein the binding domain further comprises an anchoring reagent;
(ii) a first detection reagent that specifically binds to the biomarker and is linked to a first nucleic acid probe; and
(iii) a second detection reagent that specifically binds to the biomarker and is linked to a second nucleic acid probe,
thereby forming a complex on the surface comprising the capture reagent, the first biomarker, and the first and second detection reagents;
(b) using an extension process that requires the first and second probes to be in proximity, extending the second probe to form an extended sequence comprising an anchoring region that binds the anchoring reagent;
(c) binding the extended sequence to the anchoring reagent; and
(d) measuring the extended sequence bound to the binding domain, thereby detecting, quantifying, or both, the biomarkers.

100. An assay method comprising detecting, quantifying, or both, at least three human biomarkers in a biological sample, wherein the at least three biomarkers comprise (a) anti-Smith IgG, (b) anti-RoSSA60 IgG, (c) anti-U1 RNPA IgG, (d) anti-insulin IgM and (e) anti-RoSSA52 IgG, wherein the quantifying, detecting, or both, comprises
(a) contacting the biological sample with
(i) a capture reagent that specifically binds to the biomarker in a binding domain on a surface, wherein the binding domain further comprises an anchoring reagent;
(ii) a first detection reagent that specifically binds to the biomarker and is linked to a first nucleic acid probe; and
(iii) a second detection reagent that specifically binds to the biomarker and is linked to a second nucleic acid probe,
thereby forming a complex on the surface comprising the capture reagent, the first biomarker, and the first and second detection reagents;
(b) using an extension process that requires the first and second probes to be in proximity, extending the second probe to form an extended sequence comprising an anchoring region that binds the anchoring reagent;
(c) binding the extended sequence to the anchoring reagent; and
(d) measuring the extended sequence bound to the binding domain, thereby detecting, quantifying, or both, the biomarkers.

101. An assay method comprising detecting, quantifying, or both, at least five human biomarkers in a biological sample, wherein the at least five biomarkers are selected from: (a) anti-insulin IgM, (b) anti-MPO IgA, (c) anti-Jo1 IgA, (d) anti-ZnT8 IgM, (e) anti-GAD65 IgG, (f) anti-Smith IgA, (g) anti-IA2 IgA, (h) anti-GAD65 IgM, (i) anti-Jo1 IgG, (j) anti-beta2glycoprotein IgA, (k) anti-ZnT8 IgG, and (1) anti-insulin IgA,
wherein the quantifying, detecting, or both, comprises
(a) contacting the biological sample with
(i) a capture reagent that specifically binds to the biomarker in a binding domain on a surface, wherein the binding domain further comprises an anchoring reagent;
(ii) a first detection reagent that specifically binds to the biomarker and is linked to a first nucleic acid probe; and
(iii) a second detection reagent that specifically binds to the biomarker and is linked to a second nucleic acid probe,
thereby forming a complex on the surface comprising the capture reagent, the first biomarker, and the first and second detection reagents;
(b) using an extension process that requires the first and second probes to be in proximity, extending the second probe to form an extended sequence comprising an anchoring region that binds the anchoring reagent;
(c) binding the extended sequence to the anchoring reagent; and
(d) measuring the extended sequence bound to the binding domain, thereby detecting, quantifying, or both, the biomarkers.

102. A multiplexed assay method comprising, simultaneously detecting at least five human biomarkers in a biological sample in a multiplexed assay format, wherein the at least two biomarkers comprise (a) anti-TGM2, (b) anti-GAD65, (c) anti-ZnT8, (d) anti-insulin, and (e) anti-IA2, wherein the multiplexed assay comprises:
a. combining, in one or more steps:
i. the biological sample;
ii. at least a first and second binding reagent, wherein the first and second binding reagent is a binding partner of (a) anti-TGM2, (b) anti-GAD65, (c) anti-ZnT8, (d) anti-insulin, and (e) anti-IA-2, respectively;
b. forming at least a first and second binding complex comprising the binding reagents and the biomarkers; and
c. detecting the biomarkers in each of the binding complexes.

103. The multiplexed assay method of claim 102, wherein the assay cut-points are from about 10.0-13.0 U/mL for anti-TGM2, from about 12.0-15.0 IU/mL for anti-GAD65, from about 6.0-9.0 U/mL for anti-ZnT8, from about 0.1-2.0 U/mL for anti-insulin or from about 0.5-3.0 IU/mL for anti-IA2.

104. The multiplexed assay method of claim 102, wherein the assay cut-points are from about 7.0-10.0 U/mL for anti-TGM2, from about 10.0-15.0 U/mL for anti-GAD65, from about 5.0-9.0 U/mL for anti-ZnT8, from about 1.0-3.0 U/mL for anti-insulin or from about 1.5-3.5 U/mL for anti-IA2.

105. A kit comprising, in one or more vials, containers, or compartments:
(a) a surface comprising at least a first, second, third, fourth and fifth binding reagent immobilized on an associated first, second, third, fourth and fifth binding domain, wherein the first, second, third, fourth and fifth binding reagent is a binding partner of TGM2, GAD65, ZnT8, insulin, and IA-2, respectively;
(b) a detection reagent that specifically binds to TGM2;
(c) a detection reagent that specifically binds to GAD65;
(d) a detection reagent that specifically binds to ZnT8;
(e) a detection reagent that specifically binds to insulin; and
(f) a detection reagent that specifically binds to IA-2.

106. The method of any one of claims 1-44, wherein the biomarkers are located on separate plates.

107. The method of any one of claims 1-44, wherein the biomarkers are located on the same plate.

108. An assay method comprising detecting at least two human biomarkers in a biological sample, wherein the at least two biomarkers comprise (a) anti-IA2 IgG and (b) anti-beta2glycoprotein IgG, wherein the assay comprises:
a. contacting, in one or more steps:
i. the biological sample;
ii. at least a first and second binding reagent, wherein the first and second binding reagent is a binding partner of anti-IA2 IgG and anti-beta2glycoprotein IgG, respectively;
b. forming at least a first and second binding complex comprising the binding reagents and the biomarkers; and
c. detecting the biomarkers in each of the binding complexes.

109. An assay method comprising detecting at least four human biomarkers in a biological sample, wherein the at least four biomarkers comprise (a) anti-IA2 IgG, (b) anti-beta2glycoprotein IgG, (c) anti-DGP IgG and (d) anti-IA2 IgM, wherein the assay comprises:
a. contacting, in one or more steps:
i. the biological sample;
ii. at least a first, second, third and fourth binding reagent, wherein the first, second, third and fourth binding reagent is a binding partner of anti-IA2 IgG, anti-beta2glycoprotein IgG, anti-DGP IgG and anti-IA2 IgM, respectively;
b. forming at least a first, second, third and fourth binding complex comprising the binding reagents and the biomarkers; and
c. detecting the biomarkers in each of the binding complexes.

110. An assay method comprising detecting at least three human biomarkers in a biological sample, wherein the at least three biomarkers comprise (a) anti-Smith IgG, (b) anti-RoSSA60 IgG, (c) anti-U1 RNPA IgG, (d) anti-insulin IgM and (e) anti-RoSSA52 IgG, wherein the assay comprises:
a. contacting, in one or more steps:
i. the biological sample;
ii. at least a first, second and third binding reagent, wherein the first, second and third binding reagent is a binding partner selected from anti-Smith IgG, anti-RoSSA60 IgG, anti-U1 RNPA IgG, anti-insulin IgM and anti-RoSSA52 IgG, respectively;
b. forming at least a first, second and third binding complex comprising the binding reagents and the biomarkers; and
c. detecting the biomarkers in each of the binding complexes.

111. An assay method comprising detecting at least five human biomarkers in a biological sample, wherein the at least five biomarkers are selected from: (a) anti-insulin IgM, (b) anti-MPO IgA, (c) anti-Jo1 IgA, (d) anti-ZnT8 IgM, (e) anti-GAD65 IgG, (f) anti-Smith IgA, (g) anti-IA2 IgA, (h) anti-GAD65 IgM, (i) anti-Jo1 IgG, (j) anti-beta2glycoprotein IgA, (k) anti-ZnT8 IgG, and (1) anti-insulin IgA,
wherein the assay comprises:
a. contacting, in one or more steps:
i. the biological sample;
ii. at least a first, second, third, fourth and fifth binding reagent, wherein the first, second, third, fourth and fifth binding reagent is a binding partner of a biomarker selected from anti-insulin IgM, anti-MPO IgA, anti-Jo1 IgA, anti-ZnT8 IgM, anti-GAD65 IgG, anti-Smith IgA, anti-IA2 IgA, anti-GAD65 IgM, anti-Jo1 IgG, anti-beta2glycoprotein IgA, anti-ZnT8 IgG, and anti-insulin IgA, respectively;
b. forming at least a first, second, third, fourth and fifth binding complex comprising the binding reagents and the biomarkers; and
c. detecting the biomarkers in each of the binding complexes.

112. An assay method comprising detecting at least two human biomarkers in a biological sample, wherein the at least two biomarkers are selected from: (a) anti-insulin IgM and (b) anti-MPO IgA, wherein the assay comprises:
a. contacting, in one or more steps:
i. the biological sample;
ii. at least a first and second binding reagent, wherein the first and second, binding reagent is a binding partner of a biomarker selected from anti-insulin IgM and anti-MPO IgA, respectively;
b. forming at least a first and second binding complex comprising the binding reagents and the biomarkers; and
c. detecting the biomarkers in each of the binding complexes.

113. An assay method comprising detecting at least two human biomarkers in a biological sample, wherein the at least three biomarkers are selected from: (a) anti-DGP IgA, (b) anti-DGP IgG, (c) anti-DGP IgM, (d) anti-TGM2 IgA, (e) anti-TGM2 IgG and (f) anti-TGM2 IgM,
wherein the assay comprises:
a. contacting, in one or more steps:
i. the biological sample;
ii. at least a first and second binding reagent, wherein the first and second binding reagent is a binding partner of a biomarker selected from anti-DGP IgA, anti-DGP IgG, anti-DGP IgM, anti-TGM2 IgA, anti-TGM2 IgG and anti-TGM2 IgM, respectively;
b. forming at least a first and second binding complex comprising the binding reagents and the biomarkers; and
c. detecting the biomarkers in each of the binding complexes.

114. The multiplexed assay method of claim 102, wherein the biological sample is from a same human subject at ages selected from the group consisting of about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, and combinations thereof.

115. The multiplexed assay method of claim 102, wherein the biological sample is from a same human subject every 2, 3, 4, 5, 6, 7, 8, 9, 10, 11, 12, 13, 14, 15, 16, 17, 18, 19, 20, 21, 22, 23 or 24 months.
